(19) United States
(12) Patent Application Publication
(10) Pub. No.: US 2024/0115712 A1
(43) Pub. Date: Apr. 11, 2024

(54) SEMICARBAZONE-BASED SAPONIN CONJUGATE

(71) Applicant: SAPREME TECHNOLOGIES B.V., Bilthoven (NL)

(72) Inventors: Guy HERMANS, Merelbeke (BE); Ruben POSTEL, Bilthoven (NL); Helmus VAN DE LANGEMHEEN, Putten (NL); Mazdak ASADIAN BIRJAND, Berlin (DE)

(21) Appl. No.: 18/262,994

(22) PCT Filed: Jan. 26, 2022
(86) PCT No.: PCT/NL2022/050039

§ 371 Date: Jul. 26, 2023

(30) Foreign Application Priority Data
Jan. 26, 2021 (NL) 2027405
Sep. 9, 2021 (WO) PCT/NL2021/050550

(51) Int. Cl. A61K 47/55; A61K 47/54

(52) U.S. Cl. CPC A61K 47/55; A61K 47/554

(57) ABSTRACT

The invention relates to a saponin conjugate comprising a saponin derivative based on a saponin comprising a triterpene aglycone and at least one of a first saccharide chain and a second saccharide chain linked to the aglycone core structure, wherein the saponin derivative comprises an aglycone core structure comprising an aldehyde functional group which aldehyde functional group has been transformed to a semicarbazone functional group, the saponin conjugate further comprising a proteinaceous molecule capable of binding to a cell-surface molecule. The invention also relates to a composition comprising the saponin conjugate. In addition, the invention relates to a pharmaceutical combination comprising said composition comprising the saponin conjugate and a pharmaceutical composition comprising for example an ADC or an antibody-oligonucleotide conjugate (AOC). The invention also relates to a pharmaceutical composition comprising the saponin conjugate and comprising for example an ADC or an AOC. The invention also relates to the pharmaceutical combination or pharmaceutical composition, for use as a medicament. The invention also relates to the saponin conjugate comprising the saponin derivative, a proteinaceous molecule capable of binding to a cell surface molecule (endocytic receptor), and further comprising an effector moiety such as an oligonucleotide. The invention also relates to an in vitro or ex vivo method for transferring a molecule from outside a cell to inside said cell.

### TECHNOLOGICAL FIELD

The invention relates to a saponin conjugate comprising a saponin derivative based on a saponin comprising a triterpene aglycone and at least one of a first saccharide chain and a second saccharide chain linked to the aglycone core structure, wherein the saponin derivative comprises an aglycone core structure comprising an aldehyde functional group which aldehyde functional group has been transformed to a semicarbazone functional group, the saponin conjugate further comprising a proteinaceous molecule capable of binding to a cell-surface molecule. The invention also relates to a composition comprising the saponin conjugate. In addition, the invention relates to a pharmaceutical combination comprising said composition comprising the saponin conjugate and a pharmaceutical composition comprising for example an ADC or an antibody-oligonucleotide conjugate (AOC). The invention also relates to a pharmaceutical composition comprising the saponin conjugate and comprising for example an ADC or an AOC. The invention also relates to the pharmaceutical combination or pharmaceutical composition, for use as a medicament. The invention also relates to the saponin conjugate comprising the saponin derivative, a proteinaceous molecule capable of binding to a cell surface molecule (endocytic receptor), and further comprising an effector moiety such as an oligonucleotide. The invention also relates to an in vitro or ex vivo method for transferring a molecule from outside a cell to inside said cell.

### BACKGROUND OF THE INVENTION

Targeted tumor therapy is a cancer treatment that uses drugs to target specific genes and proteins that are involved in the growth and survival of cancer cells. Immunotoxins, which are targeted toxins that contain an antibody as targeting moiety, are very promising because they combine the specificity of an antibody against tumor-specific antigens, which enables them to channel the toxin to the aimed point of action, and can introduce additionally cell killing mechanisms such as antibody-dependent cell-mediated cytotoxicity and complement-dependent cytotoxicity. To exhibit its effect, the toxin needs to be released into the cytosol after internalization. A major drawback is that the targeting moiety which bears the payload is often not fully internalized, directly recycled to the surface after internalization, or degraded in lysosomes, therewith hampering the sufficient delivery of the payload into the cell cytosol. To ensure a toxic payload concentration for tumor cells and to overcome insufficient cytosolic entry, high serum levels of the targeted toxin are required often resulting in severe side effects, in particular including immunogenicity and vascular leak syndrome. Thus, a sufficiently wide therapeutic window remains a concern when treating cancer patients with antibody-drug conjugates (ADCs).

To cope with the drawback of insufficient cytosolic entry, several strategies were developed relating to for example the redirection of toxins to endogenous cellular membrane transport complexes of the biosynthetic pathway, disruption of endosomes, attenuation of the membrane integrity of endosomal membranes, or use of cell penetrating peptides.

For example, glycosylated triterpenes such as saponins were found to act as endosomal escape enhancers for targeted toxins, such as ribosome-inactivating proteins (RIPs), in tumor therapy. Structural-activity relationship analysis of saponins revealed that the presence of inter alia an aldehyde at the C-4 position appears to be beneficial for the ability of saponins to enhance the cytotoxicity of RIPs (see Formula (1) with A¹=H or OH and A²=a polysaccharide moiety).

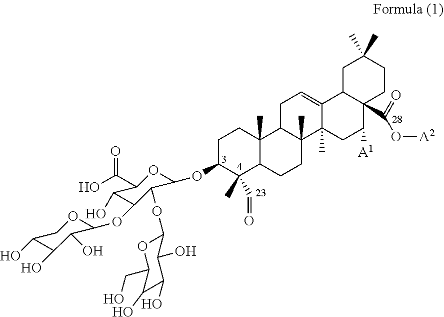

Especially, saponin SO1861 (Formula (2), sometimes also referred to as SPT001 or SPT1 or SPT), a triterpenoid saponin, was identified as a potent molecule in order to enhance the endosomal escape of tumor-cell targeted toxins. A dual effect for the enhancer mechanism is postulated: first, a direct increase of the endosomal escape resulting in caspase-dependent apoptosis that is, second, combined with lysosomal-mediated cell death pathways, which are triggered after the release of cathepsins and other hydrolytic enzymes following destruction of lysosomal membranes.

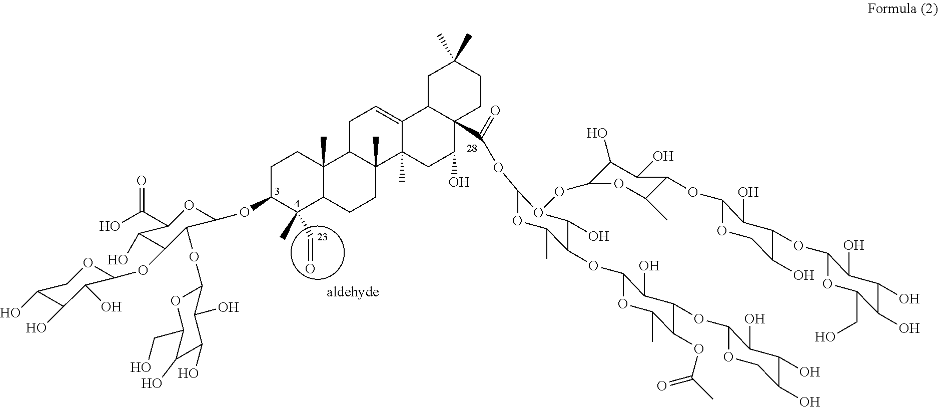

The application of saponins as endosomal escape enhancers is based on the recognition that these saponins have the ability to rupture erythrocyte membranes. However, at the very same time, cell rupturing activity of saponins contribute to (the risk for) side effects when a subject is treated with such saponins, therewith influencing optimal therapeutic windows in view of limiting therapeutic index. Indeed, toxicity of such saponins, extracellularly and/or intracellularly, when administered to a patient in need of anti-tumor therapy, is of concern when for example the optimal dosing regimen and route and frequency of administration are considered.

All characteristics of the chemical composition of the saponins themselves, including the structure of the triterpene backbone, a pentacyclic C30 terpene skeleton (also known as sapogenin or aglycone), number and length of saccharide side chains as well as type and linkage variants of the sugar residues linked to the backbone, contribute to the hemolytic potential and/or cytotoxicity of such saponins.

The saponins are per se not target-specific when the endosome and the cytosol of cells are considered, and saponins expectedly and most often distribute in a (human) subject with other kinetics than the targeted toxins, even when the same route of administration would be considered for a combination of a saponin and e.g. an ADC. Thus, after application to a patient in need thereof of a therapeutic combination comprising e.g. an ADC and a saponin, the saponin molecules can be found in any organ connoting that specificity is only mediated by the targeted toxin. Distribution of saponins in the whole body (systemic distribution) requires higher concentrations for a successful treatment when compared to specific accumulation in target cells as is achieved for the ADC. Hence, the toxicity of the modified saponins needs to be low enough for a successful application in view of the systemic application of saponins in the body, in order to achieve a suitable therapeutic window.

Therefore, there is a still a need to improve the therapeutic index when co-administration of a saponin together with e.g. an ADC is considered: need for better controlling (or better: lowering) the cytotoxicity of saponins while at the same time maintaining sufficient efficacy when potentiation of the cytotoxic effect of an ADC is considered.

ADCs are mainly composed of an antibody, a cytotoxic moiety such as a payload, and a linker. Several novel strategies have been proposed and carried out in the design and development of new ADCs to overcome the existing problems, targeting each of the components of ADCs. For example, by identification and validation of adequate antigenic targets for the antibody component, by selecting antigens which have high expression levels in tumor and little or no expression in normal tissues, antigens which are present on the cell surface to be accessible to the circulating ADCs, and antigens which allow internalizing of ADCs into the cell after binding; and alternative mechanisms of activity; design and optimize linkers which enhance the solubility and the drug-to-antibody ratio (DAR) of ADCs and overcome resistance induced by proteins that can transport the chemotherapeutic agent out of the cells; enhance the DAR ratio by inclusion of more payloads, select and optimize antibodies to improve antibody homogeneity and developability. In addition to the technological development of ADCs, new clinical and translational strategies are also being deployed to maximize the therapeutic index, such as, change dosing schedules through fractionated dosing; perform bio-distribution studies; include biomarkers to optimize patient selection, to capture response signals early and monitor the duration and depth of response, and to inform combination studies.

An example of ADCs with clinical potential are those ADCs such as brentuximab vedotin, inotuzumab ozogamicin, moxetumomab pasudotox, and polatuzumab vedotin, which are evaluated as a treatment option for lymphoid malignancies and multiple myeloma. Polatuzumab vedotin, binding to CD79b on (malignant) B-cells, and pinatuzumab vedotin, binding to CD22, are tested in clinical trials wherein the ADCs each were combined with co-administered rituximab, a monoclonal antibody binding to CD20 and not provided with a payload [B. Yu and D. Liu, Antibody-drug conjugates in clinical trials for lymphoid malignancies and multiple myeloma; Journal of Hematology & Oncology (2019) 12:94]. Combinations of monoclonal antibodies such as these examples are yet a further approach and attempt to arrive at the ‘magic bullet’ which combines many or even all of the aforementioned desired characteristics of ADCs.

Meanwhile in the past few decades, nucleic acid-based therapeutics are under development. Therapeutic nucleic acids can be based on deoxyribonucleic acid (DNA) or ribonucleic acid (RNA), anti-sense oligonucleotides (ASOs, AONs), and short interfering RNAs (siRNAs), microRNAs, and DNA and RNA aptamers, for approaches such as gene therapy, RNA interference (RNAi). Many of them share the same fundamental basis of action by inhibition of either DNA or RNA expression or degradation of mRNA, thereby preventing expression of disease-related abnormal proteins. The largest number of clinical trials is being carried out in the field of gene therapy, with almost 2600 ongoing or completed clinical trials worldwide but with only about 4% entering phase 3. This is followed by clinical trials with ASOs. Similarly to ADCs, despite the large number of techniques being explored, therapeutic nucleic acids share two major issues during clinical development: delivery into cells and off-target effects (often resulting in intolerable adverse events and side effects). For instance, ASOs such as peptide nucleic acid (PNA), phosphoramidate morpholino oligomer (PMO), locked nucleic acid (LNA) and other bridged nucleic acids (BNA), are being investigated as an attractive strategy to inhibit specifically target genes and especially those genes that are difficult to target with small molecule inhibitors or neutralizing antibodies. Currently, the efficacy of different ASOs is being studied in many neurodegenerative diseases such as Huntington's disease, Parkinson's disease, Alzheimer's disease, and amyotrophic lateral sclerosis and also in several cancer stages. The application of ASOs as potential therapeutic agents requires safe and effective methods for their delivery to the cytoplasm and/or nucleus of the target cells and tissues. Although the clinical relevance of ASOs has been demonstrated, inefficient cellular uptake, both in vitro and in vivo, limit the efficacy of ASOs and has been a barrier to therapeutic development. Cellular uptake can be <2% of the dose resulting in too low ASO concentration at the active site for an effective and sustained outcome. This consequently requires an increase of the administered dose which induces off-target effects. Most common side-effects are activation of the complement cascade, the inhibition of the clotting cascade and toll-like receptor mediated stimulation of the immune system.

Chemotherapeutics are most commonly small molecules, however, their efficacy is hampered by the severe off-target side toxicity, as well as their poor solubility, rapid clearance and limited tumor exposure. Scaffold-small-molecule drug conjugates such as polymer-drug conjugates (PDCs) are macromolecular constructs with pharmacologically activity, which comprises one or more molecules of a small-molecule drug bound to a carrier scaffold (e.g. polyethylene glycol (PEG)).

Such conjugate principle has attracted much attention and has been under investigation for several decades. The majority of conjugates of small-molecule drugs under pre-clinical or clinical development are for oncological indications. However, up-to-date only one drug not related to cancer has been approved (Movantik, a PEG oligomer conjugate of opioid antagonist naloxone, AstraZeneca) for opioid-induced constipation in patients with chronic pain in 2014, which is a non-oncology indication. Translating application of drug-scaffold conjugates into treatment of human subjects provided little clinical success so far. For example, PK1 (N-(2-hydroxypropyl)methacrylamide (HPMA) copolymer doxorubicin; development by Pharmacia, Pfizer) showed great anti-cancer activity in both solid tumors and leukemia in murine models, and was under clinical investigation for oncological indications. Despite that it demonstrated significant reduction of nonspecific toxicity and improved pharmacokinetics in man, improvements in anticancer efficacy turned out to be marginal in patients, and as a consequence further development of PK1 was discontinued.

The failure of scaffold-small-molecule drug conjugates is at least partially attributed to its poor accumulation at the tumor site. For example, while in murine models PK1 showed 45-250 times higher accumulation in the tumor than in healthy tissues (liver, kidney, lung, spleen, and heart), accumulation in tumor was only observed in a small subset of patients in the clinical trial.

A potential solution to the aforementioned problems is application of nanoparticle systems for drug delivery such as liposomes. Liposomes are sphere-shaped vesicles consisting of one or more phospholipid bilayers, which are spontaneously formed when phospholipids are dispersed in water. The amphiphilicity characteristics of the phospholipids provide it with the properties of self-assembly, emulsifying and wetting characteristics, and these properties can be employed in the design of new drugs and new drug delivery systems. Drug encapsulated in a liposomal delivery system may convey several advantages over a direct administration of the drug, such as an improvement and control over pharmacokinetics and pharmacodynamics, tissue targeting property, decreased toxicity and enhanced drug activity. An example of such success is liposome-encapsulated form of a small molecule chemotherapy agent doxorubicin (Doxil: a pegylated liposome-encapsulated form of doxorubicin; Myocet: a non-pegylated liposomal doxorubicin), which have been approved for clinical use.

Therefore, a solution still needs to be found that allows for drug therapies such as anti-tumor therapies and anti-auto-immune disease therapies (e.g. rheumatoid arthritis treatment options) and, generally, gene-silencing therapies, applicable for non-systemic use when desired, wherein the drug has for example an acceptable safety profile, little to no off-target activity, sufficient efficacy, sufficiently low clearance rate from the patient's body, a sufficiently wide therapeutic window, etc.

### SUMMARY OF THE INVENTION

According to the inventors the saponin derivatives according to the invention, such as those comprised by the saponin conjugates of the invention, wherein the aldehyde functional group is transformed to a semicarbazone functional group according to formula (I) are not known in the art.

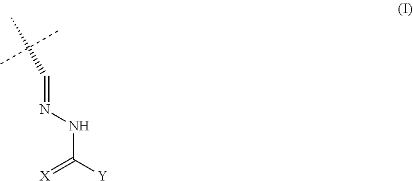

Here, X=O, P or S, and Y=NR³R⁴, wherein R³ and R⁴ independently represent H, an unsubstituted C1-C10 straight chain, branched or cyclic alkyl, an unsubstituted C2-C10 straight chain, branched or cyclic alkenyl or an unsubstituted C2-C10 straight chain or branched alkynyl, or a covalently bound linker, preferably one of R³ and R⁴ is H; or

Y=

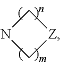

wherein n and m each are an integer independently selected from 1, 2, or 3,
Z=CH₂, O, S, P or NR S, and
wherein R⁵ represents H, an unsubstituted C1-C10 straight chain, branched or cyclic alkyl, an unsubstituted C2-C10 straight chain, branched or cyclic alkenyl, an unsubstituted C₂-C10 straight chain or branched alkynyl, or a covalently bound linker, or a maleimide moiety according to formula (II)a or formula (II)b

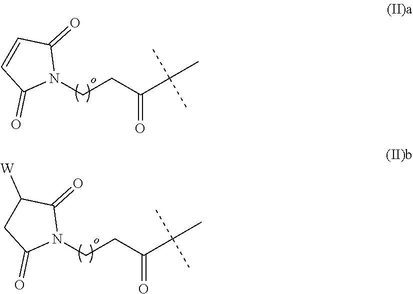

or an azide moiety according to formula (II)c

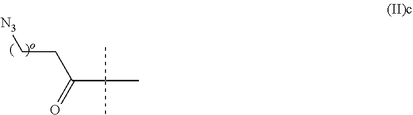

wherein o is an integer selected from 0-10, preferably 2-7, more preferably 4-6, and
W is a thiol functional group according to formula (III)

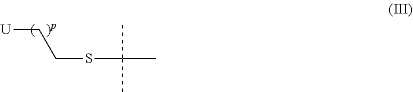

wherein U=SH, NH₂ or OH and
p is an integer selected from 0-4, preferably 1-3, more preferably 1 or 2.

Surprisingly, the inventors have found that these saponin derivatives have one or more of the following benefits:
i) a reduced toxicity when cell viability is considered of cells contacted with the saponin derivatives,
ii) increased activity when potentiation of an effector moiety, e.g. toxin cytotoxicity or AON (e.g. BNA) mediated gene silencing, is considered (without wishing to be bound by any theory: relating to similar or improved endosomal escape enhancing activity of the modified saponin) and/or
iii) reduced hemolytic activity,
   when compared with the toxicity, activity and haemolytic activity of unmodified saponin (non-derivatized saponin). Therewith, the inventors provide saponin derivatives and saponin conjugates comprising such saponin derivatives with an improved therapeutic window, since the ratio between IC50 values for cell toxicity and e.g. IC50 values for toxin potentiation or IC50 values for gene silencing is increased, and/or since the ratio between IC50 values for saponin haemolytic activity and e.g. IC50 values for toxin potentiation or IC50 values for gene silencing is increased.

In addition, the inventors surprisingly established (tumor) cell killing by contacting such cells with a saponin conjugate of the current invention based on a saponin derivative of the current invention e.g. comprising a ligand such as an antibody, for a cell-surface molecule such as a receptor such as CD71 (when the ligand is a CD71 binding antibody in the saponin conjugate), together with an AOC such as an antibody— BNA conjugate, despite the medium to low expression of the cell-surface receptor targeted by the cell-surface molecule binding-molecule (e.g. antibody, such as anti-CD71 antibody) comprised by the saponin conjugate and/or despite the medium to low expression of the cell-surface receptor targeted by the AOC. The saponin derivative and, preferably, also the saponin conjugate of the invention comprise the semicarbazone functional group. For example, in a comparative example, such cell killing when cells are contacted with an ADC or AOC could not be established or only to a lower extent, when a saponin conjugate comprising the same cell-surface molecule binding-molecule (e.g. antibody) but comprising a hydrazone functional group (═N—N(H)—C(O)—) instead of the semicarbazone functional group, was contacted with the cells. Therewith, the inventors now provide for a more potent saponin derivative and a more potent saponin conjugate, when the activation or potentiating of an effector moiety such as an effector moiety comprised by an ADC or AOC, is considered, relating to the presence of the semicarbazone functional group with which the saponin is linked to the cell-surface molecule (such as a cell-surface receptor) binding molecule, for example an antibody or at least one sdAb capable of binding to said cell-surface molecule.

Furthermore, the inventors have found that the saponin derivatives according to the invention and for example comprised by the saponin conjugate of the invention, comprising the semicarbazone functional group hydrolyses more rapidly and in a higher amount towards the corresponding native saponin comprising a “free” aldehyde functional group and on which the saponin derivative is based, as compared to saponin derivatives comprising e.g. a hydrazone functional group (═N—N(H)—C(O)—) known in the art, under acidic conditions which are the conditions present in endosomes and/or lysosomes of mammalian cells, in particular of human cells. This has the benefit that a lower amount of the saponin conjugate comprising the saponin derivative according to the invention should be administered to a patient in need of potentiation of the effector moiety part of e.g. an ADC or an AOC, to obtain the same amount of native saponin comprising the “free” aldehyde to act as endosomal escape enhancer for targeted toxins or targeted oligonucleotides, compared to the required amount of saponin conjugate comprising the saponin derivative comprising the hydrazone functional group (═N—N(H)—C(O)—). Without wishing to be bound by any theory, faster release of the saponin comprising the aldehyde functional group from the saponin conjugate comprising the saponin derivative that comprises the semicarbazone functional group according to the invention, when compared to release of the saponin comprising the aldehyde functional group from the saponin conjugate comprising the saponin derivative that comprises the hydrazone functional group, is at the basis for the improved activity of the saponin conjugate of the invention comprising a saponin derivative of the invention, wherein the activity is the potentiation of the effector moiety activity inside the cytosol or nucleus of the targeted cell by endosomal escape enhancement.

An aspect of the invention relates to a saponin conjugate comprising a first proteinaceous molecule (‘proteinaceous molecule 1’) comprising a cell-surface molecule binding-molecule comprising a first binding site for binding to a first epitope of a first cell-surface molecule and further comprising at least one thiol functional group, according to formula (X)

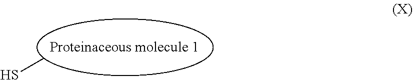

the first proteinaceous molecule covalently bound with at least one saponin derivative, wherein the at least one saponin derivative is based on a saponin comprising a triterpene aglycone core structure and at least one of a first saccharide chain ‘R¹’ and a second saccharide chain ‘R²’ linked to the aglycone core structure, wherein the saponin derivative comprises an aglycone core structure comprising an aldehyde group, wherein the aldehyde group is transformed into a semicarbazone functional group according to formula (I)

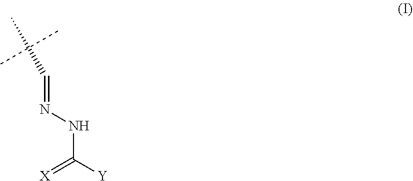

wherein R¹ and R² are independently selected from hydrogen, a monosaccharide, a linear oligosaccharide and a branched oligosaccharide,
X=O, P or S, and
Y=

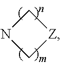

wherein n and m each are an integer independently selected from 1, 2, or 3,
Z=NR⁵, and
wherein R⁵ represents a maleimide moiety according to formula (II)

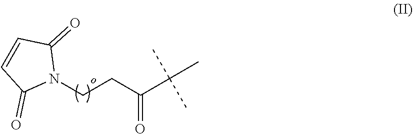

wherein o is an integer selected from 0-10, preferably 2-7, more preferably 4-6, and wherein the maleimide moiety (according to formula) (II) of the saponin derivative is further transformed into a thioether bond through reaction
either, with the at least one thiol functional group of the first proteinaceous molecule,
or, with at least one thiol functional group of an oligomeric molecule which oligomeric molecule comprises a maleimide moiety that is transformed into a thioether bond through reaction with the at least one thiol functional group of the first proteinaceous molecule.

A typical example of the saponin conjugate is the saponin conjugate according to formula (XII)

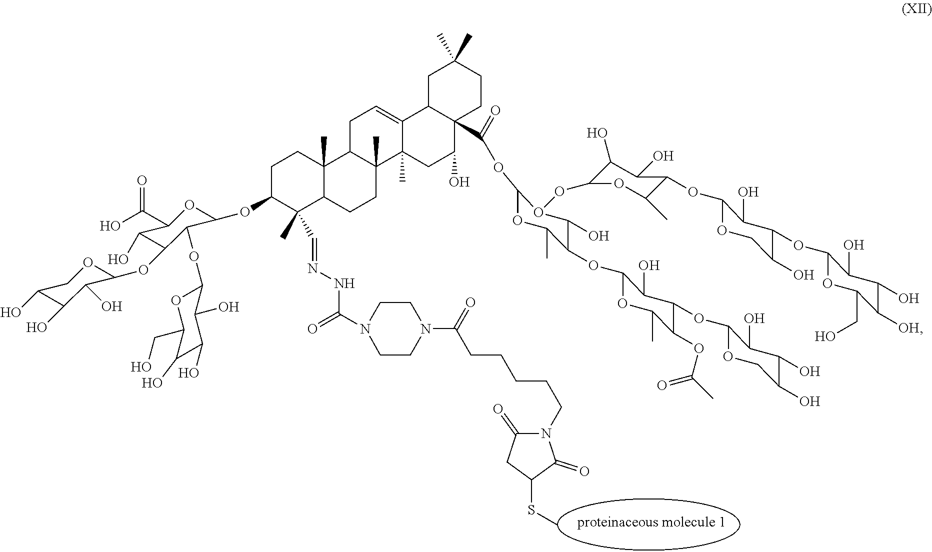

the saponin conjugate according to formula (XII)a

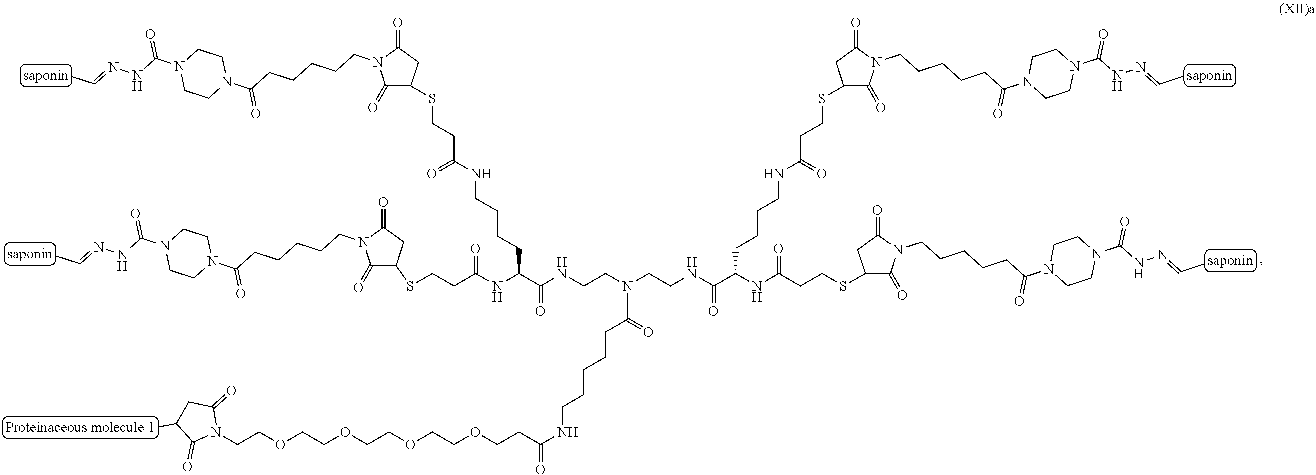 [SMILES: C/C=N/NC(=O)N1CCN(C(=O)CCCCCN2C(=O)CC(SCCC(=O)NCCCC[C@H](NC(=O)CCSC3CC(=O)N(CCCCCC(=O)N4CCN(C(=O)N/N=C/C)CC4)C3=O)C(=O)NCCN(CCNC(=O)[C@H](CCCCNC(=O)CCSC3CC(=O)N(CCCCCC(=O)N4CCN(C(=O)N/N=C/C)CC4)C3=O)NC(=O)CCSC3CC(=O)N(CCCCCC(=O)N4CCN(C(=O)N/N=C/C)CC4)C3=O)C(=O)CCCCCNC(=O)CCOCCOCCOCCOCCN3C(=O)CC(C)C3=O)C2=O)CC1]

and the saponin conjugate according to formula (XII)b

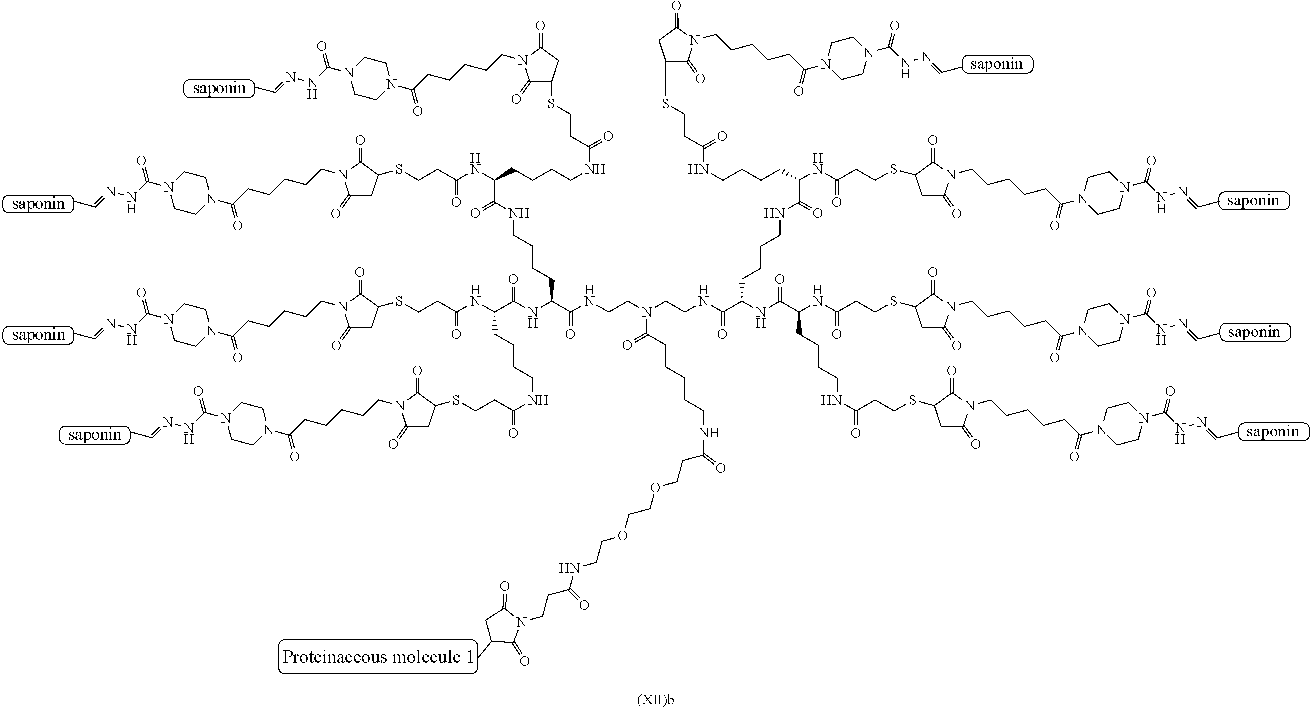

The saponin derivative comprised by the saponin conjugate according to formula (XII)a or formula (XII)b is preferably based on
a) saponin selected from any one or more of list A:
   Quillaja saponaria saponin mixture, or a saponin isolated from Quillaja saponaria, for example Quil-A, QS-17-api, QS-17-xyl, QS-21, QS-21A, QS-21B, QS-7-xyl;
   Saponinum album saponin mixture, or a saponin isolated from Saponinum album; Saponaria officinalis saponin mixture, or a saponin isolated from Saponaria officinalis; and
   Quillaja bark saponin mixture, or a saponin isolated from Quillaja bark, for example Quil-A, QS-17-api, QS-17-xyl, QS-21, QS-21A, QS-21B, QS-7-xyl; or
b) a saponin comprising a gypsogenin aglycone core structure, selected from list B:
   SA1641, gypsoside A, NP-017772, NP-017774, NP-017777, NP-017778, NP-018109, NP-017888, NP-017889, NP-018108, SO1658 and Phytolaccagenin; or
c) a saponin comprising a quillaic acid aglycone core structure, selected from list C:
   AG1856, AG1, AG2, Agrostemmoside E, GE1741, Gypsophila saponin 1 (Gyp1), NP-017674, NP-017810, NP-003881, NP-017676, NP-017677, NP-017705, NP-017706, NP-017773, NP-017775, SA1657, Saponarioside B, SO1542, SO1584, SO1674, SO1700, SO1730, SO1772, SO1832, SO1861, SO1862, SO1904, QS1861, QS1862, QS-7, QS-7 api, QS-17, QS-18, QS-21 A-apio, QS-21 A-xylo, QS-21 B-apio and QS-21 B-xylo.

Preferably, the at least one saponin (four saponin derivative moieties for the saponin conjugate according to formula (XII)a, eight for formula (XII)b) on which the saponin derivative comprised by the saponin conjugate is based, is any one or more of a saponin selected from list B or C, more preferably from list C. Preferably, the saponin is SO1861 or SO1832, more preferably SO1861. FIG. 8B displays a typical example of a saponin conjugate according to the formula (XII)b. The Proteinaceous molecule 1 is here an antibody, preferably a human anti-CD71 antibody. Two G3 dendrons each comprising eight copies of a derivative of SO1861 comprising the semicarbazone functional group, are linked to Cysteine residues of the antibody, via a linker.

Preferably, the first proteinaceous molecule (the proteinaceous molecule 1) is a monoclonal antibody, preferably an anti-CD71-antibody or at least one single domain antibody (sdAb) capable of binding to CD71, wherein the sdAb preferably is a V_{HH} domain.

An embodiment is the saponin conjugate according to formula (SapCon1)

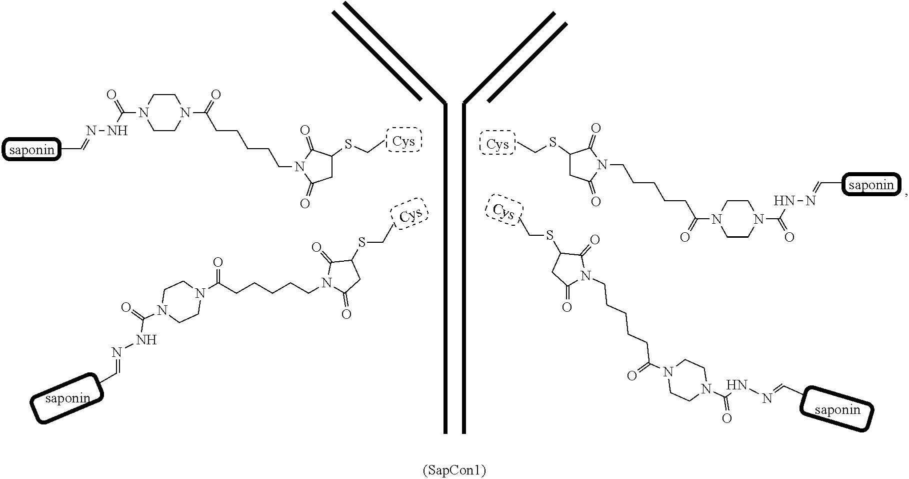 [SMILES: C/C=N/NC(=O)N1CCN(C(=O)CCCCCN2C(=O)CC(SCC)C2=O)CC1 ; C/C=N/NC(=O)N1CCN(C(=O)CCCCCN2C(=O)CC(SCC)C2=O)CC1 ; C/C=N/NC(=O)N1CCN(C(=O)CCCCCN2C(=O)CC(SCC)C2=O)CC1 ; C/C=N/NC(=O)N1CCN(C(=O)CCCCCN2C(=O)CC(SCC)C2=O)CC1]

wherein the saponin derivative comprised by the saponin conjugate is preferably based on
a) saponin selected from any one or more of list A:
   Quillaja saponaria saponin mixture, or a saponin isolated from Quillaja saponaria, for example Quil-A, QS-17-api, QS-17-xyl, QS-21, QS-21A, QS-21B, QS-7-xyl;
   Saponinum album saponin mixture, or a saponin isolated from Saponinum album;
   Saponaria officinalis saponin mixture, or a saponin isolated from Saponaria officinalis; and
   Quillaja bark saponin mixture, or a saponin isolated from Quillaja bark, for example Quil-A, QS-17-api, QS-17-xyl, QS-21, QS-21A, QS-21B, QS-7-xyl; or
b) a saponin comprising a gypsogenin aglycone core structure, selected from list B:
   SA1641, gypsoside A, NP-017772, NP-017774, NP-017777, NP-017778, NP-018109, NP-017888, NP-017889, NP-018108, SO1658 and Phytolaccagenin; or
c) a saponin comprising a quillaic acid aglycone core structure, selected from list C:
   AG1856, AG1, AG2, Agrostemmoside E, GE1741, Gypsophila saponin 1 (Gyp1), NP-017674, NP-017810, NP-003881, NP-017676, NP-017677, NP-017705, NP-017706, NP-017773, NP-017775, SA1657, Saponarioside B, SO1542, SO1584, SO1674, SO1700, SO1730, SO1772, SO1832, SO1861, SO1862, SO1904, QS1861, QS1862, QS-7, QS-7 api, QS-17, QS-18, QS-21 A-apio, QS-21 A-xylo, QS-21 B-apio and QS-21 B-xylo,
   wherein the first proteinaceous molecule of the saponin conjugate is an antibody, such as an anti-CD71 antibody. The saponin conjugate according to formula (SapCon1) comprises four covalently bound saponin derivatives comprising a semicarbazone functional group. Preferably, the four saponins on which the saponin derivatives are based, are the same.

Preferably, the at least one saponin (here, four saponin copies) on which the saponin derivative comprised by the saponin conjugate is based, is any one or more of a saponin selected from list B or C, more preferably from list C. Preferably, the saponin is SO1861 or SO1832, more preferably SO1861. FIG. 6 displays an example of a saponin conjugate according to the formula (SapCon1): four copies of a saponin derivative based on SO1861 comprise the semicarbazone functional group and are linked, via a linker, to Cysteine residues of the monoclonal antibody, typically anti-CD71 antibody.

An aspect of the invention relates to a composition (SapCon) comprising the saponin conjugate of the invention, and optionally comprising a pharmaceutically acceptable diluent and/or a pharmaceutically acceptable excipient.

An aspect of the invention relates to a first pharmaceutical combination comprising:
(a) the composition (SapCon) of the invention comprising the saponin conjugate of the invention; and
(b) a first pharmaceutical composition comprising a (covalently bound) conjugate comprising a cell-surface molecule binding-molecule, such as a second proteinaceous molecule (‘proteinaceous molecule 2’), and an effector moiety, wherein the proteinaceous molecule 2 is the same or different from the proteinaceous molecule 1 present in the saponin conjugate, and if the proteinaceous molecule 2 is different from the proteinaceous molecule 1, the proteinaceous molecule 2 comprising a second binding site for binding to a second epitope of a second cell-surface molecule, wherein the second cell-surface molecule is the same as or different from the first cell surface molecule, and if the second cell-surface molecule is different from the first cell surface molecule, the second cell-surface molecule and the first cell surface molecule are preferably present on the same cell,
the first pharmaceutical composition optionally further comprising a pharmaceutically acceptable excipient and/or a pharmaceutically acceptable diluent.

An aspect of the invention relates to a second pharmaceutical combination, comprising:
(a) the composition (SapCon) of the invention comprising the saponin conjugate of the invention; and
(b) a second pharmaceutical composition comprising a covalently bound conjugate comprising a cell-surface molecule binding-molecule, such as a third proteinaceous molecule (‘proteinaceous molecule 3’), and an effector moiety, wherein the proteinaceous molecule 3 comprises the first binding site for binding to the first epitope on the cell-surface molecule according to the invention, the second pharmaceutical composition optionally further comprising a pharmaceutically acceptable excipient and/or a pharmaceutically acceptable diluent,
wherein the first binding site of the proteinaceous molecule 1 and the first binding site of the proteinaceous molecule 3 are the same, and wherein the first cell-surface molecule and the first epitope on the first cell-surface molecule, to which the proteinaceous molecule 1 can bind, and the first cell-surface molecule and the first epitope on the first cell-surface molecule, to which the proteinaceous molecule 3 can bind, are the same.

An aspect of the invention relates to a third pharmaceutical composition comprising:
(a) the saponin conjugate of the invention; and comprising
   either
   (b1) the conjugate comprising proteinaceous molecule 2 and an effector moiety, or
   (b2) the conjugate comprising proteinaceous molecule 3 and an effector moiety, and the third pharmaceutical composition optionally comprising a pharmaceutically acceptable excipient and/or a pharmaceutically acceptable diluent.

A preferred effector moiety is an oligonucleotide, such as an AON. An AON is a preferred oligonucleotide.

As said, one of the many advantages of the saponin conjugate of the invention is that the semicarbazone functional group

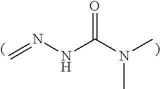

is hydrolysable under acidic conditions, such as at pH 4.0-6.5, wherein hydrolysis of said semicarbazone functional group provides the aldehyde group on the aglycone core structure of the saponin on which the saponin derivative comprised by the saponin conjugate is based, and/or
wherein said semicarbazone functional group is subject to cleavage in vivo under acidic conditions such as for example present in endosomes and/or lysosomes of a mammalian cell, preferably a human cell such as a diseased cell, an aberrant cell or a tumor cell or an autoimmune cell, preferably at pH 4.0-6.5, and more preferably at pH≤5.5, wherein hydrolysis of said semicarbazone functional group provides the aldehyde group on the aglycone core structure of the saponin on which the saponin derivative comprised by the saponin conjugate is based.

An aspect of the invention relates to the first pharmaceutical combination, the second pharmaceutical combination, or the third pharmaceutical composition, for use as a medicament.

An aspect of the invention relates to the first pharmaceutical combination, the second pharmaceutical combination, or the third pharmaceutical composition, for use in the treatment or prevention of a disease or health problem related to presence of a diseased cell according to the invention.

An aspect of the invention relates to the first pharmaceutical combination, the second pharmaceutical combination, or the third pharmaceutical composition, for use in the treatment or prevention of a disease or health problem related to the presence of the aberrant cell according to the invention.

An aspect of the invention relates to the first pharmaceutical combination, the second pharmaceutical combination, or the third pharmaceutical composition, for use in the treatment or prevention of a cancer.

An aspect of the invention relates to the first pharmaceutical combination, the second pharmaceutical combination, or the third pharmaceutical composition, for use in the treatment or prevention of an autoimmune disease.

An aspect of the invention relates to the first pharmaceutical combination, the second pharmaceutical combination, or the third pharmaceutical composition, for use according to the invention, preferably in a human patient, wherein the first cell surface molecule and the third cell surface molecule are CD71 and/or the second cell surface molecule is CD71, and/or the first proteinaceous molecule comprised by the saponin conjugate and the third proteinaceous molecule are a monoclonal antibody capable of binding to CD71 or at least one sdAb capable of binding to CD71, and/or the second proteinaceous molecule is a monoclonal antibody capable of binding to CD71 or at least one sdAb capable of binding to CD71, and/or the effector moiety is an oligonucleotide,
preferably, the first, second and third cell surface molecule is CD71, the first, second and third proteinaceous molecule is a monoclonal antibody capable of binding to CD71 or at least one sdAb capable of binding to CD71, and the effector moiety is an oligonucleotide.

An aspect of the invention relates to an antibody-drug conjugate, antibody-oligonucleotide conjugate, ligand-drug conjugate or ligand-oligonucleotide conjugate, comprising the saponin conjugate of the invention and an effector moiety according to the invention, preferably an antibody-oligonucleotide conjugate comprising the saponin conjugate of the invention and an effector moiety according to the invention.

An aspect of the invention relates to the antibody-drug conjugate, antibody-oligonucleotide conjugate, ligand-drug conjugate or ligand-oligonucleotide conjugate of the invention, preferably the antibody-oligonucleotide conjugate of the invention, for use as a medicament.

An aspect of the invention relates to the antibody-drug conjugate, antibody-oligonucleotide conjugate, ligand-drug conjugate or ligand-oligonucleotide conjugate of the invention, preferably the antibody-oligonucleotide conjugate of the invention, for use in the treatment or prevention of a disease or health problem related to presence of the diseased cell according to the invention, for use in the treatment or prevention of a disease or health problem related to the presence of the aberrant cell according to the invention, for use in the treatment or prevention of a cancer, for use in the treatment or prevention of an autoimmune disease such as rheumatoid arthritis, preferably in a human patient, wherein preferably, the first, second and third cell surface molecule is CD71, the first, second and third proteinaceous molecule is a monoclonal antibody capable of binding to CD71 or at least one sdAb capable of binding to CD71, and the effector moiety is an oligonucleotide.

An aspect of the invention is an in vitro or ex vivo method for transferring a molecule from outside a cell to inside said cell, preferably into the cytosol of said cell is provided, the method comprising the steps of:
a) providing a cell, preferably selected from: an aberrant cell, a diseased cell, a tumor cell and an auto-immune cell;
b) providing the molecule for transferring from outside the cell into the cell provided in step a), the molecule preferably selected from any one of the effector molecules of the invention preferably an oligonucleotide, wherein preferably the molecule for transferring from outside the cell into the cell is provided as a conjugate according to the invention, such conjugate comprising the second or third proteinaceous molecule;
c) providing a saponin conjugate according to the invention;
d) contacting the cell of step a) in vitro or ex vivo with the molecule of step b) and the saponin conjugate of step c), therewith establishing the transfer of the molecule from outside the cell into said cell.

An aspect of the invention relates to the antibody-drug conjugate, antibody-oligonucleotide conjugate, ligand-drug conjugate or ligand-oligonucleotide conjugate of the invention, preferably the antibody-oligonucleotide conjugate of the invention, for use in an in vitro or ex vivo method for transferring a molecule from outside a cell to inside said cell, preferably into the cytosol of said cell is provided, the method comprising the steps of:
a) providing a cell, preferably selected from: an aberrant cell, a diseased cell, a tumor cell and an auto-immune cell;
b) providing the molecule for transferring from outside the cell into the cell provided in step a), the molecule preferably selected from any one of the effector molecules of the invention preferably an oligonucleotide, wherein preferably the molecule for transferring from outside the cell into the cell is provided as a conjugate according to the invention, such conjugate comprising the second or third proteinaceous molecule;
c) providing the antibody-drug conjugate, antibody-oligonucleotide conjugate, ligand-drug conjugate or ligand-oligonucleotide conjugate of the invention, preferably the antibody-oligonucleotide conjugate of the invention;
d) contacting the cell of step a) in vitro or ex vivo with the molecule of step b) and the antibody-drug conjugate, antibody-oligonucleotide conjugate, ligand-drug conjugate or ligand-oligonucleotide conjugate of the invention, preferably the antibody-oligonucleotide conjugate of the invention of step c),
therewith establishing the transfer of the molecule from outside the cell into said cell.

### Definitions

The term “saponin” has its regular scientific meaning and here refers to a group of amphipatic glycosides which comprise one or more hydrophilic glycone moieties combined with a lipophilic aglycone core which is a sapogenin. The saponin may be naturally occurring or synthetic (i.e. non-naturally occurring). The term “saponin” includes naturally-occurring saponins, derivatives of naturally-occurring saponins as well as saponins synthesized de novo through chemical and/or biotechnological synthesis routes.

The term “cell-surface molecule” has its regular scientific meaning and here refers to a molecule that is present and exposed at the outside surface of a cell such as a blood cell or an organ cell, such as a mammalian cell, such as a human cell.

The term “saponin derivative” has its regular scientific meaning and here refers to a saponin, i.e. a modified saponin, which has a chemical modification at a position where previously an aldehyde group was present in the non-derivatised saponin before being subjected to chemical modification for provision of the saponin derivative. For example, the saponin derivative is provided by chemical modification of an aldehyde group, in a saponin upon which the saponin derivative is based, i.e. the saponin is provided and an aldehyde group is chemically modified therewith providing the saponin derivative. For example, the saponin that is derivatised for provision of the saponin derivative is a naturally occurring saponin. Typically, the saponin derivative is a synthetic saponin, typically the saponin derivative is a derivatisation of a natural saponin, and is thus derived from a natural saponin, although a saponin derivative can also be derived from a synthetic saponin which may or may not have a natural counterpart. Typically, the saponin derivative has not a natural counterpart, i.e. the saponin derivative is not produced naturally by e.g. plants or trees. Optionally, the saponin derivative further has one or more chemical modifications at positions where previously any of a carboxyl group, an acetate group and/or an acetyl group was present in the non-derivatised or derivatised saponin before being subjected to chemical modification for provision of the saponin derivative. For example, the saponin derivative is provided by chemical modification of any one or more of an a carboxyl group, an acetate group and/or an acetyl group in a saponin upon which the saponin derivative is based, i.e. the saponin is provided and an aldehyde group, a carboxyl group, an acetate group and/or an acetyl group is chemically modified therewith providing the saponin derivative.

The term “mono-desmosidic saponin” has its regular scientific meaning and here refers to a triterpenoid saponin containing a single saccharide chain bound to the aglycone core, wherein the saccharide chain consists of one or more saccharide moieties.

The term “bi-desmosidic saponin” (also referred to as “bisdesmosidic saponin”) has its regular scientific meaning and here refers to a triterpenoid saponin containing two saccharide chains bound to the aglycone core, wherein each of the two saccharide chains consists of one or more saccharide moieties.

The term “triterpenoid saponin” has its regular scientific meaning and here refers to a saponin having a triterpenoid-type of aglycone core structure. The triterpenoid saponin differs from a saponin based on a steroid glycoside such as sapogenol in that such saponin comprising steroid glycoside has a steroid core structure, and the triterpenoid saponin differs from a saponin based on an alkaloid glycoside such as tomatidine in that such saponin comprising alkaloid glycoside has a alkaloid core structure.

The term “conjugate” has its regular scientific meaning and here refers to at least a first molecule that is covalently bound through chemical bonds to at least a second molecule, therewith forming a covalently coupled assembly comprising or consisting of the first molecule and the second molecule. Typical conjugates are an ADC, an AOC, and SO1861-EMCH (EMCH linked to the aldehyde group of the aglycone core structure of the saponin).

The term “tumor cell-specific surface molecule” and the term “tumor cell-specific receptor” have their regular scientific meaning and here refer to a molecule or a receptor that is expressed and exposed at the surface of a tumor cell and not at the surface of a healthy, non-cancerous cell, or is expressed at the surface of a healthy, non-cancerous cell to a lower extent than the level of expression (number of molecules/receptors) at the surface of the tumor cell.

The term “oligonucleotide” has its regular scientific meaning and here refers to a string of two or more nucleotides, i.e. an oligonucleotides is a short oligomer composed of ribonucleotides or deoxyribonucleotides. Examples are RNA and DNA, and any modified RNA or DNA, such as a string of nucleic acids comprising a nucleotide analogue such as a bridged nucleic acid (BNA), also known as locked nucleic acid (LNA) or a 2′-O,4′-C-aminoethylene or a 2′-O,4′-C-aminomethylene bridged nucleic acid (BNA^{NC}), wherein the nucleotide is a ribonucleotide or a deoxyribonucleotide. As used herein, the terms “nucleic acid”, “oligonucleotide” and “polynucleotide” are synonymous to one another and are to be construed as encompassing any polymeric molecule made of units, wherein a unit comprises a nucleobase (or simply “base” e.g. being a canonical nucleobase like adenine (A), cytosine (C), guanine (G), thymine (T), or uracil (U), or any known non-canonical, modified, or synthetic nucleobase like 5-methylcytosine, 5-hydroxymethylcytosine, xanthine, hypoxanthine, 7-methylguanine; 5,6-dihydrouracil etc.) or a functional equivalent thereof, which renders said polymeric molecule capable of engaging in hydrogen bond-based nucleobase pairing (such as Watson—Crick base pairing) under appropriate hybridisation conditions with naturally-occurring nucleic acids such as deoxyribonucleic acid (DNA) or ribonucleic acid (RNA), which naturally-occurring nucleic acids are to be understood being polymeric molecules made of units being nucleotides.

Hence, from a chemistry perspective, the term nucleic acid under the present definition can be construed as encompassing polymeric molecules that chemically are DNA or RNA, as well as polymeric molecules that are nucleic acid analogues, also known as xeno nucleic acids (XNA) or artificial nucleic acids, which are polymeric molecules wherein one or more (or all) of the units are modified nucleotides or are functional equivalents of nucleotides. Nucleic acid analogues are well known in the art and due to various properties, such as improved specificity and/or affinity, higher binding strength to their target and/or increased stability in vivo, they are extensively used in research and medicine. Typical examples of nucleic acid analogues include but are not limited to locked nucleic acid (LNA) (that is also known as bridged nucleic acid (BNA)), phosphorodiamidate morpholino oligomer (PMO also known as Morpholino), peptide nucleic acid (PNA), glycol nucleic acid (GNA), threose nucleic acid (TNA), hexitol nucleic acid (HNA), 2′-deoxy-2′-fluoroarabinonucleic acid (FANA or FNA), 2′-deoxy-2′-fluororibonucleic acid (2′-F RNA or FRNA); altritol nucleic acids (ANA), cyclohexene nucleic acids (CeNA) etc.

In accordance with the canon, length of a nucleic acid is expressed herein the number of units from which a single strand of a nucleic acid is build. Because each unit corresponds to exactly one nucleobase capable of engaging in one base pairing event, the length is frequently expressed in so called “base pairs” or “bp” regardless whether the nucleic acid in question is a single stranded (ss) or double stranded (ds) nucleic acid. In naturally-occurring nucleic acids 1 bp corresponds to 1 nucleotide, abbreviated to 1 nt. For example, a single stranded nucleic acid made of 1000 nucleotides (or a double stranded nucleic acid made of two complementary strands each of which is made of 1000 nucleotides) is described as having a length of 1000 base pairs or 1000 bp, which length can also be expressed as 1000 nt or as 1 kilobase that is abbreviated to 1 kb. 2 kilobases or 2 kb are equal to the length of 2000 base pair which equates 2000 nucleotides of a single stranded RNA or DNA. To avoid confusion however, in view of the fact the nucleic acids as defined herein may comprise or consist of units not only chemically being nucleotides but also being functional equivalents thereof, the length of nucleic acids will preferentially be expressed herein in “bp” or “kb” rather than in the equally common in the art denotation “nt”.

In advantageous embodiments, the nucleic acid as disclosed herein is no longer than 1 kb, preferably no longer than 500 bp, most preferably no longer than 250 bp.

In particularly advantageous embodiments, the nucleic acid is an oligonucleotide (or simply an oligo) defined as nucleic acid being no longer than 100 bp, i.e. in accordance with the above provided definition, being any polymeric molecule made of no more than 100 units, wherein each unit comprises a nucleobase or a functional equivalent thereof, which renders said oligonucleotide capable of engaging in hydrogen bond-based nucleobase pairing under appropriate hybridisation conditions with DNA or RNA. Within the ambit of said definition, it will immediately be appreciated that the disclosed herein oligonucleotides can comprise or consist of units not only being nucleotides but also being synthetic equivalents thereof. In other words, from a chemistry perspective, as used herein the term oligonucleotide will be construed as possibly comprising or consisting of RNA, DNA, or a nucleic acid analogue such as but not limited to LNA (BNA), PMO (Morpholino), PNA, GNA, TNA, HNA, FANA, FRNA, ANA, CeNA and/or the like.

The term “BNA” refers to BNA^{NC} or 2′,4′-BNA^{NC} (2′-O,4′-aminoethylene bridged nucleic acid) and has its regular scientific meaning and here refers to an oligonucleotide that contains one or more nucleotide building blocks with a six-member bridged structure with an N—O linkage, and with an (N—H) or (N-Me) residue.

The term “payload” has its regular scientific meaning and here refers to a biologically active molecule such as for example a cytotoxic (anti-cancer) drug molecule.

The term “proteinaceous” has its regular scientific meaning and here refers to a molecule comprising at least two amino acid residues linked via a peptide bond with each other so that the molecule is of, relates to, resembles, or is a polypeptide or a protein, meaning that the molecule possesses, to some degree, the physicochemical properties characteristic of a protein, is of protein, relating to protein, containing protein, pertaining to protein, consisting of protein, resembling protein, or being a protein. The term “proteinaceous” as used in for example ‘proteinaceous molecule’ refers to the presence of at least two amino acid residues linked via a peptide bond with each other so that at least a part of the molecule that resembles or is a protein, wherein ‘protein’ is to be understood to include a chain of amino-acid residues at least two residues long, thus including a peptide, a polypeptide and a protein and an assembly of proteins or protein domains. In the proteinaceous molecule, the at least two amino-acid residues are for example linked via (an) amide bond(s), such as (a) peptide bond(s). In the proteinaceous molecule, the amino-acid residues are natural amino-acid residues and/or artificial amino-acid residues such as modified natural amino-acid residues. It is preferred that a proteinaceous molecule is a molecule comprising at least two amino-acid residues, preferably between 2 and about 2,000 amino-acid residues. Also preferred is a proteinaceous molecule that is a molecule comprising from 2 to 20 (typical for a peptide) amino acids. Also preferred is a proteinaceous molecule that is a molecule comprising from 21 to 1,000 amino acid residues (typical for a polypeptide, a protein, a protein domain, such as an antibody, a Fab, an scFv, a ligand for a receptor such as EGF). Preferably, the amino-acid residues are (typically) linked via (a) peptide bond(s). According to the invention, said amino-acid residues are or comprise (modified) (non-)natural amino acid residues.

The term “binding molecule” has its regular scientific meaning and here refers to a molecule capable of specifically binding to another molecule such as a cell-surface molecule, e.g. a cell-surface receptor. Typical binding molecules are peptides, proteins, non-protein molecules, cell-surface receptor ligands, protein ligands, that can bind to e.g. a protein, a lipid, a (poly)saccharide, such as a cell-surface receptor or a cell-surface molecule. “Specifically binding” here refers to specific and selective binding with higher affinity than non-specific background binding.

The term “moiety” has its regular scientific meaning and here refers to a molecule that is bound, linked, conjugated to a further molecule, linker, assembly of molecules, etc., and therewith forming part of a larger molecule, conjugate, assembly of molecules. Typically, a moiety is a molecule that is covalently bound to another molecules, involving one or more chemical groups initially present on the effector molecule. For example, saporin is a typical effector molecule. As part of an antibody-drug conjugate, the saporin is a typical effector moiety in the ADC. As part of an antibody-oligonucleotide conjugate, an AON such as a BNA or an siRNA is a typical effector moiety in the AOC.

The term “aglycone core structure” has its regular scientific meaning and here refers to the aglycone core of a saponin without the one or two carbohydrate antenna or saccharide chains (glycans) bound thereto. For example, quillaic acid is the aglycone core structure for SO1861, QS-7 and QS-21. Typically, the glycans of a saponin are mono-saccharides or oligo-saccharides, such as linear or branched glycans.

The term “QS-21” (also referred to as “QS21”), unless further specified, refers to any one of the isomers of QS-21. As will be understood by the skilled person, a typical natural extract comprising QS-21 will comprise a mixture of the different isomers of QS-21. However, through purification or (semi-)synthetic routes, a single isomer can be isolated.

The term “Saponinum album” has its normal meaning and here refers to a mixture of saponins produced by Merck KGaA (Darmstadt, Germany) containing saponins from Gypsophila paniculata and Gypsophila arostii, containing SA1657 and mainly SA1641.

The term “Quillaja saponin” has its normal meaning and here refers to the saponin fraction of Quillaja saponaria and thus the source for all other QS saponins, mainly containing QS-18 and QS-21.

“QS-21” or “QS21” has its regular scientific meaning and here refers to a mixture of QS-21 A-apio (˜63%), QS-21 A-xylo (˜32%), QS-21 B-apio (˜3.3%), and QS-21 B-xylo (˜1.7%).

Similarly, “QS-21A” has its regular scientific meaning and here refers to a mixture of QS-21 A-apio (˜65%) and QS-21 A-xylo (˜35%).

Similarly, “QS-21 B” has its regular scientific meaning and here refers to a mixture of QS-21 B-apio (˜65%) and QS-21 B-xylo (˜35%).

The term “Quil-A” refers to a commercially available semi-purified extract from Quillaja saponaria and contains variable quantities of more than 50 distinct saponins, many of which incorporate the triterpene-trisaccharide substructure GaI-(1→2)-[XyI-(1→3)]-GlcA- at the C-3beta-OH group found in QS-7, QS-17, QS18, and QS-21. The saponins found in Quil-A are listed in van Setten (1995), Table 2 [Dirk C. van Setten, Gerrit van de Werken, Gijsbert Zomer and Gideon F. A. Kersten, Glycosyl Compositions and Structural Characteristics of the Potential Immuno-adjuvant Active Saponins in the Quillaja saponaria Molina Extract Quil A, RAPID COMMUNICATIONS IN MASS SPECTROMETRY, VOL. 9, 660-666 (1995)]. Quil-A and also Quillaja saponin are fractions of saponins from Quillaja saponaria and both contain a large variety of different saponins with largely overlapping content. The two fractions differ in their specific composition as the two fractions are gained by different purification procedures.

The term “QS1861” and the term “QS1862” refer to QS-7 and QS-7 api. QS1861 has a molecular mass of 1861 Dalton, QS1862 has a molecular mass of 1862 Dalton. QS1862 is described in Fleck et al. (2019) in Table 1, row no. 28 [Juliane Deise Fleck, Andresa Heemann Betti, Francini Pereira da Silva, Eduardo Artur Troian, Cristina Olivaro, Fernando Ferreira and Simone Gasparin Verza, Saponins from Quillaja saponaria and Quillaja brasiliensis: Particular Chemical Characteristics and Biological Activities, Molecules 2019, 24, 171; doi:10.3390/molecules24010171]. The described structure is the api-variant QS1862 of QS-7. The molecular mass is 1862 Dalton as this mass is the formal mass including proton at the glucuronic acid. At neutral pH, the molecule is deprotonated. When measuring in mass spectrometry in negative ion mode, the measured mass is 1861 Dalton.

The term “saccharide chain” has its regular scientific meaning and here refers to any of a glycan, a carbohydrate antenna, a single saccharide moiety (mono-saccharide) or a chain comprising multiple saccharide moieties (oligosaccharide, polysaccharide). The saccharide chain can consist of only saccharide moieties or may also comprise further moieties such as any one of 4E-Methoxycinnamic acid, 4Z-Methoxycinnamic acid, and 5-O-[5-O-Ara/Api-3,5-dihydroxy-6-methyl-octanoyl]-3,5-dihydroxy-6-methyl-octanoic acid), such as for example present in QS-21.

The term “transformation” has its regular scientific meaning and here refers to the chemical transformation of modification of a first functional group or first chemical group or first chemical moiety such that a second functional group or second chemical group or second chemical moiety is provided. An example is the transformation of an aldehyde group carbonyl group into a semicarbazone functional group through reaction with a semicarbazide.

The term “Api/XyI-” or “Api- or XyI-” in the context of the name of a saccharide chain has its regular scientific meaning and here refers to the saccharide chain either comprising an apiose (Api) moiety, or comprising a xylose (Xyl) moiety.

The term “antibody” as used herein is used in the broadest sense, which may refer to an immunoglobulin (Ig) defined as a protein belonging to the class IgG, IgM, IgE, IgA, or IgD (or any subclass thereof), or a functional binding fragment or binding domain of an immunoglobulin. In the context of the present invention, a “binding fragment” ora “binding domain” of an immunoglobulin or of an antibody is defined as antigen-binding fragment or—domain or other derivative of a parental immunoglobulin that essentially maintains the antigen binding activity of such parental immunoglobulin. Functional fragments and functional domains are antibodies in the sense of the present invention even if their affinity to the antigen is lower than that of the parental immunoglobulin. “Functional fragments and—domains” in accordance with the invention include, but are not limited to, F(ab′)2 fragments, Fab′ fragments, Fab fragments, scFv, dsFv, single-domain antibody (sdAb), monovalent IgG, scFv-Fc, reduced IgG (rIgG), minibody, diabodies, triabodies, tetrabodies, Fc fusion proteins, nanobodies, variable V domains such as V_{HH}, Vh, and other types of antigen recognizing immunoglobulin fragments and domains. The fragments and domains may be engineered to minimize or completely remove the intermolecular disulphide interactions that occur between the CH1 and CL domains. Functional fragment and—domains offer the advantage of greater tumor penetration because of their smaller size. In addition, the functional fragment or—domain can be more evenly distributed throughout the tumor mass as compared to whole immunoglobulin.

The term “antibody-drug conjugate” or “ADC” has its regular scientific meaning and here refers to any conjugate of an antibody such as an IgG, an immunoglobulin, an immunoglobulin binding fragment, a binding derivative or binding fragment or binding domain of an antibody such as a F(ab′)2 fragment, Fab′ fragment, Fab fragment, scFv, dsFv, scFv-Fc, reduced IgG (rIgG), minibody, diabody, triabody, tetrabody, Fc fusion protein, nanobody, variable V domain, a single-domain antibody (sdAb), preferably a V_{HH}, a ligand for a cell-surface molecule such as a receptor such as EGF and a cytokine, multiple V_{H} domains, single-domain antibodies, V_{HH}, or camelid V_{H}, etc., and any molecule that can exert a therapeutic effect when contacted with cells of a subject such as a human patient, such as an active pharmaceutical ingredient, a toxin, an oligonucleotide, an enzyme, a small molecule drug compound, etc.

The term “single domain antibody”, or “sdAb”, in short, or ‘nanobody’, has its regular scientific meaning and here refers to an antibody fragment consisting of a single monomeric variable antibody domain, unless referred to as more than one monomeric variable antibody domain such as for example in the context of a bivalent sdAb, which comprises two of such monomeric variable antibody domains in tandem. In the conjugates of the invention, more than one sdAb can be present, which sdAb's can be the same (multivalent and mono-specific) or can be different (multivalent and/or for example multi-paratope, bi-paratope, multi-specific, bi-specific). In addition, for example the more than two sdAb's are for example a combination of mono-specific and multivalent sdAb's and at least one further sdAb that binds to a different epitope (e.g. multispecific or biparatope).

A bivalent nanobody is a molecule comprising two single domain antibodies targeting epitopes on molecules present at the extracellular side of a cell, such as epitopes on the extracellular domain of a cell surface molecule that is present on the cell. Preferably the cell-surface molecule is a cell-surface receptor. A bivalent nanobody is also named a bivalent single domain antibody. Preferably the two different single domain antibodies are directly covalently bound or covalently bound through an intermediate molecule that is covalently bound to the two different single domain antibodies. Preferably the intermediate molecule of the bivalent nanobody has a molecular weight of less than 10,000 Dalton, more preferably less than 5000 Dalton, even more preferably less than 2000 Dalton, most preferably less than 1500 Dalton.

Preferably the two single domain antibodies of the bivalent nanobody do not bind to the same copy of the cell surface molecule present on a cell but bind to different copies of that cell surface molecule present on the same cell. It is believed that binding of the bivalent nanobody to different copies further enhances the uptake (endocytosis) of the nanobody in the cell, or when comprised in a conjugate, it is believed that binding of the bivalent nanobody to different copies on the same cell further enhances the uptake of the conjugate in the cell. This further enhancement may be due to the cross-linking of two cell surface molecules by the bivalent nanobody, which cross-linking is believed to stimulate the uptake. Furthermore, it is believed that improved uptake and internalization enhances endosomal and lysosomal delivery of the bivalent nanobody or the conjugate comprising the bivalent nanobody.

In a different embodiment preferably the two different single domain antibodies of the hetero-bivalent nanobody bind to the same copy of the cell surface molecule present on a cell.

A homo-bivalent nanobody is a bivalent nanobody wherein each of the two single domain antibodies target the same epitope on the extracellular cell-surface molecule, such the extracellular domain of a cell surface molecule that is present on a cell. A homo-bivalent nanobody is also named a homo-bivalent single domain antibody.

A hetero-bivalent nanobody, here also named a biparatopic nanobody, is a bivalent nanobody wherein the two single domain antibodies target different, non-overlapping epitopes on the extracellular domain of a cell surface molecule that is present on a cell. A hetero-bivalent nanobody is also named a hetero-bivalent single domain antibody and is also named a biparatopic single domain antibody or biparatopic nanobody.

A conjugate is a combination of two or more different molecules that have been and are covalently bound. The different molecules of the conjugate for this invention comprise one or more saponins, one or more effector molecules, one or more (bivalent) nanobodies, preferably a single bivalent nanobody molecule comprising two single domain antibodies, more preferably a bi-paratopic sdAb, and optionally though preferably one or more intermediate molecules such as linkers linking the two or more different molecules covalently together, such as for example via linking to a central further linker. In a conjugate, not all of the two or more, such as three, different molecules need to be directly covalently bound to each other. Different molecules in the conjugate may also be covalently bound by being both covalently bound to the same intermediate molecule such as a linker or each by being covalently bound to an intermediate molecule such as a further linker wherein these two intermediate molecules such as two (different) linkers, are covalently bound to each other. According to this definition even more intermediate molecules, such as linkers, may be present between the two different molecules in the conjugate as long as there is a chain of covalently bound atoms in between.

The term “antibody-oligonucleotide conjugate” or “AOC” has its regular scientific meaning and here refers to any conjugate of an antibody such as an IgG, an immunoglobulin, an immunoglobulin binding fragment, a binding derivative or binding fragment or binding domain of an antibody such as a F(ab′)2 fragment, Fab′ fragment, Fab fragment, scFv, dsFv, scFv-Fc, reduced IgG (rIgG), minibody, diabody, triabody, tetrabody, Fc fusion protein, nanobody, variable V domain, a single-domain antibody (sdAb), preferably a V_{HH}, a ligand for a cell-surface molecule such as a receptor such as EGF and a cytokine, multiple V_{H} domains, single-domain antibodies, V_{HH}, or camelid V_{H}, etc., and any oligonucleotide molecule that can exert a therapeutic effect when contacted with cells of a subject such as a human patient, such as an oligonucleotide selected from a natural or synthetic string of nucleic acids encompassing DNA, modified DNA, RNA, mRNA, modified RNA, synthetic nucleic acids, presented as a single-stranded molecule or a double-stranded molecule, such as a BNA, an antisense oligonucleotide (ASO), a short or small interfering RNA (siRNA; silencing RNA), an anti-sense DNA, anti-sense RNA, etc.

The term “linker” has its regular scientific meaning, and linkers are commonly known in the art of bioconjugation. Common linkers are for example described in G. T. Hermanson (Bioconjugation Techniques, Third edition, Elsevier, 2013, ISBN: 978-0-12-382239-0). Here, the term linker refers to a chemical moiety or a linear stretch of amino-acid residues complexed through peptide bonds, which is suitable for covalently attaching (binding) a first molecule, such as a saponin, to another molecule, e.g. to a (proteinaceous) ligand or to an effector molecule or to a scaffold, for example composed of or comprising amino-acid residues, nucleic acids, etc. Typically, the linker comprises a chain of atoms linked by chemical bonds. Any linker molecule or linker technology known in the art can be used in the present disclosure. Where indicated, the linker is a linker for covalently binding of molecules through a chemical group on such a molecule suitable for forming a covalent linkage or bond with the linker. The linker may be a non-cleavable linker, e.g., the linker is stable in physiological conditions. The linker may be a cleavable linker, e.g. a linker that is cleavable, in the presence of an enzyme or at a particular pH range or value, or under physiological conditions such as intracellular conditions in the endosomes such as the late endosomes and the lysosomes of mammalian cells such as human cells. Exemplary linkers that can be used in the context of the present disclosure include, but is not limited to, N-E-maleimidocaproic acid hydrazide (EMCH), succinimidyl 3-(2-pyridyldithio)propionate or 3-(2-Pyridyldithio)propionic acid N-hydroxysuccinimide ester (SPDP), a linker represented by a maleimide moiety according to formula a Da or formula (II)b,

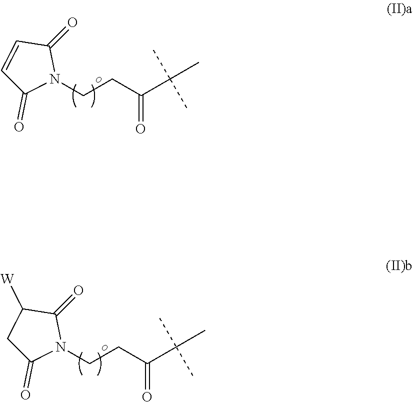

or an azide moiety according to formula (II)c

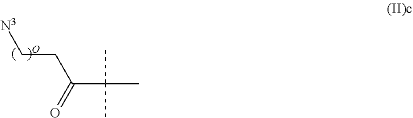

wherein o is an integer selected from 0-10, preferably 2-7, more preferably 4-6, and
W is a thiol functional group according to formula (III),

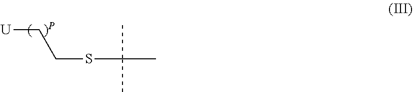

wherein U=SH, NH₂ or OH and
p is an integer selected from 0-4, preferably 1-3, more preferably 1 or 2, and 1-[Bis(dimethylamino)methylene]-1H-1,2,3-triazolo[4,5-b]pyridinium 3-oxid hexafluorophosphate (HATU).

The term “effector molecule”, or “effector moiety” when referring to the effector molecule as part of e.g. a covalent conjugate, has its regular scientific meaning and here refers to a molecule or moiety that has an effect on any one or more of a target molecule and/or proximally to any one or more of a target molecule and/or that can selectively bind to any one or more of a target molecule, wherein the target molecules are for example: a protein, a peptide, a carbohydrate, a saccharide such as a glycan, a (phospho)lipid, a nucleic acid such as DNA, RNA, an enzyme, and regulates the biological activity of such one or more target molecule(s). The effector molecule is for example a molecule selected from any one or more of a small molecule such as a drug molecule, a toxin such as a protein toxin, an oligonucleotide such as an AON such as a BNA, a xeno nucleic acid or an siRNA, an enzyme, a peptide, a protein, or any combination thereof. Thus, for example, an effector molecule or an effector moiety is a molecule or moiety selected from any one or more of a small molecule such as a drug molecule, a toxin such as a protein toxin, an oligonucleotide such as an AON such as a BNA, a xeno nucleic acid or an siRNA, an enzyme, a peptide, a protein, or any combination thereof, that can selectively bind to any one or more of the target molecules: a protein, a peptide, a carbohydrate, a saccharide such as a glycan, a (phospho)lipid, a nucleic acid such as DNA, RNA, an enzyme, and that upon binding to the target molecule regulates the biological activity of such one or more target molecule(s). An effect can include, but is not limited to, biological effect, a therapeutic effect, an imaging effect, and/or a cytotoxic effect. Typically, an effector molecule or moiety can exert a biological effect inside a cell such as a mammalian cell such as a human cell, such as in the cytosol of said cell. At a molecular or cellular level, an effect can include, but is not limited to, promotion or inhibition of the target's activity, labelling of the target, and/or cell death. Typical effector molecules and effector moieties are thus protein toxins, drug molecules, plasmid DNA, toxins such as toxins comprised by antibody-drug conjugates (ADCs), oligonucleotides such as an AON such as an siRNA, BNA, nucleic acids comprised by an antibody-oligonucleotide conjugate (AOC), enzymes. For example, an effector molecule or moiety is a molecule which can act as a ligand that can increase or decrease (intracellular) enzyme activity, gene expression, or cell signalling. The effector moiety is not a saponin on which the saponin derivative or the saponin conjugate of the invention are based. The effector moiety is not the saponin derivative or the saponin conjugate of the invention.

The term “HSP27” relates to a BNA oligonucleotide molecule which silences the expression of HSP27 in the cells. The term “ApoB”, or “ApoBBNA”, or “ApoB #02” relates to a BNA oligonucleotide molecule which silences the expression of apoB in the cells.

The term “bridged nucleic acid”, or “BNA” in short, or “locked nucleic acid” or “LNA” in short or 2′-O,4′-C-aminoethylene or 2′-O,4′-C-aminomethylene bridged nucleic acid (BNA Nc), has its regular scientific meaning and here refers to a modified RNA nucleotide. The term “BNA-based antisense oligonucleotide”, or in short “BNA-AON”, has its regular scientific meaning and here refers to a string of antisense nucleotides wherein at least one of said nucleotides is a BNA. A BNA is also referred to as ‘constrained RNA molecule’ or ‘inaccessible RNA molecule’. A BNA monomer can contain a five-membered, six-membered or even a seven-membered bridged structure with a “fixed” C3′-endo sugar puckering. The bridge is synthetically incorporated at the 2′, 4′-position of the ribose to afford a 2′, 4′-BNA monomer. A BNA monomer can be incorporated into an oligonucleotide polymeric structure using standard phosphoramidite chemistry known in the art. A BNA is a structurally rigid oligonucleotide with increased binding affinity and stability.

The terms first, second, third and the like in the description and in the claims, are used for distinguishing between for example similar elements, compositions, constituents in a composition, or separate method steps, and not necessarily for describing a sequential or chronological order. The terms are interchangeable under appropriate circumstances and the embodiments of the invention can operate in other sequences than described or illustrated herein, unless specified otherwise.

The term ‘L’ as used such as in an saponin derivative or an antibody-saponin conjugate or construct comprising a linker, represents ‘labile linker’ which is cleaved under slightly acid conditions (pH<6.6, such as pH 4.0-5.5) in the endosome, endolysosome and in the lysosome of mammalian cells, such as human cells, such as a human tumor cell.

The embodiments of the invention described herein can operate in combination and cooperation, unless specified otherwise.

Furthermore, the various embodiments, although referred to as “preferred” or “e.g.” or “for example” or “in particular” and the like are to be construed as exemplary manners in which the invention may be implemented rather than as limiting the scope of the invention.

The term “comprising”, used in the claims, should not be interpreted as being restricted to for example the elements or the method steps or the constituents of a compositions listed thereafter; it does not exclude other elements or method steps or constituents in a certain composition. It needs to be interpreted as specifying the presence of the stated features, integers, (method) steps or components as referred to, but does not preclude the presence or addition of one or more other features, integers, steps or components, or groups thereof. Thus, the scope of the expression “a method comprising steps A and B” should not be limited to a method consisting only of steps A and B, rather with respect to the present invention, the only enumerated steps of the method are A and B, and further the claim should be interpreted as including equivalents of those method steps. Thus, the scope of the expression “a composition comprising components A and B” should not be limited to a composition consisting only of components A and B, rather with respect to the present invention, the only enumerated components of the composition are A and B, and further the claim should be interpreted as including equivalents of those components.

The term “DAR” normally stands for Drug Antibody Ratio and refers to the average drug to antibody ratio for a given preparation of antibody drug conjugate (ADC) and here refers to a ratio of the number of bound saponin copies or moieties such as SO1861 moieties, or SPT001 moieties, or bound payload, e.g. an AON such as ApoB BNA with respect to the conjugate molecule, or refers to the number of oligomeric molecules comprising saponin moieties with respext to the conjugate molecule.

An “aberrant cell” has its regular scientific meaning and is here defined as a cell that deviates from its usual and healthy normal counterparts and for example an aberrant cell can show uncontrolled growth characteristics. Typical aberrant cells are autoimmune cells and tumor cells that (over)express a tumor-cell related antigen such as a cell-surface receptor, at the surface of the autoimmune cell or tumor cell. The tumor-cell related antigen can be unique for the aberrant cell or can be over-expressed relatively to the expression level of the antigen on the usual and healthy normal counterparts of the aberrant cell.

A “diseased cell” has its regular scientific meaning and is here defined as a cell that comprises a gene defect causing or contributing to a disease and/or a health problem, and/or as a cell that exhibits deviating transcription of a gene relative to transcription of the gene in a healthy normal cell causing or contributing to a disease and/or a health problem, wherein ‘deviating transcription’ refers to upregulated (increased) transcription or down-regulated (decreased) transcription, and/or as a cell that exhibits deviating expression of a protein relative to expression of the protein in a healthy normal cell causing or contributing to a disease and/or a health problem, wherein ‘deviating expression’ refers to upregulated (increased) protein expression or down-regulated (decreased) protein expression. Typical diseased cells can be autoimmune cells and tumor cells overexpressing a tumor-cell related antigen or expressing HSP27, or liver cells displaying too high expression levels of ApoB.

In addition, reference to an element or a component by the indefinite article “a” or “an” does not exclude the possibility that more than one of the element or component are present, unless the context clearly requires that there is one and only one of the elements or components. The indefinite article “a” or “an” thus usually means “at least one”.

The terms “SO1861” and “SO1862” refer to the same saponin of Saponaria officinalis, though in deprotonated form or api form, respectively. The molecular mass is 1862 Dalton as this mass is the formal mass including a proton at the glucuronic acid. At neutral pH, the molecule is deprotonated. When measuring the mass using mass spectrometry in negative ion mode, the measured mass is 1861 Dalton.

### BRIEF DESCRIPTION OF THE DRAWINGS

The present invention will be discussed in more detail below, with reference to the attached drawings, in which

FIG. 1: SO1861-SC-Mal synthesis

FIG. 2A-B: SO1861-semicarbazone (‘SO1861-SC’) synthesis (FIG. 2A); SO1861-SC-Mal (block) synthesis (FIG. 2B)

FIG. 3: SO1861-EMCH synthesis

FIG. 4A-C: Dendron4-amine synthesis. Synthesis of intermediate 5 and intermediate 6 (FIG. 4A); Synthesis of intermediate 7 and intermediate 9 (FIG. 4B); Synthesis of intermediate 10 (FIG. 4C)

FIG. 4D: 501861-EMCH and 501861-SC-Mal structure

FIG. 4E: Dendron-(EMCH-501861)4-amine and Dendron-(SC-501861)4-amine synthesis

FIG. 4F: Dendron-(EMCH-501861)4-azide and Dendron-(SC-501861)4-azide synthesis

FIG. 4G: Dendron-(EMCH-501861)4-maleimide1 and Dendron-(SC-501861)4-maleimide1 synthesis

FIG. 4H: Dendron-(EMCH-501861)4-maleimide2 and Dendron-(SC-501861)4-maleimide2 synthesis

FIG. 5A-D: Dendron8-amine synthesis. Synthesis of intermediate 13 (FIG. 5A); Synthesis of intermediate 14 (FIG. 5B); Synthesis of intermediate 15 (FIG. 5C); Synthesis of intermediate 16 (FIG. 5D)

FIG. 5E: Dendron-(EMCH-501861)8-amine and Dendron-(SC-501861)8-amine synthesis

FIG. 5F: Dendron-(EMCH-501861)8-azide and Dendron-(SC-501861)8-azide synthesis

FIG. 5G: Dendron-(EMCH-SO1861)8-maleimide1 and Dendron-(SC-SO1861)8-maleimide1 synthesis

FIG. 5H: Dendron-(EMCH-SO1861)8-maleimide2 and Dendron-(SC-SO1861)8-maleimide2 synthesis

FIG. 6: Structure of hCD71mab-SC-501861 conjugate. Graphical representation of mAb-semicarbazone-501861 (DAR4). The derivatised saponin 501861-SC-Mal is covalently linked to the central monoclonal antibody (e.g. an IgG) via covalent bond formation between Cysteine residues of the antibody and the maleimide group of the derivatised SO1861. Four SO1861 moieties are bound to the antibody

FIG. 7A-B: Structure of hCD71mab-dendron(EMCH-SO1861)4 conjugate. 501861-hydrazone moiety (FIG. 7A); antibody-dendron(EMCH-501861)4 conjugate (FIG. 7B)

FIG. 8A-B: Structure of hCD71mab-dendron(SC-501861)8 conjugate. SO1861-semicarbazone moiety and dendron(SC-501861)8-maleimide1 (FIG. 8A); antibody-dendron(SC-SO1861)8 conjugate (FIG. 8B). ‘SPT001’ is SO1861.

FIG. 9A-B: Release kinetic assay of 501861-EMCH (formula (4)) (FIG. 9A) and SO1861-SC-Mal (blocked) (Molecule VIII) (FIG. 9B) at various pH

FIG. 10A-B: Cell killing assay (MTS) of 501861-SC-Mal+5 pM EGF-dianthin on HeLa (FIG. 10 A) and A431 (FIG. 10B) cell lines. Note: the legend to FIG. 10A and FIG. 10B is the same and is displayed next to the graphs in FIG. 10A.

FIG. 11A-B: Cell killing assay (MTS) of 501861-SC-Mal+10 pM Cetuximab-saporin on HeLa

(FIG. 11A) and A431 (FIG. 11B) cell lines. Note: the legend to FIG. 11A and FIG. 11B is the same and is displayed next to the graphs in FIG. 11A.

FIG. 12A-B: Cell killing assay (MTS) of 501861-SC-Mal+50 pM Trastuzumab-saporin on HeLa (FIG. 12A) and A431 (FIG. 12B) cell lines. Note: the legend to FIG. 12A and FIG. 12B is the same and is displayed next to the graphs in FIG. 12A.

FIG. 13A-B: HSP27 gene silencing of 501861-SC-Mal+Tmab-HSP27BNA on A431 cell lines. (FIG. 13A) Relative HSP27 expression in the absence of saponin. (FIG. 13B) Relative HSP27 expression under influence of saponin analogues 501861-EMCH and 501861-SC-Mal and of wild type (non-modified, non-derivatised) saponin SO1861.

FIG. 14A-B: Cell killing assay (MTS) of 501861-SC-Mal, SO1861-SC, 501861-EMCH and non-derivatised SO1861 on HeLa (FIG. 14A) and A431 (FIG. 14B) cell lines. Note: the legend to FIG. 14A and FIG. 14B is the same and is displayed next to the graphs in FIG. 14A. ‘SC’ is ‘semicarbazone’

FIG. 15: Hemolytic activity of modified SO1861 (SO1861 derivatives) and non-derivatised SO1861 on human red blood cells (RBC).

FIG. 16A-B: EGFR/CD71 targeted cell killing in A431 cells (FIG. 16A) and CaSKi cells (FIG. 16B). Cetuximab-(SC-501861)4, also called Cetuximab-SC-501861 (DAR⁴) and Cetuximab-(EMCH-501861)4 titration+fixed concentration 10 pM CD71mab-saporin and controls on A431 cells (EGFR++/CD71+) and CaSKi cells(EGFR++/CD71+). Note: the legend to FIG. 16A and FIG. 16B is the same and is displayed next to the graphs in FIG. 16B.

FIG. 17A-B: EGFR/CD71 targeted cell killing in HeLa cells (FIG. 17A) and A2058 cells (FIG. 17B). Cetuximab-(SC-SO1861)4 and Cetuximab-(EMCH-SO1861)4 titration+fixed concentration 10 pM CD71mab-saporin and controls on HeLa cells (EGFR+/−/CD71+) and A2058 cells (EGFR−/CD71+). Note: the legend to FIG. 17A and FIG. 17B is the same and is displayed next to the graphs in FIG. 17B.

FIG. 18: HER2/CD71 targeted cell killing. Trastuzumab-(SC-SO1861)4 or Trastuzumab-(EMCH-SO1861)4 titration+fixed concentration 10 pM CD71mab-saporin and controls on SK-BR3 cells (HER2++/CD71+).

FIG. 19A-B: HER2/CD71 targeted cell killing. (FIG. 19A, FIG. 19B) Trastuzumab-(SC-SO1861)4 or Trastuzumab-(Cys-EMCH-SO1861)4 titration+fixed concentration 10 pM CD71mab-saporin and controls on JIMT-1 cells (HER2+/−/CD71+) (FIG. 19A), and MDA-MB-468 (HER2-/CD71+) (FIG. 19B). Note: the legend to FIG. 19A and FIG. 19B is the same and is displayed next to the graphs in FIG. 19B.

FIG. 20: EGFR/HER2 targeted gene silencing in A431 cells. Cetuximab-(SC-SO1861)4 and Cetuximab-(EMCH-SO1861)4 titration+fixed concentration 50 pM Trastuzumab-S-HSP27BNA and controls on A431 cells (EGFR++/HER2+/−). The term ‘S’ as used such as in an antibody-oligonucleotide conjugate, represents ‘stable linker’ which is a non-cleavable linker in the endosome, endolysosome and in the lysosome of mammalian cells, such as human cells, such as a human tumor cell, thus under slightly acidic conditions (pH<6.6, such as pH 4.0-5.5).

FIG. 21: Cell killing assay (MTS) of various cell lines treated with 5 pM EGFdianthin (Dia-EGF), 10 pM Cetuximab-saporin (Cet-SPRN), 50 pM Trastuzumab-saporin (Tras-SPRN) or 10 pM CD71-saporin (CD71-SPRN).

FIG. 22: HSP27 mRNA expression levels in A431 cells. Displayed is the relative HSP27 expression in A431 cells contacted with Cetuximab-(SC-SO1861)-HSP27 (DAR4/DAR2) and Cetuximab-(EMCH-SO1861)-HSP27 (DAR4/DAR2). The conjugates comprise two BNA molecules (DAR²) and four SO1861 moieties (DAR⁴).

FIG. 23A-F: CD71/EGFR targeted cell killing in SK-BR3 (FIG. 23A), JIMT-1 (FIG. 23B), HeLa (FIG. 23C), and MDA-MB-468 cells (FIG. 23D) A431 (FIG. 23E) and A2058 (FIG. 23F). SO1861-SC-Mal, CD71mAb-(SC-SO1861)⁴, CD71mAb-(EMCH-SO1861)⁴, CD71mAb-dendron(SC-SO1861)8^{1.5} (average of ˜12 SO1861 molecules) and CD71mAb-dendron(EMCH-SO1861)4^{3.2} (on average of −12 SO1861 molecules) titration+fixed concentration 5 pM EGFdianthin (fusion protein toxin).

### DETAILED DESCRIPTION

The present invention will be described with respect to particular embodiments but the invention is not limited thereto but only by the claims.

Surprisingly, the inventors have found that a saponin derivative based on a saponin comprising a triterpene aglycone core structure and at least one of a first saccharide chain ‘R¹’ and a second saccharide chain ‘R²’ linked to the aglycone core structure, wherein the saponin derivative comprises an aglycone core structure comprising an aldehyde group, wherein the aldehyde group is transformed into a semicarbazone functional group according to formula (I)

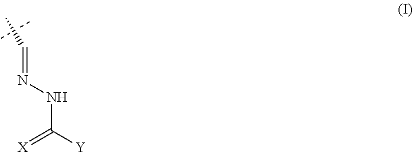

wherein R¹ and R² are independently selected from hydrogen, a monosaccharide, a linear oligosaccharide and a branched oligosaccharide,
X=O, P or S, and
Y=NR³R⁴, wherein R³ and R⁴ independently represent H, an unsubstituted C1-C10 straight chain, branched or cyclic alkyl, an unsubstituted C2-C10 straight chain, branched or cyclic alkenyl or an unsubstituted C2-C10 straight chain or branched alkynyl, or a covalently bound linker, preferably one of R³ and R⁴ is H; or
Y=

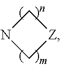

wherein n and m each are an integer independently selected from 1, 2, or 3,
Z=CH₂, O, S, P or NR⁵, and
wherein R⁵ represent H, an unsubstituted C1-C10 straight chain, branched or cyclic alkyl, an unsubstituted C2-C10 straight chain, branched or cyclic alkenyl, an unsubstituted C2-C10 straight chain or branched alkynyl, or a covalently bound linker, or a maleimide moiety according to formula (II)a or formula (II)b,

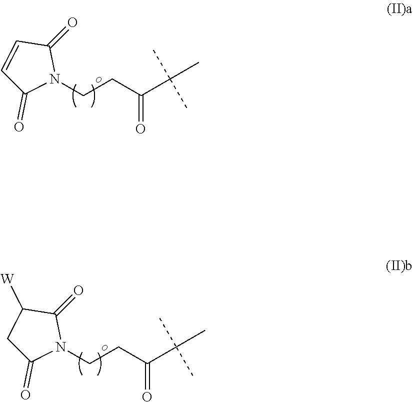

or an azide moiety according to formula (II)c

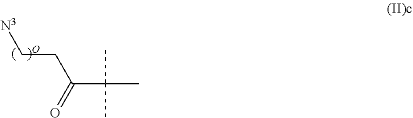

wherein o is an integer selected from 0-10, preferably 2-7, more preferably 4-6, and
W is a thiol functional group according to formula (III),

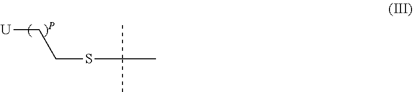

wherein U=SH, NH₂ or OH and
p is an integer selected from 0-4, preferably 1-3, more preferably 1 or 2, has a reduced toxicity when cell viability is considered of cells contacted with the saponin derivatives; has activity when potentiation of e.g. toxin cytotoxicity or AON-, such as BNA, mediated gene silencing is considered (without wishing to be bound by any theory: relating to similar or improved endosomal escape enhancing activity of the modified saponin), if the aldehyde functional group is transformed to a semicarbazone functional group according to formula (I); and/or has reduced hemolytic activity, when compared with the toxicity, activity and haemolytic activity of unmodified saponin. That is to say, the saponin derivative of the invention has at least one, preferably to, more preferably all three of:
(i) a reduced toxicity when cell viability is considered of cells contacted with the saponin derivatives;
(ii) enhanced activity when potentiation of e.g. toxin cytotoxicity or AON-mediated gene silencing such as BNA-mediated gene silencing is considered (without wishing to be bound by any theory: relating to similar or improved endosomal escape enhancing activity of the modified saponin), if the aldehyde functional group is transformed to a semicarbazone functional group according to formula (I); and/or
(iii) reduced hemolytic activity,
when compared with the toxicity, activity and haemolytic activity of unmodified saponin on which the saponin derivative is based. Therewith, the inventors provide saponin derivatives with an improved therapeutic window, since for the saponin derivatives, the cytotoxicity is lower than cytotoxicity determined for their naturally occurring counterparts, the haemolytic activity is lower than haemolytic activity determined for the naturally occurring counterparts of the saponin derivatives, the ratio between IC50 values for cell toxicity and e.g. IC50 values for toxin potentiation or IC50 values for gene silencing is similar or increased, and/or since the ratio between IC50 values for saponin haemolytic activity and e.g. IC50 values for toxin potentiation or IC50 values for gene silencing is similar or increased. Reference is made to Table 3 and Table 4 of the examples for an overview of exemplified saponin derivatives for an overview of the cytotoxicity, haemolytic activity and endosomal escape enhancing activity (activity), as well as an overview of the ratio between IC50 for cytotoxicity and IC50 for activity, and the ratio between IC50 for haemolytic activity and IC50 for activity.

In addition, the inventors surprisingly established (tumor) cell killing by contacting such cells with a saponin conjugate of the current invention based on a saponin derivative of the current invention, together with an ADC such as an antibody—protein toxin conjugate, despite the medium to low expression of the cell-surface receptor targeted by the cell-surface molecule binding-molecule (e.g. antibody) comprised by the saponin conjugate and/or despite the medium to low expression of the cell-surface receptor targeted by the ADC. The saponin derivative and the saponin conjugate of the invention comprise the semicarbazone functional group. For example, in a comparative example, such cell killing could not be established or only to a lower extent, when a saponin conjugate comprising the same cell-surface molecule binding-molecule (e.g. antibody) but comprising a hydrazone functional group (═N—N(H)—C(O)—) instead of the semicarbazone functional group, was contacted with the cells. Therewith, the inventors now provide for a more potent saponin derivative and saponin conjugate, when the activation or potentiating of an effector moiety such as an effector moiety comprised by an ADC or AOC, is considered.

Furthermore, the inventors have found that the saponin derivatives according to the invention comprising the semicarbazone functional group hydrolyses more rapidly and in an higher amount towards the corresponding native saponin comprising a “free” aldehyde functional group, as compared to saponin derivatives comprising a hydrazone functional group (═N—N(H)—C(O)—) known in the art, under acidic conditions which are the conditions present in endosomes and/or lysosomes of mammalian cells. This has the benefit that a lower amount of the saponin derivatives according to the invention should be administered to a patient in need of potentiation of e.g. an ADC or an AOC, to obtain the same amount of native saponin comprising the “free” aldehyde to act as endosomal escape enhancers for targeted toxins or targeted oligonucleotides, compared to the required amount of saponin derivative comprising e.g. the hydrazone functional group (═N—N(H)—C(O)—). Without wishing to be bound by any theory, faster and more efficacious release of the saponin comprising the aldehyde functional group from the saponin derivative that comprises the semicarbazone functional group according to the invention, when compared to release of the saponin comprising the aldehyde functional group from the saponin derivative that comprises e.g. the hydrazone functional group, is at the basis for the improved activity of the saponin conjugate of the invention comprising a saponin derivative of the invention, such as a conjugate comprising the saponin, an oligonucleotide and a cell-surface receptor ligand such as EGF or an antibody or an sdAb, wherein the activity is the potentiation of the effector moiety activity such as a gene-silencing oligonucleotide, inside the cytosol or nucleus of the targeted cell by endosomal escape enhancement.

Surprisingly, transformation (derivatisation) of the aldehyde group at C-23 of the aglycone of the saponin into a semicarbazone functional group according to formula (I), results in a decrease in cytotoxicity when such saponin derivatives are contacted with cells, i.e. various types of cells. It is thus part of the invention that these series of saponin derivatives with decreased cytotoxicity are provided, wherein the decrease in cytotoxicity is relative to the cytotoxicity as determined for the unmodified naturally occurring saponin counterparts. The saponin derivatives can be formed from such naturally occurring saponins, such as SO1861, equally active SO1832 and QS-21 (isoforms), preferably from SO1861 or SO1832. When the decrease in cytotoxicity is considered, saponin derivatives according to the invention, are equally suitable, when saponins with decreased cytotoxicity are to be provided.

The saponin SO1832 consists of the quillaic acid aglycone core with the carbohydrate substituent GaI-(1→2)-[XyI-(1→3)]-GlcA- at the C-3beta-OH group and with the carbohydrate substituent XyI-(1→3)-XyI-(1→4)-Rha-(1→2)-[XyI-(1→3)-4-OAc-Qui-(1→4)]-Fuc- at the C-28-OH group (See also Table A1). The chemical formula is C₈₂H₁₂₈O₄₅ and the exact mass is 1832,77 Dalton. The saponin structure of SO1832 is according to molecule (SO1832):

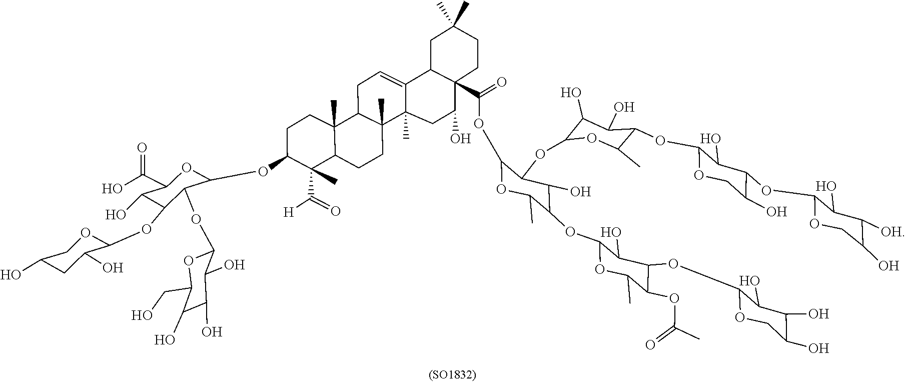

The inventors have also found that the modifications applied to provide the saponin derivatives lead to an increased critical micelle concentration (CMC) when compared with the corresponding unmodified saponin. For example, the saponin derivative according to formula (VIII) has an increased CMC when compared to their corresponding underivatised saponin. Without wishing to be bound by any theory, it is believed that an increased CMC is advantageous for several reasons. For example, an increased CMC may facilitate the use of the modified saponins in subsequent conjugation reactions since free molecules are generally more susceptible to conjugation reactions than molecules ordered in a micellar structure. Furthermore, in case the saponin derivatives need to exert a biological function (e.g. in an in vivo treatment or ex vivo method or in vitro method), for example in case the saponin derivatives are used as such or even in case they are released in-situ after cleavage from a carrier or another entity, an increased CMC when compared to unmodified saponin is advantageous since the free saponin molecules will be more readily available to interact with their biological target than in case these saponin derivatives are ordered in a micellar structure. An increased CMC may also be useful to facilitate the large scale production and concentration of the saponin derivatives since at concentrations beyond (above) the critical micellar concentration, saponins form micelles which hinder isolation (e.g. using preparative HPLC). Surprisingly, for the saponin derivatives according to the invention the observed increased CMC was not associated with increased cytotoxicity or haemolytic activity. The relationship between CMC and cytotoxicity is not predictable and complex, as can for example be seen from the data in Table 2 of de Groot et al. (“Saponin interactions with model membrane systems—Langmuir monolayer studies, hemolysis and formation of ISCOMs”, planta medica 82.18 (2016): 1496-1512.), which shows that, taking α-Hederin as the reference point, an increase in CMC may be associated with an increase in general cytotoxicity (as is the case for Digitonin) but may just as well be associated with a decrease in cytotoxicity (as is the case for Glycyrrhizin and Hederacoside C). Furthermore, for the saponin derivative according to formula (VIII) the increased CMC is also associated with an increased ratio: IC50 hemolysis/IC50 activity, compared to the corresponding free saponin. The inventors thus provide saponin derivatives with an improved therapeutic window when cytotoxicity is considered and/or when haemolytic activity is considered, and when the potentiation of e.g. toxins is considered and/or when an increased CMC compared to the corresponding underivatised saponin is considered. Such saponin derivatives of the invention are in particular suitable for application in a therapeutic regimen involving e.g. an ADC or an AOC for the prophylaxis or treatment of e.g. a cancer, or a disease or health problem related to an aberrant cell or a diseased cell, in a (human) subject in need thereof. The safety of such saponin derivatives is improved when cytotoxicity and/or haemolytic activity is considered, especially when such saponin derivatives are administered to a patient in need of e.g. treatment with an ADC or with and AOC comprising an oligonucleotide, e.g. an AON such as a BNA for silencing a gene such as HSP27 and apoB.

An aspect of the invention relates to a saponin derivative based on a saponin comprising a triterpene aglycone core structure and at least one of a first saccharide chain ‘R¹’ and a second saccharide chain ‘R²’ linked to the aglycone core structure, wherein the saponin derivative comprises an aglycone core structure comprising an aldehyde group, wherein the aldehyde group is transformed into a semicarbazone functional group according to formula (I)

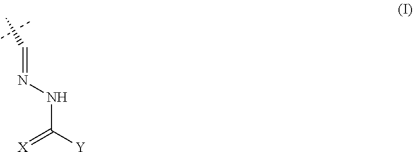

wherein R¹ and R² are independently selected from hydrogen, a monosaccharide, a linear oligosaccharide and a branched oligosaccharide,
X=O, P or S, and
Y=NR³R⁴, wherein R³ and R⁴ independently represent H or a covalently bound linker, preferably one of R³ and R⁴ is H; or
Y=

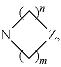

wherein n and m each are an integer independently selected from 1, 2, or 3,
Z=CH₂, O, S, P or NR S, and
wherein R⁵ represents H or a covalently bound linker, or a maleimide moiety according to formula (II)a (also referred to as ‘formula (II)’) or formula (II)b

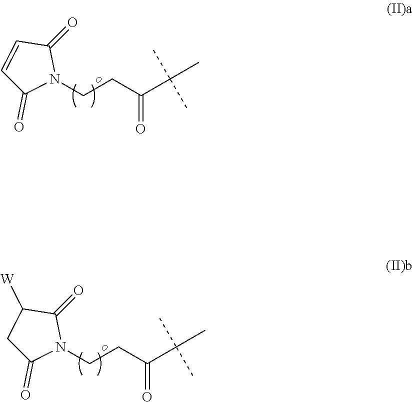

or an azide moiety according to formula (II)c

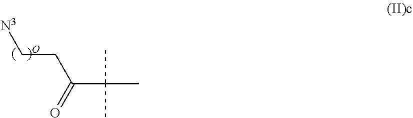

wherein o is an integer selected from 0-10, preferably 2-7, more preferably 4-6, and
W is a thiol functional group according to formula (III)

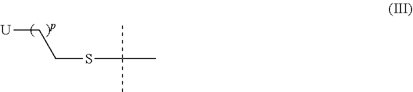

wherein U=SH, NH₂ or OH and
p is an integer selected from 0-4, preferably 1-3, more preferably 1 or 2.

An aspect of the invention relates to a saponin derivative based on a saponin comprising a triterpene aglycone core structure and at least one of a first saccharide chain ‘R¹’ and a second saccharide chain ‘R²’ linked to the aglycone core structure, wherein the saponin derivative comprises an aglycone core structure comprising an aldehyde group, wherein the aldehyde group is transformed into a semicarbazone functional group according to formula (I)

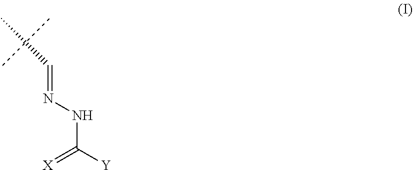

wherein R¹ and R² are independently selected from hydrogen, a monosaccharide, a linear oligosaccharide and a branched oligosaccharide,
X=O, P or S, and
Y=NR³R⁴, wherein R³ and R⁴ independently represent H, an unsubstituted C1-C10 straight chain, branched or cyclic alkyl, an unsubstituted C2-C10 straight chain, branched or cyclic alkenyl or an unsubstituted C2-C10 straight chain or branched alkynyl, preferably one of R³ and R⁴ is H; or
Y=

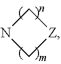

wherein n and m each are an integer independently selected from 1, 2, or 3,
Z=CH₂, O, S, P or NR S, and
wherein R⁵ represents H, an unsubstituted C1-C10 straight chain, branched or cyclic alkyl, an unsubstituted C2-C10 straight chain, branched or cyclic alkenyl, an unsubstituted C2-C10 straight chain or branched alkynyl, or a maleimide moiety according to formula a Da or formula (II)b

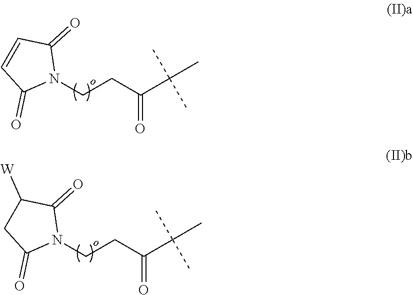

or an azide moiety according to formula (II)c

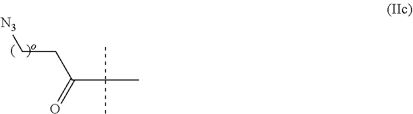

wherein o is an integer selected from 0-10, preferably 2-7, more preferably 4-6, and
W is a thiol functional group according to formula (III)

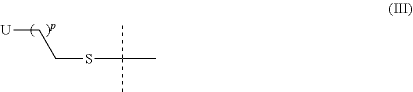

wherein U=SH, NH₂ or OH and
p is an integer selected from 0-4, preferably 1-3, more preferably 1 or 2.

An aspect of the invention relates to a saponin derivative based on a saponin comprising a triterpene aglycone core structure and at least one of a first saccharide chain ‘R¹’ and a second saccharide chain ‘R²’ linked to the aglycone core structure, wherein the saponin derivative comprises an aglycone core structure comprising an aldehyde group, wherein the aldehyde group is transformed into a semicarbazone functional group according to formula (I)

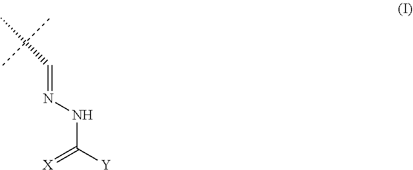

wherein R¹ and R² are independently selected from hydrogen, a monosaccharide, a linear oligosaccharide and a branched oligosaccharide,
X=O, P or S, and
Y=NR³R⁴, wherein R³ and R⁴ independently represent H, an unsubstituted C1-C10 straight chain, branched or cyclic alkyl, an unsubstituted C2-C10 straight chain, branched or cyclic alkenyl or an unsubstituted C2-C10 straight chain or branched alkynyl, or a covalently bound linker, preferably one of R³ and R⁴ is H; or
Y=

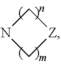

wherein n and m each are an integer independently selected from 1, 2, or 3,
Z=CH₂, O, S, P or NR S, and
wherein R⁵ represents H, an unsubstituted C1-C10 straight chain, branched or cyclic alkyl, an unsubstituted C2-C10 straight chain, branched or cyclic alkenyl, an unsubstituted C2-C10 straight chain or branched alkynyl, or a covalently bound linker, or a maleimide moiety according to formula (II)a or formula (II)b

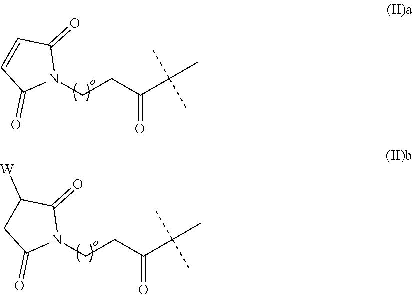

or an azide moiety according to formula (II)c

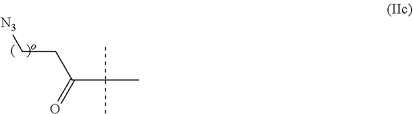

wherein o is an integer selected from 0-10, preferably 2-7, more preferably 4-6, and
W is a thiol functional group according to formula (III)

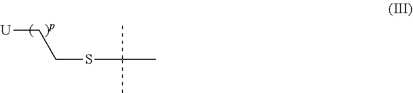

wherein U=SH, NH₂ or OH and
p is an integer selected from 0-4, preferably 1-3, more preferably 1 or 2.

Worded differently, the saponin derivative according to the invention is a saponin comprising a triterpene aglycone core structure and at least one of a first saccharide chain ‘R¹’ and a second saccharide chain ‘R²’ linked to the aglycone core structure, wherein R¹ and R² are independently selected from hydrogen, a monosaccharide, a linear oligosaccharide and a branched oligosaccharide and the aglycone core structure comprises an aldehyde group which has been derivatised by reacting with a semicarbazide according to formula (3)

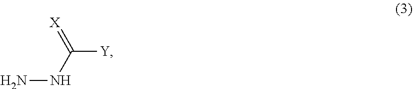

wherein X=O, P or S,
Y=NR³R⁴, wherein R³ and R⁴ independently represent H, an unsubstituted C1-C10 straight chain, branched or cyclic alkyl, an unsubstituted C2-C10 straight chain, branched or cyclic alkenyl or an unsubstituted C2-C10 straight chain or branched alkynyl, or a covalently bound linker, preferably one of R³ and R⁴ is H, or
Y=

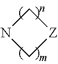

wherein n and m each are an integer independently selected from 1, 2, or 3,
Z=CH₂, O, S, P or NR S, and
wherein R⁵ represent H, an unsubstituted C1-C10 straight chain, branched or cyclic alkyl, an unsubstituted C2-C10 straight chain, branched or cyclic alkenyl, an unsubstituted C2-C10 straight chain or branched alkynyl, or a covalently bound linker, or a maleimide moiety according to formula a Da or formula (II)b,

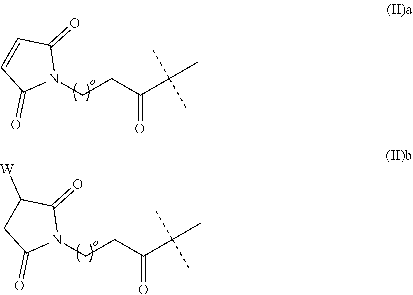

or an azide moiety according to formula (II)c

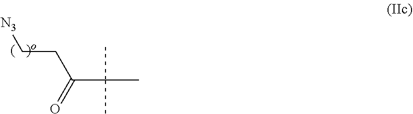

wherein o is an integer selected from 0-10, preferably 2-7, more preferably 4-6, and
W is a thiol functional group according to formula (III),

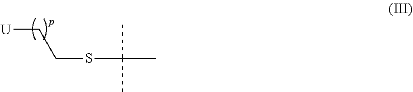

wherein U=SH, NH₂ or OH and
p is an integer selected from 0-4, preferably 1-3, more preferably 1 or 2.

An embodiment is the saponin derivative according to the invention, wherein the saponin derivative is a monodesmosidic triterpene glycoside or a bidesmosidic triterpene glycoside, more preferably a bidesmosidic triterpene glycoside. A series of saponins known in the art have shown to enhance endosomal escape of molecules, which are most often bidesmosidic triterpene glycosides, although also a series of monodesmosidic triterpene glycosides have shown such enhancing activity. Table A1 summarizes the series of saponins for which endosomal escape enhancing activity has been established. Such saponins are good starting points for synthesising the saponin derivatives of the invention. In particular, SO1861 has proven to be a suitable natural saponin for derivatisation according to the invention. Since SO1832 has similar activity when compared to SO1861, as has been established by the inventors, also SO1832 is a suitable natural saponin for derivatisation according to the invention. Similarly, QS-21 can be a suitable natural saponin for derivatisation according to the invention, although the inventors established that the cytotoxicity and haemolytic activity of QS-21 is relatively higher than cytotoxicity and haemolytic activity of e.g. SO1861.

An embodiment is the saponin derivative according to the invention, wherein the saponin derivative comprises an aglycone core structure selected from the group consisting of:
2alpha-hydroxy oleanolic acid;
16alpha-hydroxy oleanolic acid;
hederagenin (23-hydroxy oleanolic acid);
16alpha,23-dihydroxy oleanolic acid;
gypsogenin;
quillaic acid;
protoaescigenin-21(2-methylbut-2-enoate)-22-acetate;
23-oxo-barringtogenol C-21,22-bis(2-methylbut-2-enoate);
23-oxo-barringtog enol C-21 (2-methylbut-2-enoate)-16,22-diacetate;
digitogenin;
3,16,28-trihydroxy oleanan-12-en;
gypsogenic acid,
and
derivatives thereof,
preferably the saponin derivative comprises an aglycone core structure selected from quillaic acid and gypsogenin or derivatives thereof, more preferably the saponin derivative aglycone core structure is quillaic acid or a derivative thereof. Since the inventors now found that improved saponin derivatives can be provided with regard to decreased cytotoxicity and lower haemolysis of cells contacted with such derivatives, based on saponins of the triterpene glycoside type, basically any saponin with such endosomal escape enhancing activity as tested by the inventors, such as saponins having the aglycone of the afore embodiment and listed in Table A1, can be improved accordingly. Lowering toxicity and lowering haemolytic activity while preserving activity to a sufficiently high extent when potentiation of toxins and for example an AON such as BNAs is considered, is an important achievement by the inventors, when the widening of the therapeutic window of the saponin derivatives alone or in combination with e.g. an ADC or an AOC is considered. A sufficiently high dose of derivatised saponin can be applied in e.g. tumor therapy for a cancer patient in need thereof, while the (risk for) cytotoxic side-effects and the (risk for) undesired haemolytic activity exerted or induced by the saponin derivative is decreased when compared with the application of the natural saponin counterpart. Improvements of the therapeutic window of the saponin derivatives of the invention are for example apparent for the exemplified saponin derivatives in Table 3 and Table 4: the ratio between the IC50 for either cytotoxicity, or haemolytic activity and the IC50 for endosomal escape enhancing activity are listed, as well as the haemolytic activity, cytotoxicity and the activity, and the CMC.

An embodiment is the saponin derivative according to the invention, wherein the saponin derivative comprises an aglycone core structure selected from the group consisting of quillaic acid, gypsogenin, and derivatives thereof, preferably the saponin derivative comprises an aglycone core structure selected from the group consisting of quillaic acid and derivatives thereof, wherein the first saccharide chain R¹, when present, is linked to the C3 atom (also denoted as ‘C-3’ atom) or the C28 atom (also denoted as ‘C-28’ atom) of the aglycone core structure, preferably to the C3 atom, and/or wherein the second saccharide chain R², when present, is linked to the C28 atom of the aglycone core structure. Preferred are those saponin derivatives which are based on a saponin having both saccharide chains bound to the aglycone, but in general any saponin that displays endosomal escape enhancing activity is suitable for derivatisation according to the invention, for the purpose to provide single, double, or triple, preferably single or double derivatised saponins with lower cytotoxicity, lower haemolytic activity and sufficiently high endosomal escape enhancing activity.

An embodiment is the saponin derivative according to the invention, wherein the saponin on which the saponin derivative is based is a penta-cyclic triterpene saponin of the 12,13-dehydrooleanane type, preferably the saponin is a monodesmosidic or bidesmosidic penta-cyclic triterpene saponin of the 12,13-dehydrooleanane type.

An embodiment is the saponin derivative according to the invention, wherein the saponin on which the saponin derivative is based is a mono-desmosidic or bi-desmosidic triterpene saponin belonging to the type of a 12,13-dehydrooleanane with the aldehyde group in position C-23 and optionally comprising a glucuronic acid group in a carbohydrate substituent at the C-3beta-OH group of the saponin, preferably a bi-desmosidic triterpene saponin belonging to the type of a 12,13-dehydrooleanane with the aldehyde group in position C-23 and comprising a glucuronic acid group in a carbohydrate substituent at the C-3beta-OH group of the saponin.

A preferred embodiment is the saponin derivative according to the invention, wherein the saponin derivative is according to formula (IV):

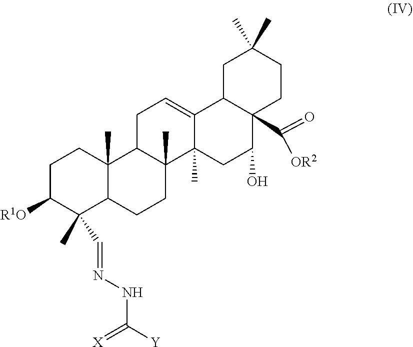

wherein R¹ and R² are independently selected from hydrogen, a monosaccharide, a linear oligosaccharide and a branched oligosaccharide,
X=O, P or S, preferably O; and
Y=NR³R⁴, wherein R³ and R⁴ independently represent H, an unsubstituted C1-C10 straight chain, branched or cyclic alkyl, an unsubstituted C2-C10 straight chain, branched or cyclic alkenyl or an unsubstituted C2-C10 straight chain or branched alkynyl, or a covalently bound linker, preferably one of R³ and R⁴ is H; or
Y=

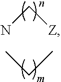

wherein n and m each are an integer independently selected from 1, 2 or 3;
Z=CH₂, O, S, P or NR S, and
wherein R⁵ represents H, an unsubstituted C1-C10 straight chain, branched or cyclic alkyl, an unsubstituted C2-C10 straight chain, branched or cyclic alkenyl or an unsubstituted C2-C10 straight chain or branched alkynyl, or a covalently bound linker, or a maleimide moiety according to formula (II)a or formula (II)b,

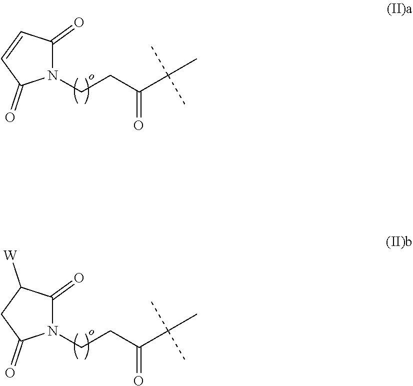

or an azide moiety according to formula (II)c

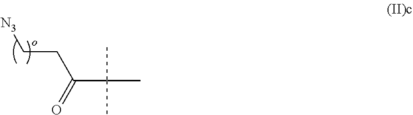

wherein o is an integer selected from 0-10, preferably 2-7, more preferably 4-6 and
W is thiol functional group according to formula (III)

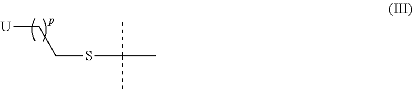

wherein U=SH, NH₂ or OH, preferably OH, and
p is an integer selected from 0-4, preferably 1-3, more preferably 1 or 2.

A preferred embodiment is the saponin derivative according to the invention, wherein the saponin derivative is according to formula (IV):

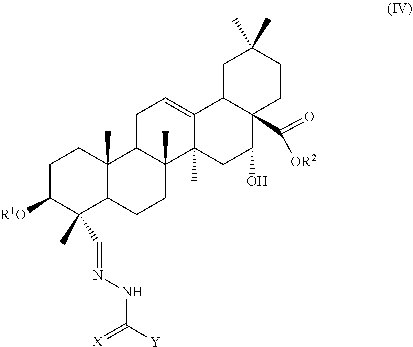

wherein R¹ and R² are independently selected from hydrogen, a monosaccharide, a linear oligosaccharide and a branched oligosaccharide,
X=O, P or S, preferably O; and
Y=

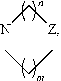

wherein n and m each are an integer independently selected from 1, 2 or 3;
Z=CH₂, O, S, P or NR⁵, preferably O or NR⁵, and
wherein R⁵ represents H, an unsubstituted C1-C10 straight chain, branched or cyclic alkyl, an unsubstituted C2-C10 straight chain, branched or cyclic alkenyl or an unsubstituted C2-C10 straight chain or a branched alkynyl, or a covalently bound linker, or a maleimide moiety according to formula (II)a or formula (II)b, preferably H or a covalently bound linker or the branched alkynyl or a maleimide moiety according to formula a Da or formula (II)b,

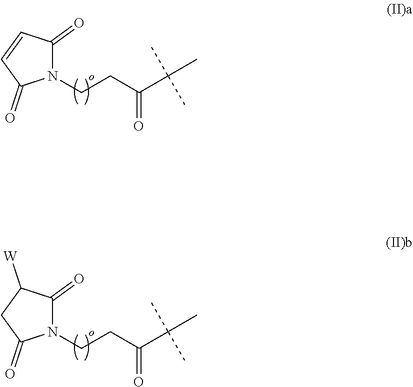

or an azide moiety according to formula (II)c

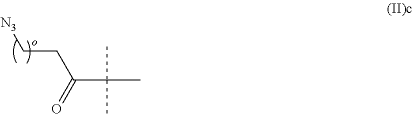

wherein o is an integer selected from 0-10, preferably 2-7, more preferably 4-6 and
W is thiol functional group according to formula (III)

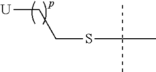

wherein U=SH, NH₂ or OH, preferably OH, and
p is an integer selected from 0-4, preferably 1-3, more preferably 1 or 2.

An embodiment is the saponin derivative according to the invention, wherein the semicarbazone functional group according to formula (I), the linear semicarbazide is according formula (3) or saponin derivative is according to formula (IV), wherein Y=

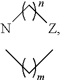

wherein n=1 and m=1, n=2 and m=1, n=2 and m=2, n=3 and m=2 or n=3 and m=3; preferably wherein n=1 and m=2, n=2 and m=2, or n=2 and m=3, more preferably n=2 and m=3 and Z is defined according to the previous embodiments. Particular saponin derivatives according to the invention are thus saponin derivatives, wherein n=1 and m=1, or n=2 and m=1, or n=2 and m=2, or n=3 and m=2, or n=3 and m=3; preferably wherein n=2 and m=2.

An embodiment is the saponin derivative according to the invention, wherein
R¹ is selected from
H,
GlcA-,
Glc-,
GaI-,
Rha-(1→2)-Ara-,
GaI-(1→2)-[XyI-(1→3)]-GlcA-,
Glc-(1→2)-[Glc-(1→4)]-GlcA-,
Glc-(1→2)-Ara-(1→3)-[GaI-(1→2)]-GlcA-,
XyI-(1→2)-Ara-(1→3)-[GaI-(1→2)]-GlcA-,
Glc-(1→3)-GaI-(1→2)-[XyI-(1→3)]-Glc-(1→4)-GaI-,
Rha-(1→2)-GaI-(1→3)-[Glc-(1→2)]-GlcA-,
Ara-(1→4)-Rha-(1→2)-Glc-(1→2)-Rha-(1→2)-GlcA-,
Ara-(1→4)-Fuc-(1→2)-Glc-(1→2)-Rha-(1→2)-GlcA-,
Ara-(1→4)-Rha-(1→2)-GaI-(1→2)-Rha-(1→2)-GlcA-,
Ara-(1→4)-Fuc-(1→2)-GaI-(1→2)-Rha-(1→2)-GlcA-,
Ara-(1→4)-Rha-(1→2)-Glc-(1→2)-Fuc-(1→2)-GlcA-,
Ara-(1→4)-Fuc-(1→2)-Glc-(1→2)-Fuc-(1→2)-GlcA-,
Ara-(1→4)-Rha-(1→2)-GaI-(1→2)-Fuc-(1→2)-GlcA-,
Ara-(1→4)-Fuc-(1→2)-GaI-(1→2)-Fuc-(1→2)-GlcA-,
XyI-(1→4)-Rha-(1→2)-Glc-(1→2)-Rha-(1→2)-GlcA-,
XyI-(1→4)-Fuc-(1→2)-Glc-(1→2)-Rha-(1→2)-GlcA-,
XyI-(1→4)-Rha-(1→2)-GaI-(1→2)-Rha-(1→2)-GlcA-,
XyI-(1→4)-Fuc-(1→2)-GaI-(1→2)-Rha-(1→2)-GlcA-,
XyI-(1→4)-Rha-(1→2)-Glc-(1→2)-Fuc-(1→2)-GlcA-,
XyI-(1→4)-Fuc-(1→2)-Glc-(1→2)-Fuc-(1→2)-GlcA-,
XyI-(1→4)-Rha-(1→2)-GaI-(1→2)-Fuc-(1→2)-GlcA-,
XyI-(1→4)-Fuc-(1→2)-GaI-(1→2)-Fuc-(1→2)-GlcA-, and
derivatives thereof,
preferably R¹ is GaI-(1→2)-[XyI-(1→3)]-GlcA-; and
R² is selected from:
H,
GaI-,
Rha-(1→2)-[XyI-(1→4)]-Rha-,
Rha-(1→2)-[Ara-(1→3)-XyI-(1→4)]-Rha-,
Ara-,
XyI-,
XyI-(1→4)-Rha-(1→2)-[R1-(→4)]-Fuc- wherein R1 is 4E-Methoxycinnamic acid,
XyI-(1→4)-Rha-(1→2)-[R2-(→4)]-Fuc- wherein R2 is 4Z-Methoxycinnamic acid,
XyI-(1→4)-[GaI-(1→3)]-Rha-(1→2)-4-OAc-Fuc-,
XyI-(1→4)-[Glc-(1→3)]-Rha-(1→2)-3,4-di-OAc-Fuc-,
XyI-(1→4)-[Glc-(1→3)]-Rha-(1→2)-[R⁶-(→4)]-3-OAc-Fuc- wherein R⁶ is 4E-Methoxycinnamic acid,
Glc-(1→3)-XyI-(1→4)-[Glc-(1→3)]-Rha-(1→2)-4-OAc-Fuc-,
Glc-(1→3)-XyI-(1→4)-Rha-(1→2)-4-OAc-Fuc-,
(Ara- or XyI-)(1→3)-(Ara- or XyI-)(1→4)-(Rha- or Fuc-)(1→2)-[4-OAc-(Rha- or Fuc-)(1→4)]-(Rha-or Fuc-),
XyI-(1→3)-XyI-(1→4)-Rha-(1→2)-[Qui-(1→4)]-Fuc-,
Api-(1→3)-XyI-(1→4)-[Glc-(1→3)]-Rha-(1→2)-Fuc-,
XyI-(1→4)-[GaI-(1→3)]-Rha-(1→2)-Fuc-,
XyI-(1→4)-[Glc-(1→3)]-Rha-(1→2)-Fuc-,
Ara/XyI-(1→4)-Rha/Fuc-(1→4)-[Glc/GaI-(1→2)]-Fuc-,
Api-(1→3)-XyI-(1→4)-[Glc-(1→3)]-Rha-(1→2)-[R⁷-(→4)]-Fuc- wherein R⁷ is 5-O-[5-O-Ara/Api-3,5-dihydroxy-6-methyl-octanoyl]-3,5-dihydroxy-6-methyl-octanoic acid),
Api-(1→3)-XyI-(1→4)-Rha-(1→2)-[R⁸-(→4)]-Fuc- wherein R⁸ is 5-O-[5-O-Ara/Api-3,5-dihydroxy-6-methyl-octanoyl]-3,5-dihydroxy-6-methyl-octanoic acid),
Api-(1→3)-XyI-(1→4)-Rha-(1→2)-[Rha-(1→3)]-4-OAc-Fuc-,
Api-(1→3)-XyI-(1→4)-[Glc-(1→3)]-Rha-(1→2)-[Rha-(1→3)]-4-OAc-Fuc-,
6-OAc-Glc-(1→3)-XyI-(1→4)-Rha-(1→2)-[3-OAc-Rha-(1→3)]-Fuc-,
Glc-(1→3)-XyI-(1→4)-Rha-(1→2)-[3-OAc-Rha-(1→3)]-Fuc-,
XyI-(1→3)-XyI-(1→4)-Rha-(1→2)-[Qui-(1→4)]-Fuc-,
Glc-(1→3)-[XyI-(1→4)]-Rha-(1→2)-[Qui-(1→4)]-Fuc-,
Glc-(1→3)-XyI-(1→4)-Rha-(1→2)-[XyI-(1→3)-4-OAc-Qui-(1→4)]-Fuc-,
XyI-(1→3)-XyI-(1→4)-Rha-(1→2)-[3,4-di-OAc-Qui-(1→4)]-Fuc-,
Glc-(1→3)-[XyI-(1→4)]-Rha-(1→2)-Fuc-,
6-OAc-Glc-(1→3)-[XyI-(1→4)]-Rha-(1→2)-Fuc-,
Glc-(1→3)-[XyI-(1→3)-XyI-(1→4)]-Rha-(1→2)-Fuc-,
XyI-(1→3)-XyI-(1→4)-Rha-(1→2)-[XyI-(1→3)-4-OAc-Qui-(1→4)]-Fuc-,
Api/XyI-(1→3)-XyI-(1→4)-[Glc-(1→3)]-Rha-(1→2)-[Rha-(1→3)]-4OAc-Fuc-, Api-(1→3)-XyI-(1→4)-[Glc-(1→3)]-Rha-(1→2)-[Rha-(1→3)]-4OAc-Fuc-,
Api/XyI-(1→3)-XyI-(1→4)-[Glc-(1→3)]-Rha-(1→2)-[R⁹-(→4)]-Fuc- wherein R⁹ is 5-O-[5-O-Rha-(1→2)-Ara/Api-3,5-dihydroxy-6-methyl-octanoyl]-3,5-dihydroxy-6-methyl-octanoic acid),
Api/XyI-(1→3)-XyI-(1→4)-[Glc-(1→3)]-Rha-(1→2)-[R¹⁹-(→4)]-Fuc- wherein R¹⁹ is 5-O-[5-O-Ara/Api-3,5-dihydroxy-6-methyl-octanoyl]-3,5-dihydroxy-6-methyl-octanoic acid),
Api/XyI-(1→3)-XyI-(1→4)-[Glc-(1→3)]-Rha-(1→2)-[R¹¹-(→4)]-Fuc- wherein R¹¹ is 5-O-[5-O-Ara/Api-3,5-dihydroxy-6-methyl-octanoyl]-3,5-dihydroxy-6-methyl-octanoic acid),
Api-(1→3)-XyI-(1→4)-Rha-(1→2)-[R¹²-(→4)]-Fuc- wherein R¹² is 5-O-[5-O-Ara/Api-3,5-dihydroxy-6-methyl-octanoyl]-3,5-dihydroxy-6-methyl-octanoic acid),
XyI-(1→3)-XyI-(1→4)-Rha-(1→2)-[R¹³-(→4)]-Fuc- wherein R¹³ is 5-O-[5-O-Ara/Api-3,5-dihydroxy-6-methyl-octanoyl]-3,5-dihydroxy-6-methyl-octanoic acid),
Api-(1→3)-XyI-(1→4)-Rha-(1→2)-[R¹⁴-(→3)]-Fuc- wherein R¹⁴ is 5-O-[5-O-Ara/Api-3,5-dihydroxy-6-methyl-octanoyl]-3,5-dihydroxy-6-methyl-octanoic acid),
XyI-(1→3)-XyI-(1→4)-Rha-(1→2)-[R¹⁵-(→3)]-Fuc- wherein R¹⁵ is 5-O-[5-O-Ara/Api-3,5-dihydroxy-6-methyl-octanoyl]-3,5-dihydroxy-6-methyl-octanoic acid)
Glc-(1→3)-[Glc-(1→6)]-GaI-, and
derivatives thereof;
preferably R² is selected from:
Glc-(1→3)-XyI-(1→4)-Rha-(1→2)-[XyI-(1→3)-4-OAc-Qui-(1→4)]-Fuc-,
XyI-(1→3)-XyI-(1→4)-Rha-(1→2)-[3,4-di-OAc-Qui-(1→4)]-Fuc-,
Api-(1→3)-XyI-(1→4)-Rha-(1→2)-[R¹²-(→4)]-Fuc- wherein R¹² is 5-O-[5-O-Ara/Api-3,5-dihydroxy-6-methyl-octanoyl]-3,5-dihydroxy-6-methyl-octanoic acid,
XyI-(1→3)-XyI-(1→4)-Rha-(1→2)-[R¹³-(→4)]-Fuc- wherein R¹³ is 5-O-[5-O-Ara/Api-3,5-dihydroxy-6-methyl-octanoyl]-3,5-dihydroxy-6-methyl-octanoic acid,
Api-(1→3)-XyI-(1→4)-Rha-(1→2)-[R¹⁴-(→3)]-Fuc- wherein R¹⁴ is 5-O-[5-O-Ara/Api-3,5-dihydroxy-6-methyl-octanoyl]-3,5-dihydroxy-6-methyl-octanoic acid, and
XyI-(1→3)-XyI-(1→4)-Rha-(1→2)-[R¹⁵-(→3)]-Fuc- wherein R¹⁵ is 5-O-[5-O-Ara/Api-3,5-dihydroxy-6-methyl-octanoyl]-3,5-dihydroxy-6-methyl-octanoic acid,
more preferably R² is Glc-(1→3)-XyI-(1→4)-Rha-(1→2)-[XyI-(1→3)-4-OAc-Qui-(1→4)]-Fuc-,
most preferably R¹ is GaI-(1→2)-[XyI-(1→3)]-GlcA- and R² is Glc-(1→3)-XyI-(1→4)-Rha-(1→2)-[XyI-(1→3)-4-OAc-Qui-(1→4)]-Fuc-.

In preferred saponin derivatives according to the invention,
R¹ is GaI-(1→2)-[XyI-(1→3)]-GlcA-; and
R² is selected from:
Glc-(1→3)-XyI-(1→4)-Rha-(1→2)-[XyI-(1→3)-4-OAc-Qui-(1→4)]-Fuc-,
XyI-(1→3)-XyI-(1→4)-Rha-(1→2)-[3,4-di-OAc-Qui-(1→4)]-Fuc-,
Api-(1→3)-XyI-(1→4)-Rha-(1→2)-[R¹²-(→4)]-Fuc- wherein R¹² is 5-O-[5-O-Ara/Api-3,5-dihydroxy-6-methyl-octanoyl]-3,5-dihydroxy-6-methyl-octanoic acid,
XyI-(1→3)-XyI-(1→4)-Rha-(1→2)-[R¹³-(→4)]-Fuc- wherein R¹³ is 5-O-[5-O-Ara/Api-3,5-dihydroxy-6-methyl-octanoyl]-3,5-dihydroxy-6-methyl-octanoic acid,
Api-(1→3)-XyI-(1→4)-Rha-(1→2)-[R¹⁴-(→3)]-Fuc- wherein R¹⁴ is 5-O-[5-O-Ara/Api-3,5-dihydroxy-6-methyl-octanoyl]-3,5-dihydroxy-6-methyl-octanoic acid, and
XyI-(1→3)-XyI-(1→4)-Rha-(1→2)-[R¹⁵-(→3)]-Fuc- wherein R¹⁵ is 5-O-[5-O-Ara/Api-3,5-dihydroxy-6-methyl-octanoyl]-3,5-dihydroxy-6-methyl-octanoic acid,
preferably, R¹ is GaI-(1→2)-[XyI-(1→3)]-GlcA- and R² is Glc-(1→3)-XyI-(1→4)-Rha-(1→2)-[XyI-(1→3)-4-OAc-Qui-(1→4)]-Fuc-.

Saponin derivatives comprising such a R¹ group and such an R² group are preferably saponin derivatives based on a saponin listed in Table A1, for which endosomal escape enhancing activity has been established and/or predicted based on similarity or analogy.

Examples of saponins that can be applied for providing the saponin derivative of the invention are:
a) saponin selected from any one or more of list A:
   Quillaja saponaria saponin mixture, or a saponin isolated from Quillaja saponaria, for example Quil-A, QS-17-api, QS-17-xyl, QS-21, QS-21A, QS-21B, QS-7-xyl;
   Saponinum album saponin mixture, or a saponin isolated from Saponinum album;
   Saponaria officinalis saponin mixture, or a saponin isolated from Saponaria officinalis; and
   Quillaja bark saponin mixture, or a saponin isolated from Quillaja bark, for example Quil-A, QS-17-api, QS-17-xyl, QS-21, QS-21A, QS-21B, QS-7-xyl; or
b) a saponin comprising a gypsogenin aglycone core structure, selected from list B:
   SA1641, gypsoside A, NP-017772, NP-017774, NP-017777, NP-017778, NP-018109, NP-017888, NP-017889, NP-018108, SO1658 and Phytolaccagenin; or
c) a saponin comprising a quillaic acid aglycone core structure, selected from list C:
   AG1856, AG1, AG2, Agrostemmoside E, GE1741, Gypsophila saponin 1 (Gyp1), NP-017674, NP-017810, NP-003881, NP-017676, NP-017677, NP-017705, NP-017706, NP-017773, NP-017775, SA1657, Saponarioside B, SO1542, SO1584, SO1674, SO1700, SO1730, SO1772, SO1832, SO1861, SO1862, SO1904, QS1861, QS1862, QS-7, QS-7 api, QS-17, QS-18, QS-21 A-apio, QS-21 A-xylo, QS-21 B-apio and QS-21 B-xylo.

Preferably, the at least one saponin is any one or more of a saponin selected from list B or C, more preferably from list C.

An embodiment is the saponin derivative of the invention wherein the saponin on which the saponin derivative is based is any one or more of AG1856, GE1741, a saponin isolated from Quillaja saponaria, Quil-A, QS-17, QS-21, QS-7, SA1641, a saponin isolated from Saponaria officinalis, Saponarioside B, SO1542, SO1584, SO1658, SO1674, SO1700, SO1730, SO1772, SO1832, SO1861, SO1862 and SO1904, preferably the at least one saponin is any one or more of QS-21, SO1832, SO1861, SA1641 and GE1741, more preferably the at least one saponin is QS-21, SO1832 or SO1861, even more preferably the at least one saponin is SO1861 or SO1832.

An embodiment is the saponin derivative of the invention wherein the saponin on which the saponin derivative is based is a saponin isolated from Saponaria officinalis, preferably the at least one saponin is any one or more of Saponarioside B, SO1542, SO1584, SO1658, SO1674, SO1700, SO1730, SO1772, SO1832, SO1861, SO1862 and SO1904, more preferably the at least one saponin is any one or more of SO1832, SO1861 and SO1862, even more preferably SO1832 and SO1861, even more preferably the at least one saponin is SO1861.

Preferably, the saponin derivative is based on a triterpenoid saponin of the 12,13-dehydrooleanane type, preferably with an aldehyde group in position C-23 of the aglycone core. In Table A1, examples of such saponins are listed. In particular, triterpenoid saponins of the 12,13-dehydrooleanane type with an aldehyde group in position C-23 of the aglycone core, wherein the aglycone core is quillaic acid or gypsogenine, are preferred. Examples of such triterpenoid saponins are listed in Table A1.

An embodiment is the saponin derivative according to the invention, wherein the saponin derivative is a derivative of a saponin selected from the group of saponins consisting of: Quillaja bark saponin, dipsacoside B, saikosaponin A, saikosaponin D, macranthoidin A, esculentoside A, phytolaccagenin, aescinate, AS6.2, NP-005236, AMA-1, AMR, alpha-Hederin, NP-012672, NP-017777, NP-017778, NP-017774, NP-018110, NP-017772, NP-018109, NP-017888, NP-017889, NP-018108, SA1641, AE X55, NP-017674, NP-017810, AG1, NP-003881, NP-017676, NP-017677, NP-017706, NP-017705, NP-017773, NP-017775, SA1657, AG2, SO1861, GE1741, SO1542, SO1584, SO1658, SO1674, SO1832, SO1904, SO1862, QS-7, QS1861, QS-7 api, QS1862, QS-17, QS-18, QS-21 A-apio, QS-21 A-xylo, QS-21 B-apio, QS-21 B-xylo, beta-Aescin, Aescin Ia, Teaseed saponin I, Teaseedsaponin J, Assamsaponin F, Digitonin, Primula acid 1 and AS64R, preferably the saponin derivative is selected from the group consisting of a QS-21 derivative, an SO1861 derivative, an SA1641 derivative and an GE1741 derivative, more preferably the saponin derivative is selected from the group consisting of a QS-21 derivative and an SO1861 derivative, most preferably the saponin derivative is an SO1861 derivative. These saponins are essentially saponins displaying endosomal escape enhancing activity as established by the inventors, or that are structurally highly similar to saponins for which the endosomal escape enhancing activity has been established. Structural outline of these saponins is summarized in Table A1. Since SO1861 and SO1832 are about equally active (when endosomal escape enhancing effects are assessed), application of SO1861 and SO1832 in the saponin derivatives and in the saponin conjugates of the invention is equally preferred.

Thus, typically, for the saponin derivative according to the invention, the saponin derivative is a derivative of a saponin selected from the group of saponins consisting of: Quillaja bark saponin, dipsacoside B, saikosaponin A, saikosaponin D, macranthoidin A, esculentoside A, phytolaccagenin, aescinate, AS6.2, NP-005236, AMA-1, AMR, alpha-Hederin, NP-012672, NP-017777, NP-017778, NP-017774, NP-018110, NP-017772, NP-018109, NP-017888, NP-017889, NP-018108, SA1641, AE X55, NP-017674, NP-017810, AG1, NP-003881, NP-017676, NP-017677, NP-017706, NP-017705, NP-017773, NP-017775, SA1657, AG2, SO1861, GE1741, SO1542, SO1584, SO1658, SO1674, SO1832, SO1904, SO1862, QS-7, QS1861, QS-7 api, QS1862, QS-17, QS-18, QS-21 A-apio, QS-21 A-xylo, QS-21 B-apio, QS-21 B-xylo, beta-Aescin, Aescin Ia, Teaseed saponin I, Teaseedsaponin J, Assamsaponin F, Digitonin, Primula acid 1 and AS64R, preferably the saponin derivative is selected from the group consisting of a QS-21 derivative, an SO1861 derivative, an SA1641 derivative and a GE1741 derivative, more preferably the saponin derivative is selected from the group consisting of a QS-21 derivative and an SO1861 derivative, most preferably the saponin derivative is an SO1861 derivative. As said before, a derivative based on SO1861 and SO1832 is equally preferred.

An embodiment is the saponin derivative according to the invention, wherein the saponin derivative is selected from the group consisting of derivatives of: SO1861, SA1657, GE1741, SA1641, QS-21, QS-21A, QS-21 A-api, QS-21 A-xyl, QS-21B, QS-21 B-api, QS-21 B-xyl, QS-7-xyl, QS-7-api, QS-17-api, QS-17-xyl, QS1861, QS1862, Quillajasaponin, Saponinum album, QS-18, Quil-A, Gyp1, gypsoside A, AG1, AG2, SO1542, SO1584, SO1658, SO1674, SO1832, SO1862, SO1904, stereoisomers thereof and combinations thereof, preferably the saponin derivative is selected from the group consisting of a SO1861 derivative, a GE1741 derivative, a SA1641 derivative, a QS-21 derivative, and a combination thereof, more preferably the saponin derivative is a SO1861 derivative or a QS-21 derivative, most preferably, the saponin derivative is a SO1861 derivative.

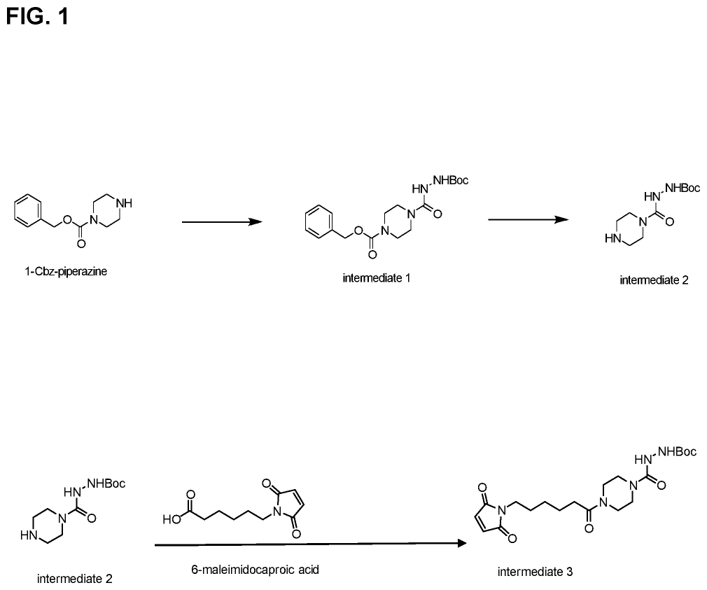

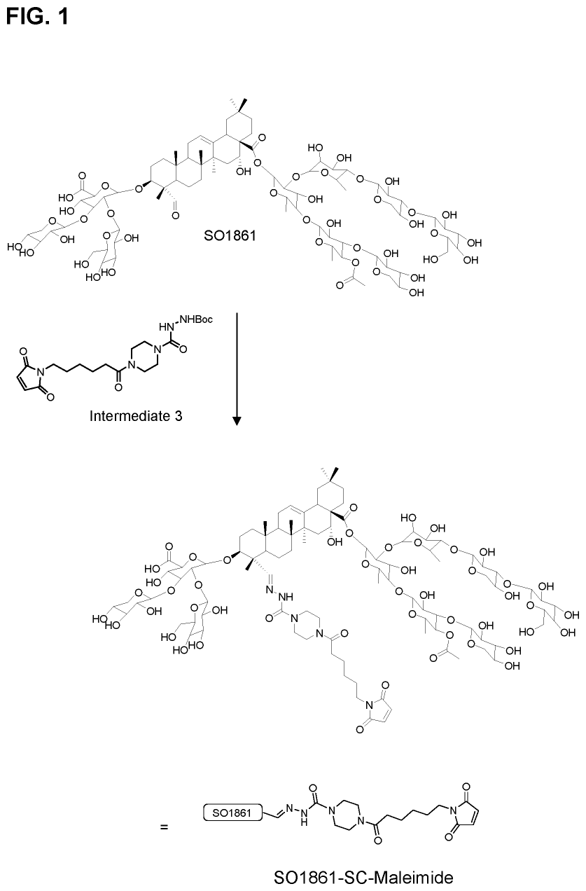

Particularly preferred is a saponin derivative of the invention, wherein the saponin derivative is according to formula (V), formula (VI), formula (VII) (See also FIG. 1) or formula (VIII)

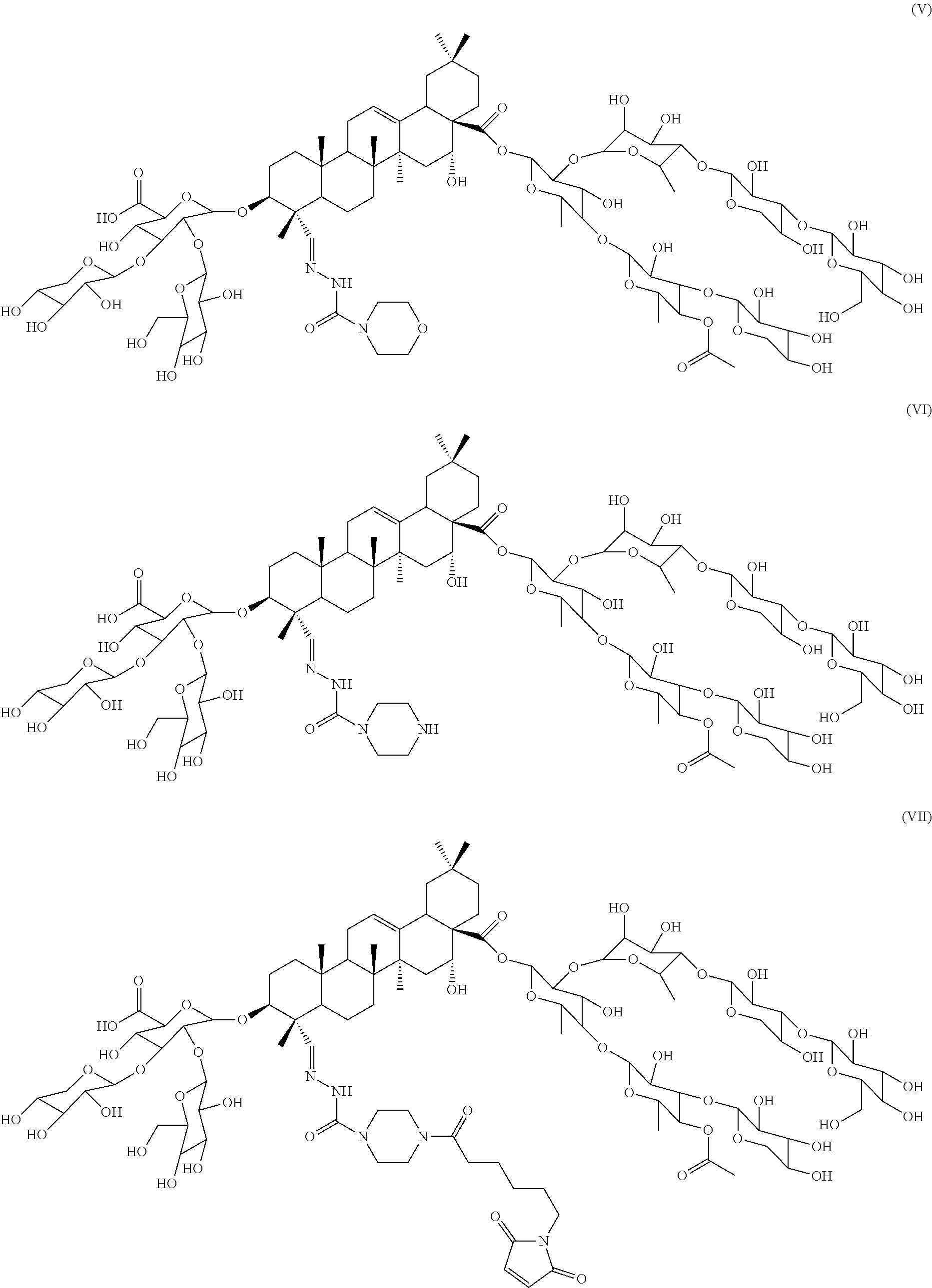

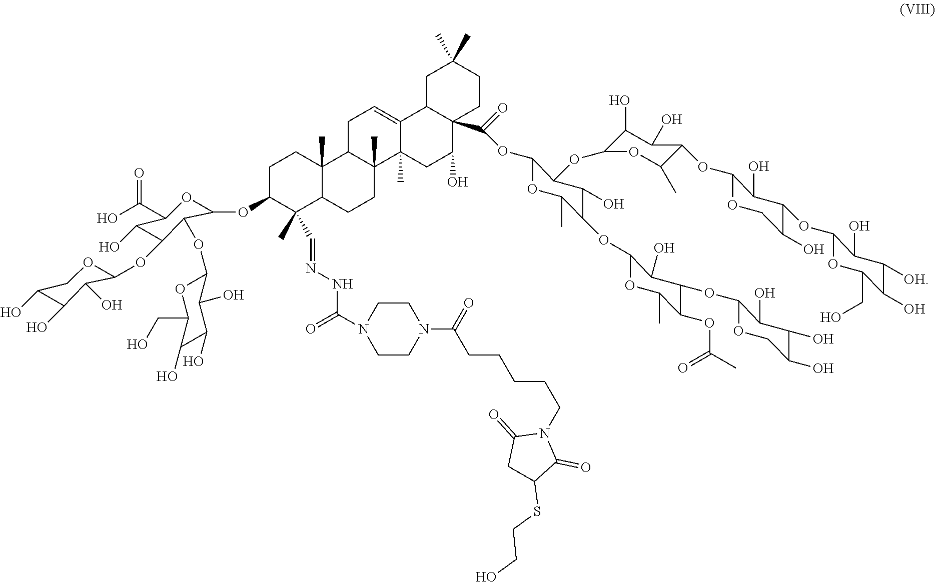

Particularly preferred is a saponin derivative of the invention, wherein the saponin derivative is according to molecule (AA):

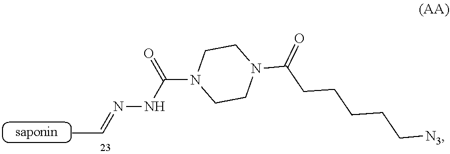 [SMILES: C/C=N/NC(=O)N1CCN(C(=O)CCCCCN=[N+]=[N-])CC1]

wherein

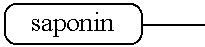

represents a saponin moiety according to formula (SM):

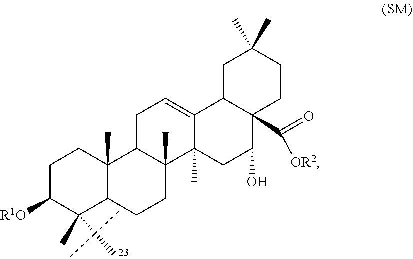

wherein R¹ and R² are independently selected from hydrogen, a monosaccharide, a linear oligosaccharide and a branched oligosaccharide, and wherein the saponin moiety according to formula (SM) is based on a saponin comprising an aldehyde group in position C-23. The saponin moiety is for example based on any one of the saponins listed here above or in Table A1, and SO1861, SO1832 and QS-21 are preferred, although SO1861 and SO1832 are most preferred.

Also preferred is the saponin derivative according to the invention wherein the saponin derivative is according to molecule (KK):

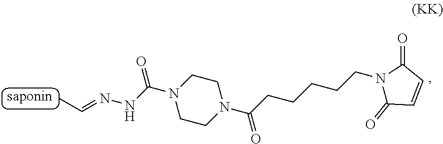 [SMILES: "saponin" ; C/C=N/NC(=O)N1CCN(C(=O)CCCCCN2C(=O)C=CC2=O)CC1]

wherein

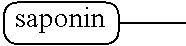

represents a saponin moiety according to formula (SM):

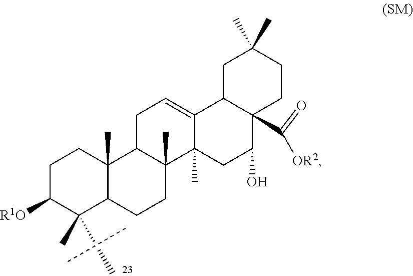

wherein R¹ and R² are independently selected from hydrogen, a monosaccharide, a linear oligosaccharide and a branched oligosaccharide according to the invention and as outlined here above, and wherein the saponin moiety according to formula (SM) is based on a saponin comprising an aldehyde group in position C-23 according to the invention. Suitable saponins are for example outlined in Table A1. SO1861, SO1832 and QS-21 are preferred, although SO1861 and SO1832 are more preferred.

The saponin derivative according to molecule (KK) is for example a derivative suitable for conjugation with e.g. a cell-targeting antibody, e.g. in the context of an ADC or AOC. Molecule (KK) is applicable as a semi-product for synthesizing such an ADC or AOC, providing a single conjugate comprising the cell-targeting moiety (e.g. an antibody or a binding domain thereof), an effector moiety such as a toxin or a (gene-silencing) oligonucleotide (e.g. an AON) such as a BNA or siRNA, and the saponin moiety with improved activity due to enhanced release from the conjugate under influence of slightly acidic pH in the endosome of a targeted cell, by virtue of the presence of the semicarbazone functional group, and as compared with a similar conjugate though comprising the hydrazone functional group instead of the semicarbazone functional group. The saponin derivative according to molecule (KK) is for example also a semi-product for producing a conjugate comprising more than one copies of the saponin moiety comprising the semicarbazone functional group. Examples are the synthesis of a conjugate comprising for example a G2 dendron or G3 dendron and four or eight covalently bound saponin derivatives comprising the semicarbazone functional group. As such, such a dendron-based conjugate with four or eight saponin moieties, is a semi-product for providing a conjugate comprising an effector moiety such as a (gene-silencing) oligonucleotide, such as a BNA or an siRNA, and/or comprising a ligand for a cell-surface molecule such as a cell-receptor binding antibody or binding fragment or domain thereof or a cell-surface receptor ligand such as EGF for binding to the EGFR. Providing such conjugates of the invention with a single saponin derivative or multiple (copies of the) saponin derivative(s) provides the opportunity to fine-tune and select the optimal number of saponin moieties most suitable for optimal enhancement of the intracellular potency of the effector molecule such as a gene-silencing oligonucleotide (an AON), e.g. a BNA. That is to say, for certain applications involving a combination of a certain target cell, a certain selected cell-surface molecule such as a receptor, a certain cell-surface molecule binding ligand such as an antibody or EGF, and a certain effector molecule such as a (gene-silencing) oligonucleotide (e.g. an AON) such as a BNA, presence of a single saponin derivative comprising the semicarbazone functional group in a conjugate of the saponin derivative, the effector molecule and the cell-surface molecule binding ligand may suffice to obtain optimal potency of the effector molecule, whereas for other combinations more than one saponin derivative moieties in the conjugate may be applied to achieve optimal potency, such as 2-32 saponin derivative moieties, preferably 2-16 moieties, more preferably 4-8 moieties, such as 4 or 8 moieties. The saponin derivatives of the invention provide for a flexible platform when adapting the number of saponin derivative moieties in a saponin conjugate of the invention such that optimal efficacy of the effector molecule is achieved, is considered. Indeed, the inventors established that gene silencing was enhanced when the number of saponin derivative moieties comprising the semicarbazone functional group was increased from 1 to 4 to 8 for a conjugate of the invention comprising a cell-targeting ligand and a BNA for silencing the apoB gene.

A preferred embodiment is the saponin derivative according to the invention, characterized in that the saponin derivative comprises a single saponin moiety. A preferred embodiment is the saponin derivative according to the invention, characterized in that the saponin derivative comprises more than one saponin moiety, such as 2-64 moieties, preferably 4-32 moieties, more preferably 4-16 moieties, most preferably 4-8 moieties such as 4 or 8 moieties. Saponin derivatives comprising more than a single saponin moiety, such as a saponin derivative based on a G2 dendron or G3 dendron, comprising four or eight saponin moieties respectively, which are provided with the semicarbazone functional group, provide the advantage of providing higher extent of endosomal escape enhancing activity by a single saponin derivative molecule compared to a saponin derivative comprising a single saponin moiety.

Part of the invention is the saponin derivative according to molecule (JJ):

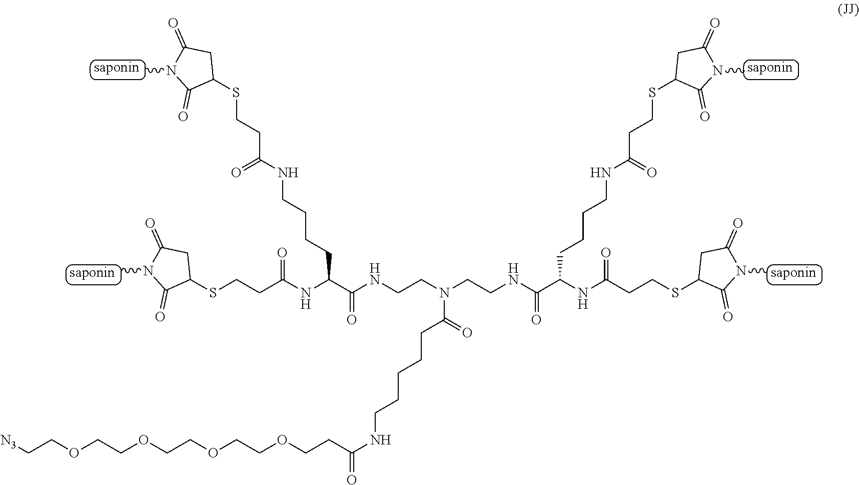

which molecule (JJ) is the conjugate product of conjugation of N,N′-((9S,19S)-14-(6-aminohexanoyl)-1-mercapto-9-(3-mercaptopropanamido)-3,10,18-trioxo-4,11,14,17-tetraazatricosane-19,23-diyl)bis(3-mercaptopropanamide first with the saponin derivative according to molecule (KK), as displayed here above, and subsequently with 2,5-dioxopyrrolidin-1-yl 1-azido-3,6,9,12-tetraoxapentadecan-15-oate.

Also part of the invention is the saponin derivative according to molecule (QQ):

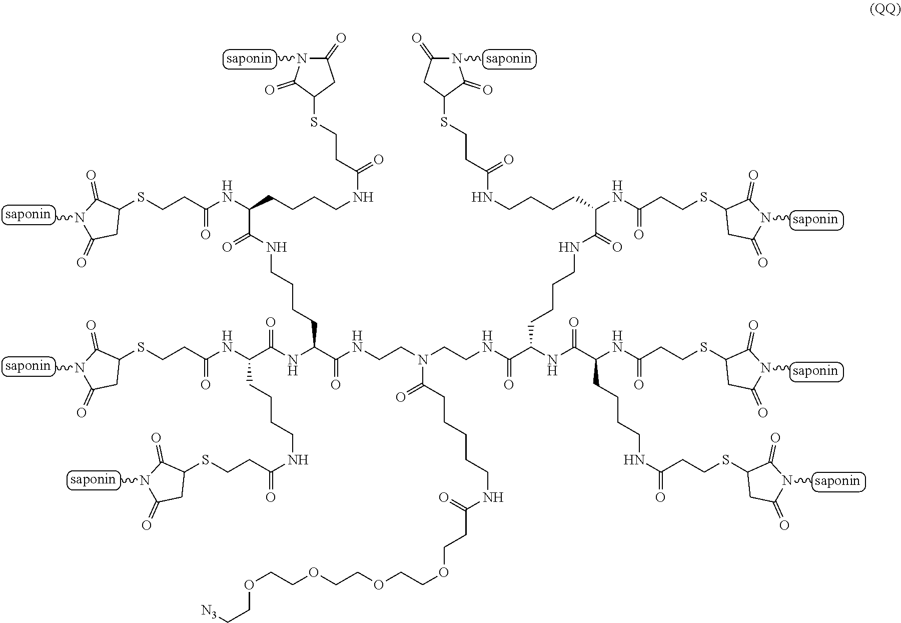

which molecule (QQ) is the conjugate product of conjugation of (2S)—N-[(1S)-1-{[2-(6-amino-N-{2-[(2S)-2,6-bis[(2S)-2,6-bis(3-sulfanylpropanamido)hexanamido]hexanamido]ethyl}hexanamido)ethyl]carbamoyl}-5-[(2S)-2,6-bis(3-sulfanylpropanamido)hexanamido]pentyl]-2,6-bis(3-sulfanylpropanamido)hexanamide formate first with saponin derivative according to the invention and according to molecule (KK) as displayed and described here above, and subsequently with 2,5-dioxopyrrolidin-1-yl 1-azido-3,6,9,12-tetraoxapentadecan-15-oate.

An embodiment is the saponin derivative according to the invention, and for example according to any one of the molecules (AA), (KK), (JJ) and (QQ), wherein the saponin derivative is based on a saponin according to any one of the saponins listed here below and in Table A1.

Preferred is the saponin derivative according to the invention, and for example according to any one of the molecules (AA), (KK), (JJ) and (QQ), wherein the saponin derivative is based on a saponin selected from SO1861, SO1832 and QS-21, preferably SO1861 and SO1832, more preferably SO1861.

Also preferred is the saponin derivative according to the invention, and for example according to any one of the molecules (AA), (KK), (JJ) and (QQ), wherein the semicarbazone functional group is subject to hydrolysis in vivo under acidic conditions as present in endosomes and/or lysosomes of mammalian cells, preferably human cells, preferably at pH 4.0-6.5, and more preferably at pH≤5.5.

An embodiment is the saponin derivative according to the invention, characterized in that the saponin derivative has a molecular weight of less than 2500 g/mol, preferably less than 2300 g/mol, more preferably less than 2150 g/mol.

An embodiment is the saponin derivative according to the invention, characterized in that the saponin derivatisation has a molecular weight of less than 400 g/mol, preferably less than 300 g/mol, more preferably less than 270 g/mol. The molecular weight of the saponin derivative corresponds to the molecular weight of the saponin derivative exclusive of the aglycone core and the one (for monodesmosidic saponins) or two (for bidesmosidic saponins) glycon (sugar) chains.

Saponin Conjugate Based on the Saponin Derivatives

Surprisingly, the inventors have found that the saponin derivatives according to the invention which are used as a component for potentiating the intracellular effect of an effector molecule or effector moiety when the effector molecule is provided as a conjugate comprising a cell-surface molecule binding-molecule such as an antibody, e.g. as an ADC or AOC, are now also suitably for (covalent) coupling to a cell-surface molecule binding-molecule such as a cell-surface molecule targeting antibody, such as an sdAb, such that by such coupling the saponin conjugate of the invention is provided, endowed with, for example, anti-tumor activity potentiating activity when used in combination with for example an ADC or an AOC.

An aspect of the invention relates to a saponin conjugate comprising a cell-surface molecule binding-molecule such as a first proteinaceous molecule (‘proteinaceous molecule 1’) that is covalently bound to the saponin derivative according to the invention, i.e. covalently linked to the saponin derivative. The cell-surface molecule binding-molecule is covalently bound to the derivatisation in the saponin derivative, i.e. the derivatised aldehyde functional group of the saponin on which the saponin derivative is based, i.e. through (via) a linker. The cell-surface molecule binding-molecule typically is a protein, such as an antibody or a binding fragment thereof, or a single-domain antibody (sdAb) or a binding molecule comprising a sdAb.

An aspect of the invention relates to a saponin conjugate based on a saponin derivative according to the invention, wherein the aldehyde group is transformed into a semicarbazone functional group according to formula (IX)

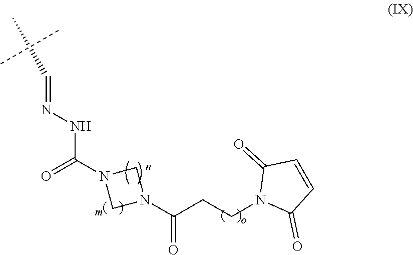

wherein n and m each are an integer independently selected from 1, 2, or 3;
is an integer selected from 0-10, preferably 2-7, more preferably 4-6;
and wherein the maleimide functional group is further transformed into a thioether bond through reaction with a first proteinaceous molecule (‘proteinaceous molecule 1’) comprising a thiol functional group according to formula (X)

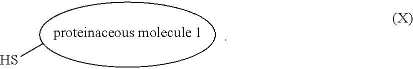

An aspect of the invention relates to a saponin conjugate comprising a first proteinaceous molecule (‘proteinaceous molecule 1’) comprising a cell-surface molecule binding-molecule comprising a first binding site for binding to a first epitope of a first cell-surface molecule and further comprising at least one thiol functional group, according to formula (X)

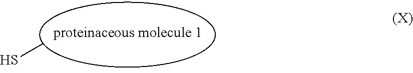

the first proteinaceous molecule covalently bound with at least one saponin derivative, wherein the at least one saponin derivative is based on a saponin comprising a triterpene aglycone core structure and at least one of a first saccharide chain ‘R¹’ and a second saccharide chain ‘R²’ linked to the aglycone core structure, wherein the saponin derivative comprises an aglycone core structure comprising an aldehyde group, wherein the aldehyde group is transformed into a semicarbazone functional group according to formula (I)

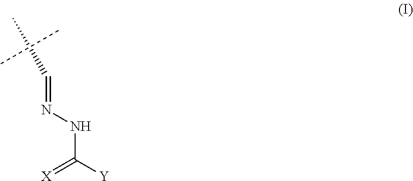

wherein R¹ and R² are independently selected from hydrogen, a monosaccharide, a linear oligosaccharide and a branched oligosaccharide,
X=O, P or S, and
Y=

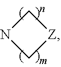

wherein n and m each are an integer independently selected from 1, 2, or 3,
Z=NR⁵, and
wherein R⁵ represents a maleimide moiety according to formula (II)

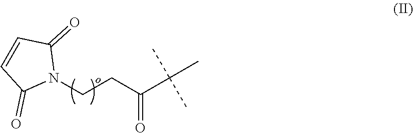

wherein o is an integer selected from 0-10, preferably 2-7, more preferably 4-6,
and wherein the maleimide moiety (II) of the saponin derivative is further transformed into a thioether bond through reaction
either, with the at least one thiol functional group of the first proteinaceous molecule, or with at least one thiol functional group of an oligomeric molecule which oligomeric molecule comprises a maleimide moiety that is transformed into a thioether bond through reaction with the at least one thiol functional group of the first proteinaceous molecule.

The saponin derivatives, wherein the aldehyde group is transformed into a semicarbazone functional group according to formula (IX)

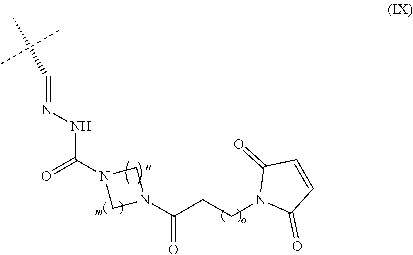

wherein n and m each are an integer independently selected from 1, 2, or 3; and
is an integer selected from 0-10, preferably 2-7, more preferably 4-6;
are suitable for application as a precursor for a conjugation reaction with a further molecule comprising a free sulfhydryl group. The maleimide functional group of the saponin derivative according to formula (IX) can form a thio-ether bond with such a free sulfhydryl group. For example, the saponin derivative according to formula (IX) can be covalently coupled to a peptide or a protein which comprises a free sulfhydryl group such as a cysteine with a free sulfhydryl group. Such a protein is for example an antibody or a binding derivative or binding fragment or binding domain thereof such as a F(ab′)2 fragment, Fab′ fragment, Fab fragment, scFv, dsFv, scFv-Fc, reduced IgG (rIgG), minibody, diabody, triabody, tetrabody, Fc fusion protein, nanobody, variable V domain, a single-domain antibody (sdAb), preferably a V_{HH}, for example camelid V_{H}, or a ligand for a cell-surface molecule such as a receptor such as EGF and a cytokine. Application of the saponin derivative according to formula (IX) in a coupling reaction with e.g. an antibody that comprises a free sulfhydryl group, provides a conjugate for targeted delivery of the saponin to and inside a cell, when the antibody (or the binding domain or fragment thereof) is an antibody for specific binding to a target cell surface molecule such as a receptor, e.g. as present on a tumor cell. Preferably, the saponin derivative is coupled to an antibody or V_{HH} capable of binding to a tumor-cell specific surface molecule such as a receptor, e.g. HER2, EGFR, CD71.

An embodiment is the saponin conjugate according to the invention, wherein the saponin is a mono-desmosidic triterpene saponin or bi-desmosidic triterpene saponin belonging to the type of a 12,13-dehydrooleanane with the aldehyde group in position C-23 and optionally comprising a glucuronic acid group in a carbohydrate substituent at the C-3beta-OH group of the saponin, preferably a bi-desmosidic triterpene saponin belonging to the type of a 12,13-dehydrooleanane with the aldehyde group in position C-23 and comprising a glucuronic acid group in a carbohydrate substituent at the C-3beta-OH group of the saponin.

An embodiment is the saponin conjugate according to the invention, wherein the triterpene aglycone core structure is selected from quillaic acid and gypsogenin, preferably the triterpene aglycone core structure is quillaic acid.

An embodiment is the saponin conjugate according to the invention, wherein the saponin derivative is according to formula (IV):

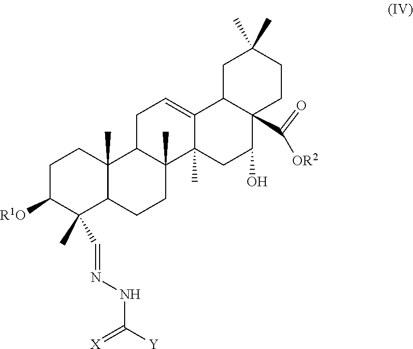

wherein R¹ and R² are independently selected from hydrogen, a monosaccharide, a linear oligosaccharide and a branched oligosaccharide,
X=O, P or S, and
Y=

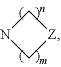

wherein n and m each are an integer independently selected from 1, 2 or 3;
Z=NR⁵, and
wherein R⁵ represents a maleimide moiety according to formula (II)

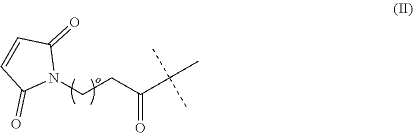

wherein o is an integer selected from 0-10, preferably 2-7, more preferably 4-6.

An embodiment is the saponin conjugate according to the invention, wherein X=O.

An embodiment is the saponin conjugate according to the invention,
wherein Y=

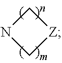

n and m each are an integer independently selected from 1, 2 or 3; and
Z=NR⁵; and
wherein R⁵ represents a maleimide moiety according to formula (II)

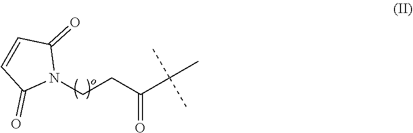

wherein o is an integer selected from 0-10, preferably 2-7, more preferably

An embodiment is the saponin conjugate according to the invention,
wherein n=1 and m=1, or
n=2 and m=1, or
n=2 and m=2, or
n=3 and m=2, or
n=3 and m=3;
preferably wherein n=2 and m=2.

An embodiment is the saponin conjugate according to the invention, wherein the first saccharide chain R¹ is selected from:
H,
GlcA-,
Glc-,
GaI-,
Rha-(1→2)-Ara-,
GaI-(1→2)-[XyI-(1→3)]-GlcA-,
Glc-(1→2)-[Glc-(1→4)]-GlcA-,
Glc-(1→2)-Ara-(1→3)-[GaI-(1→2)]-GlcA-,
XyI-(1→2)-Ara-(1→3)-[GaI-(1→2)]-GlcA-,
Glc-(1→3)-GaI-(1→2)-[XyI-(1→3)]-Glc-(1→4)-GaI-,
Rha-(1→2)-GaI-(1→3)-[Glc-(1→2)]-GlcA-,
Ara-(1→4)-Rha-(1→2)-Glc-(1→2)-Rha-(1→2)-GlcA-,
Ara-(1→4)-Fuc-(1→2)-Glc-(1→2)-Rha-(1→2)-GlcA-,
Ara-(1→4)-Rha-(1→2)-GaI-(1→2)-Rha-(1→2)-GlcA-,
Ara-(1→4)-Fuc-(1→2)-GaI-(1→2)-Rha-(1→2)-GlcA-,
Ara-(1→4)-Rha-(1→2)-Glc-(1→2)-Fuc-(1→2)-GlcA-,
Ara-(1→4)-Fuc-(1→2)-Glc-(1→2)-Fuc-(1→2)-GlcA-,
Ara-(1→4)-Rha-(1→2)-GaI-(1→2)-Fuc-(1→2)-GlcA-,
Ara-(1→4)-Fuc-(1→2)-GaI-(1→2)-Fuc-(1→2)-GlcA-,
XyI-(1→4)-Rha-(1→2)-Glc-(1→2)-Rha-(1→2)-GlcA-,
XyI-(1→4)-Fuc-(1→2)-Glc-(1→2)-Rha-(1→2)-GlcA-,
XyI-(1→4)-Rha-(1→2)-GaI-(1→2)-Rha-(1→2)-GlcA-,
XyI-(1→4)-Fuc-(1→2)-GaI-(1→2)-Rha-(1→2)-GlcA-,
XyI-(1→4)-Rha-(1→2)-Glc-(1→2)-Fuc-(1→2)-GlcA-,
XyI-(1→4)-Fuc-(1→2)-Glc-(1→2)-Fuc-(1→2)-GlcA-,
XyI-(1→4)-Rha-(1→2)-GaI-(1→2)-Fuc-(1→2)-GlcA-,
XyI-(1→4)-Fuc-(1→2)-GaI-(1→2)-Fuc-(1→2)-GlcA-, and
derivatives thereof, and
wherein the second saccharide chain R² is selected from:
H,
Glc-,
GaI-,
Rha-(1→2)-[XyI-(1→4)]-Rha-,
Rha-(1→2)-[Ara-(1→3)-XyI-(1→4)]-Rha-,
Ara-,
XyI-,
XyI-(1→4)-Rha-(1→2)-[R1-(→4)]-Fuc- wherein R1 is 4E-Methoxycinnamic acid,
XyI-(1→4)-Rha-(1→2)-[R2-(→4)]-Fuc- wherein R2 is 4Z-Methoxycinnamic acid,
XyI-(1→4)-[GaI-(1→3)]-Rha-(1→2)-4-OAc-Fuc-,
XyI-(1→4)-[Glc-(1→3)]-Rha-(1→2)-3,4-di-OAc-Fuc-,
XyI-(1→4)-[Glc-(1→3)]-Rha-(1→2)-[R⁶-(→4)]-3-OAc-Fuc- wherein R⁶ is 4E-Methoxycinnamic acid,
Glc-(1→3)-XyI-(1→4)-[Glc-(1→3)]-Rha-(1→2)-4-OAc-Fuc-,
Glc-(1→3)-XyI-(1→4)-Rha-(1→2)-4-OAc-Fuc-,
(Ara- or XyI-)(1→3)-(Ara- or XyI-)(1→4)-(Rha- or Fuc-)(1→2)-[4-OAc-(Rha- or Fuc-)(1→4)]-(Rha- or Fuc-),
XyI-(1→3)-XyI-(1→4)-Rha-(1→2)-[Qui-(1→4)]-Fuc-,
Api-(1→3)-XyI-(1→4)-[Glc-(1→3)]-Rha-(1→2)-Fuc-,
XyI-(1→4)-[GaI-(1→3)]-Rha-(1→2)-Fuc-,
XyI-(1→4)-[Glc-(1→3)]-Rha-(1→2)-Fuc-,
Ara/XyI-(1→4)-Rha/Fuc-(1→4)-[Glc/GaI-(1→2)]-Fuc-,
Api-(1→3)-XyI-(1→4)-[Glc-(1→3)]-Rha-(1→2)-[R⁷-(→4)]-Fuc- wherein R⁷ is 5-O-[5-O-Ara/Api-3,5-dihydroxy-6-methyl-octanoyl]-3,5-dihydroxy-6-methyl-octanoic acid),
Api-(1→3)-XyI-(1→4)-Rha-(1→2)-[R⁸-(→4)]-Fuc- wherein R⁸ is 5-O-[5-O-Ara/Api-3,5-dihydroxy-6-methyl-octanoyl]-3,5-dihydroxy-6-methyl-octanoic acid),
Api-(1→3)-XyI-(1→4)-Rha-(1→2)-[Rha-(1→3)]-4-OAc-Fuc-,
Api-(1→3)-XyI-(1→4)-[Glc-(1→3)]-Rha-(1→2)-[Rha-(1→3)]-4-OAc-Fuc-,
6-OAc-Glc-(1→3)-XyI-(1→4)-Rha-(1→2)-[3-OAc-Rha-(1→3)]-Fuc-,
Glc-(1→3)-XyI-(1→4)-Rha-(1→2)-[3-OAc--Rha-(1→3)]-Fuc-,
XyI-(1→3)-XyI-(1→4)-Rha-(1→2)-[Qui-(1→4)]-Fuc-,
Glc-(1→3)-[XyI-(1→4)]-Rha-(1→2)-[Qui-(1→4)]-Fuc-,
Glc-(1→3)-XyI-(1→4)-Rha-(1→2)-[XyI-(1→3)-4-OAc-Qui-(1→4)]-Fuc-,
XyI-(1→3)-XyI-(1→4)-Rha-(1→2)-[3,4-di-OAc-Qui-(1→4)]-Fuc-,
Glc-(1→3)-[XyI-(1→4)]-Rha-(1→2)-Fuc-,
6-OAc-Glc-(1→3)-[XyI-(1→4)]-Rha-(1→2)-Fuc-,
Glc-(1→3)-[XyI-(1→3)-XyI-(1→4)]-Rha-(1→2)-Fuc-,
XyI-(1→3)-XyI-(1→4)-Rha-(1→2)-[XyI-(1→3)-4-OAc-Qui-(1→4)]-Fuc-,
Api/XyI-(1→3)-XyI-(1→4)-[Glc-(1→3)]-Rha-(1→2)-[Rha-(1→3)]-4OAc-Fuc-,
Api-(1→3)-XyI-(1→4)-[Glc-(1→3)]-Rha-(1→2)-[Rha-(1→3)]-4OAc-Fuc-,
Api/XyI-(1→3)-XyI-(1→4)-[Glc-(1→3)]-Rha-(1→2)-[R⁹-(→4)]-Fuc- wherein R⁹ is 5-O-[5-O-Rha-(1→2)-Ara/Api-3,5-dihydroxy-6-methyl-octanoyl]-3,5-dihydroxy-6-methyl-octanoic acid),
Api/XyI-(1→3)-XyI-(1→4)-[Glc-(1→3)]-Rha-(1→2)-[R¹⁹-(→4)]-Fuc- wherein R¹⁹ is 5-O-[5-O-Ara/Api-3,5-dihydroxy-6-methyl-octanoyl]-3,5-dihydroxy-6-methyl-octanoic acid),
Api/XyI-(1→3)-XyI-(1→4)-[Glc-(1→3)]-Rha-(1→2)-[R¹¹-(→4)]-Fuc- wherein R¹¹ is 5-O-[5-O-Ara/Api-3,5-dihydroxy-6-methyl-octanoyl]-3,5-dihydroxy-6-methyl-octanoic acid),
Api-(1→3)-XyI-(1→4)-Rha-(1→2)-[R¹²-(→4)]-Fuc- wherein R¹² is 5-O-[5-O-Ara/Api-3,5-dihydroxy-6-methyl-octanoyl]-3,5-dihydroxy-6-methyl-octanoic acid),
XyI-(1→3)-XyI-(1→4)-Rha-(1→2)-[R¹³-(→4)]-Fuc- wherein R¹³ is 5-O-[5-O-Ara/Api-3,5-dihydroxy-6-methyl-octanoyl]-3,5-dihydroxy-6-methyl-octanoic acid),
Api-(1→3)-XyI-(1→4)-Rha-(1→2)-[R¹⁴-(→3)]-Fuc- wherein R¹⁴ is 5-O-[5-O-Ara/Api-3,5-dihydroxy-6-methyl-octanoyl]-3,5-dihydroxy-6-methyl-octanoic acid),
XyI-(1→3)-XyI-(1→4)-Rha-(1→2)-[R¹⁵-(→3)]-Fuc- wherein R¹⁵ is 5-O-[5-O-Ara/Api-3,5-dihydroxy-6-methyl-octanoyl]-3,5-dihydroxy-6-methyl-octanoic acid)
Glc-(1→3)-[Glc-(1→6)]-GaI-, and
derivatives thereof.

An embodiment is the saponin conjugate according to the invention, wherein the first saccharide chain R¹ is GaI-(1→2)-[XyI-(1→3)]-GlcA-; and wherein the second saccharide chain R² is selected from:
Glc-(1→3)-XyI-(1→4)-Rha-(1→2)-[XyI-(1→3)-4-OAc-Qui-(1→4)]-Fuc-,
XyI-(1→3)-XyI-(1→4)-Rha-(1→2)-[3,4-di-OAc-Qui-(1→4)]-Fuc-,
Api-(1→3)-XyI-(1→4)-Rha-(1→2)-[R¹²-(→4)]-Fuc- wherein R¹² is 5-O-[5-O-Ara/Api-3,5-dihydroxy-6-methyl-octanoyl]-3,5-dihydroxy-6-methyl-octanoic acid,
XyI-(1→3)-XyI-(1→4)-Rha-(1→2)-[R¹³-(→4)]-Fuc- wherein R¹³ is 5-O-[5-O-Ara/Api-3,5-dihydroxy-6-methyl-octanoyl]-3,5-dihydroxy-6-methyl-octanoic acid,
Api-(1→3)-XyI-(1→4)-Rha-(1→2)-[R¹⁴-(→3)]-Fuc- wherein R¹⁴ is 5-O-[5-O-Ara/Api-3,5-dihydroxy-6-methyl-octanoyl]-3,5-dihydroxy-6-methyl-octanoic acid, and
XyI-(1→3)-XyI-(1→4)-Rha-(1→2)-[R¹⁵-(→3)]-Fuc- wherein R¹⁵ is 5-O-[5-O-Ara/Api-3,5-dihydroxy-6-methyl-octanoyl]-3,5-dihydroxy-6-methyl-octanoic acid,
preferably, R¹ is GaI-(1→2)-[XyI-(1→3)]-GlcA- and R² is Glc-(1→3)-XyI-(1→4)-Rha-(1→2)-[XyI-(1→3)-4-OAc-Qui-(1→4)]-Fuc-.

Preferably, the saponin conjugate comprises a saponin derivative, wherein the saponin derivative is based on a triterpenoid saponin of the 12,13-dehydrooleanane type, preferably with an aldehyde group in position C-23 of the aglycone core. In Table A1, examples of such saponins are listed. In particular, triterpenoid saponins of the 12,13-dehydrooleanane type with an aldehyde group in position C-23 of the aglycone core, wherein the aglycone core is quillaic acid or gypsogenine, are preferred. Examples of such triterpenoid saponins are listed in Table A1.

An embodiment is the saponin conjugate according to the invention, wherein the at least one saponin on which the saponin derivative is based is any one or more of:
a) saponin selected from any one or more of list A:
   Quillaja saponaria saponin mixture, or a saponin isolated from Quillaja saponaria, for example Quil-A, QS-17-api, QS-17-xyl, QS-21, QS-21A, QS-21B, QS-7-xyl;
   Saponinum album saponin mixture, or a saponin isolated from Saponinum album;
   Saponaria officinalis saponin mixture, or a saponin isolated from Saponaria officinalis; and
   Quillaja bark saponin mixture, or a saponin isolated from Quillaja bark, for example Quil-A, QS-17-api, QS-17-xyl, QS-21, QS-21A, QS-21B, QS-7-xyl; or
b) a saponin comprising a gypsogenin aglycone core structure, selected from list B:
   SA1641, gypsoside A, NP-017772, NP-017774, NP-017777, NP-017778, NP-018109, NP-017888, NP-017889, NP-018108, SO1658 and Phytolaccagenin; or
c) a saponin comprising a quillaic acid aglycone core structure, selected from list C:
   AG1856, AG1, AG2, Agrostemmoside E, GE1741, Gypsophila saponin 1 (Gyp1), NP-017674, NP-017810, NP-003881, NP-017676, NP-017677, NP-017705, NP-017706, NP-017773, SO1832, SO1861, SO1862, SO1904, QS1861, QS1862, QS-7, QS-7 api, QS-17, QS-18, QS-21 A-apio, QS-21 A-xylo, QS-21 B-apio and QS-21 B-xylo,
preferably, the at least one saponin is any one or more of a saponin selected from list B or C, more preferably from list C.

An embodiment is the saponin conjugate according to the invention, wherein the at least one saponin on which the saponin derivative is based is any one or more of AG1856, GE1741, a saponin isolated from Quillaja saponaria, Quil-A, QS-17, QS-21, QS-7, SA1641, a saponin isolated from Saponaria officinalis, Saponarioside B, SO1542, SO1584, SO1658, SO1674, SO1700, SO1730, SO1772, SO1832, SO1861, SO1862 and SO1904, preferably the at least one saponin is any one or more of QS-21, SO1832, SO1861, SA1641 and GE1741, more preferably the at least one saponin is QS-21, SO1832 or SO1861, even more preferably the at least one saponin is SO1861 or SO1832.

An embodiment is the saponin conjugate according to the invention, wherein the at least one saponin on which the saponin derivative is based is a saponin isolated from Saponaria officinalis, preferably the at least one saponin is any one or more of Saponarioside B, SO1542, SO1584, SO1658, SO1674, SO1700, SO1730, SO1772, SO1832, SO1861, SO1862 and SO1904, more preferably the at least one saponin is any one or more of SO1832, SO1861 and SO1862, even more preferably SO1832 and SO1861, even more preferably the at least one saponin is SO1861.

An embodiment is the saponin conjugate according to the invention, wherein the saponin derivative is according to formula (VII)

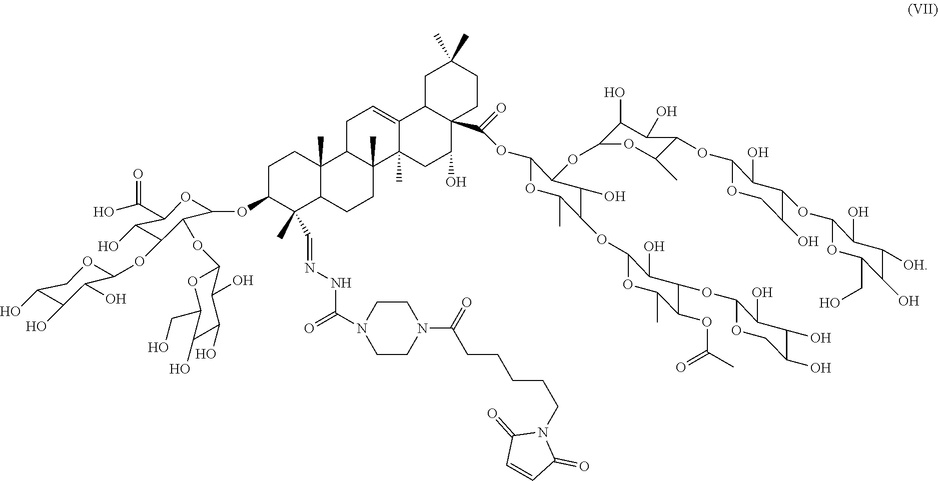

It will be appreciated by the skilled person that since the saponin conjugate of the invention comprises (is based on) the saponin derivative according to the invention, all embodiments referring to the saponin derivative applies mutatis mutandis to the saponin conjugate. That is to say, the saponin conjugates of the invention are (also) based on the saponin derivatives according to the invention, detailed here above.

A preferred embodiment is the saponin conjugate according to the invention, wherein the saponin conjugate is according to formula (XI)

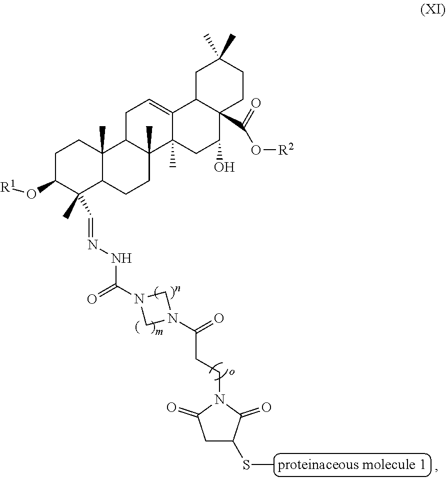

wherein R¹ and R² are as defined here above for the saponin conjugate;
n and m each are an integer independently selected from 1, 2 or 3; and
is an integer selected from 0-10, preferably 2-7, more preferably 4-6.

A preferred embodiment is the saponin conjugate according to the invention, wherein the semicarbazone functional group according to formula (IX) or the saponin conjugate is according to formula (XI), wherein n=1 and m=1, n=2 and m=1, n=2 and m=2, n=3 and m=2, n=3 and m=3; preferably wherein n=1 and m=2, n=2 and m=2, or n=2 and m=3; more preferably wherein n=2 and m=2.

A highly preferred embodiment is the saponin conjugate according to formula (XII)

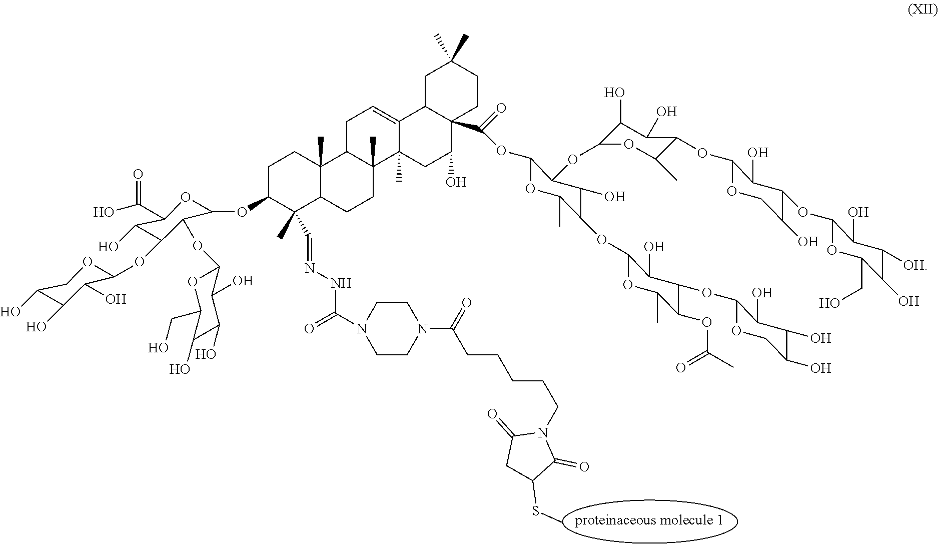

The saponin conjugate according to formula (XII) is based on the SO1861 derivative. The saponin conjugate that is equally preferred is based on the SO1832 derivate, providing a saponin conjugate reminiscent to the saponin conjugate according to formula (XII) though comprising the glycans of SO1832 (see Table A1).

An embodiment is the saponin conjugate of the invention, wherein the oligomeric molecule to which the at least one saponin (derivative) is covalently bound, is selected from: a dendron, a poly-ethylene glycol such as any one of PEG₃-PEG₃₀, preferably any one of PEG₄-PEG₁₂, preferably the oligomeric molecule is a dendron such as a poly-amidoamine (PAMAM) dendrimer.

An embodiment is the saponin conjugate of the invention, wherein the oligomeric molecule is a dendron, preferably a G2 dendron, a G3 dendron, a G4 dendron or a G5 dendron, more preferably a G2 dendron or a G3 dendron.

An embodiment is the saponin conjugate of the invention, wherein the semicarbazone functional group

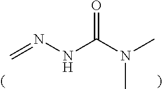

is hydrolysable under acidic conditions, preferably at pH 4.0-6.5, wherein hydrolysis of said semicarbazone functional group provides the aldehyde group on the aglycone core structure of the saponin on which the saponin derivative is based,
and/or
wherein the semicarbazone functional group is subject to cleavage in vivo under acidic conditions such as for example present in endosomes and/or lysosomes of a mammalian cell, preferably a human cell such as a diseased cell, an aberrant cell or a tumor cell, preferably at pH 4.0-6.5, and more preferably at pH 5.5, wherein hydrolysis of said semicarbazone functional group provides the aldehyde group on the aglycone core structure of the saponin on which the saponin derivative is based.

An embodiment is the saponin conjugate of the invention, comprising more than one copy of the saponin, preferably any number of saponin copies selected from 1-64 copies of the saponin, more preferably 2-32 copies of the saponin, even more preferably 3-16 copies of the saponin, even more preferably 4-12 copies of the saponin, such as 1, 2, 3, 4, 5, 6, 7, 8, 9, 10, 11, 12, 13, 14, 15 or 16 copies of the saponin, preferably 2, 4 or 8 copies of the saponin.

An embodiment is the saponin conjugate of the invention, wherein the saponin conjugate is according to formula (XII)a

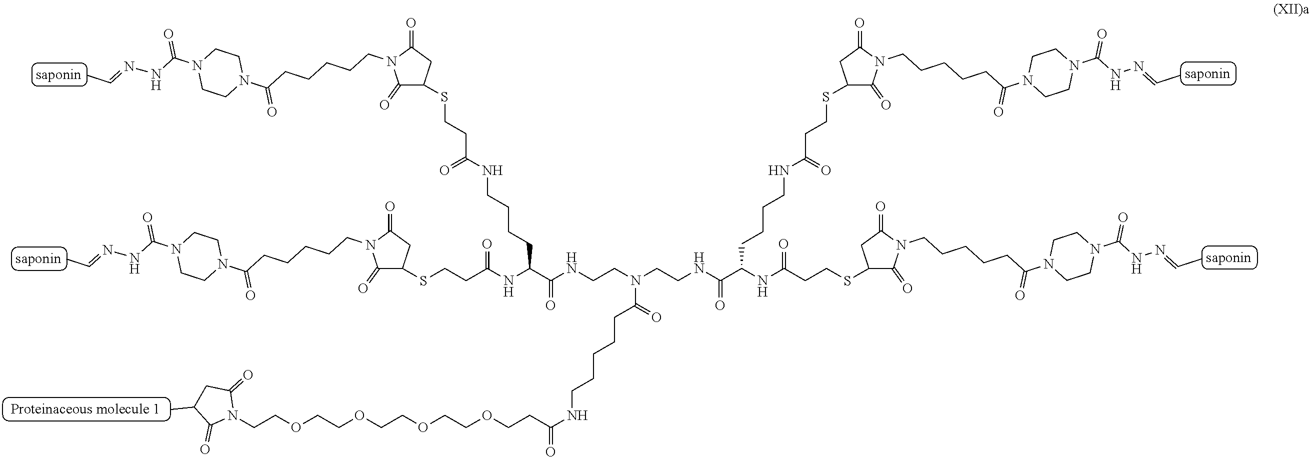 [SMILES: C/C=N/NC(=O)N1CCN(C(=O)CCCCCN2C(=O)CC(SCCC(=O)NCCCC[C@H](NC(=O)CCSC3CC(=O)N(CCCCCC(=O)N4CCN(C(=O)N/N=C/C)CC4)C3=O)C(=O)NCCN(CCNC(=O)[C@H](CCCCNC(=O)CCSC3CC(=O)N(CCCCCC(=O)N4CCN(C(=O)N/N=C/C)CC4)C3=O)NC(=O)CCSC3CC(=O)N(CCCCCC(=O)N4CCN(C(=O)N/N=C/C)CC4)C3=O)C(=O)CCCCCNC(=O)CCOCCOCCOCCOCCN3C(=O)CC(C)C3=O)C2=O)CC1]

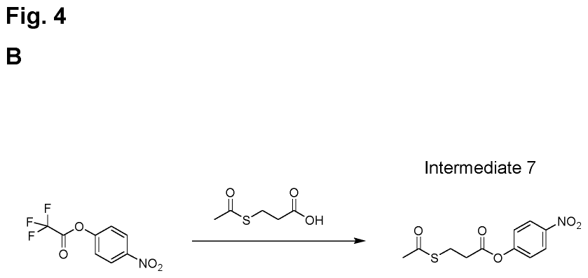

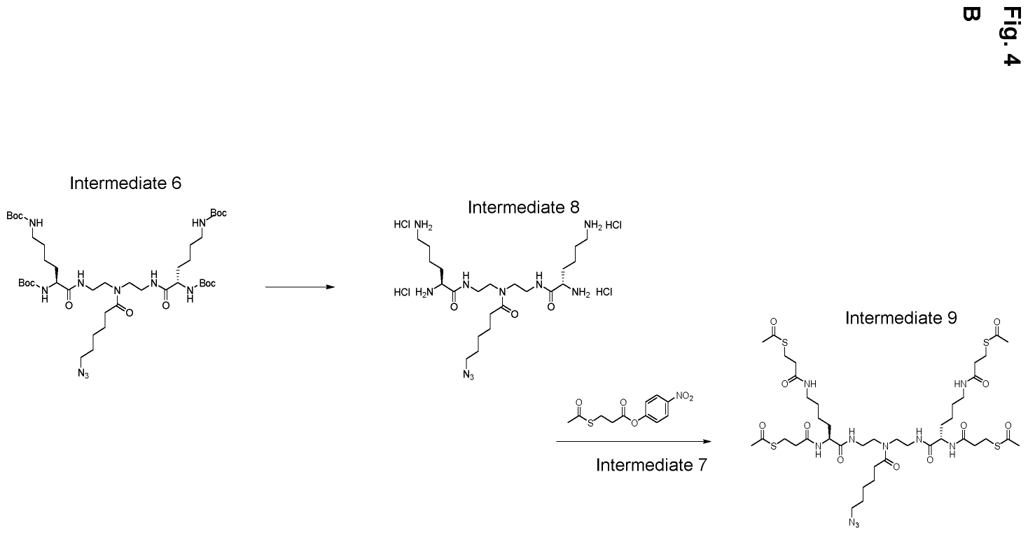

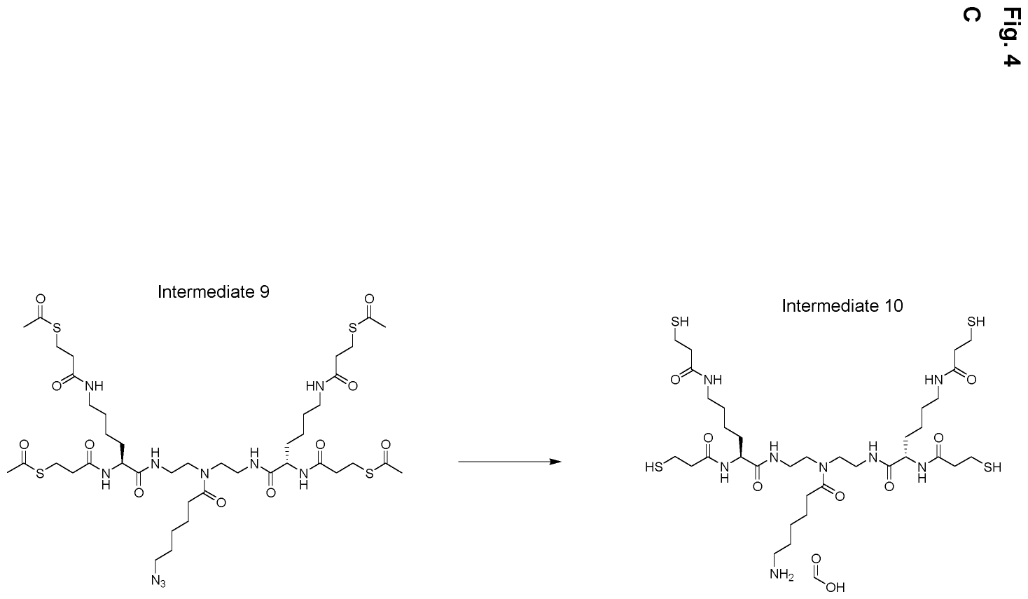

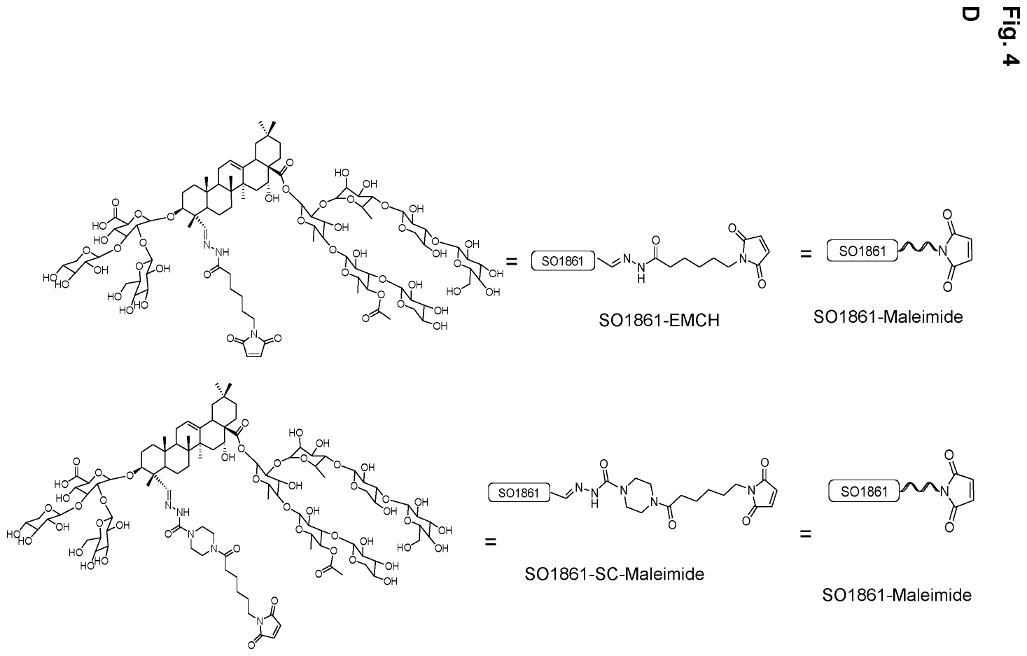

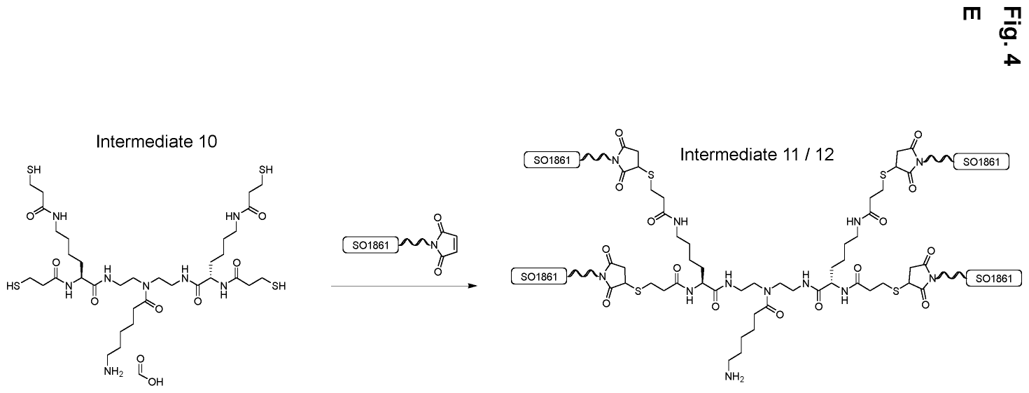

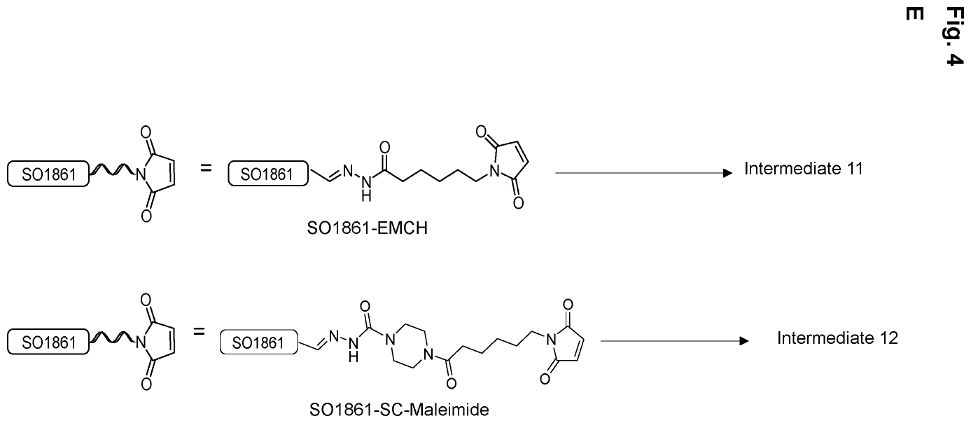

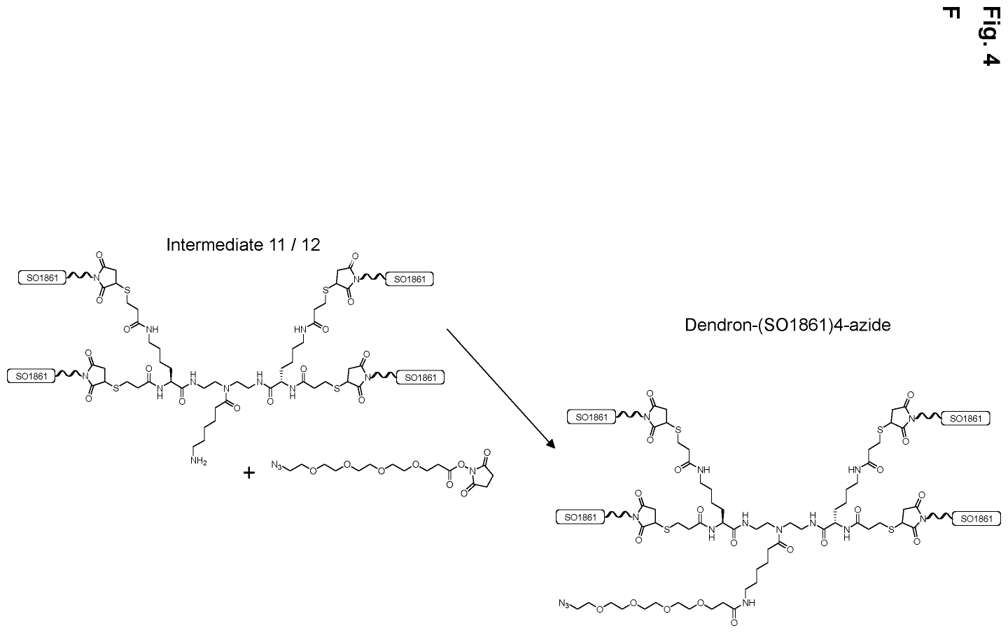

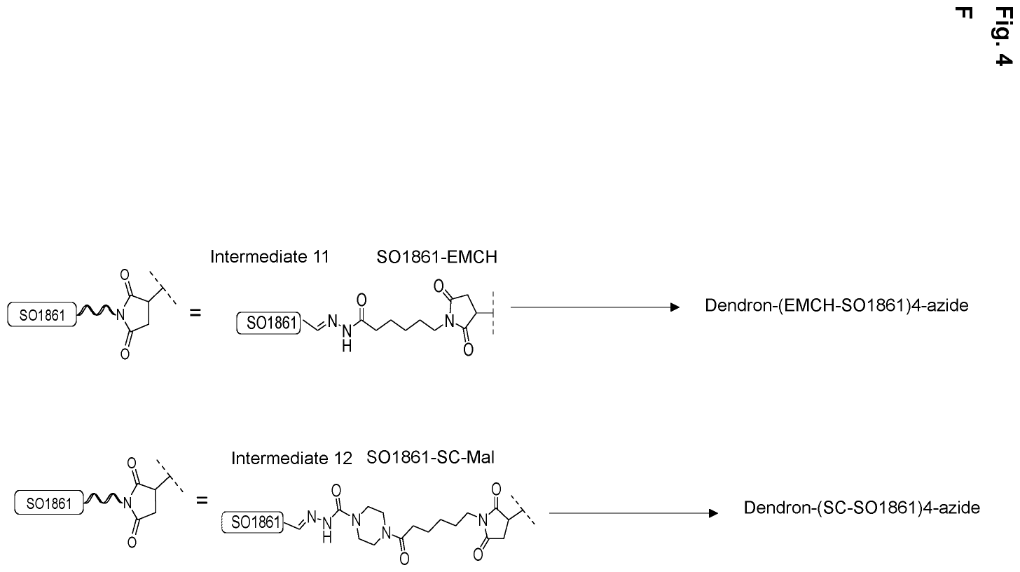

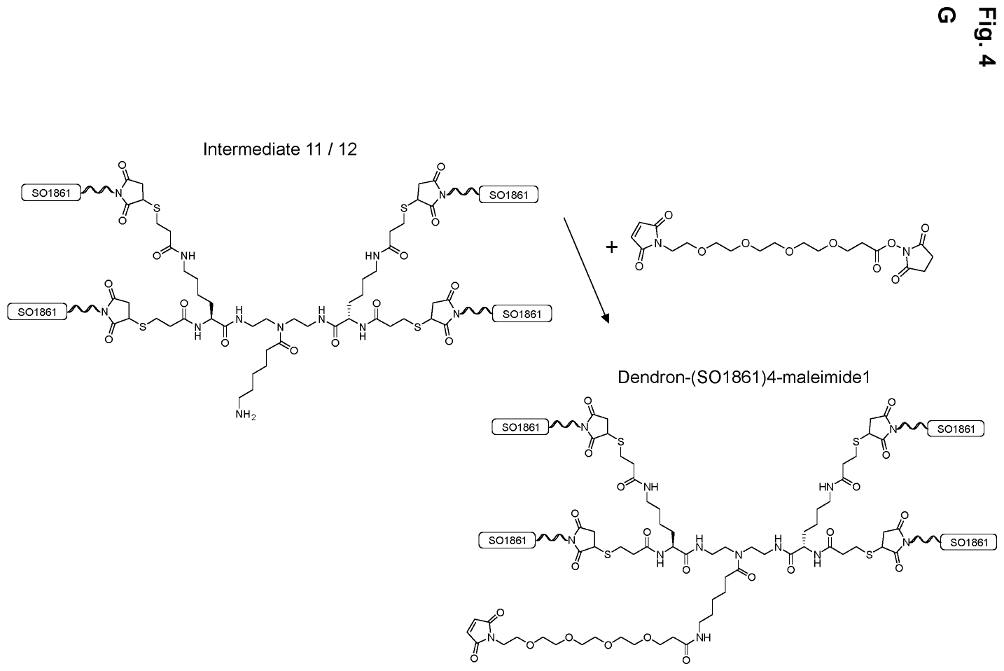

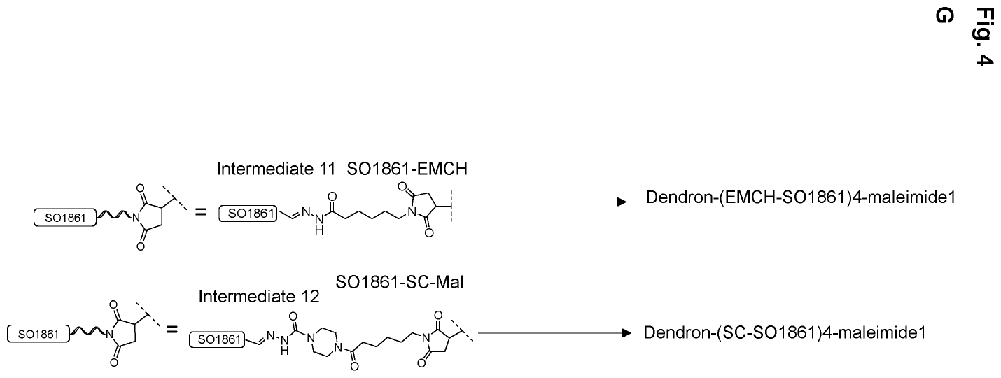

Preferably, the proteinaceous molecule 1 is an antibody; preferably an anti-CD71 antibody or at least one sdAb capable of binding to CD71. Preferably, the saponin is SO1861 or SO1832. Preferably, the saponin derivative moieties comprised by the saponin conjugate are the same. When the first proteinaceous molecule (proteinaceous molecule 1) comprised by the saponin conjugate is an antibody, the saponin conjugate comprises preferably 1 or 2 conjugated copies of the dendron-(saponin)4-maleimide2, for which dendron-(saponin)4-maleimide2 as an example dendron-(SO1861)4-maleimide2 is displayed in FIG. 4H. More preferred is a single conjugated copy of the dendron-(saponin)4-maleimide2 per antibody molecule. Preferably, the saponin is SO1861 or SO1832, more preferably SO1861.

An embodiment is the saponin conjugate of the invention, wherein the saponin conjugate is according to formula (XII)b

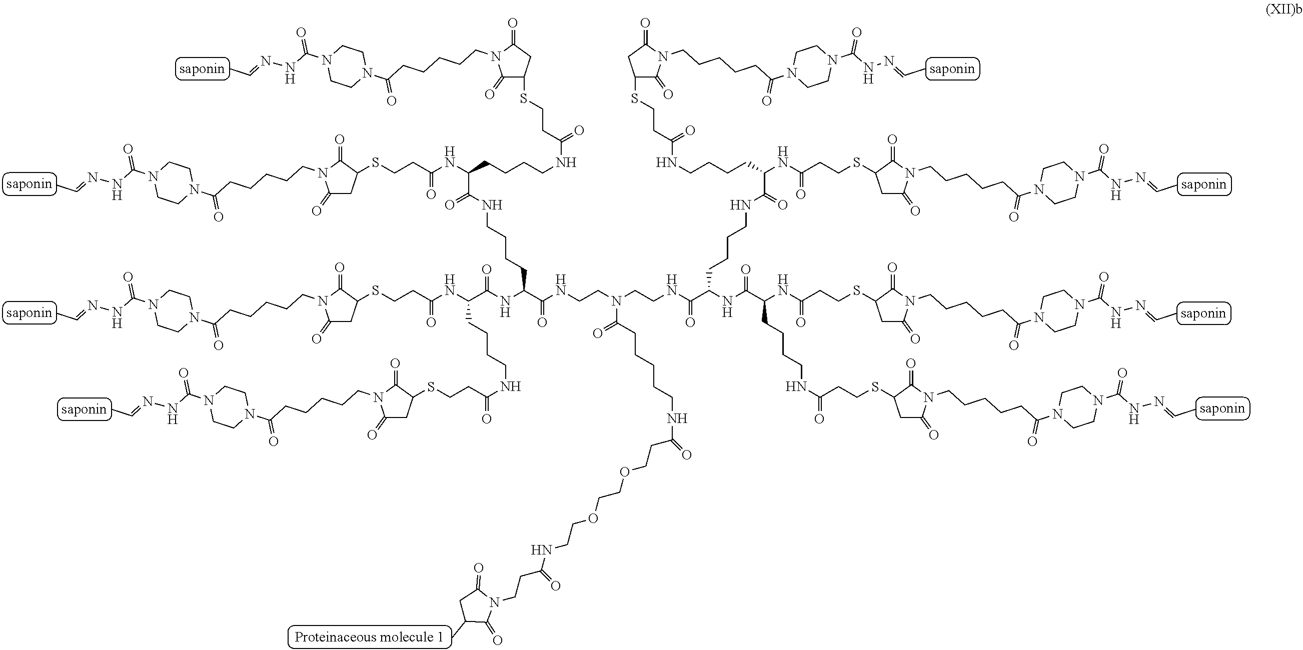

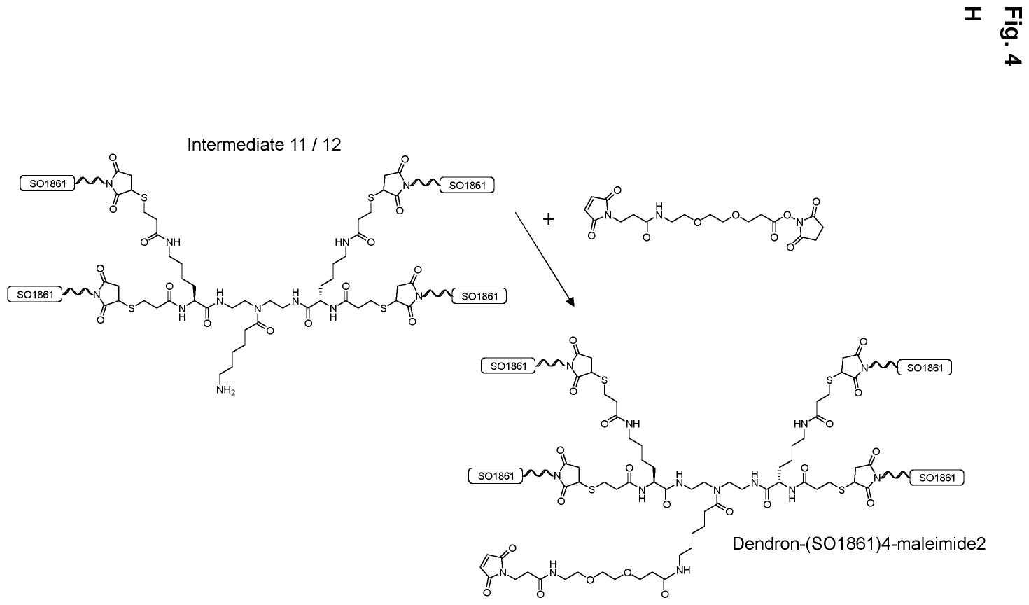

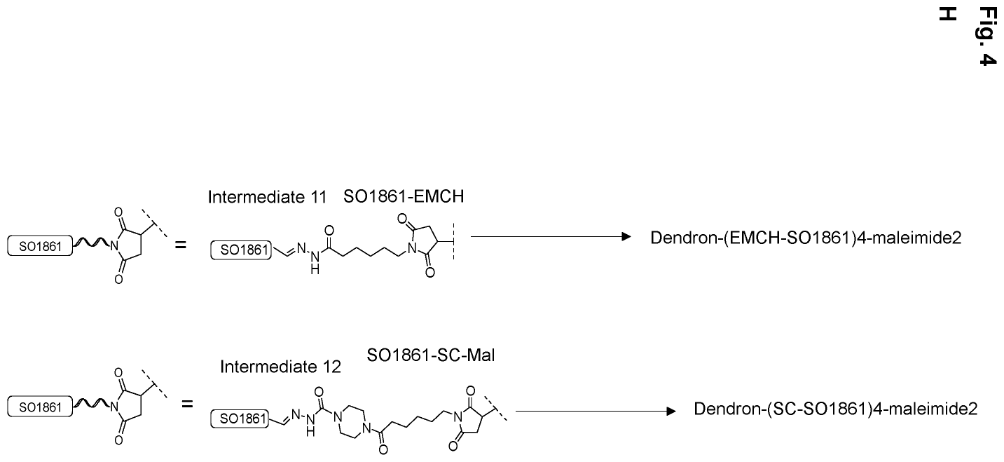

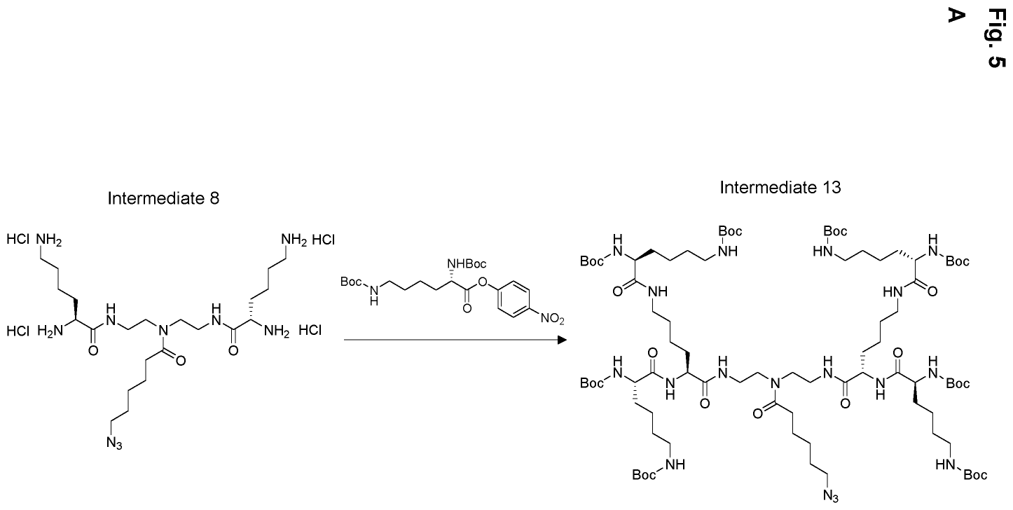

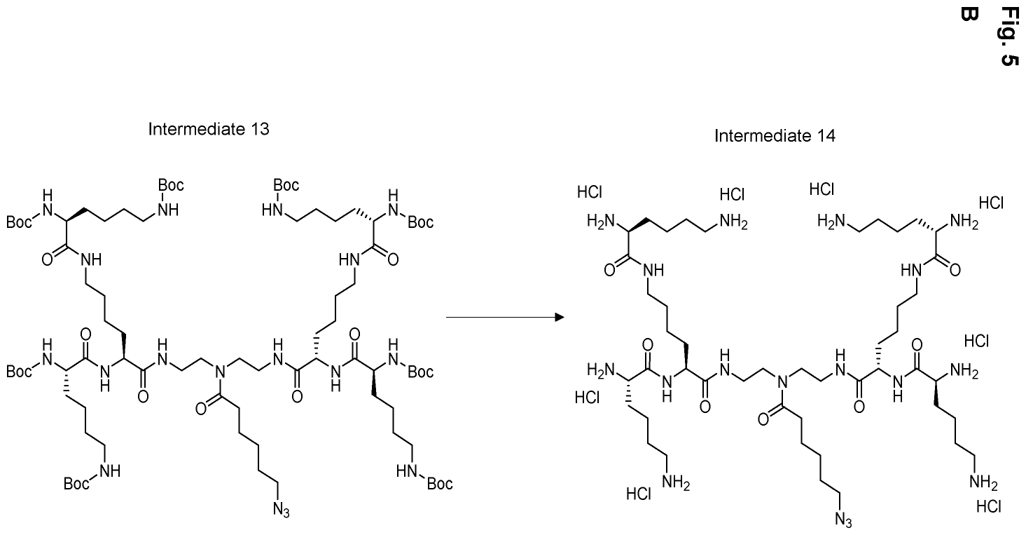

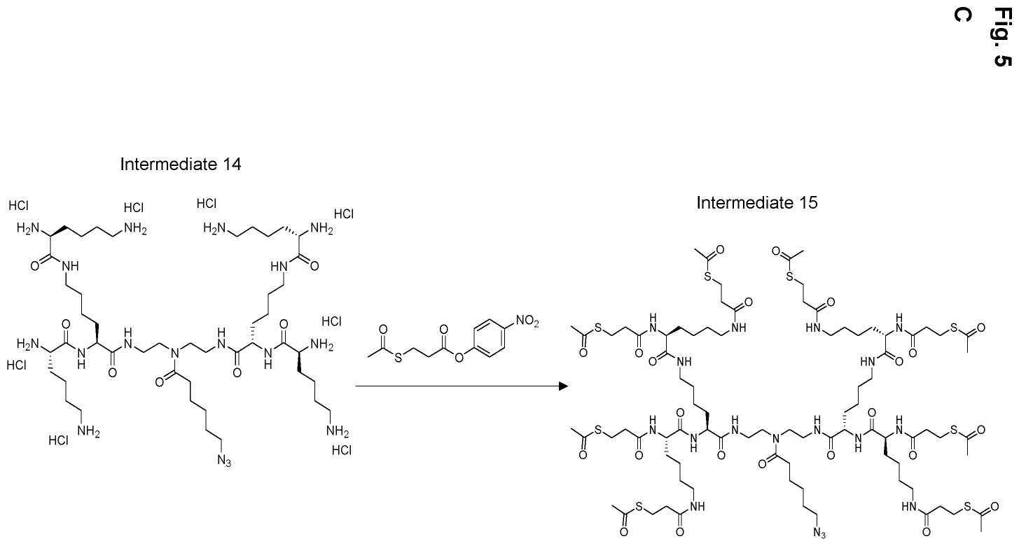

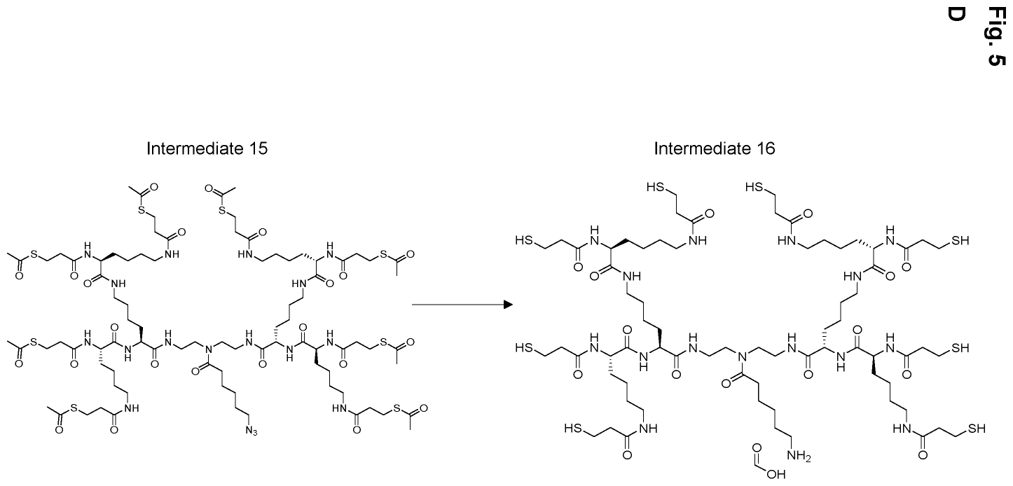

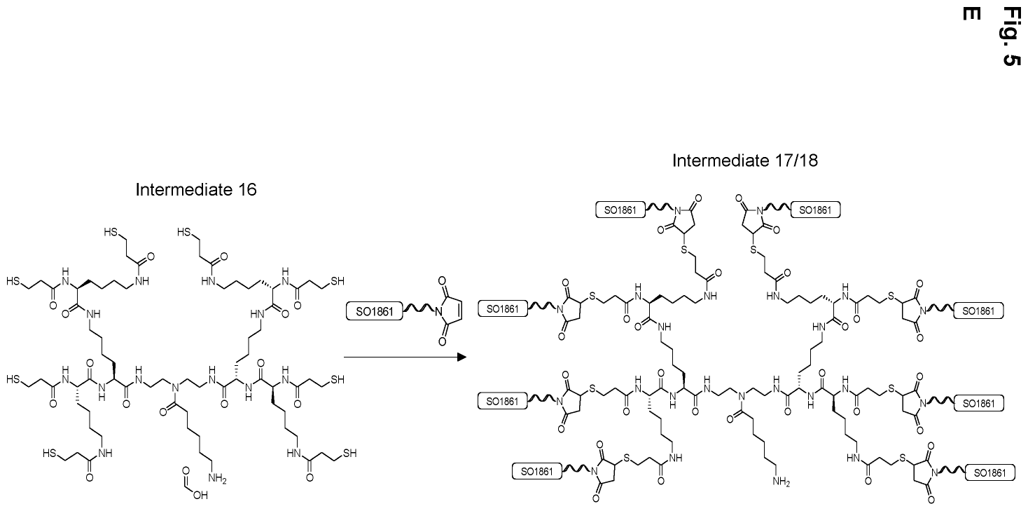

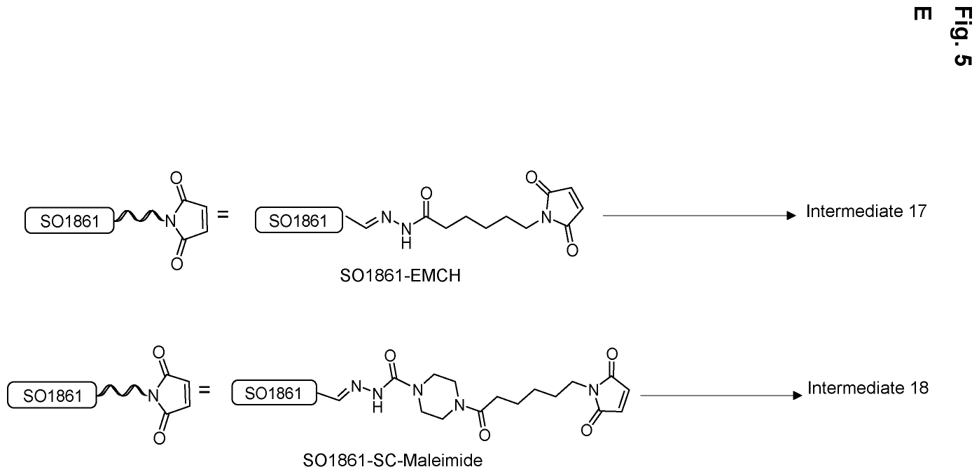

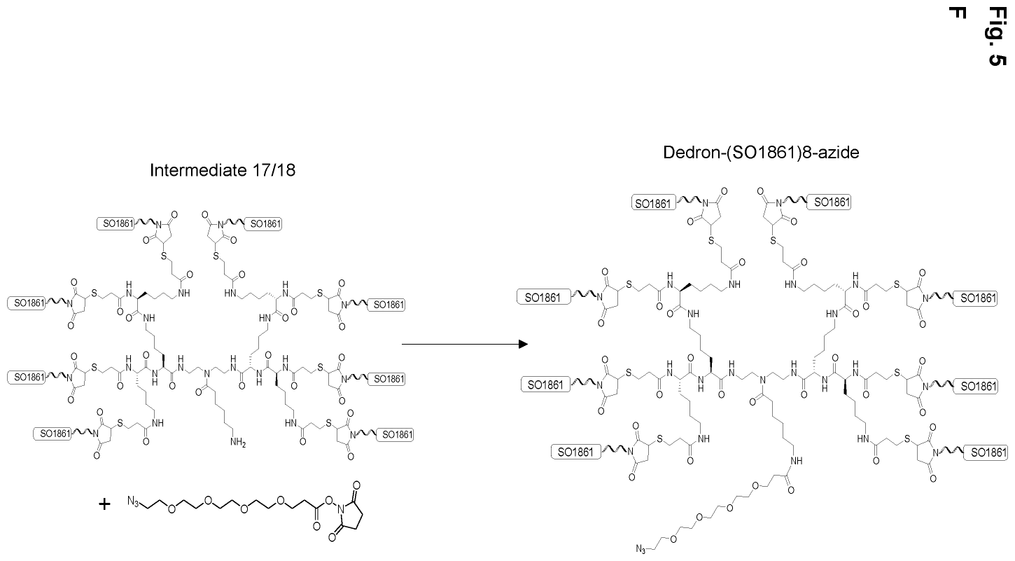

Preferably, the proteinaceous molecule 1 is an antibody; preferably an anti-CD71 antibody or at least one sdAb capable of binding to CD71. Preferably, the saponin is SO1861 or SO1832. Preferably, the saponin derivative moieties comprised by the saponin conjugate are the same. When the first proteinaceous molecule (proteinaceous molecule 1) comprised by the saponin conjugate is an antibody, the saponin conjugate comprises preferably 1 or 2 conjugated copies of the dendron-(saponin)8-maleimide1, for which dendron-(saponin)8-maleimide1 as an example dendron-(SO1861)8-maleimide1 is displayed in FIG. 5H. More preferred is a single conjugated copy of the dendron-(saponin)8-maleimide1 per antibody molecule. Preferably, the saponin is SO1861 or SO1832, more preferably SO1861.

The saponin conjugate according to formula (XII)a or formula (XII)b comprises for example a saponin derivative moiety according to formula (XII)c

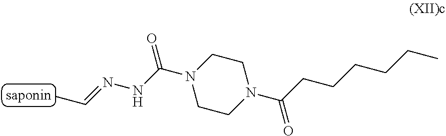 [SMILES: C/C=N/NC(=O)N1CCN(C(=O)CCCCCC)CC1]

which saponin derivative moiety is for example the moiety resulting from the coupling of the saponin derivative according to formula (KK)

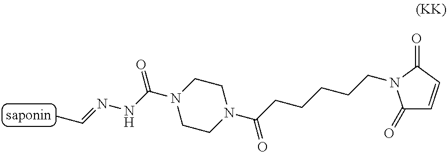 [SMILES: C/C=N/NC(=O)N1CCN(C(=O)CCCCCN2C(=O)C=CC2=O)CC1]

with a thiol-group bearing dendron such as a G2 dendron or G3 dendron. The moiety

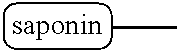

here represents a saponin moiety according to formula (SM):

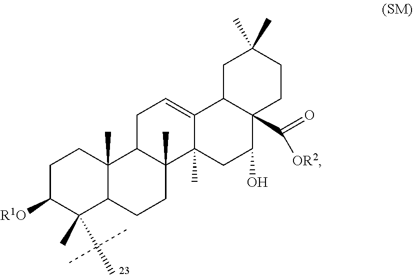

wherein R¹ and R² are independently selected from hydrogen, a monosaccharide, a linear oligosaccharide and a branched oligosaccharide according to any one of the oligosaccharides as listed here above, and wherein the saponin moiety according to formula (SM) is based on a saponin comprising an aldehyde group in position C-23 according to any one of the saponins listed here above, and in Table A1. Preferred are SO1861 and SO1832.

An embodiment is the saponin conjugate according to the invention, wherein the proteinaceous molecule 1 comprises a first binding site for binding to a first epitope of a first cell-surface molecule. Preferably, in the saponin conjugate according to the invention, the proteinaceous molecule 1 comprises a cell-surface molecule binding-molecule comprising a first binding site for binding to a first epitope of a first cell-surface molecule.

Preferred is the saponin conjugate of the invention, wherein the first binding site of the proteinaceous molecule 1 is or comprises any one or more of: an amino acid, a peptide, a protein, an antibody such as an IgG, preferably a monoclonal antibody, or a binding derivative of said antibody or binding fragment of said antibody or binding domain of said antibody such as a F(ab′)2 fragment, Fab′ fragment, Fab fragment, scFv, dsFv, scFv-Fc, reduced IgG (rIgG), minibody, diabody, triabody, tetrabody, Fc fusion protein, nanobody, variable V domain, a single-domain antibody (sdAb), wherein the sdAb preferably is a V_{HH}, for example a camelid V_{H}, or wherein said first binding site is or comprises a ligand for a cell-surface molecule preferably a receptor, preferably wherein the ligand is a proteinaceous ligand such as EGF or a cytokine, an adnectin, an affibody, an anticalin, or binding molecules comprising one or more of any of these cell-surface molecule binding-molecules.

Such a receptor ligand can be a receptor ligand known in the art of cell-targeting therapy, such as EGF. Typically, such a receptor ligand targets a receptor on a diseased cell such as a tumor cell or an auto-immune cell such as in rheumatoid arthritis. Typically, such a receptor ligand is a proteinaceous molecule such as a peptide or a protein, although non-proteinaceous receptor ligands known in the art are equally suitable. Such receptor ligands can also be selected from molecules such as adnectins, anticalins, affibodies, etc., etc. The common denominator for the receptor ligands is their specificity for an epitope on a target cell, for targeted delivery of an effector moiety bound to the receptor ligand, reminiscent to the targeted delivery of effector moieties such as toxins, enzymes, oligonucleotides, drug molecules, etc., linked to e.g. an antibody or a single domain antibody, etc. Suitable cell-surface molecule binding-molecules are proteinaceous molecules such as antibodies, sdAb, etc., and non-proteinaceous molecules, suitable for targeting a selected cell (type) and therewith suitable for bringing a saponin derivative of the invention and/or a payload, effector molecule, effector moiety such as a drug, toxin, oligonucleotide, enzyme, in close proximity of the target cell surface to which the cell-surface molecule binding-molecule can bind. Typically, the cell-surface molecule is a receptor. The binding of the cell-surface molecule binding-molecule to the target cell is followed by transfer of the saponin derivate and/or the payload, effector molecule, effector moiety, to the endosome of the cell to which the cell-surface molecule binding-molecule is bound.

An embodiment is the saponin conjugate according to the invention, wherein the first binding site of the proteinaceous molecule 1 is selected from any one or more of cell-surface molecule binding-molecules: an amino acid, a peptide, a protein, an antibody or a binding derivative or binding fragment or binding domain thereof such as a F(ab′)2 fragment, Fab′ fragment, Fab fragment, scFv, dsFv, scFv-Fc, reduced IgG (rIgG), minibody, diabody, triabody, tetrabody, Fc fusion protein, nanobody, variable V domain, a single-domain antibody (sdAb), preferably a V_{HH}, for example camelid V_{H}, or a ligand for a cell-surface molecule such as a receptor such as EGF and a cytokine, an adnectin, an affibody, an anticalin, or binding molecules comprising one or more of any of these cell-surface molecule binding-molecules. Preferred is the saponin conjugate of the invention, wherein the first binding site of the proteinaceous molecule 1 is or comprises an antibody, preferably a monoclonal antibody, such as an IgG, or a binding derivative of said antibody or binding fragment of said antibody or binding domain of said antibody, preferably the first binding site is an antibody.

Preferred is the saponin conjugate of the invention, wherein the proteinaceous molecule 1 comprises a cell-surface molecule binding-molecule comprising a first binding site for binding to a first epitope of a first cell-surface molecule, wherein said first binding site is or comprises any one or more of: a single-domain antibody (sdAb), preferably a V_{H} domain derived from a heavy chain of an antibody, preferably of immunoglobulin G origin, preferably of human origin, a V_{L} domain derived from a light chain of an antibody, preferably of immunoglobulin G origin, preferably of human origin, a V_{HH} domain such as derived from a heavy-chain only antibody (HCAb) such as from Camelidae origin or Ig-NAR origin such as a variable heavy chain new antigen receptor (VNAR) domain, preferably the HCAb is from Camelidae origin, preferably the sdAb is a V_{HH} domain derived from an HCAb from Camelidae origin (camelid V_{H}) such as derived from an HCAb from camel, lama, alpaca, dromedary, vicuna, guanaco and Bactrian camel. An embodiment is the saponin conjugate according to the invention, wherein the first binding site of the proteinaceous molecule 1 is or comprises a single-domain antibody (sdAb), preferably V_{H} domain derived from a heavy chain of an antibody, preferably of immunoglobulin G origin, preferably of human origin, a V_{L} domain derived from a light chain of an antibody, preferably of immunoglobulin G origin, preferably of human origin, a V_{HH} domain such as derived from a heavy-chain only antibody (HCAb) such as from Camelidae origin or Ig-NAR origin such as a variable heavy chain new antigen receptor (VNAR) domain, preferably the HCAb is from Camelidae origin, preferably the sdAb is a V_{HH} domain derived from an HCAb from Camelidae origin (camelid V_{H}) such as derived from an HCAb from camel, lama, alpaca, dromedary, vicuna, guanaco and Bactrian camel.

The antibodies (immunoglobulins) comprised by the saponin conjugate of the present invention may be bi- or multifunctional. For example, a bifunctional antibody has one arm having a specificity for one receptor or antigen, while the other arm recognizes a different receptor or antigen. Alternatively, each arm of the bifunctional antibody may have specificity for a different epitope of the same receptor or antigen of the target cell.

The antibodies (immunoglobulins) comprised by the saponin conjugate of the present invention may be, but are not limited to, polyclonal antibodies, monoclonal antibodies, human antibodies, humanized antibodies, chimeric antibodies, resurfaced antibodies, anti-idiotypic antibodies, mouse antibodies, rat antibodies, rat/mouse hybrid antibodies, llama antibodies, llama heavy-chain only antibodies, heavy-chain only antibodies, and veterinary antibodies. Preferably, the antibody (immunoglobulin) of the present invention is a monoclonal antibody. The resurfaced, chimeric, humanized and fully human antibodies are also more preferred because they are less likely to cause immunogenicity in humans. The antibodies of the saponin conjugate, ADC or the AOC of the present invention preferably specifically binds to an antigen expressed on the surface of a cancer cell, an autoimmune cell, a diseased cell, an aberrant cell, while leaving any healthy cell essentially unaltered (e.g. by not binding to such normal cell, or by binding to a lesser extent in number and/or affinity to such healthy cell).

An embodiment is the saponin conjugate according to the invention wherein the first epitope of the first cell-surface molecule is any one or more of: a diseased cell specific first epitope of a cell-surface receptor, a first epitope of a cell-surface receptor over-expressed on a diseased cell, an aberrant cell specific first epitope of a cell-surface receptor, a first epitope of a cell-surface receptor overexpressed on an aberrant cell, a tumor-cell specific first epitope of a first tumor-cell surface receptor, preferably of a first tumor-cell surface receptor specifically present on a tumor cell and/or overexpressed on the tumor cell. An embodiment is the saponin conjugate according to the invention, wherein the first epitope of the first cell-surface molecule to which the first binding site can bind is a tumor-cell specific first epitope of a first tumor-cell surface molecule, more preferably a tumor-cell specific first epitope of a first tumor-cell surface receptor specifically present on a tumor cell. Thus, in some embodiments, in the saponin conjugate according to the invention, the first epitope of the first cell-surface molecule, to which the proteinaceous molecule 1 can preferably bind, is a tumor-cell specific first epitope of a first tumor-cell surface molecule, more preferably a tumor-cell specific first epitope of a first tumor-cell surface receptor specifically present on a tumor cell.

Typically, in the saponin conjugate of the invention, wherein the first cell surface molecule is a first cell surface receptor, preferably an endocytic cell-surface receptor, preferably a diseased cell specific receptor or an aberrant cell specific receptor or a tumor-cell specific receptor, or a receptor overexpressed at a diseased cell, aberrant cell or tumor cell, more preferably the cell surface molecule is selected from any one or more of: CD71, CA125, EpCAM(17-1A), CD52, CEA, CD44v6, FAP, EGF-IR, integrin, syndecan-1, vascular integrin alpha-V beta-3, HER2, EGFR, CD20, CD22, Folate receptor 1, CD146, CD56, CD19, CD138, CD27L receptor, prostate specific membrane antigen (PSMA), CanAg, integrin-alphaV, CA6, CD33, mesothelin, Cripto, CD3, CD30, CD239, CD70, CD123, CD352, DLL3, CD25, ephrinA4, MUC-1, Trop2, CEACAM5, CEACAM6, HER3, CD74, PTK7, Notch3, FGF2, C4.4A, FLT3, CD38, FGFR3, CD7, PD-L1, CTLA-4, CD52, PDGFRA, VEGFR1, VEGFR2, c-Met (HGFR), EGFR1, RANKL, ADAMTS5, CD16, CXCR7 (ACKR3), glucocorticoid-induced TNFR-related protein (GITR), even more preferably the cell surface molecule is selected from: CD71, HER2, c-Met, VEGFR2, CXCR7, CD71, EGFR and EGFR1, even more preferably the cell surface molecule is any one of CD71, HER2 and EGFR, most preferably the cell surface molecule is cell surface receptor CD71.

An embodiment is the saponin conjugate according to the invention, wherein the antibody is selected from, or the sdAb is derived from or based on, any one or more of immunoglobulins: an anti-CD71 antibody such as IgG type OKT-9, an anti-HER2 antibody such as trastuzumab (Herceptin), pertuzumab, an anti-CD20 antibody such as rituximab, ofatumumab, tositumomab, obinutuzumab ibritumomab, an anti-CA125 antibody such as oregovomab, an anti-EpCAM (17-1A) antibody such as edrecolomab, an anti-EGFR antibody such as cetuximab, matuzumab, panitumumab, nimotuzumab, an anti-CD30 antibody such as brentuximab, an anti-CD33 antibody such as gemtuzumab, huMy9-6, an anti-vascular integrin alpha-v beta-3 antibody such as etaracizumab, an anti-CD52 antibody such as alemtuzumab, an anti-CD22 antibody such as epratuzumab, pinatuzumab, binding fragment (Fv) of anti-CD22 antibody moxetumomab, humanized monoclonal antibody inotuzumab, an anti-CEA antibody such as labetuzumab, an anti-CD44v6 antibody such as bivatuzumab, an anti-FAP antibody such as sibrotuzumab, an anti-CD19 antibody such as huB4, an anti-CanAg antibody such as huC242, an anti-CD56 antibody such as huN901, an anti-CD38 antibody such as daratumumab, OKT-10 anti-CD38 monoclonal antibody, an anti-CA6 antibody such as DS6, an anti-IGF-1R antibody such as cixutumumab, 3B7, an anti-integrin antibody such as CNTO 95, an anti-syndecan-1 antibody such as B-134, an anti-CD79b such as polatuzumab, an anti-HIVgp41 antibody, or an immunoglobulin with at least 95% amino-acid sequence identity with any one of these immunoglobulins, preferably at least 96%, more
preferably at least 97%, even more preferably at least 98%, even more preferably at least 99%, preferably the antibody is selected from, or the sdAb is derived from or based on any one or more of immunoglobulins: an anti-HIVgp41 antibody, an anti-CD71 antibody, an anti-HER2 antibody and an anti-EGFR antibody, or an immunoglobulin with at least 95% amino-acid sequence identity with any one of these immunoglobulins, preferably at least 96%, more preferably at least 97%, even more preferably at least 98%, even more preferably at least 99%,
more preferably the antibody is, or the sdAb is derived from or based on any one or more of: trastuzumab, pertuzumab, cetuximab, matuzumab, an anti-CD71 antibody, OKT-9, or an immunoglobulin with at least 95% amino-acid sequence identity with any one of these immunoglobulins, preferably at least 96%, more preferably at least 97%, even more preferably at least 98%, even more preferably at least 99%,
even more preferably the antibody is, or the sdAb is derived from or based on any one or more of: an anti-CD71 antibody, trastuzumab, cetuximab, the anti-CD71 antibody OKT-9, or an immunoglobulin with at least 95% amino-acid sequence identity with any one of these immunoglobulins, preferably at least 96%, more preferably at least 97%, even more preferably at least 98%, even more preferably at least 99%,
more preferably the antibody is, or the sdAb is derived from or based on any one or more of: an anti-CD71 antibody such as OKT-9, or an immunoglobulin with at least 95% amino-acid sequence identity with such immunoglobulin, preferably at least 96%, more preferably at least 97%, even more preferably at least 98%, even more preferably at least 99%, and preferably the proteinaceous molecule 1 is a monoclonal antibody, preferably an anti-CD71-antibody

An embodiment is the saponin conjugate according to the invention, wherein the cell-surface molecule targeting (binding) molecule can bind to a tumor-cell surface molecule, preferably a tumor-cell receptor such as a tumor-cell specific receptor, more preferably a receptor selected from CD71, CD63, CA125, EpCAM(17-1A), CD52, CEA, CD44v6, FAP, EGF-IR, integrin, syndecan-1, vascular integrin alpha-V beta-3, HER2, EGFR, CD20, CD22, Folate receptor 1, CD146, CD56, CD19, CD138, CD27L receptor, PSMA, CanAg, integrin-alphaV, CA6, CD33, mesothelin, Cripto, CD3, CD30, CD239, CD70, CD123, CD352, DLL3, CD25, ephrinA4, MUC1, Trop2, CEACAM5, CEACAM6, HER3, CD74, PTK7, Notch3, FGF2, C4.4A, FLT3, CD38, FGFR3, CD7, PD-L1, CTLA4, CD52, PDGFRA, VEGFR1, VEGFR2, preferably selected from CD71, HER2 and EGFR, more preferably CD71; more preferably wherein the cell-surface molecule targeting (binding) molecule is or comprises a monoclonal antibody or at least one cell-surface molecule binding fragment or—domain thereof, and preferably comprises or consists of any one of cetuximab, daratumumab, gemtuzumab, trastuzumab, panitumumab, brentuximab, inotuzumab, moxetumomab, polatuzumab, obinutuzumab, OKT-9 anti-CD71 monoclonal antibody of the IgG type, pertuzumab, rituximab, ofatumumab, Herceptin, alemtuzumab, pinatuzumab, OKT-10 anti-CD38 monoclonal antibody, an antibody of Table A2, preferably cetuximab or trastuzumab or OKT-9, or at least one cell-surface molecule binding fragment or—domain thereof.

An embodiment is the saponin conjugate wherein the first proteinaceous molecule is an antibody and wherein the saponin conjugate comprises four saponin derivative moieties according to formula (SapCon1)

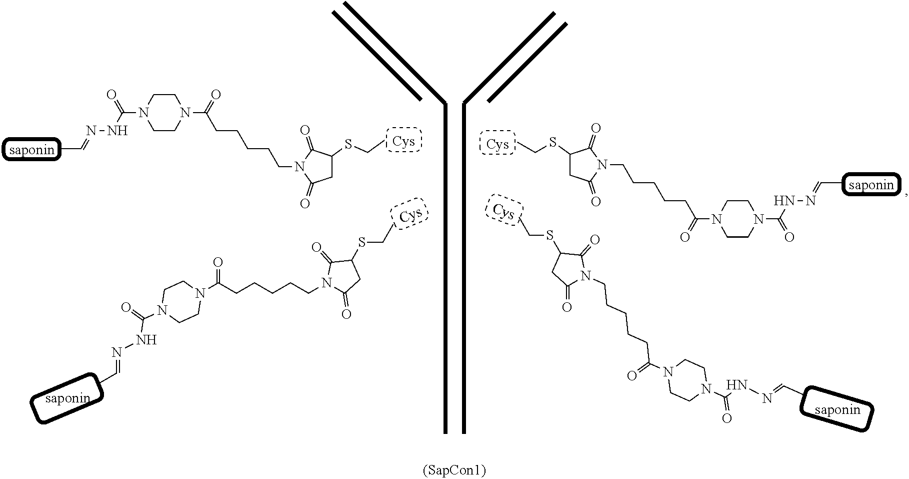 [SMILES: C/C=N/NC(=O)N1CCN(C(=O)CCCCCN2C(=O)CC(SCC)C2=O)CC1 ; C/C=N/NC(=O)N1CCN(C(=O)CCCCCN2C(=O)CC(SCC)C2=O)CC1 ; C/C=N/NC(=O)N1CCN(C(=O)CCCCCN2C(=O)CC(SCC)C2=O)CC1 ; C/C=N/NC(=O)N1CCN(C(=O)CCCCCN2C(=O)CC(SCC)C2=O)CC1]

wherein the saponin derivative comprised by the saponin conjugate is preferably based on
a) saponin selected from any one or more of list A:
   Quillaja saponaria saponin mixture, or a saponin isolated from Quillaja saponaria, for example Quil-A, QS-17-api, QS-17-xyl, QS-21, QS-21A, QS-21B, QS-7-xyl;
   Saponinum album saponin mixture, or a saponin isolated from Saponinum album;
   Saponaria officinalis saponin mixture, or a saponin isolated from Saponaria officinalis; and
   Quillaja bark saponin mixture, or a saponin isolated from Quillaja bark, for example Quil-A, QS-17-api, QS-17-xyl, QS-21, QS-21A, QS-21B, QS-7-xyl; or
b) a saponin comprising a gypsogenin aglycone core structure, selected from list B:
   SA1641, gypsoside A, NP-017772, NP-017774, NP-017777, NP-017778, NP-018109, NP-017888, NP-017889, NP-018108, SO1658 and Phytolaccagenin; or
c) a saponin comprising a quillaic acid aglycone core structure, selected from list C:
   AG1856, AG1, AG2, Agrostemmoside E, GE1741, Gypsophila saponin 1 (Gyp1), NP-017674, NP-017810, NP-003881, NP-017676, NP-017677, NP-017705, NP-017706, NP-017773, NP-017775, SA1657, Saponarioside B, SO1542, SO1584, SO1674, SO1700, SO1730, SO1772, SO1832, SO1861, SO1862, SO1904, QS1861, QS1862, QS-7, QS-7 api, QS-17, QS-18, QS-21 A-apio, QS-21 A-xylo, QS-21 B-apio and QS-21 B-xylo.

Preferably, the at least one saponin (here, four saponins) on which the saponin derivative comprised by the saponin conjugate is based, is any one or more of a saponin selected from list B or C, more preferably from list C. Preferably, the saponin is SO1861 or SO1832, more preferably SO1861. The antibody is preferably an anti-CD71 antibody or at least one sdAb capable of binding to CD71. It is preferred that the four saponin derivative moieties of the saponin conjugate are the same. The saponin derivative comprises a semicarbazone functional group.

An embodiment is the saponin conjugate or the saponin derivative according to the invention, wherein the semicarbazone functional group

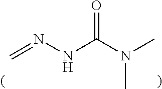

is cleavable or hydrolysable under acidic conditions, preferably at pH 4.0-6.5, such that the aldehyde group of the saponin on which the saponin derivative is based is formed upon cleavage of the semicarbazone functional group. It is specifically preferred that for the saponin derivative according to the invention and/or the saponin conjugate according to the invention, the semicarbazone functional group

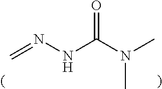

is hydrolysable (cleavable) under acidic conditions, preferably at pH 4.0-6.5, wherein hydrolysis of said semicarbazone functional group provides the aldehyde group on the aglycone core structure of the saponin on which the saponin derivative is based.

Since the semicarbazone functional group is not hydrolysed at physiological pH as apparent in e.g. the circulation and in tissue of mammals, e.g. human subjects, the saponin in the saponin conjugate is not split off from the conjugate while in the circulation or in tissue. Once the cell-surface molecule binding-molecule of the saponin conjugate, e.g. an antibody, bound to the cell-surface molecule on the target cell, the saponin conjugate is endocytosed by the cell and the saponin conjugate is transferred to and delivered into the cell endosome. In the endosome, the pH is suitable for hydrolysis (cleavage) of the semicarbazone functional group, such that the aldehyde functional group of the saponin on which the saponin conjugate is based, is again formed and the free saponin is provided. The free saponin in the endosome facilitates endosomal escape of any effector molecule or effector moiety into the cytosol, when such effector molecule or effector moiety is co-localized in the endosome. Without wishing to be bound by any theory, conjugating the saponin to a binding molecule through the semicarbazone functional group (e.g. via a maleimide linker) efficiently and sufficiently masks the cytotoxic activity of the free saponin on which the conjugate is based, by altering the initial aldehyde functional group of the saponin into the semicarbazone functional group. Once in the endosome, the semicarbazone functional group is susceptible to hydrolysis at the endosomal acidic pH, stimulating formation of the aldehyde functional group in the saponin, therewith providing the ‘active’ form of the saponin, when endosomal escape enhancing activity is concerned. Once inside the endosome of e.g. an auto-immune cell or a tumor cell, any cytotoxicity of the free saponin comprising the aldehyde functional group is not anymore hampering the benefits of the improved delivery of any effector moiety or molecule in the cytosol. That is to say, in the saponin conjugate, cytotoxicity of the saponin is efficiently blocked, inhibited or diminished, when the conjugate circulates or is present in the body extracellularly, and once inside the endosome, the formed free saponin comprising the aldehyde functional group is an efficient molecule for potentiating a desired effect of an effector molecule or moiety (that is for example co-administered to a subject in the form of for example an ADC, AOC, and capable of binding to the same cell as to which the saponin-conjugate of the invention can bind).

An embodiment is the saponin conjugate or the saponin derivative according to the invention, wherein the semicarbazone functional group is cleavable or hydrolysable in vivo under acidic conditions as present in endosomes and/or lysosomes of mammalian cells, preferably human cells, preferably at pH 4.0-6.5, and more preferably at pH 5.5, such that the aldehyde group of the saponin on which the saponin derivative is based is formed upon hydrolysis of the semicarbazone functional group. Thus, a specifically preferred embodiment is the saponin derivative according to the invention and/or the saponin conjugate according to the invention, wherein the semicarbazone functional group is subject to hydrolysis in vivo under acidic conditions as present in endosomes and/or lysosomes of mammalian cells, preferably human cells, preferably at pH 4.0-6.5, and more preferably at pH 5.5.

Surprisingly, the inventors have found that the saponin derivative according to the invention, in particular the saponin derivative according to formula (XII), (XII)a, (XII)b comprising the semicarbazone functional group hydrolyses more rapidly and in an higher amount towards the corresponding saponin comprising a “free” aldehyde functional group, i.e. native saponin, such as SO1861 or SO1832, as compared to saponin derivatives comprising a hydrazone functional group (═N—N(H)—C(O)—), such as the saponin derivative 501861-EMCH-blocked according to formula (4) (see formula (4) here below), under acidic conditions, such as at pH 4.0-6.5 which are the conditions present in endosomes and/or lysosomes of mammalian cells, such as human cells (see also the Examples section, here below). This has the benefit that a lower amount of the saponin derivatives according to the invention should be administered to obtain the same amount of saponin comprising the “free” aldehyde to act as endosomal escape enhancers for targeted toxins.

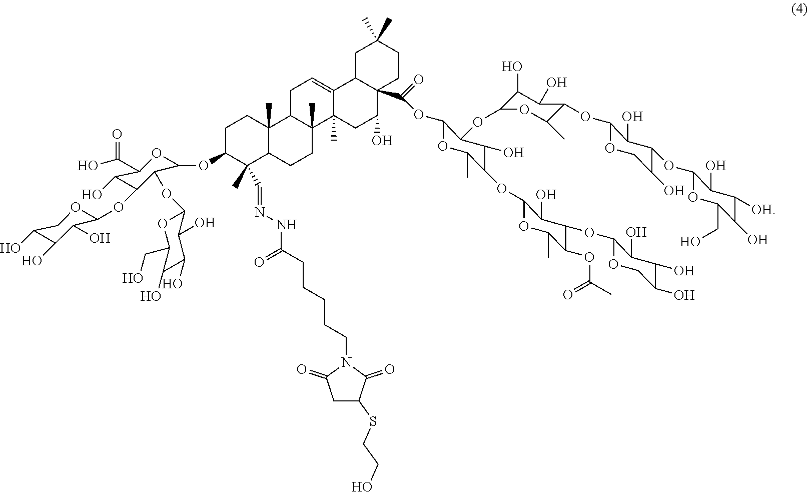

### Saponin Conjugate Comprising an Effector Moiety: 1-Component Conjugate

Preferred is the saponin conjugate according to the invention, wherein the proteinaceous molecule 1 comprises a covalently bound effector moiety, and preferably, said effector moiety is an oligonucleotide. Such a saponin conjugate comprising an effector moiety is referred to as a 1-component conjugate.

An aspect of the invention relates to a so called ‘1-component conjugate’ comprising pharmaceutical composition comprising the saponin conjugate, wherein the proteinaceous molecule 1 comprised by said conjugate comprises a covalently bound effector moiety, and preferably, said effector moiety is an oligonucleotide, and said pharmaceutical composition optionally comprising a pharmaceutically acceptable excipient and/or a pharmaceutically acceptable diluent. That is to say, the saponin conjugate comprising at least one saponin derivative, the proteinaceous molecule 1 and at least one copy of an effector molecule is referred to as a 1-component conjugate.

According to the invention is the 1-component conjugate or the pharmaceutical composition comprising the 1-component conjugate, for use as a medicament.

According to the invention is the 1-component conjugate or the pharmaceutical composition comprising the 1-component conjugate, for use in the treatment of a cardiovascular disease or hypercholesterolemia or for use in a method for lowering LDL-cholesterol in the blood of a subject, wherein the effector moiety is an oligonucleotide capable of, for example when present inside a mammalian cell and preferably when present inside a human cell, silencing gene apolipoprotein B (apoB), wherein preferably the oligonucleotide is any one of an AON such as a BNA, a xeno nucleic acid, an siRNA, an antisense oligonucleotide.

The inventors established that combining in a single conjugate, i.e. the 1-component conjugate, at least one saponin provided with a semicarbazone functional group according to the invention, a binding partner for a cell receptor such as an antibody or a binding domain or fragment thereof or such as an anti-CD71 antibody, and an effector moiety such as an oligonucleotide e.g. an AON such as a BNA for silencing the HSP27 gene or the apoB gene, provides a conjugate with a higher potency when the activity and effect of the effector moiety in the target cell is considered and compared with the potency and activity achieved with a similar conjugate, though comprising at least one saponin provided with a hydrazone functional group. Without wishing to be bound by any theory, the improved potency is related to more efficacious release of the saponin moiety from the conjugate due to higher susceptibility for bond cleavage under the slightly acidic pH condition in the endosome of the target cell, when the at least one saponin is provided with the semicarbazone functional group. Examples with an antibody-oligonucleotide conjugate (AOC) comprising at least one saponin provided with the semicarbazone functional group covalently bound to the AOC indeed show the improved gene-silencing effect of this conjugate, compared to the similar conjugate though comprising at least one saponin provided with the hydrazone functional group covalently bound to the AOC.

### Pharmaceutical Combination and Pharmaceutical Composition Comprising a Saponin Conjugate, Composition Comprising a Saponin Conjugate

An aspect of the invention relates to a composition comprising the saponin conjugate of any one of the previous aspects and embodiments relating to the saponin conjugate of the invention, the composition optionally comprising a pharmaceutically acceptable diluent and/or a pharmaceutically acceptable excipient.

Surprisingly, the inventors have found that the saponin derivatives according to the invention which are used as a component for potentiating the intracellular effect of an effector molecule or effector moiety when the effector molecule is provided as a conjugate comprising a cell-surface molecule binding-molecule such as an antibody, e.g. as an ADC or AOC, are now also suitably for (covalent) coupling to a cell-surface molecule binding-molecule such as a cell-surface molecule targeting antibody, such as an sdAb, such that by such coupling the saponin conjugate of the invention is provided, endowed with, for example, anti-tumor activity potentiating activity when used in combination with for example an ADC or an AOC.

Typically, such a composition comprising the saponin conjugate is suitable for use in combination with e.g. an ADC or an AOC. For example, the composition comprising the saponin conjugate is administered to a patient in need of administration of the ADC or AOC, before the ADC or AOC is administered, together with the ADC or AOC, or (shortly) after administration of the ADC or the AOC to the patient in need of such ADC or AOC therapy. For example, the composition comprising the saponin conjugate is mixed with a pharmaceutical composition comprising the ADC or the AOC, and a suitable dose of the mixture obtained is administered to a patient in need of ADC or AOC therapy. According to the invention, the saponin derivative comprised by the composition comprising the saponin conjugate enhances the efficacy and potency of the effector molecule comprised by such an ADC or AOC, when the saponin derivative and the ADC or AOC co-localize inside a target cell such as a tumor cell. Under influence of the saponin derivative, the effector molecule is released into the cytosol of the target cell to a higher extent, compared to contacting the same cells with the same dose of ADC or AOC in the absence of the saponin derivative. Thus, similar efficacy can be obtained at lower ADC or AOC dose when the effector molecule co-localizes inside a target cell together with the saponin derivative of the composition comprising the saponin conjugate, compared to the dose required to achieve the same efficacy in the absence of the saponin derivative inside the cell where the ADC or the AOC comprising the effector molecule is delivered.

An embodiment is the composition comprising the saponin conjugate according to the invention comprising the saponin derivative according to the invention, preferably a pharmaceutically acceptable diluent, and further comprising:
a pharmaceutically acceptable salt, preferably a pharmaceutically acceptable inorganic salt, such as an ammonium, calcium, copper, iron, magnesium, manganese, potassium, sodium, strontium, or zinc salt, preferably NaCl; and/or
a pharmaceutically acceptable buffer system, such as a phosphate, a borate, a citrate, a carbonate, a histidine, a lactate, a tromethamine, a gluconate, an aspartate, a glutamate, a tartarate, a succinate, a malate, a fumarate, an acetate and/or a ketoglutarate containing buffer system.

An embodiment is the composition comprising the saponin conjugate according to the invention comprising the saponin derivative according to the invention and a pharmaceutically acceptable diluent, preferably water, wherein the composition is liquid at a temperature of 25° C. and has a pH within the range of 2-11, preferably within the range of 4-9, more preferably within the range of 6-8.

An embodiment is the composition comprising the saponin conjugate according to the invention comprising a saponin derivative according to the invention and a pharmaceutically acceptable diluent, preferably water, wherein the composition is liquid at a temperature of 25° C. and wherein the concentration of the saponin derivative is within the range of 10⁻¹² to 1 mol/l, preferably within the range of 10⁻⁹ to 0.1 mol/l, more preferably within the range of 10⁻⁶ to 0.1 mol/l.

An aspect of the invention relates to a first pharmaceutical combination comprising:
(a) the composition of the invention comprising the saponin conjugate of any one of the previous aspects and embodiments relating to the saponin conjugate of the invention; and
(b) a first pharmaceutical composition comprising a covalently bound conjugate comprising a cell-surface molecule binding-molecule, such as a second proteinaceous molecule (′proteinaceous molecule 2′), and an effector moiety, wherein the proteinaceous molecule 2 is the same or different from the proteinaceous molecule 1 present in the saponin conjugate, and if the proteinaceous molecule 2 is different from the proteinaceous molecule 1, the proteinaceous molecule 2 comprising a second binding site for binding to a second epitope of a second cell-surface molecule, wherein the second cell-surface molecule is the same as or different from the first cell surface molecule, and if the second cell-surface molecule is different from the first cell surface molecule, the second cell-surface molecule and the first cell surface molecule are preferably present on the same cell, the first pharmaceutical composition optionally further comprising a pharmaceutically acceptable excipient and/or a pharmaceutically acceptable diluent.

The effector moiety is not a saponin on which the saponin derivative or the saponin conjugate of the invention are based. The effector moiety is not the saponin derivative or the saponin conjugate of the invention.

An embodiment is the first pharmaceutical combination according to the invention comprising the composition comprising the saponin conjugate according to the invention and the first pharmaceutical composition, wherein said composition comprising the saponin conjugate and/or said first pharmaceutical composition preferably comprises a pharmaceutically acceptable diluent, and further comprising:
a pharmaceutically acceptable salt, preferably a pharmaceutically acceptable inorganic salt, such as an ammonium, calcium, copper, iron, magnesium, manganese, potassium, sodium, strontium, or zinc salt, preferably NaCl; and/or
a pharmaceutically acceptable buffer system, such as a phosphate, a borate, a citrate, a carbonate, a histidine, a lactate, a tromethamine, a gluconate, an aspartate, a glutamate, a tartarate, a succinate, a malate, a fumarate, an acetate and/or a ketoglutarate containing buffer system.

An embodiment is the first pharmaceutical combination according to the invention comprising the composition comprising the saponin conjugate according to the invention and the first pharmaceutical composition, wherein said composition comprising the saponin conjugate and/or said first pharmaceutical composition preferably comprises a pharmaceutically acceptable diluent, preferably water, wherein the composition(s) is/are liquid at a temperature of 25° C. and has/have a pH within the range of 2-11, preferably within the range of 4-9, more preferably within the range of 6-8.

An embodiment is the first pharmaceutical composition of the invention, wherein the second proteinaceous molecule is selected from the proteinaceous molecules according to the invention and as listed here above.

An embodiment is the first pharmaceutical composition of the invention, wherein the saponin conjugate is the saponin conjugate according to formula (XII).

An aspect of the invention relates to a second pharmaceutical combination, comprising:
(a) the composition of the invention comprising the saponin conjugate of any one of the previous aspects and embodiments relating to the saponin conjugate of the invention; and
(b) a second pharmaceutical composition comprising a covalently bound conjugate comprising a cell-surface molecule binding-molecule, such as a third proteinaceous molecule (′proteinaceous molecule 3′), and an effector moiety, wherein the proteinaceous molecule 3 comprises the first binding site for binding to the first epitope on the cell-surface molecule according to the invention, the second pharmaceutical composition optionally further comprising a pharmaceutically acceptable excipient and/or a pharmaceutically acceptable diluent,
wherein the first binding site of the proteinaceous molecule 1 and the first binding site of the proteinaceous molecule 3 are the same, and wherein the first cell-surface molecule and the first epitope on the first cell-surface molecule, to which the proteinaceous molecule 1 can bind, and the first cell-surface molecule and the first epitope on the first cell-surface molecule, to which the proteinaceous molecule 3 can bind, are the same.

An embodiment is the second pharmaceutical combination of the invention, wherein the third proteinaceous molecule is selected from the proteinaceous molecules according to the invention. That is to say, the third proteinaceous molecule and the first proteinaceous molecule can be the same.

The effector moiety is not a saponin on which the saponin derivative or the saponin conjugate of the invention are based. The effector moiety is not the saponin derivative or the saponin conjugate of the invention.

An embodiment is the second pharmaceutical combination according to the invention comprising the composition comprising the saponin conjugate according to the invention and the second pharmaceutical composition, wherein said composition comprising the saponin conjugate and/or said second pharmaceutical composition preferably comprises a pharmaceutically acceptable diluent, and further comprising:
a pharmaceutically acceptable salt, preferably a pharmaceutically acceptable inorganic salt, such as an ammonium, calcium, copper, iron, magnesium, manganese, potassium, sodium, strontium, or zinc salt, preferably NaCl; and/or
a pharmaceutically acceptable buffer system, such as a phosphate, a borate, a citrate, a carbonate, a histidine, a lactate, a tromethamine, a gluconate, an aspartate, a glutamate, a tartarate, a succinate, a malate, a fumarate, an acetate and/or a ketoglutarate containing buffer system.

An embodiment is the second pharmaceutical combination according to the invention comprising the composition comprising the saponin conjugate according to the invention and the second pharmaceutical composition, wherein said composition comprising the saponin conjugate and/or said second pharmaceutical composition preferably comprises a pharmaceutically acceptable diluent, preferably water, wherein the composition(s) is/are liquid at a temperature of 25° C. and has/have a pH within the range of 2-11, preferably within the range of 4-9, more preferably within the range of 6-8.

An aspect of the invention relates to a third pharmaceutical composition comprising:
(a) the saponin conjugate of the invention;
   and comprising
   either
(b1) the conjugate comprising proteinaceous molecule 2 and an effector moiety, as detailed here above (i.e., conjugate comprising a cell-surface molecule binding-molecule, such as a second proteinaceous molecule (‘proteinaceous molecule 2’), and an effector moiety, wherein the proteinaceous molecule 2 is the same or different from the proteinaceous molecule 1 present in the saponin conjugate, and if the proteinaceous molecule 2 is different from the proteinaceous molecule 1, the proteinaceous molecule 2 comprising a second binding site for binding to a second epitope of a second cell-surface molecule, wherein the second cell-surface molecule is the same as or different from the first cell surface molecule, and if the second cell-surface molecule is different from the first cell surface molecule, the second cell-surface molecule and the first cell surface molecule are preferably present on the same cell, wherein optionally the second proteinaceous molecule is selected from the proteinaceous molecules according to the invention and as listed here above)
   or
(b2) the conjugate comprising proteinaceous molecule 3 and an effector moiety, as detailed here above (i.e., conjugate comprising a cell-surface molecule binding-molecule, such as a third proteinaceous molecule (‘proteinaceous molecule 3’), and an effector moiety, wherein the proteinaceous molecule 3 comprises the first binding site for binding to the first epitope on the cell-surface molecule according to the invention, wherein the first binding site of the proteinaceous molecule 1 and the first binding site of the proteinaceous molecule 3 are the same, and wherein the first cell-surface molecule and the first epitope on the first cell-surface molecule, to which the proteinaceous molecule 1 can bind, and the first cell-surface molecule and the first epitope on the first cell-surface molecule, to which the proteinaceous molecule 3 can bind, are the same, wherein optionally the third proteinaceous molecule is selected from the proteinaceous molecules according to the invention and as listed here above),
   and the third pharmaceutical composition optionally comprising a pharmaceutically acceptable excipient and/or a pharmaceutically acceptable diluent.

The effector moiety is not a saponin on which the saponin derivative or the saponin conjugate of the invention are based. The effector moiety is not the saponin derivative or the saponin conjugate of the invention.

An embodiment is the third pharmaceutical composition according to the invention comprising a pharmaceutically acceptable diluent, and further comprising:
a pharmaceutically acceptable salt, preferably a pharmaceutically acceptable inorganic salt, such as an ammonium, calcium, copper, iron, magnesium, manganese, potassium, sodium, strontium, or zinc salt, preferably NaCl; and/or
a pharmaceutically acceptable buffer system, such as a phosphate, a borate, a citrate, a carbonate, a histidine, a lactate, a tromethamine, a gluconate, an aspartate, a glutamate, a tartarate, a succinate, a malate, a fumarate, an acetate and/or a ketoglutarate containing buffer system.

An embodiment is the third pharmaceutical composition according to the invention preferably comprising a pharmaceutically acceptable diluent, preferably water, wherein the composition(s) is/are liquid at a temperature of 25° C. and has/have a pH within the range of 2-11, preferably within the range of 4-9, more preferably within the range of 6-8.

An embodiment is the first pharmaceutical combination or the second pharmaceutical combination or the third pharmaceutical composition according to the invention, wherein the proteinaceous molecule 2 and the proteinaceous molecule 3 is of a type as detailed for the proteinaceous molecule 1, e.g. an antibody or an antigen binding fragment or antigen binding domain thereof, a ligand for a cell-surface molecule or cell-surface receptor such as EGF or a cytokine, an scFv, a Fab, a binding molecule comprising an sdAb, such as a V_{HH}, a binding molecule comprising any of an adnectin, an anticalin, an affibody. The skilled person will appreciate that for the proteinaceous molecule 1 and proteinaceous molecule 2 and proteinaceous molecule 3 any cell-surface molecule binding-molecule can be selected and is suitable for application in the saponin conjugates of the invention and for application in the pharmaceutical compositions and pharmaceutical combinations of the invention, that is known today in the technological field of (specifically) targeting a mammalian (diseased-, aberrant-, tumor-, auto-immune-, etc.) cell with a binding molecule, e.g. for (targeted and specific) delivery of an effector molecule or effector moiety conjugated to the cell-surface molecule binding-molecule. Typical examples of such suitable cell-surface molecule binding-molecules are the antibodies and binding fragments, binding domains and binding derivatives thereof and ligands such as EGF or cytokines used today to deliver drug molecules, payloads, effector moieties, protein toxins, small-molecule toxins, enzymes, oligonucleotides, etc., etc., e.g. in ADCs and AOCs. Any cell-surface molecule binding-molecule applicable for e.g. designing and providing ADCs and AOCs is typically also applicable for providing the saponin conjugate of the invention (e.g. proteinaceous molecule 1). Any cell-surface molecule binding-molecule applicable for e.g. designing and providing ADCs and AOCs is typically also applicable for providing the cell-surface molecule binding-molecule conjugated with an effector moiety, e.g. the proteinaceous molecule 3 and proteinaceous molecule 2 conjugated to an effector moiety. The skilled person will also appreciate that it is within the scope of the invention that for the proteinaceous molecule 1 and proteinaceous molecule 2 and proteinaceous molecule 3 any cell-surface molecule binding-molecule can be selected and is suitable for application in the saponin conjugates of the invention and for application in the pharmaceutical compositions and pharmaceutical combinations of the invention, that is known today in the technological field of (specifically) targeting a mammalian (diseased-, aberrant-, tumor-, auto-immune-, etc.) cell with a binding molecule, e.g. for (targeted and specific) delivery of an effector molecule or effector moiety conjugated to the cell-surface molecule binding-molecule, wherein the cell-surface molecule binding-molecule is either a proteinaceous molecule or a non-proteinaceous molecule. That is to say, according to the invention, any of the cell-surface molecule binding-molecules proteinaceous molecule 1, proteinaceous molecule 2, proteinaceous molecule 3 can be replaced by a non-proteinaceous cell-surface molecule binding-molecule suitable for targeting (binding to) a selected and desired cell such as a mammalian cell, e.g. a tumor cell or an auto-immune cell and/or a diseased cell and/or an aberrant cell. According to the invention, it is preferred that the cell-surface molecules to which combinations of the proteinaceous molecule 1, proteinaceous molecule 2, proteinaceous molecule 3 bind, are present at the surface of the same target cell, when combinations of proteinaceous molecule 1, proteinaceous molecule 2 and proteinaceous molecule 3 bind to different cell-surface molecules. For example, according to the invention, if in pharmaceutical combinations and pharmaceutical compositions proteinaceous molecule 1 binds to HER2 and proteinaceous molecule 2 binds to CD71 or EGFR, HER2 and CD71 or HER2 and EGFR are typically present at the same cell.

An embodiment is the first pharmaceutical combination or the second pharmaceutical combination or the third pharmaceutical composition according to the invention, wherein the second binding site of the proteinaceous molecule 2 and the first binding site of the proteinaceous molecule 3 is/are or comprise(s) a single-domain antibody (sdAb), preferably V_{H} domain derived from a heavy chain of an antibody, preferably of immunoglobulin G origin, preferably of human origin, a V_{L} domain derived from a light chain of an antibody, preferably of immunoglobulin G origin, preferably of human origin, a V_{HH} domain such as derived from a heavy-chain only antibody (HCAb) such as from Camelidae origin or Ig-NAR origin such as a variable heavy chain new antigen receptor (VNAR) domain, preferably the HCAb is from Camelidae origin, preferably the sdAb is a V_{HH} domain derived from an HCAb from Camelidae origin (camelid V_{H}) such as derived from an HCAb from camel, lama, alpaca, dromedary, vicuna, guanaco and Bactrian camel.

An embodiment is the first pharmaceutical combination or the second pharmaceutical combination or the third pharmaceutical composition according to the invention, wherein the second epitope of the second cell-surface molecule to which the second binding site can bind is a tumor-cell specific second epitope of a second tumor-cell surface molecule, more preferably a tumor-cell specific second epitope of a second tumor-cell surface receptor specifically present on a tumor cell, and wherein the first epitope of the first cell-surface molecule is a tumor-cell specific first epitope of a first tumor-cell surface molecule, more preferably a tumor-cell specific first epitope of a first tumor-cell surface receptor specifically present on a tumor cell, wherein the second cell-surface molecule and the first cell-surface molecule are present on the same cell.

An embodiment is the first pharmaceutical combination or the second pharmaceutical combination or the third pharmaceutical composition according to the invention, wherein the second and third cell-surface molecule binding (targeting) molecule can bind to a tumor-cell surface molecule, preferably a tumor-cell receptor such as a tumor-cell specific receptor, more preferably a receptor selected from CD71, CD63, CA125, EpCAM(17-1A), CD52, CEA, CD44v6, FAP, EGF-IR, integrin, syndecan-1, vascular integrin alpha-V beta-3, HER2, EGFR, CD20, CD22, Folate receptor 1, CD146, CD56, CD19, CD138, CD27L receptor, PSMA, CanAg, integrin-alphaV, CA6, CD33, mesothelin, Cripto, CD3, CD30, CD239, CD70, CD123, CD352, DLL3, CD25, ephrinA4, MUC1, Trop2, CEACAM5, CEACAM6, HER3, CD74, PTK7, Notch3, FGF2, C4.4A, FLT3, CD38, FGFR3, CD7, PD-L1, CTLA4, CD52, PDGFRA, VEGFR1, VEGFR2, preferably selected from CD71, HER2 and EGFR, more preferably wherein the second and third cell-surface molecule binding molecule is or comprises a monoclonal antibody or at least one cell-surface molecule binding fragment or—domain thereof, and preferably comprises or consists of any one of cetuximab, daratumumab, gemtuzumab, trastuzumab, panitumumab, brentuximab, inotuzumab, moxetumomab, polatuzumab, obinutuzumab, OKT-9 anti-CD71 monoclonal antibody of the IgG type, pertuzumab, rituximab, ofatumumab, Herceptin, alemtuzumab, pinatuzumab, OKT-10 anti-CD38 monoclonal antibody, an antibody of Table A2, preferably cetuximab or trastuzumab or OKT-9, or at least one cell-surface molecule binding fragment or—domain thereof.

Preferred are the first pharmaceutical combination or second pharmaceutical combination or the third pharmaceutical composition according to the invention, wherein the second binding site of the proteinaceous molecule 2 and/or the first binding site of the proteinaceous molecule 3 comprises or consists of an antibody or a binding derivative or binding fragment or binding domain thereof such as a F(ab′)2 fragment, Fab′ fragment, Fab fragment, scFv, dsFv, scFv-Fc, reduced IgG (rIgG), minibody, diabody, triabody, tetrabody, Fc fusion protein, nanobody, variable V domain, a single-domain antibody (sdAb), preferably a V_{HH}, for example camelid V_{H}, or a ligand for a cell-surface molecule such as a receptor such as EGF and a cytokine, preferably V_{H} domain derived from a heavy chain of an antibody, preferably of immunoglobulin G origin, preferably of human origin, a V_{L} domain derived from a light chain of an antibody, preferably of immunoglobulin G origin, preferably of human origin, a V_{HH} domain such as derived from a heavy-chain only antibody (HCAb) such as from Camelidae origin or Ig-NAR origin such as a variable heavy chain new antigen receptor (VNAR) domain, preferably the HCAb is from Camelidae origin, preferably the sdAb is a V_{HH} domain derived from an HCAb from Camelidae origin (camelid V_{H}) such as derived from an HCAb from camel, lama, alpaca, dromedary, vicuna, guanaco and Bactrian camel.

An aspect of the invention relates to a pharmaceutical composition comprising:
(a) the saponin conjugate of the invention; and
(b) the conjugate comprising proteinaceous molecule 2 and an effector moiety of the invention, or the conjugate comprising proteinaceous molecule 3 and an effector moiety of the invention, and optionally further comprising a pharmaceutically acceptable excipient and/or a pharmaceutically acceptable diluent. The effector moiety is not a saponin on which the saponin derivative or the saponin conjugate of the invention are based. The effector moiety is not the saponin derivative or the saponin conjugate of the invention.

Preferred are the first pharmaceutical combination or second pharmaceutical combination or the third pharmaceutical composition of the invention, or the pharmaceutical composition comprising the 1-component conjugate of the invention, wherein the effector moiety is an oligonucleotide.

An embodiment is the first pharmaceutical combination or second pharmaceutical combination or the third pharmaceutical composition of the invention, or the pharmaceutical composition comprising the 1-component conjugate of the invention, wherein the effector moiety that is comprised by the conjugate comprising a cell-surface molecule binding-molecule, such as a second or third proteinaceous molecule (‘proteinaceous molecule 2’, ‘proteinaceous molecule 3’), and an effector moiety or that is comprised by the conjugate comprising a cell-surface molecule binding-molecule, such as a third proteinaceous molecule (‘proteinaceous molecule 3’), and an effector moiety, comprises or consists of any one or more of: an oligonucleotide, a nucleic acid and a xeno nucleic acid, preferably selected from any one or more of a vector, a gene, a cell suicide inducing transgene, deoxyribonucleic acid (DNA), ribonucleic acid (RNA), anti-sense oligonucleotide (ASO, AON), short interfering RNA (siRNA), microRNA (miRNA), DNA aptamer, RNA aptamer, mRNA, mini-circle DNA, peptide nucleic acid (PNA), phosphoramidate morpholino oligomer (PMO), locked nucleic acid (LNA), bridged nucleic acid (BNA), 2′-deoxy-2′-fluoroarabino nucleic acid (FANA), 2′-O-methoxyethyl-RNA (MOE), 2′-O,4′-aminoethylene bridged nucleic acid, 3′-fluoro hexitol nucleic acid (FHNA), a plasmid, glycol nucleic acid (GNA) and threose nucleic acid (TNA), or a derivative thereof, more preferably a BNA, for example a BNA for silencing HSP27 or apoB protein expression.

An embodiment is the first pharmaceutical combination or second pharmaceutical combination or the third pharmaceutical composition of the invention, or the pharmaceutical composition comprising the 1-component conjugate of the invention, wherein the effector moiety is an oligonucleotide selected from deoxyribonucleic acid (DNA) oligomer, ribonucleic acid (RNA) oligomer, anti-sense oligonucleotide (ASO, AON), short interfering RNA (siRNA), microRNA (miRNA), anti-microRNA (anti-miRNA), DNA aptamer, RNA aptamer, mRNA, mini-circle DNA, peptide nucleic acid (PNA), phosphoramidate morpholino oligomer (PMO), locked nucleic acid (LNA), bridged nucleic acid (BNA), 2′-deoxy-2′-fluoroarabino nucleic acid (FANA), 2′-O-methoxyethyl-RNA (MOE), 3′-fluoro hexitol nucleic acid (FHNA), glycol nucleic acid (GNA), xeno nucleic acid oligonucleotide and threose nucleic acid (TNA).

An embodiment is the first pharmaceutical combination or second pharmaceutical combination or the third pharmaceutical composition of the invention, or the pharmaceutical composition comprising the 1-component conjugate of the invention, wherein the oligonucleotide is selected from any one or more of a(n): short interfering RNA (siRNA), short hairpin RNA (shRNA), anti-hairpin-shaped microRNA (miRNA), single-stranded RNA, aptamer RNA, double-stranded RNA (dsRNA), microRNA (miRNA), anti-microRNA (anti-miRNA, anti-miR), antisense oligonucleotide (ASO), mRNA, DNA, antisense DNA, locked nucleic acid (LNA), bridged nucleic acid (BNA), 2′-O,4′-aminoethylene bridged nucleic Acid (BNA Nc), BNA-based siRNA, and BNA-based antisense oligonucleotide (BNA-AON).

An embodiment is the first pharmaceutical combination or second pharmaceutical combination or the third pharmaceutical composition of the invention, or the pharmaceutical composition comprising the 1-component conjugate of the invention, wherein the effector moiety is an oligonucleotide selected from any one of an anti-miRNA, a BNA-AON or an siRNA, such as BNA-based siRNA, preferably selected from chemically modified siRNA, metabolically stable siRNA and chemically modified, metabolically stable siRNA.

An embodiment is the first pharmaceutical combination or second pharmaceutical combination or the third pharmaceutical composition of the invention, or the pharmaceutical composition comprising the 1-component conjugate of the invention, wherein the oligonucleotide is an oligonucleotide that is capable of silencing a gene, when present in a cell comprising such gene, and/or is capable of targeting an aberrant miRNA when present in a cell comprising such aberrant miRNA.

An embodiment is the first pharmaceutical combination or second pharmaceutical combination or the third pharmaceutical composition of the invention, or the pharmaceutical composition comprising the 1-component conjugate of the invention, wherein the oligonucleotide is an oligonucleotide that is capable of targeting an mRNA, when present in a cell comprising such mRNA, or wherein the oligonucleotide is an oligonucleotide that is capable of antagonizing or restoring an miRNA function such as inhibiting an oncogenic miRNA (onco-miR) or suppression of expression of an onco-miR, when present in a cell comprising such an miRNA.

An embodiment is the first pharmaceutical combination or second pharmaceutical combination or the third pharmaceutical composition of the invention, or the pharmaceutical composition comprising the 1-component conjugate of the invention, wherein the effector moiety that is comprised by the conjugate comprising a cell-surface molecule binding-molecule, such as a second proteinaceous molecule (‘proteinaceous molecule 2’), and an effector moiety or that is comprised by the conjugate comprising a cell-surface molecule binding-molecule, such as a third proteinaceous molecule (‘proteinaceous molecule 3’), and an effector moiety, comprises or consists of any one or more of: at least one proteinaceous molecule, preferably selected from any one or more of a peptide, a protein, protein toxin, an enzyme such as urease and Cre-recombinase, a ribosome-inactivating protein, and more preferably selected from any one or more of a viral toxin such as apoptin; a bacterial toxin such as Shiga toxin, Shiga-like toxin, Pseudomonas aeruginosa exotoxin (PE) or exotoxin A of PE, full-length or truncated diphtheria toxin (DT), cholera toxin; a fungal toxin such as alpha-sarcin; a plant toxin including ribosome-inactivating proteins and the A chain of type 2 ribosome-inactivating proteins such as dianthin e.g. dianthin-30 or dianthin-32, saporin e.g. saporin-S3 or saporin-S6, bouganin or de-immunized derivative debouganin of bouganin, shiga-like toxin A, pokeweed antiviral protein, ricin, ricin A chain, modeccin, modeccin A chain, abrin, abrin A chain, volkensin, volkensin A chain, viscumin, viscumin A chain; or an animal or human toxin such as frog RNase, or granzyme B or angiogenin from humans, or any fragment or derivative thereof; preferably the protein toxin is dianthin and/or saporin.

An embodiment is the first pharmaceutical combination or second pharmaceutical combination or the third pharmaceutical composition of the invention, or the pharmaceutical composition comprising the 1-component conjugate of the invention, wherein the effector moiety is a toxin.

An embodiment is the first pharmaceutical combination or second pharmaceutical combination or the third pharmaceutical composition of the invention, or the pharmaceutical composition comprising the 1-component conjugate of the invention, wherein the toxin is selected from: a viral toxin, a bacterial toxin, a plant toxin including ribosome-inactivating proteins and the A chain of type 2 ribosome-inactivating proteins, an animal toxin, a human toxin and a fungal toxin, more preferably the toxin is a plant toxin including ribosome-inactivating proteins and the A chain of type 2 ribosome-inactivating proteins.

An embodiment is the first pharmaceutical combination or second pharmaceutical combination or the third pharmaceutical composition of the invention, or the pharmaceutical composition comprising the 1-component conjugate of the invention, wherein the toxin is selected from the list consisting of: apoptin, Shiga toxin, Shiga-like toxin, Pseudomonas aeruginosa exotoxin (PE), full-length or truncated diphtheria toxin (DT), cholera toxin, alpha-sarcin, dianthin, saporin, bouganin, de-immunized derivative debouganin of bouganin, shiga-like toxin A, pokeweed antiviral protein, ricin, ricin A chain, modeccin, modeccin A chain, abrin, abrin A chain, volkensin, volkensin A chain, viscumin, viscumin A chain, frog RNase, granzyme B, human angiogenin; preferably the toxin is dianthin and/or saporin.

An embodiment is the first pharmaceutical combination or second pharmaceutical combination or the third pharmaceutical composition of the invention, or the pharmaceutical composition comprising the 1-component conjugate of the invention, wherein the effector moiety that is comprised by the conjugate comprising a cell-surface molecule binding-molecule, such as a second proteinaceous molecule (‘proteinaceous molecule 2’), and an effector moiety or that is comprised by the conjugate comprising a cell-surface molecule binding-molecule, such as a third proteinaceous molecule (‘proteinaceous molecule 3’), and an effector moiety, comprises or consists of any one or more of: at least one payload, preferably selected from any one or more of a toxin targeting ribosomes, a toxin targeting elongation factors, a toxin targeting tubulin, a toxin targeting DNA and a toxin targeting RNA, more preferably any one or more of emtansine, pasudotox, maytansinoid derivative DM1, maytansinoid derivative DM4, monomethyl auristatin E (MMAE, vedotin), monomethyl auristatin F (MMAF, mafodotin), a Calicheamicin, N-Acetyl-γ-calicheamicin, a pyrrolobenzodiazepine (PBD) dimer, a benzodiazepine, a CC-1065 analogue, a duocarmycin, Doxorubicin, paclitaxel, docetaxel, cisplatin, cyclophosphamide, etoposide, docetaxel, 5-fluorouracyl (5-FU), mitoxantrone, a tubulysin, an indolinobenzodiazepine, AZ13599185, a cryptophycin, rhizoxin, methotrexate, an anthracycline, a camptothecin analogue, SN-38, DX-8951f, exatecan mesylate, truncated form of Pseudomonas aeruginosa exotoxin (PE38), a Duocarmycin derivative, an amanitin, α-amanitin, a spliceostatin, a thailanstatin, ozogamicin, tesirine, Amberstatin269 and soravtansine, or a derivative thereof.

An embodiment is the first pharmaceutical combination or second pharmaceutical combination or the third pharmaceutical composition of the invention, or the pharmaceutical composition comprising the 1-component conjugate of the invention, wherein the payload or the toxin is selected from the list consisting of: apoptin, Shiga toxin, Shiga-like toxin, Pseudomonas aeruginosa exotoxin (PE), full-length or truncated diphtheria toxin (DT), cholera toxin, alpha-sarcin, dianthin, saporin, bouganin, de-immunized derivative debouganin of bouganin, shiga-like toxin A, pokeweed antiviral protein, ricin, ricin A chain, modeccin, modeccin A chain, abrin, abrin A chain, volkensin, volkensin A chain, viscumin, viscumin A chain, frog RNase, granzyme B, human angiogenin; preferably the toxin is dianthin and/or saporin.

An embodiment is the first pharmaceutical combination or second pharmaceutical combination or the third pharmaceutical composition of the invention, or the pharmaceutical composition comprising the 1-component conjugate of the invention, wherein the payload or the toxin is selected from: a toxin targeting ribosomes, a toxin targeting elongation factors, a toxin targeting tubulin, a toxin targeting DNA and a toxin targeting RNA, more preferably the toxin is selected from the list consisting of: emtansine, pasudotox, maytansinoid derivative DM1, maytansinoid derivative DM4, monomethyl auristatin E (MMAE, vedotin), monomethyl auristatin F (MMAF, mafodotin), a Calicheamicin, N-Acetyl-γ-calicheamicin, a pyrrolobenzodiazepine (PBD) dimer, a benzodiazepine, a CC-1065 analogue, a duocarmycin, Doxorubicin, paclitaxel, docetaxel, cisplatin, cyclophosphamide, etoposide, docetaxel, 5-fluorouracyl (5-FU), mitoxantrone, a tubulysin, an indolinobenzodiazepine, AZ13599185, a cryptophycin, rhizoxin, methotrexate, an anthracycline, a camptothecin analogue, SN-38, DX-8951f, exatecan mesylate, truncated form of Pseudomonas aeruginosa exotoxin (PE38), a Duocarmycin derivative, an amanitin, α-amanitin, a spliceostatin, a thailanstatin, ozogamicin, tesirine, Amberstatin269 and soravtansine.

An embodiment is the first pharmaceutical combination or second pharmaceutical combination or the third pharmaceutical composition of the invention, or the pharmaceutical composition comprising the 1-component conjugate of the invention, wherein the effector moiety is an enzyme, such as urease or Cre-recombinase.

An embodiment is the first pharmaceutical combination or second pharmaceutical combination or the third pharmaceutical composition of the invention, or the pharmaceutical composition comprising the 1-component conjugate of the invention, wherein the effector molecule is a drug molecule.

An embodiment is the first pharmaceutical combination or second pharmaceutical combination or the third pharmaceutical composition of the invention, or the pharmaceutical composition comprising the 1-component conjugate of the invention, wherein the covalently bound conjugate or the 1-component conjugate comprises 1-16 effector moieties, preferably oligonucleotide(s), preferably 1-4 effector moieties, most preferably 1 effector moiety, wherein the effector moiety is preferably covalently bound in the conjugate via a cleavable bond, preferably an acid-labile cleavable bond that is cleaved under acidic conditions such as for example present in endosomes and/or lysosomes of mammalian cells, preferably human cells such as a diseased cell, an aberrant cell and a tumor cell, preferably at pH 4.0-6.5, and more preferably at pH<5.5, wherein preferably the cleavable bond is a hydrazone bond or a semicarbazone bond, more preferably a semicarbazone bond.

An aspect of the invention relates to the first pharmaceutical combination of the invention, the second pharmaceutical combination of the invention or the third pharmaceutical composition of the invention, or to the so-called ‘1-component’ conjugate comprising the saponin derivative of the invention, the cell-targeting ligand (antibody) according to the invention and an effector moiety according to the invention, for use as a medicament, preferably in a human patient.

An aspect of the invention relates to the first pharmaceutical combination of the invention, the second pharmaceutical combination of the invention or the third pharmaceutical composition of the invention, or to the 1-component conjugate of the invention, for use in the treatment or prevention of a disease or health problem related to presence of the diseased cell according to the invention, preferably in a human patient, preferably wherein the disease or health problem related to presence of the diseased cell is related to a gene defect in the diseased cell and/or is related to expression or overexpression of a protein in the diseased cell. Examples of suitable target cell-surface receptors to which the saponin conjugate of the invention can bind are HER2, EGFR and CD71. Therefore, these targets are preferred. More preferred is CD71.

An aspect of the invention relates to the first pharmaceutical combination of the invention, the second pharmaceutical combination of the invention or the third pharmaceutical composition of the invention, or to the 1-component conjugate of the invention, for use in the treatment or prevention of a disease or health problem related to the presence of the aberrant cell according to the invention, preferably in a human patient, preferably wherein the disease or health problem related to presence of the aberrant cell is related to a gene defect in the aberrant cell and/or is related to expression or overexpression of a protein in the aberrant cell. Examples of suitable target cell-surface receptors to which the saponin conjugate of the invention can bind are HER2, EGFR and CD71. Therefore, these targets are preferred. More preferred is CD71.

The first pharmaceutical combination of the invention, the second pharmaceutical combination of the invention or the third pharmaceutical composition of the invention, or to the 1-component conjugate of the invention, are suitable for treatment of a cancer or for prophylaxis of a cancer. An example of such a cancer is a carcinoma. An example of such a cancer is a melanoma. An example of such a cancer is a melanoma selected from any one or more of: a breast cancer such as a breast carcinoma such as adenocarcinoma or metastatic adenocarcinoma in the breast. A further example of such a cancer is a cervical cancer such as a cervical carcinoma such as cervical epidermoid carcinoma. A further example of such a cancer is a skin cancer such as a skin carcinoma such as epidermoid carcinoma, or such as skin melanoma

An aspect of the invention relates to the first pharmaceutical combination of the invention, the second pharmaceutical combination of the invention or the third pharmaceutical composition of the invention, or to the 1-component conjugate of the invention, for use in the treatment or prevention of a cancer, preferably in a human patient, such as a cancer selected from any one or more of a carcinoma and a melanoma, for example selected from any one or more of: a breast cancer such as a breast carcinoma such as adenocarcinoma or metastatic adenocarcinoma in the breast; a cervical cancer such as a cervical carcinoma such as cervical epidermoid carcinoma; a skin cancer such as a skin carcinoma such as epidermoid carcinoma, or such as skin melanoma, preferably wherein the cancer is related to a gene defect in a tumor cell and/or is related to expression or overexpression of a protein in a tumor cell. Examples of suitable target cell-surface receptors to which the saponin conjugate of the invention can bind are HER2, EGFR and CD71. Therefore, these targets are preferred. More preferred is CD71.

An aspect of the invention relates to the first pharmaceutical combination of the invention, the second pharmaceutical combination of the invention or the third pharmaceutical composition of the invention, or to the 1-component conjugate of the invention, for use in the treatment or prevention of an autoimmune disease such as rheumatoid arthritis, preferably in a human patient, preferably wherein the autoimmune disease is related to a gene defect in an aberrant cell and/or is related to expression or overexpression of a protein in an aberrant cell.

An aspect of the invention relates to the first pharmaceutical combination of the invention, the second pharmaceutical combination of the invention or the third pharmaceutical composition of the invention, or to the 1-component conjugate of the invention, for use in the treatment or prevention of a disease or health problem relating to any one or more of: expression or over-expression of a protein, presence of a mutant gene, a gene defect, a mutant protein, absence of a functional protein, presence of a dys-functional protein and a functional protein deficiency.

An aspect of the invention relates to the first pharmaceutical combination of the invention, the second pharmaceutical combination of the invention or the third pharmaceutical composition of the invention, or to the 1-component conjugate of the invention, for use according to the invention, i.e., for use in the treatment or prevention of a disease or health problem related to presence of a diseased cell according to the invention, for use in the treatment or prevention of a disease or health problem related to the presence of the aberrant cell according to the invention, for use in the treatment or prevention of a cancer, and/or for use in the treatment or prevention of an autoimmune disease, preferably in a human patient, wherein the first cell surface molecule and the third cell surface molecule are CD71 and/or the second cell surface molecule is CD71, and/or the first proteinaceous molecule and the third proteinaceous molecule are a monoclonal antibody capable of binding to CD71 or at least one sdAb capable of binding to CD71, and/or the second proteinaceous molecule is a monoclonal antibody capable of binding to CD71 or at least one sdAb capable of binding to CD71, and/or the effector moiety is an oligonucleotide, preferably, the first, second and third cell surface molecule is CD71, the first, second and third proteinaceous molecule is a monoclonal antibody capable of binding to CD71 or at least one sdAb capable of binding to CD71, and the effector moiety is an oligonucleotide.

1-Component Conjugate Comprising a Saponin Conjugate of the Invention and an Effector Moiety, for Medical Use

An aspect of the invention relates to an antibody-drug conjugate, antibody-oligonucleotide conjugate, ligand-drug conjugate or ligand-oligonucleotide conjugate, comprising the saponin conjugate of the invention and an effector moiety according to the invention, preferably an antibody-oligonucleotide conjugate comprising the saponin conjugate of the invention and an effector moiety according to the invention. Such an antibody-drug conjugate, antibody-oligonucleotide conjugate, ligand-drug conjugate or ligand-oligonucleotide conjugate is a so-called ‘1-component’ conjugate comprising the saponin derivative of the invention, the cell-targeting ligand (antibody) according to the invention and an effector moiety according to the invention.

An aspect of the invention relates to the antibody-drug conjugate, antibody-oligonucleotide conjugate, ligand-drug conjugate or ligand-oligonucleotide conjugate, preferably the antibody-oligonucleotide conjugate, according to the invention, for use as a medicament.

An aspect of the invention relates to the antibody-drug conjugate, antibody-oligonucleotide conjugate, ligand-drug conjugate or ligand-oligonucleotide conjugate, preferably the antibody-oligonucleotide conjugate, according to the invention, for use according to the invention, i.e., for use in the treatment or prevention of a disease or health problem related to presence of a diseased cell according to the invention, for use in the treatment or prevention of a disease or health problem related to the presence of the aberrant cell according to the invention, for use in the treatment or prevention of a cancer, and/or for use in the treatment or prevention of an autoimmune disease, preferably in a human patient.

An aspect of the invention relates to a 1-component pharmaceutical composition comprising the 1-component conjugate of the invention, the 1-component conjugate comprising a liver-cell targeting ligand such as an antibody and comprising an oligonucleotide and comprising a saponin derivative of the invention, the pharmaceutical composition optionally comprising a pharmaceutically acceptable excipient and/or optionally a pharmaceutically acceptable diluent.

An aspect of the invention relates to the ninth pharmaceutical composition of the invention or to the oligonucleotide conjugate of the invention and according to any one of the molecules (EE), (PP) and (SS), for use as a medicament.

An aspect of the invention relates to the 1-component pharmaceutical composition of the invention or to the first pharmaceutical combination of the invention, the second pharmaceutical combination of the invention or the third pharmaceutical composition of the invention, or to the 1-component conjugate of the invention, for use in the treatment or prophylaxis of a disease or health problem in which an expression product is involved of any one or more of genes: HSP27, apoB, TTR, PCSK9, TMPRSS6, ALAS1, AT3, GO, CCS, X gene of HBV, S gene of HBV, AAT, miR-122, hepatitis B virus HbsAg, LDHA, CEBPA and LDH, and/or for use in the treatment or prophylaxis of a disease or health problem which involves any one or more of genes: HSP27, apoB, TTR, PCSK9, TMPRSS6, ALAS1, AT3, GO, CCS, X gene of HBV, S gene of HBV, AAT, miR-122, hepatitis B virus HbsAg, LDHA, CEBPA and LDH.

An aspect of the invention relates to the 1-component pharmaceutical composition of the invention or to the first pharmaceutical combination of the invention, the second pharmaceutical combination of the invention or the third pharmaceutical composition of the invention, or to the 1-component conjugate of the invention, for use in the treatment or prophylaxis of a disease or health problem in which an expression product is involved of any one or more of genes: HSP27, apoB, TTR, PCSK9, TMPRSS6, ALAS1, AAT, miR-122, hepatitis B virus HbsAg, LDHA and CEBPA, and/or for use in the treatment or prophylaxis of a disease or health problem which involves any one or more of genes: HSP27, apoB, TTR, PCSK9, TMPRSS6, ALAS1, AAT, miR-122, hepatitis B virus HbsAg, LDHA and CEBPA.

An embodiment is the 1-component pharmaceutical composition of the invention or to the first pharmaceutical combination of the invention, the second pharmaceutical combination of the invention or the third pharmaceutical composition of the invention, or to the 1-component conjugate of the invention, for use as here above outlined, wherein said use is in the treatment or prophylaxis of a disease or health problem in which an expression product is involved of any one or more of genes: HSP27 and apoB, preferably apoB, and/or for use in the treatment or prophylaxis of a disease or health problem which involves any one or more of genes: HSP27 and apoB, preferably apoB.

An embodiment is the 1-component pharmaceutical composition of the invention or to the first pharmaceutical combination of the invention, the second pharmaceutical combination of the invention or the third pharmaceutical composition of the invention, or to the 1-component conjugate of the invention, for use as here above outlined, for use in the treatment or prophylaxis of a cancer, an infectious disease, a viral infection, hypercholesterolemia, cardiovascular disease, primary hyperoxaluria, haemophilia A, haemophilia B, AAT related liver disease, acute hepatic porphyria, TTR-mediated amyloidosis, hereditary TTR amyloidosis (hATTR), complement-mediated disease, hepatitis B infection, hepatitis C infection, al-antitrypsin deficiency, 8-thalassaemia, or an auto-immune disease.

An embodiment is the 1-component pharmaceutical composition of the invention or to the first pharmaceutical combination of the invention, the second pharmaceutical combination of the invention or the third pharmaceutical composition of the invention, or to the 1-component conjugate of the invention, for use as here above outlined, wherein said use is in the treatment or prophylaxis of a cancer such as endometrial carcinoma, breast cancer, lung cancer or hepatocellular carcinoma, and/or a cardiovascular disease such as hypercholesterolemia, preferably hypercholesterolemia.

An aspect of the invention relates to the 1-component pharmaceutical composition of the invention or to the first pharmaceutical combination of the invention, the second pharmaceutical combination of the invention or the third pharmaceutical composition of the invention, or to the 1-component conjugate of the invention, for use in the lowering of LDL-cholesterol in a subject.

### Method for Delivering an Effector Molecule Inside a Cell, Using a Saponin Derivative or a Saponin Conjugate

An aspect of the invention relates to an in vitro or ex vivo method for transferring a molecule from outside a cell to inside said cell, preferably into the cytosol of said cell, comprising the steps of:
a) providing a cell;
b) providing the molecule for transferring from outside the cell into the cell provided in step a);
c) providing a saponin derivative or a saponin conjugate according to the invention;
d) contacting the cell of step a) in vitro or ex vivo with the molecule of step b) and the saponin derivative or the saponin conjugate of step c), therewith establishing the transfer of the molecule from outside the cell into said cell.

More in particular, an in vitro or ex vivo method for transferring a molecule from outside a cell to inside said cell, preferably into the cytosol of said cell is provided, the method comprising the steps of:
a) providing a cell, preferably selected from: an aberrant cell, a diseased cell, a tumor cell and an auto-immune cell;
b) providing the molecule for transferring from outside the cell into the cell provided in step a), the molecule preferably selected from any one of the effector molecules of the invention preferably an oligonucleotide, wherein preferably the molecule for transferring from outside the cell into the cell is provided as a conjugate according to the invention, such conjugate comprising the second or third proteinaceous molecule;
c) providing a saponin conjugate according to the invention;
d) contacting the cell of step a) in vitro or ex vivo with the molecule of step b) and the saponin conjugate of step c), therewith establishing the transfer of the molecule from outside the cell into said cell.

An embodiment is the method of the invention, wherein the cell is a human cell such as a T-cell, an NK-cell, a tumor cell, and/or wherein the molecule of step b) is any one of: an antibody-drug conjugate, a receptor-ligand—drug conjugate, an antibody-oligonucleotide conjugate or a receptor-ligand—oligonucleotide conjugate, wherein the drug is for example a toxin and wherein the oligonucleotide is for example an AON such as an siRNA or a BNA, and/or wherein the saponin derivative is selected from the group consisting of derivatives of: SO1861, SA1657, GE1741, SA1641, QS-21, QS-21A, QS-21 A-api, QS-21 A-xyl, QS-21B, QS-21 B-api, QS-21 B-xyl, QS-7-xyl, QS-7-api, QS-17-api, QS-17-xyl, QS1861, QS1862, Quillajasaponin, Saponinum album, QS-18, Quil-A, Gyp1, gypsoside A, AG1, AG2, SO1542, SO1584, SO1658, SO1674, SO1832, SO1862, SO1904, stereoisomers thereof and combinations thereof, preferably the saponin derivative is selected from the group consisting of an SO1861 derivative, an SO1832 derivative, a GE1741 derivative, an SA1641 derivative, a QS-21 derivative, and a combination thereof, more preferably the saponin derivative is an SO1861 derivative, an SO1832 derivative or a QS-21 derivative, most preferably, the saponin derivative is an SO1861 derivative; or wherein the saponin derivative is according to any one of formula (V), formula (VI), formula (VII) and formula (VIII), preferably the saponin derivative is according to formula (V) or formula (VIII).

The saponin derivative is a saponin derivative of the invention. In particular embodiments the in vitro or ex vivo method for transferring a molecule from outside a cell to inside said cell, preferably into the cytosol of said cell as described herein is provided wherein the saponin derivative comprises, preferably consists of the saponin derivative according to formula (V), the saponin derivative according to formula (VI), the saponin derivative according to formula (VII), the saponin derivative according to formula (VIII) or any combination thereof.

An embodiment is the method of the invention, wherein the saponin conjugate is selected from any one of the saponin conjugates of the invention, including the 1-component conjugate. It is preferred that the cell-targeting ligand is an antibody such as a monoclonal antibody or at least an sdAb, capable of binding to CD71. It is preferred that the cell with which the method is applied (over)expresses CD71. It is preferred that the molecule selected for transferring into the selected cell by applying the method, is an oligonucleotide according to the invention. Preferably, the saponin on which the saponin derivative comprised by the saponin conjugate is based, is selected from SO1861, SO1832 and QS-21, preferably SO1861 and SO1832, more preferably SO1861.

TABLE A1
Saponins displaying (late) endosomal/lysosomal escape enhancing activity,
and with an aglycone core of the 12,13-dehydrooleanane type⁴⁾

| | | Carbohydrate | |
|---|---|---|---|
| | Aglycone core with | substituent at | |
| | an aldehyde group at | the C-3beta- | Carbohydrate substituent at the C- |
| Saponin Name | the C-23 position | OH group | 28-OH group |
| NP-017777 | Gypsogenin | Gal-(1→2)-[Xyl- | Xyl-(1→4)-Rha-(1→2)-[R-(→4)]-Fuc- |
| | | (1→3)]-GlcA- | (R = 4E-Methoxycinnamic acid) |
| NP-017778 | Gypsogenin | Gal-(1→2)-[Xyl- | Xyl-(1→4)-Rha-(1→2)-[R-(→4)]-Fuc- |
| | | (1→3)]-GlcA- | (R = 4Z-Methoxycinnamic acid) |
| NP-017774 | Gypsogenin | Gal-(1→2)-[Xyl- | Xyl-(1→4)-[Gal-(1→3)]-Rha-(1→2)-4- |
| | | (1→3)]-GlcA- | OAc-Fuc- |
| NP-018110^{c}, | Gypsogenin | Gal-(1→2)-[Xyl- | Xyl-(1→4)-[Glc-(1→3)]-Rha-(1→2)-3,4- |
| NP-017772^{d} | | (1→3)]-GlcA- | di-OAc-Fuc- |
| NP-018109 | Gypsogenin | Gal-(1→2)-[Xyl- | Xyl-(1→4)-[Glc-(1→3)]-Rha-(1→2)-[R- |
| | | (1→3)]-GlcA- | (→4)]-3-OAc-Fuc- (R = 4E- |
| | | | Methoxycinnamic acid) |
| NP-017888 | Gypsogenin | Gal-(1→2)-[Xyl- | Glc-(1→3)-Xyl-(1→4)-[Glc-(1→3)]- |
| | | (1→3)]-GlcA- | Rha-(1→2)-4-OAc-Fuc- |
| NP-017889 | Gypsogenin | Gal-(1→2)-[Xyl- | Glc-(1→3)-Xyl-(1→4)-Rha-(1→2)-4- |
| | | (1→3)]-GlcA- | OAc-Fuc- |
| NP-018108 | Gypsogenin | Gal-(1→2)-[Xyl- | Ara/Xyl-(1→3)-Ara/Xyl-(1→4)- |
| | | (1→3)]-GlcA- | Rha/Fuc-(1→2)-[4-OAc-Rha/Fuc- |
| | | | (1→4)]-Rha/Fuc- |
| SA1641^{a}, | Gypsogenin | Gal-(1→2)-[Xyl- | Xyl-(1→3)-Xyl-(1→4)-Rha-(1→2)-[Qui- |
| AEX55^{b} | | (1→3)]-GlcA- | (1→4)]-Fuc- |
| SO1658 | Gypsogenin | Gal-(1→2)-[Xyl- | Glc-(1→3)-[Xyl-(1→3)-Xyl-(1→4)]-Rha- |
| | | (1→3)]-GlcA- | (1→2)-Fuc- |
| gypsoside A⁶⁾ | Gypsogenin | Gal-(1→4)-Glc | Xyl-(1→3)-Fuc-(1→4)-[Xyl-(1→3)-Xyl- |
| | | (1→4)-[Ara- | (1→3)]-Rha- |
| | | (1→3)]-GlcA- | |
| phytolaccagenin | Gypsogenin | absent | absent |
| Gypsophila | Quillaic acid | Gal-(1→2)-[Xyl- | Glc-(1→3)-[Xyl-(1→4)]-Rha-(1→2)- |
| saponin 1 | | (1→3)]-GlcA- | Fuc- |
| (Gyp1) | | | |
| NP-017674 | Quillaic acid | Gal-(1→2)-[Xyl- | Api-(1→3)-Xyl-(1→4)-[Glc-(1→3)]- |
| | | (1→3)]-GlcA- | Rha-(1→2)-Fuc- |
| NP-017810 | Quillaic acid | Gal-(1→2)-[Xyl- | Xyl-(1→4)-[Gal-(1→3)]-Rha-(1→2)- |
| | | (1→3)]-GlcA- | Fuc- |
| AG1 | Quillaic acid | Gal-(1→2)-[Xyl- | Xyl-(1→4)-[Glc-(1→3)]-Rha-(1→2)- |
| | | (1→3)]-GlcA- | Fuc- |
| NP-003881 | Quillaic acid | Gal-(1→2)-[Xyl- | Ara/Xyl-(1→4)-Rha/Fuc-(1→4)- |
| | | (1→3)]-GlcA- | [Glc/Gal-(1→2)]-Fuc- |
| NP-017676 | Quillaic acid | Gal-(1→2)-[Xyl- | Api-(1→3)-Xyl-(1→4)-[Glc-(1→3)]- |
| | | (1→3)]-GlcA- | Rha-(1→2)-[R-(→4)]-Fuc- |
| | | | (R = 5-O-[5-O-Ara/Api-3,5-dihydroxy-6- |
| | | | methyl-octanoyl]-3,5-dihydroxy-6- |
| | | | methyl-octanoic acid) |
| NP-017677 | Quillaic acid | Gal-(1→2)-[Xyl- | Api-(1→3)-Xyl-(1→4)-Rha-(1→2)-[R- |
| | | (1→3)]-GlcA- | (→4)]-Fuc- |
| | | | (R = 5-O-[5-O-Ara/Api-3,5-dihydroxy-6- |
| | | | methyl-octanoyl]-3,5-dihydroxy-6- |
| | | | methyl-octanoic acid) |
| NP-017706 | Quillaic acid | Gal-(1→2)-[Xyl- | Api-(1→3)-Xyl-(1→4)-Rha-(1→2)- |
| | | (1→3)]-GlcA- | [Rha-(1→3)]-4-OAc-Fuc- |
| NP-017705 | Quillaic acid | Gal-(1→2)-[Xyl- | Api-(1→3)-Xyl-(1→4)-[Glc-(1→3)]- |
| | | (1→3)]-GlcA- | Rha-(1→2)-[Rha-(1→3)]-4-OAc-Fuc- |
| NP-017773 | Quillaic acid | Gal-(1→2)-[Xyl- | 6-OAc-Glc-(1→3)-Xyl-(1→4)-Rha- |
| | | (1→3)]-GlcA- | (1→2)-[3-OAc-Rha-(1→3)]-Fuc- |
| NP-017775 | Quillaic acid | Gal-(1→2)-[Xyl- | Glc-(1→3)-Xyl-(1→4)-Rha-(1→2)-[3- |
| | | (1→3)]-GlcA- | OAc--Rha-(1→3)]-Fuc- |
| SA1657 | Quillaic acid | Gal-(1→2)-[Xyl- | Xyl-(1→3)-Xyl-(1→4)-Rha-(1→2)-[Qui- |
| | | (1→3)]-GlcA- | (1→4)]-Fuc- |
| AG2 | Quillaic acid | Gal-(1→2)-[Xyl- | Glc-(1→3)-[Xyl-(1→4)]-Rha-(1→2)- |
| | | (1→3)]-GlcA- | [Qui-(1→4)]-Fuc- |
| GE1741 | Quillaic acid | Gal-(1→2)-[Xyl- | Xyl-(1→3)-Xyl-(1→4)-Rha-(1→2)-[3,4- |
| | | (1→3)]-GlcA- | di-OAc-Qui-(1→4)]-Fuc- |
| SO1542 | Quillaic acid | Gal-(1→2)-[Xyl- | Glc-(1→3)-[Xyl-(1→4)]-Rha-(1→2)- |
| | | (1→3)]-GlcA- | Fuc- |
| SO1584 | Quillaic acid | Gal-(1→2)-[Xyl- | 6-OAc-Glc-(1→3)-[Xyl-(1→4)]-Rha- |
| | | (1→3)]-GlcA- | (1→2)-Fuc- |
| SO1674 | Quillaic acid | Gal-(1→2)-[Xyl- | Glc-(1→3)-[Xyl-(1→3)-Xyl-(1→4)]-Rha- |
| | | (1→3)]-GlcA- | (1→2)-Fuc- |
| SO1700³⁾ | Quillaic acid | Gal-(1→2)-[Xyl- | Xyl-(1→4)-Rha-(1→2)-[Xyl-(1→3)-4- |
| | | (1→3)]-GlcA- | OAc-Qui-(1→4)]-Fuc- |
| Saponarioside | Quillaic acid | Gal-(1→2)-[Xyl- | Xyl-(1→3)-Xyl-(1→4)-Rha-(1→2)-[4- |
| B¹⁾ | | (1→3)]-GlcA- | OAc-Qui-(1→4)]-Fuc- |
| SO1730³⁾ | Quillaic acid | Gal-(1→2)-[Xyl- | Glc-(1→3)-Xyl-(1→4)-Rha-(1→2)-[-4- |
| | | (1→3)]-GlcA- | OAc-Qui-(1→4)]-Fuc- |
| SO1772³⁾ | Quillaic acid | Gal-(1→2)-[Xyl- | 6-OAc-Glc-(1→3)-[Xyl-(1→4)]-Rha- |
| | | (1→3)]-GlcA- | (1→2)-[4-OAc-Qui-(1→4)]-Fuc-- |
| SO1832¹⁾ | Quillaic acid | Gal-(1→2)-[Xyl- | Xyl-(1→3)-Xyl-(1→4)-Rha-(1→2)-[Xyl- |
| (protonated | | (1→3)]-GlcA- | (1→3)-4-OAc-Qui-(1→4)]-Fuc- |
| SO1831) = | | | |
| Saponarioside A | | | |
| SO1861 | Quillaic acid | Gal-(1→2)-[Xyl- | Glc-(1→3)-Xyl-(1→4)-Rha-(1→2)-[Xyl- |
| (deprotonated | | (1→3)]-GlcA- | (1→3)-4-OAc-Qui-(1→4)]-Fuc- |
| SO1862) | | | |
| SO1862 | Quillaic acid | Gal-(1→2)-[Xyl- | Glc-(1→3)-Xyl-(1→4)-Rha-(1→2)-[Xyl- |
| (protonated | | (1→3)]-GlcA- | (1→3)-4-OAc-Qui-(1→4)]-Fuc- |
| SO1861), also | | | |
| referred to as | | | |
| Sapofectosid⁵⁾ | | | |
| SO1904³⁾ | Quillaic acid | Gal-(1→2)-[Xyl- | 6-OAc-Glc-(1→3)-[Xyl-(1→4)]-Rha- |
| | | (1→3)]-GlcA- | (1→2)-[Xyl-(1→3)-4-OAc-Qui-(1→4)]- |
| | | | Fuc- |
| QS-7 (also | Quillaic acid | Gal-(1→2)-[Xyl- | Api/Xyl-(1→3)-Xyl-(1→4)-[Glc-(1→3)]- |
| referred to as | | (1→3)]-GlcA- | Rha-(1→2)-[Rha-(1→3)]-4OAc-Fuc- |
| QS1861) | | | |
| QS-7 api (also | Quillaic acid | Gal-(1→2)-[Xyl- | Api-(1→3)-Xyl-(1→4)-[Glc-(1→3)]- |
| referred to as | | (1→3)]-GlcA- | Rha-(1→2)-[Rha-(1→3)]-4OAc-Fuc- |
| QS1862) | | | |
| QS-17 | Quillaic acid | Gal-(1→2)-[Xyl- | Api/Xyl-(1→3)-Xyl-(1→4)-[Glc-(1→3)]- |
| | | (1→3)]-GlcA- | Rha-(1→2)-[R-(→4)]-Fuc- |
| | | | (R = 5-O-[5-O-Rha-(1→2)-Ara/Api-3,5- |
| | | | dihydroxy-6-methyl-octanoyl]-3,5- |
| | | | dihydroxy-6-methyl-octanoic acid) |
| QS-18 | Quillaic acid | Gal-(1→2)-[Xyl- | Api/Xyl-(1→3)-Xyl-(1→4)-[Glc-(1→3)]- |
| | | (1→3)]-GlcA- | Rha-(1→2)-[R-(→4)]-Fuc- |
| | | | (R = 5-O-[5-O-Ara/Api-3,5-dihydroxy-6- |
| | | | methyl-octanoyl]-3,5-dihydroxy-6- |
| | | | methyl-octanoic acid) |
| QS-21 A-apio | Quillaic acid | Gal-(1→2)-[Xyl- | Api-(1→3)-Xyl-(1→4)-Rha-(1→2)-[R- |
| | | (1→3)]-GlcA- | (→4)]-Fuc- |
| | | | (R = 5-O-[5-O-Ara/Api-3,5-dihydroxy-6- |
| | | | methyl-octanoyl]-3,5-dihydroxy-6- |
| | | | methyl-octanoic acid) |
| QS-21 A-xylo | Quillaic acid | Gal-(1→2)-[Xyl- | Xyl-(1→3)-Xyl-(1→4)-Rha-(1→2)-[R- |
| | | (1→3)]-GlcA- | (→4)]-Fuc- |
| | | | (R = 5-O-[5-O-Ara/Api-3,5-dihydroxy-6- |
| | | | methyl-octanoyl]-3,5-dihydroxy-6- |
| | | | methyl-octanoic acid) |
| QS-21 B-apio | Quillaic acid | Gal-(1→2)-[Xyl- | Api-(1→3)-Xyl-(1→4)-Rha-(1→2)-[R- |
| | | (1→3)]-GlcA- | (→3)]-Fuc- |
| | | | (R = 5-O-[5-O-Ara/Api-3,5-dihydroxy-6- |
| | | | methyl-octanoyl]-3,5-dihydroxy-6- |
| | | | methyl-octanoic acid) |
| QS-21 B-xylo | Quillaic acid | Gal-(1→2)-[Xyl- | Xyl-(1→3)-Xyl-(1→4)-Rha-(1→2)-[R- |
| | | (1→3)]-GlcA- | (→3)]-Fuc- |
| | | | (R = 5-O-[5-O-Ara/Api-3,5-dihydroxy-6- |
| | | | methyl-octanoyl]-3,5-dihydroxy-6- |
| | | | methyl-octanoic acid) |
| QS-21 | Quillaic acid | Gal-(1→2)-[Xyl- | Combination of the carbohydrate |
| | | (1→3)]-GlcA- | chains depicted for QS-21 A-apio, A- |
| | | | xylo, B-apio, B-xylo, for this position at |
| | | | the aglycone (see also the structure |
| | | | depicted as (Scheme Q)) |
| Agrostemmoside E | Quillaic acid | Gal-(1→2)-[Xyl- | [4,6-di-OAc-Glc-(1→3)]-[Xyl-(1→4)]- |
| (AG1856, | | (1→3)]-GlcA- | Rha-(1→2)-[3,4-di-OAc-Qui-(1→4)]- |
| AG2.8)²⁾ | | | Fuc- |
| | Aglycone core without | | |
| | an aldehyde group at | | Carbohydrate substituent at the C- |
| Saponin Name | the C-23 position | | 28-OH group |
| | | Carbohydrate | |
| | | substituent at | |
| | | the C-3beta- | |
| | | OH group | |
| NP-005236 | 2alpha- | GlcA- | Glc/Gal- |
| | Hydroxyoleanolic | | |
| | acid | | |
| AMA-1 | 16alpha- | Glc- | Rha-(1→2)-[Xyl-(1→4)]-Rha- |
| | Hydroxyoleanolic | | |
| | acid | | |
| AMR | 16alpha- | Glc- | Rha-(1→2)-[Ara-(1→3)-Xyl-(1→4)]- |
| | Hydroxyoleanolic | | Rha- |
| | acid | | |
| alpha-Hederin | Hederagenin (23- | Rha-(1→2)- | Not present |
| | Hydroxyoleanolic | Ara- | |
| | acid) | | |
| NP-012672 | 16alpha,23- | Ara/Xyl-(1→4)- | Ara/Xyl- |
| | Dihydroxyoleanolic | Rha/Fuc- | |
| | acid | (1→2)-Glc/Gal- | |
| | | (1→2)- | |
| | | Rha/Fuc- | |
| | | (1→2)-GlcA- | |
| beta-Aescin | Protoaescigenin- | Glc-(1→2)-[Glc- | Not present |
| (described: | 21(2-methylbut-2- | (1→4)]-GlcA- | |
| Aescin Ia) | enoate)-22-acetat | | |
| aescinate | Aglycone core | present | Not present |
| | without an | | |
| | aldehyde group at | | |
| | the C-23 position | | |
| dipsacoside B | Aglycone core | present | present |
| | without an | | |
| | aldehyde group at | | |
| | the C-23 position | | |
| esculentoside A | Aglycone core | present | Not present |
| | without an | | |
| | aldehyde group at | | |
| | the C-23 position | | |
| Teaseed | 23-Oxo- | Glc-(1→2)-Ara- | Not present |
| saponin I | barringtogenol C - | (1→3)-[Gal- | |
| | 21,22-bis(2- | (1→2)]-GlcA- | |
| | methylbut-2- | | |
| | enoate) | | |
| Teaseedsaponin J | 23-Oxo- | Xyl-(1→2)-Ara- | Not present |
| | barringtogenol C - | (1→3)-[Gal- | |
| | 21,22-bis(2- | (1→2)]-GlcA- | |
| | methylbut-2- | | |
| | enoate) | | |
| Assamsaponin F | 23-Oxo- | Glc-(1→2)-Ara- | Not present |
| | barringtogenol C - | (1→3)-[Gal- | |
| | 21(2-methylbut-2- | (1→2)]-GlcA- | |
| | enoate)-16,22- | | |
| | diacetat | | |
| Primula acid 1 | 3,16,28- | Rha-(1→2)- | Not present |
| | Trihydroxyoleanan- | Gal-(1→3)- | |
| | 12-en | [Glc-(1→2)]- | |
| | | GlcA- | |
| AS64R | Gypsogenic acid | absent | Glc-(1→3)-[Glc- |
| | | | (1→6)]-Gal- |
| Macranthoidin A | Aglycone core | present | present |
| | without an | | |
| | aldehyde group at | | |
| | the C-23 position | | |
| saikosaponin A | Aglycone core | present | absent |
| | without an | | |
| | aldehyde group at | | |
| | the C-23 position | | |
| saikosaponin D | Aglycone core | present | absent |
| | without an | | |
| | aldehyde group at | | |
| | the C-23 position | | |
| | | Carbohydrate | |
| | | substituent at | |
| | | the C-23-OH | |
| | | group | |
| AS6.2 | Gypsogenic acid | Gal- | Glc-(1→3)-[Glc-(1→6)]-Gal- |

^{a}, ^{b}Different names refer to different isolates of the same structure
^{c}, ^{d}Different names refer to different isolates of the same structure
¹⁾Jia et al., Major Triterpenoid Saponins from Saponaria officinalis, J. Nat. Prod. 1998, 61, 11, 1368-1373, Publication Date: Sep. 19, 1998, https://doi.org/10.1021/np980167u
²⁾The structure of Agrostemmoside E (also referred to as AG1856 or AG2.8) is given in FIG. 4 of J. Clochard et al, A new acetylated triterpene saponin from Agrostemma githago L. modulates gene delivery efficiently and shows a high cellular tolerance, International Journal of Pharmaceutics, Volume 589, 15 Nov. 2020, 119822.
³⁾Structures of SO1700, SO1730, SO1772, SO1904 are given in Moniuszko-Szajwaj et al., Highly Polar Triterpenoid Saponins from the Roots of Saponaria officinalis L., Helv. Chim. Acta, V99, pp. 347-354, 2016 (doi.org/10.1002/hlca.201500224).
⁴⁾See for example:
thesis by Dr Stefan Böttger (2013): Untersuchungen zur synergistischen Zytotoxizität zwischen Saponinen und Ribosomen inaktivierenden Proteinen Typ I, and
Sama et al., Structure-Activity Relationship of Transfection-Modulating Saponins - A Pursuit for the Optimal Gene Trafficker, Planta Med. Volume 85, pp. 513-518, 2019 (doi: 10.1055/a-0863-4795) and
Fuchs et al., Glycosylated Triterpenoids as Endosomal Escape Enhancers in Targeted Tumor Therapies, Biomedicine, Volume 5, issue 14, 2017 (doi: 10.3390/biomedicines5020014).
⁵⁾Sama et al., Sapofectosid - Ensuring non-toxic and effective DNA and RNA delivery, International Journal of Pharmaceutics, Volume 534, Issues 1-2, 20 Dec. 2017, Pages 195-205 (dx.doi.org/10.1016/j.ijpharm.2017.10.016) & Moniuszko-Szajwaj et al., Highly Polar Triterpenoid Saponins from the Roots of Saponaria officinalis L., Helv. Chim. Acta, V99, pp. 347-354, 2016 (doi.org/10.1002/hlca.201500224).
⁶⁾See for example: doi: 10.1016/s0040-4039(01)90658-6, Tetrahedron Letters No. 8, pp. 477-482, 1963 and pubchem.ncbi.nlm.nih.gov/compound/Gipsoside

Suitable sources for isolating saponins according to the invention, i.e. those that display endosomal escape enhancing activity, are Quillaja saponaria, Saponinum album, Saponaria officinalis, and Quillaja bark. Saponin suitable for the saponin derivatives of the invention and for the saponin conjugate of the invention are thus for example:
Quillaja saponaria saponin, saponin isolated from Quillaja saponaria, for example Quil-A, QS-17-api, QS-17-xyl, QS-21, QS-21A, QS-21B, QS-7-xyl,
Saponinum album, saponin isolated from Saponinum album
Saponaria officinalis saponin, saponin isolated from Saponaria officinalis (preferred),
Quillaja bark saponin, saponin isolated from Quillaja bark saponin, for example Quil-A, QS-17-api, QS-17-xyl, QS-21, QS-21A, QS-21 B, QS-7-xyl.

In addition, apart from QS-21, also the individual saponins present in QS-21 are suitable saponins for the saponin conjugate of the invention, i.e. the saponins depicted as the saponins of SCHEME Q:

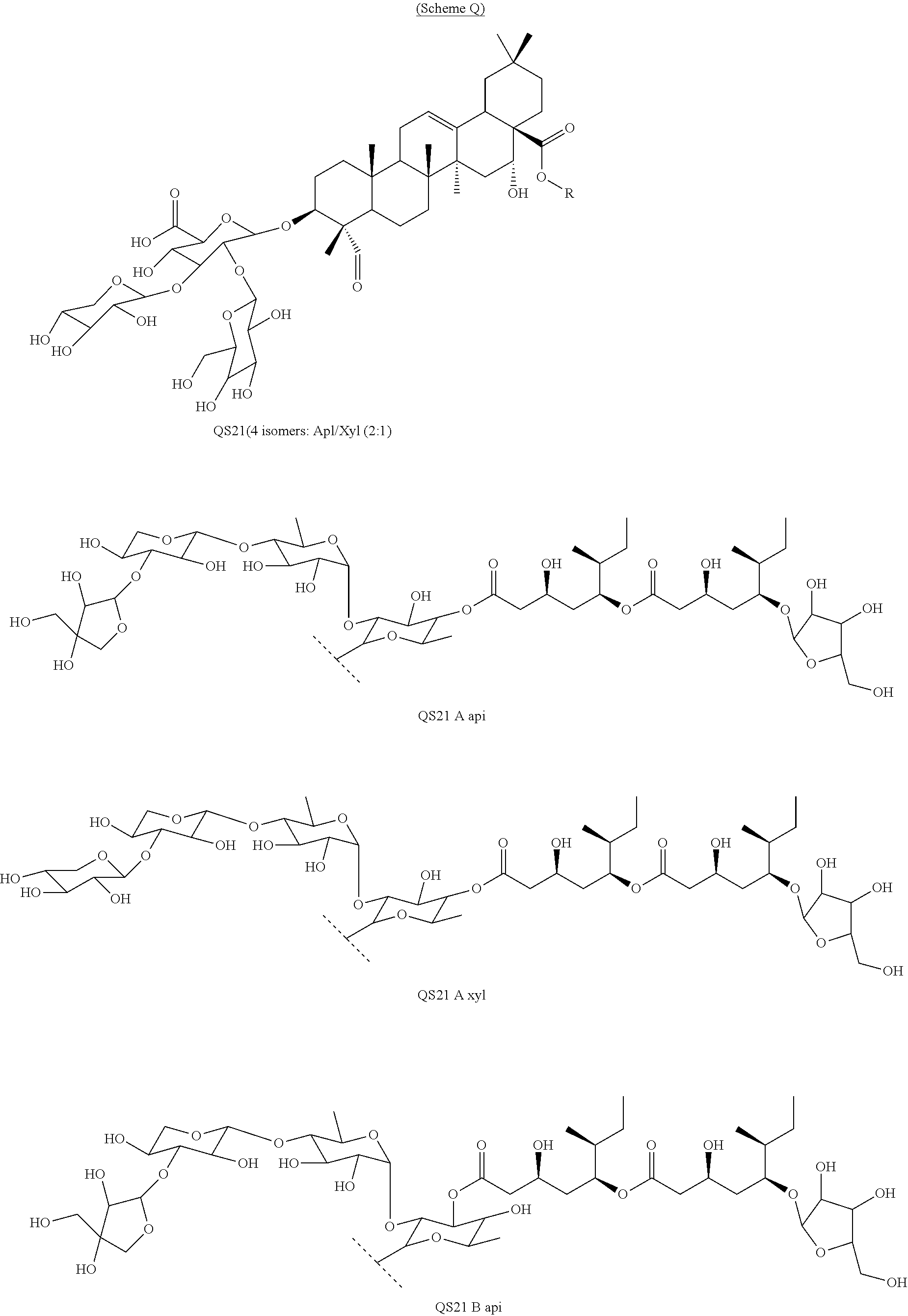

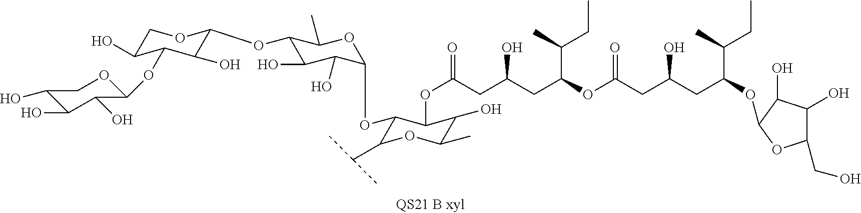

TABLE A2
Tumor-specific cell-surface receptor targets which can be targeted
by a cell-surface molecule targeting (binding) molecule such as
immunoglobulins, and antibodies that can be used for the saponin
conjugates of the invention (not presented as a limitation; further
immunoglobulins are equally suitable for the invention)

| Target | |
|---|---|
| cell- | |
| surface | |
| receptor | Example monoclonal antibodies |
| HER2 | anti-HER2 monoclonal antibody such as trastuzumab and |
| | pertuzumab |
| CD20 | anti-CD20 monoclonal antibody such as rituximab, |
| | ofatumumab, tositumomab and ibritumomab |
| CA125 | anti-CA125 monoclonal antibody such as oregovomab |
| EpCAM | anti-EpCAM (17-1A) monoclonal antibody such as |
| (17-1A) | edrecolomab |
| EGFR | anti-EGFR monoclonal antibody such as cetuximab, |
| | panitumumab and nimotuzumab |
| CD30 | anti-CD30 monoclonal antibody such brentuximab |
| CD33 | anti-CD33 monoclonal antibody such as gemtuzumab and |
| | huMy9-6 |
| vascular | anti-vascular integrin alpha-v beta-3 monoclonal antibody |
| integrin | such as etaracizumab |
| alpha-v | |
| beta-3 | |
| CD52 | anti-CD52 monoclonal antibody such as alemtuzumab |
| CD22 | anti-CD22 monoclonal antibody such as epratuzumab |
| CEA | anti-CEA monoclonal antibody such as labetuzumab |
| CD44v6 | anti-CD44v6 monoclonal antibody such as bivatuzumab |
| FAP | anti- FAP monoclonal antibody such as sibrotuzumab |
| CD19 | anti-CD19 monoclonal antibody such as huB4 |
| CanAg | anti-CanAg monoclonal antibody such as huC242 |
| CD56 | anti-CD56 monoclonal antibody such huN901 |
| CD38 | anti-CD38 monoclonal antibody such as daratumumab |
| CA6 | anti-CA6 monoclonal antibody such as DS6 |
| IGF-IR | anti-IGF-IR monoclonal antibody such as cixutumumab |
| | and 3B7 |
| integrin | anti-integrin monoclonal antibody such as CNTO 95 |
| syndecan-1 | anti-syndecan-1 monoclonal antibody such as B-B4 |

The invention is further illustrated by the following examples, which should not be interpreted as limiting the present invention in any way.

### EXAMPLES

Materials and Methods

### Abbreviations

Ab Antibody
AEM N-(2-Aminoethyl)maleimide trifluoroacetate salt
AMPD 2-Amino-2-methyl-1,3-propanediol
BOC tert-Butyloxycarbonyl
BOP (Benzotriazol-1-yloxy)tris(dimethylamino)phosphonium hexafluorophosphate
DCM dichloromethane
DIPEA N,N-diisopropylethylamine
DMF N,N-dimethylformamide
DMSO Dimethylsulfoxid
DTME dithiobismaleimidoethane
DTT dithiothreitol
EDCl·HCl 3-((Ethylimino)methyleneamino)-N,N-dimethylpropan-1-aminium chloride
EDTA ethylenediaminetetraacetic acid
EMCH·TFA N-(ε-maleimidocaproic acid) hydrazide, trifluoroacetic acid salt
HATU 1-[Bis(dimethylamino)methylene]-1H-1,2,3-triazolo[4,5-13]pyridinium 3-oxid hexafluorophosphate
mab monoclonal antibody
min minutes
NEM N-Ethylmaleimide
NMM 4-methylmorpholine
r.t. retention time
SC semicarbazone
SEC size exclusion chromatography
TBEU (Tris-(hydroxymethyl)-aminomethan)-Borat-EDTA-Urea
TCEP tris(2-carboxyethyl)phosphine hydrochloride
Temp temperature
TFA trifluoroacetic acid
THF tetrahydrofuran

Materials

Trastuzumab (Herceptin®, Roche), cetuximab (Erbitux®, Merck KGaA) were purchased from the pharmacy (Chart& Berlin). CD71 monoclonal antibody was purchased from BioCell (Okt9, #BE0023). SO1861 was isolated and purified by Analyticon Discovery GmbH from raw plant extract obtained from Saponaria officinalis L. EGFdianthin was produced from E. coli according to standard procedures. HSP27BNA oligo and ApoB and ApoB #02 were produced by Bio-Synthesis Inc, (Lewisville). Tris(2-carboxyethyl)phosphine hydrochloride (TCEP, 98%, Sigma-Aldrich), 5,5-Dithiobis(2-nitrobenzoic acid) (DTNB, Ellman's reagent, 99%, Sigma-Aldrich), Zeba™ Spin Desalting Columns (2 mL, Thermo-Fisher), NuPAGE™ 4-12% Bis-Tris Protein Gels (Thermo-Fisher), NuPAGE™ MES SDS Running Buffer (Thermo-Fisher), Novex™ Sharp Pre-stained Protein Standard (Thermo-Fisher), PageBlue™ Protein Staining Solution (Thermo-Fischer), Pierce™ BCA Protein Assay Kit (Thermo-Fisher), N-Ethylmaleimide (NEM, 98%, Sigma-Aldrich), 1,4-Dithiothreitol (DTT, 98%, Sigma-Aldrich), Sephadex G25 (GE Healthcare), Sephadex G50 M (GE Healthcare), Superdex 200P (GE Healthcare), Isopropyl alcohol (IPA, 99.6%, VWR), Tris(hydroxymethyl)aminomethane (Tris, 99%, Sigma-Aldrich), Tris(hydroxymethyl)aminomethane hydrochloride (Tris·HCL, Sigma-Aldrich), L-Histidine (99%, Sigma-Aldrich), D-(+)-Trehalose dehydrate (99%, Sigma-Aldrich), Polyethylene glycol sorbitan monolaurate (TWEEN 20, Sigma-Aldrich), Dulbecco's Phosphate-Buffered Saline (DPBS, Thermo-Fisher), Guanidine hydrochloride (99%, Sigma-Aldrich), Ethylenediaminetetraacetic acid disodium salt dihydrate (EDTA-Naz, 99%, Sigma-Aldrich), sterile filters 0.2 μm and 0.45 μm (Sartorius), Succinimidyl 4-(N-maleimidomethyl)cyclohexane-1-carboxylate (SMCC, Thermo-Fisher), Vivaspin T4 and T15 concentrator (Sartorius), Superdex 200PG (GE Healthcare), Tetra(ethylene glycol) succinimidyl 3-(2-pyridyldithio)propionate (PEG₄-SPDP, Thermo-Fisher), [0-(7-Azabenzotriazol-1-yl)-N,N,N,N-tetramethyluronium-hexafluorphosphat] (HATU, 97%, Sigma-Aldrich), Dimethyl sulfoxide (DMSO, 99%, Sigma-Aldrich), N-(2-Aminoethyl)maleimide trifluoroacetate salt (AEM, 98%, Sigma-Aldrich), L-Cysteine (98.5%, Sigma-Aldrich), deionized water (DI) was freshly taken from Ultrapure Lab Water Systems (MilliQ, Merck), Nickel-nitrilotriacetic acid agarose (Ni-NTA agarose, Protino), Glycine (99.5%, VWR), 5,5-Dithiobis(2-nitrobenzoic acid (Ellman's reagent, DTNB, 98%, Sigma-Aldrich), S-Acetylmercaptosuccinic anhydride Fluorescein (SAMSA reagent, Invitrogen) Sodium bicarbonate (99.7%, Sigma-Aldrich), Sodium carbonate (99.9%, Sigma-Aldrich), PD MiniTrap desalting columns with Sephadex G-25 resin (GE Healthcare), PD10 G25 desalting column (GE Healthcare), Zeba Spin Desalting Columns in 0.5, 2, 5, and 10 mL (Thermo-Fisher), Vivaspin Centrifugal Filters T4 10 kDa MWCO, T4 100 kDa MWCO, and T15 (Sartorius), Biosep s3000 aSEC column (Phenomenex), Vivacell Ultrafiltration Units 10 and 30 kDa MWCO (Sartorius), Nalgene Rapid-Flow filter (Thermo-Fisher), dichlormethan (Sigma-Aldrich), methanol (Sigma-Aldrich), diethyl ether (Sigma-Aldrich), acetonitrile (Sigma-Aldrich), Pyridine 2-thione (Sigma-Aldrich), Goat anti-Human IgG-HRP (Southern Biotech), Goat anti-Human Kappa-HRP (Southern Biotech), Tris concentrate (Thermo-Fisher), MOPS running buffer (20×, Thermo-Fisher), LDS sample buffer (4×, Thermo-Fisher), TBS Blocking Buffer (Thermo-Fisher), Tris (Tris(hydroxymethyl)aminomethane, Merck), Tris HCl (Sigma-Aldrich), Minisart RC15 0.2 μm filter (Sartorius), Minisart 0.45 μm filter (Sartorius), PD Minitrap G25 (Cytiva), TNBS (2,4,6-trinitrobenzene sulfonic acid, Sigma-Aldrich), Sodium Dodecyl Sulfate (SDS, Sigma-Aldrich), SMCC (succinimidyl 4-(N-maleimidomethyl)cyclohexane-1-carboxylate, Thermo-Fisher), THPP (Tris(hydroxypropyl)phosphine, Sigma-Aldrich), DBCO-NHS (CAS 1353016-71-3, BroadPharm), PEG4-SPDP (2-Pyridyldithiol-tetraoxatetradecane-N-hydroxysuccinimide, Thermo-Fisher), Novex™ TBE-Urea Gels, 15% (Thermo-Fisher), TBE buffer (Tris-Borat-EDTA, Thermo-Fisher).

Methods

Analytical Methods

LC-MS Method 1,¹

Apparatus: Waters IClass; Bin. Pump: UPIBSM, SM: UPISMFTN with SO; UPCMA, PDA: UPPDATC, 210-320 nm, SQD: ACQ-SQD2 ESI, pos/neg 100-800; ELSD: gas pressure 40 psi, drift tube temp: 50° C.; column: Acquity C18, 50×2.1 mm, 1.7 μm Temp: 60° C., Flow: 0.6 mL/min, Gradient: t₀=5% B, t_{2.0min}=98% B, t_{2.7min}=98% B, Post time: 0.3 min, Eluent A: 0.1% formic acid in water, Eluent B: 0.1% formic acid in acetonitrile.

LC-MS Method 1

Apparatus: Waters IClass; Bin. Pump: UPIBSM, SM: UPISMFTN with SO; UPCMA, PDA: UPPDATC, 210-320 nm, SQD: ACQ-SQD2 ESI, mass ranges depending on the molecular weight of the product:

neg or neg/pos within in a range of 1500-2400 or 2000-3000; ELSD: gas pressure 40 psi, drift tube temp: 50° C.; column: Acquity C18, 50×2.1 mm, 1.7 μm Temp: 60° C., Flow: 0.6 mL/min, lin. Gradient depending on the polarity of the product:
^{A} t₀=2% A, t_{5.0min}=50% A, t_{6.0min}=98% A
^{B} t₀=2% A, t_{5.0min}=98% A, t_{6.0min}=98% A

Posttime: 1.0 min, Eluent A: acetonitrile, Eluent B: 10 mM ammonium bicarbonate in water (pH=9.5).

LC-MS Method 1,^{1,2}

Apparatus: Agilent 1200 Bin. Pump: G1312A, degasser; autosampler, ColCom, DAD: Agilent G1316A, 210, 220 and 220-320 nm, PDA: 210-320 nm, MSD: Agilent LC/MSD G6130B ESI, pos/neg 100-1000; ELSD Alltech 3300 gas flow 1.5 ml/min, gas temp: 40° C.; column: Waters XSelect™ CSH C18, 30×2.1 mm, 3.5 μm, Temp: 35° C., Flow: 1 mL/min, Gradient: t₀=5% A, t_{1.6min}=98% A, t₃ₘᵢₙ=98% A, Posttime: 1.3 min, Eluent A: 0.1% formic acid in acetonitrile, Eluent B: 0.1% formic acid in water.

LC-MS Method 1,²

Apparatus: Waters IClass; Bin. Pump: UPIBSM, SM: UPISMFTN with SO; UPCMA, PDA: UPPDATC, 210-320 nm, SQD: ACQ-SQD2 ESI, neg 2000-3000; ELSD: gas pressure 40 psi, drift tube temp: 50° C.; column: Acquity C18, 50×2.1 mm, 1.7 μm Temp: 60° C., Flow: 0.6 mL/min, Gradient: t₀=5% B, t_{5.0min}=98% B, t_{6.0min}=98% B, Post time: 1.0 min, Eluent A: 0.1% formic acid in water, Eluent B: 0.1% formic acid in acetonitrile.

LC-MS Method 2

Apparatus: Waters IClass; Bin. Pump: UPIBSM, SM: UPISMFTN with SO; UPCMA, PDA: UPPDATC, 210-320 nm, SQD: ACQ-SQD2 ESI, mass ranges depending on the molecular weight of the product: pos/neg 100-800 or neg 2000-3000; ELSD: gas pressure 40 psi, drift tube temp: 50° C.; column: Waters XSelect™ CSH C18, 50×2.1 mm, 2.5 μm, Temp: 25° C., Flow: 0.5 mL/min, Gradient: t₀ₘᵢₙ=5% A, t_{2.0min}=98% A, t_{2.7min}=98% A, Posttime: 0.3 min, Eluent A: acetonitrile, Eluent B: 10 mM ammonium bicarbonate in water (pH=9.5).

LC-MS Method 2,²

Apparatus: Agilent 1260 Bin. Pump: G7112B, Multisampler, Column Comp, DAD: Agilent G7115A, 210, 220 and 220-320 nm, PDA: 210-320 nm, MSD: Agilent LC/MSD G6130B ESI, mass ranges depending on the molecular weight of the product:
^{A} pos/neg 100-1000
^{B} pos/neg 100-1400;
   ELSD Alltech 3300 gas flow 1.5 ml/min, gas temp: 40° C.; column: Waters XSelect™ C18, 30×2.1 mm, 3.5 μm, Temp: 40° C., Flow: 1 mL/min, Gradient: t₀=5% A, t_{1.6min}=98% A, t₃ₘᵢₙ=98% A, Posttime: 1.3 min, Eluent A: 0.1% formic acid in acetonitrile, Eluent B: 0.1% formic acid in water.

LC-MS Method 1,³

Apparatus: Waters IClass; Bin. Pump: UPIBSM, SM: UPISMFTN with SO; UPCMA, PDA: UPPDATC, 210-320 nm, SQD: ACQ-SQD2 ESI, neg/pos 1500-2400; ELSD: gas pressure 40 psi, drift tube temp: 50° C.; column: Acquity C18, 50×2.1 mm, 1.7 μm Temp: 60° C., Flow: 0.6 mL/min, lin. Gradient depending on the polarity of the product: t₀=2% B, t_{5.0min}=98% B, t_{6.0min}=98% B, Post time: 1.0 min, Eluent A: 10 mM ammonium bicarbonate in water (pH=9.5), Eluent B: acetonitrile.

LC-MS Method 3

Apparatus: Waters IClass; Bin. Pump: UPIBSM, SM: UPISMFTN with SO; UPCMA, PDA: UPPDATC, 210-320 nm, SQD: ACQ-SQD2 ESI, mass ranges depending on the molecular weight of the product pos/neg 105-800, 500-1200 or 1500-2500; ELSD: gas pressure 40 psi, drift tube temp: 50° C.; column: Waters XSelect™ CSH C18, 50×2.1 mm, 2.5 μm, Temp: 40° C., Flow: 0.5 mL/min, Gradient: t₀ₘᵢₙ=5% A, t_{2.0min}=98% A, t_{2.7min}=98% A, Posttime: 0.3 min, Eluent A: 0.1% formic acid in acetonitrile, Eluent B: 0.1% formic acid in water.

LC-MS Method 3,³

Apparatus: Waters IClass; Bin. Pump: UPIBSM, SM: UPISMFTN with SO; UPCMA, PDA: UPPDATC, 210-320 nm, SQD: ACQ-SQD2 ESI, pos/neg 800-1500; ELSD: gaspressure 40 psi, drift tube temp: 50° C.; column: Waters XSelect™ CSH C18, 50×2.1 mm, 2.5 μm Temp: 25° C., Flow: 0.6 mL/min, Gradient: t₀=5% A, t_{2.0min}=98% A, t_{2.7min}=98% A, Posttime: 0.3 min, Eluent A: acetonitrile, Eluent B: 10 mM ammonium bicarbonate in water (pH=9.5).

LC-MS Method 1,⁴ Apparatus: Waters IClass; Bin. Pump: UPIBSM, SM: UPISMFTN with SO; UPCMA, PDA:

UPPDATC, 210-320 nm, SQD: ACQ-SQD2 ESI, neg/pos 1500-2400; ELSD: gas pressure 40 psi, drift tube temp: 50° C.; column: Acquity C18, 50×2.1 mm, 1.7 μm Temp: 60° C., Flow: 0.6 mL/min, lin. Gradient depending on the polarity of the product: t₀=2% B, t_{5.0min}=50% B, t_{6.0min}=98% B, Post time: 1.0 min, Eluent A: 10 mM ammonium bicarbonate in water (pH=9.5), Eluent B: acetonitrile.

LC-MS Method 4

Apparatus: Waters IClass; Bin. Pump: UPIBSM, SM: UPISMFTN with SO; UPCMA, PDA: UPPDATC, 210-320 nm, SQD: ACQ-SQD2 ESI, mass ranges depending on the molecular weight of the product: pos/neg 100-800 or neg 2000-3000; ELSD: gas pressure 40 psi, drift tube temp: 50° C. column: Waters Acquity Shield RP18, 50×2.1 mm, 1.7 μm, Temp: 25° C., Flow: 0.5 mL/min, Gradient: t₀ₘᵢₙ=5% A, t_{2.0min}=98% A, t_{2.7min}=98% A, Posttime: 0.3 min, Eluent A: acetonitrile, Eluent B: 10 mM ammonium bicarbonate in water (pH=9.5).

LC-MS Method 4,⁴

Apparatus: Waters IClass; Bin. Pump: UPIBSM, SM: UPISMFTN with SO; UPCMA, PDA: UPPDATC, 210-320 nm, SQD: ACQ-SQD2 ESI, pos/neg 1500-2500; ELSD: gas pressure 40 psi, drift tube temp: 50° C.; column: Waters XSelect™ CSH C18, 50×2.1 mm, 2.5 μm Temp: 25° C., Flow: 0.6 mL/min, Gradient: t₀=15% A, t_{2.0min}=60% A, t_{2.7min}=98% A, Posttime: 0.3 min, Eluent A: acetonitrile, Eluent B: 10 mM ammonium bicarbonate in water (pH=9.5).

LC-MS Method 5,⁵

Apparatus: Waters IClass; Bin. Pump: UPIBSM, SM: UPISMFTN with SO; UPCMA, PDA: UPPDATC, 210-320 nm, SQD: ACQ-SQD2 ESI, mass ranges depending on the molecular weight of the product:
^{A} pos/neg 1500-2500
^{B} neg 2000-3000;
   ELSD: gaspressure 40 psi, drift tube temp: 50° C.; column: Acquity C18, 50×2.1 mm, 1.7 μm Temp: 60° C., Flow: 0.6 mL/min, Gradient: t₀=2% A, t_{5.0min}=50% A, t_{6.0min}=98% A, Posttime: 1.0 min, Eluent A: acetonitrile, Eluent B: 10 mM ammonium bicarbonate in water (pH=9.5).

Preparative Methods

Preparative MP-LC method 1,¹

Instrument type: Reveleris™ prep MPLC; Column: Phenomenex LUNA C18(3) (150×25 mm, 10 μm); Flow: 40 mL/min; Column temp: room temperature; Eluent A: 0.1% (v/v) Formic acid in water, Eluent B: 0.1% (v/v) Formic acid in acetonitrile; Gradient: t₀ₘᵢₙ=5% B, t₁ₘᵢₙ=5% B, t₂ₘᵢₙ=20% B, t₁₇ₘᵢₙ=60% B, t₁₈ₘᵢₙ=100% B, t₂₃ₘᵢₙ=100% B; Detection UV: 210, 235, 254 nm and ELSD.

Preparative MP-LC Method 2,²

Instrument type: Reveleris™ prep MPLC; column: Waters XSelect™ CSH C18 (145×25 mm, 10 μm); Flow: 40 mL/min; Column temp: room temperature; Eluent A: 10 mM ammoniumbicarbonate in water pH=9.0); Eluent B: 99% acetonitrile+1% 10 mM ammoniumbicarbonate in water; Gradient: t₀ₘᵢₙ=5% B, t₁ₘᵢₙ=5% B, t₂ₘᵢₙ=10% B, t₁₇ₘᵢₙ=50% B, t₁₈ₘᵢₙ=100% B, t₂₃ₘᵢₙ=100% B; Detection UV: 210, 235, 254 nm and ELSD.

Preparative MP-LC Method 1

Instrument type: Reveleris™ prep MPLC; column: Waters XSelect™ CSH C18 (145×25 mm, 10 μm); Flow: 40 mL/min; Column temp: room temperature; Eluent A: 10 mM ammoniumbicarbonate in water pH=9.0); Eluent B: 99% acetonitrile+1% 10 mM ammoniumbicarbonate in water; Gradient:
^{A}t₀ₘᵢₙ=5% B, t₁ₘᵢₙ=5% B, t₂ₘᵢₙ=10% B, t₁₇ₘᵢₙ=50% B, t₁₈ₘᵢₙ=100% B, t₂₃ₘᵢₙ=100% B
^{A} t₀ₘᵢₙ=5% B, t₁ₘᵢₙ=5% B, t₂ₘᵢₙ=20% B, t₁₇ₘᵢₙ=60% B, t₁₈ₘᵢₙ=100% B, t₂₃ₘᵢₙ=100% B;

### Detection UV: 210, 235, 254 nm and ELSD.

Preparative MP-LC Method 2

Instrument type: Reveleris™ prep MPLC; Column: Phenomenex LUNA C18(3) (150×25 mm, 10 μm); Flow: 40 mL/min; Column temp: room temperature; Eluent A: 0.1% (v/v) Formic acid in water, Eluent B: 0.1% (v/v) Formic acid in acetonitrile; Gradient:
^{A} t₀ₘᵢₙ=5% B, t₁ₘᵢₙ=5% B, t₂ₘᵢₙ=20% B, t₁₇ₘᵢₙ=60% B, t₁₈ₘᵢₙ=100% B, t₂₃ₘᵢₙ=100% B
^{B}t₀ₘᵢₙ=2% B, t₁ₘᵢₙ=2% B, t₂ₘᵢₙ=2% B, t₁₇ₘᵢₙ=30% B, t₁₈ₘᵢₙ=100% B, t₂₃ₘᵢₙ=100% B
^{C} t₀ₘᵢₙ=5% B, t₁ₘᵢₙ=5% B, t₂ₘᵢₙ=10% B, t₁₇ₘᵢₙ=50% B, t₁₈ₘᵢₙ=100% B, t₂₃ₘᵢₙ=100% B
^{D} t₀ₘᵢₙ=5% B, t₁ₘᵢₙ=5% B, t₂ₘᵢₙ=5% B, t₁₇ₘᵢₙ=40% B, t₁₈ₘᵢₙ=100% B, t₂₃ₘᵢₙ=100% B

### ; Detection UV: 210, 235, 254 nm and ELSD.

Preparative LC-MS Method 3

MS instrument type: Agilent Technologies G6130B Quadrupole; HPLC instrument type: Agilent Technologies 1290 preparative LC; Column: Waters XSelect™ CSH (C18, 150×19 mm, 10 μm); Flow: 25 ml/min; Column temp: room temperature; Eluent A: 100% acetonitrile; Eluent B: 10 mM ammonium bicarbonate in water pH=9.0; Gradient:
^{A} t₀=20% A, t_{2.5min}=20% A, t₁₁ₘᵢₙ=60% A, t₁₃ₘᵢₙ=100% A, t₁₇ₘᵢₙ=100% A
^{B}t₀=5% A, t_{2.5min}=5% A, t₁₁ₘᵢₙ=40% A, t₁₃ₘᵢₙ=100% A, t₁₇ₘᵢₙ=100% A;
   Detection: DAD (210 nm); Detection: MSD (ESI pos/neg) mass range: 100-800; Fraction collection based on DAD.

Preparative LC-MS Method 4

MS instrument type: Agilent Technologies G6130B Quadrupole; HPLC instrument type: Agilent

Technologies 1290 preparative LC; Column: Waters XBridge Protein (C4, 150×19 mm, 10 μm); Flow: 25 ml/min; Column temp: room temperature; Eluent A: 100% acetonitrile; Eluent B: 10 mM ammonium bicarbonate in water pH=9.0; Gradient:
^{A}t₀=2% A, t_{2.5min}=2% A, t₁₁ₘᵢₙ=30% A, t₁₃ₘᵢₙ=100% A, t₁₇ₘᵢₙ=100% A
^{B}t₀=10% A, t_{2.5min}=10% A, t₁₁ₘᵢₙ=50% A, t₁₃ₘᵢₙ=100% A, t₁₇ₘᵢₙ=100% A
_{C}t₀=5% A, t_{2.5min}=5% A, t₁₁ₘᵢₙ=40% A, t₁₃ₘᵢₙ=100% A, t₁₇ₘᵢₙ=100% A;
   Detection: DAD (210 nm); Detection: MSD (ESI pos/neg) mass range: 100-800; Fraction collection based on DAD

Preparative MP-LC Method 1,¹

Instrument type: Reveleris™ prep MPLC; column: Waters XSelect™ CSH C18 (145×25 mm, 10 μm); Flow: 40 mL/min; Column temp: room temperature; Eluent A: 10 mM ammoniumbicarbonate in water pH=9.0); Eluent B: 99% acetonitrile+1% 10 mM ammoniumbicarbonate in water; Gradient: t₀ₘᵢₙ=5% B, t₁ₘᵢₙ=5% B, t₂ₘᵢₙ=10% B, t₁₇ₘᵢₙ=50% B, t₁₈ₘᵢₙ=100% B, t₂₃ₘᵢₙ=100% B; Detection UV: 210, 225, 285 nm.

Preparative MP-LC Method 2,²

Instrument type: Reveleris™ prep MPLC; Column: Phenomenex LUNA C18(3) (150×25 mm, 10 μm); Flow: 40 mL/min; Column temp: room temperature; Eluent A: 0.1% (v/v) Formic acid in water, Eluent B: 0.1% (v/v) Formic acid in acetonitrile; Gradient: t₀ₘᵢₙ=5% B, t₁ₘᵢₙ=5% B, t₂ₘᵢₙ=10% B, t₁₇ₘᵢₙ=50% B, t₁₈ₘᵢₙ=100% B, t₂₃ₘᵢₙ=100% B; Detection UV: 210, 225, 285 nm.

Preparative LC-MS Method 1,³

MS instrument type: Agilent Technologies G6130B Quadrupole; HPLC instrument type: Agilent Technologies 1290 preparative LC; Column: Waters XSelect™ CSH (C18, 100×30 mm, 10 μm); Flow: 25 ml/min; Column temp: room temperature; Eluent A: 100% acetonitrile; Eluent B: 10 mM ammonium bicarbonate in water pH=9.0; lin. gradient depending on the polarity of the product:
^{A}t₀=20% A, t₂ₘᵢₙ=20% A, t_{8.5min}=60% A, t₁₀ₘᵢₙ=100% A, t₁₃ₘᵢₙ=100% A
^{B}t₀=5% A, t₂ₘᵢₙ=5% A, t_{8.5min}=40% A, t₁₀ₘᵢₙ=100% A, t₁₃ₘᵢₙ=100% A
^{C}t₀=10% A, t₂ₘᵢₙ=10% A, t_{8.5min}=50% A, t₁₀ₘᵢₙ=100% A, t₁₃ₘᵢₙ=100% A;
   Detection: DAD (220-320 nm); Detection: MSD (ESI pos/neg) mass range: 100-800; Fraction collection based on DAD.

Preparative LC-MS Method 2,⁴

MS instrument type: Agilent Technologies G6130B Quadrupole; HPLC instrument type: Agilent Technologies 1290 preparative LC; column: Waters XBridge Shield (C18, 150×19 mm, 5 μm); Flow: 25 ml/min; Column temp: room temperature; Eluent A: 100% acetonitrile; Eluent B: 10 mM ammonium bicarbonate in water pH=9.0; lin. gradient: t₀=5% A, t_{2.5min}=5% A, film, t₁₁ₘᵢₙ=40% A, t₁₃ₘᵢₙ=100% A, t₁₇ₘᵢₙ=100% A; Detection: DAD (220-320 nm); Detection: MSD (ESI pos/neg) mass range: 100-800; Fraction collection based DAD

Flash Chromatopraphy

Grace Reveleris X2® C-815 Flash; Solvent delivery system: 3-piston pump with auto-priming, 4 independent channels with up to 4 solvents in a single run, auto-switches lines when solvent depletes; maximum pump flow rate 250 mL/min; maximum pressure 50bar (725 psi); Detection: UV 200-400 nm, combination of up to 4 UV signals and scan of entire UV range, ELSD; Column sizes: 4-330 g on instrument, luer type, 750 g up to 3000 g with optional holder.

Further Methods

UV-Vis Spectrophotometry

Protein concentrations were determined using a Thermo Nanodrop 2000 spectrometer and the following mass E280 values ((mg/ml)-1 cm-1); Oligo concentrations were determined using a molar ε260 value of 153,000 M−1 cm−1.

Ellman's assay was carried out using a Perkin Elmer Lambda 25 Spectrophotometer and a literature molar ε412 value of 14150 M−1 cm−1 for TNB. Experimentally determined molar ε495=58,700 M−1 cm−1 and Rz280:495=0.428 were used for SAMSA-fluorescein.

TNBS Assay

Glycine standards (0, 2.5, 5, 10, 15 and 20 μg/ml) were freshly prepared using DPBS pH 7.5. TNBS assay reagent was prepared by combining TNBS (40 μl) and DPBS pH 7.5 (9.96 ml). 10% w/v SDS prepared using DI water. For the assay; 60 μl of each sample (singlicate) and standard (triplicate) plated out. To each well was added TNBS reagent (60 μl) and the plate shaker-incubated for 3 hours at 37° C. and 600 rpm. Afterwards, 50 μl of 10% SDS and 25 μl 1M HCl was added and the plate was analysed at 340 nm. Lysine-targeting molecule incorporation determined by depletion of lysine concentration of conjugate with respect to unmodified protein.

SEC

The conjugates were analysed by SEC using an Akta purifier 10 system and Biosep SEC-s3000 column eluting with DPBS:IPA (85:15). Conjugate purity was determined by integration of the Conjugate peak with respect to impurities/aggregate forms.

SDS-PAGE and Western Blotting

Native proteins and conjugates were analysed under heat denaturing non-reducing and reducing conditions by SDS-PAGE against a protein ladder using a 4-12% bis-tris gel and MES as running buffer (200V, ˜40 minutes). Samples were prepared to 0.5 mg/ml, comprising LDS sample buffer and MOPS running buffer as diluent. For reducing samples, DTT was added to a final concentration of 50 mM. Samples were heat treated for 2 minutes at 90-95° C. and 5 μg (10 μl) added to each well. Protein ladder (10 μl) was loaded without pre-treatment. Empty lines were filled with 1× LDS sample buffer (10 μl). After the gel was run, it was washed thrice with DI water (100 ml) with shaking (15 minutes, 200 rpm). Coomassie staining was performed by shaker-incubating the gel with PAGEBlue protein stain (30 ml) (60 minutes, 200 rpm). Excess staining solution was removed, rinsed twice with DI water (100 ml) and destained with DI water (100 ml) (60 minutes, 200 rpm). The resulting gel was imaged and processed using ImageJ (Rasband, W. S., ImageJ, U. S. National Institutes of Health, Bethesda, Maryland, USA).

TBEU-PAGE

Native protein, conjugates and BNA standard were analysed under heat denaturing non-reducing and reducing conditions by TBEU-PAGE against an oligo ladder using a 15% TBE-Urea gel and TBE as running buffer (180V, ˜60 minutes). Samples were prepared to 0.5 mg/ml, and BNA standard was prepared to 20 μg/ml, respectively, all comprising TBE Urea sample buffer and purified H₂O as diluent. Samples and standards were heat treated for 3 minutes at 70° C. and 10 μl added to each well, equating to 5 μg of protein and conjugate samples, and 0.2 μg of BNA, per lane. Oligo ladder reconstituted to 0.1 μg/band/ml in TE pH 7.5 (2 μl) was loaded without pre-treatment. After the gel was run, it was stained with freshly prepared ethidium bromide solution (1 μg/ml) with shaking (40 minutes, 200 rpm). The resulting gel was visualised by UV epi-illumination (254 nm), imaged and processed using ImageJ.

MALDI-TOF-MS

MALDI-TOF spectra were recorded on a MALDI-Mass Spectrometer (Bruker Ultrafex III). Typically, the sample dissolved in MilliQ water in nanomolar to micromolar range was spotted on the target (MTP 384 target plate polished steel T F, Bruker Daltons) using either super-DHB (99%, Fluka) or sinapinic acid (SA, 99%, Sigma-Aldrich) as the matrix dissolved in acetonitrile (MADLI-TOF-MS tested, Sigma)/0.1% TFA (7:3 v/v) via the dried-droplet-method. PepMix (Peptide Calibration Standard, Bruker Daltons) or ProteMass (Protein Calibration Standard, Sigma-Aldrich) served as calibration standards.

Release Kinetics Assay

The SO1861 conjugate of interest was dissolved in an acetate buffer (20 mM, pH 4.0 or pH 5.0), a phosphate buffer (20 mM) or in a PBS buffer (pH 7.4) with a final concentration of 10 μM at 20° C. or at 37° C. The release of SO1861 was followed overtime by monitoring the decrease of the UPLC-UV 4 peak area of the SO1861 conjugate of interest at different time points.

Cell Viability Assay

Cell viability was determined by an MTS-assay, performed according to the manufacturer's instruction (CellTiter 96® AQueous One Solution Cell Proliferation Assay, Promega). Briefly, the MTS solution was diluted 20× in DMEM without phenol red (PAN-Biotech GmbH) supplemented with 10% FBS (PAN-Biotech GmbH). The cells were washed once with 200 μL PBS per well, after which 100 μL diluted MTS solution was added per well. The plate was incubated for approximately 20-30 minutes at 37° C. Subsequently, the optical density at 492 nm was measured on a Thermo Scientific Multiskan FC plate reader (Thermo Scientific). For quantification the background signal of ‘medium only’ wells was subtracted from the signal from all other wells, before the ratio of untreated/treated cells was calculated, by dividing the background corrected signal of untreated wells by the background corrected signal of the treated wells.

FACS Analysis

Cells were seeded in DMEM (PAN-Biotech GmbH) supplemented with 10% fetal calf serum (PAN-Biotech GmbH) and 1% penicillin/streptomycin (PAN-Biotech GmbH), at 500,000 c/plate in 10 cm dishes and incubated for 48 hrs (5% CO₂, 37° C.), until a confluency of 90% was reached. Next, the cells were trypsinized (TrypIE Express, Gibco Thermo Scientific) to single cells. 0.75×10⁶ Cells were transferred to a 15 mL Falcon tube and centrifuged (1,400 rpm, 3 min). The supernatant was discarded while leaving the cell pellet submerged. The pellet was dissociated by gentle tapping the Falcon tube on a vortex shaker and the cells were washed with 4 mL cold PBS (Mg²⁺ and Ca²⁺ free, 2% FBS). After washing, the cells were resuspended in 3 mL cold PBS (Mg²⁺ and Ca²⁺ free, 2% FBS) and divided equally over 3 round bottom FACS tubes (1 mL/tube). The cells were centrifuged again and resuspended in 200 μL cold PBS (Mg²⁺ and Ca²⁺ free, 2% FBS) or 200 μL antibody solution containing 5 μL antibody in 195 μL cold PBS (Mg²⁺ and Ca²⁺ free, 2% FBS). APC Mouse IgG1, κ APC anti-human EGFR (#352906, Biolegend) was used to stain the EGFR receptor. PE anti-human HER2 APC anti-human CD340 (erbB2/HER-2) (#324408 Biolegend) was used to stain the HER2 receptor, PE Mouse IgG2a, K Isotype Ctrl FC (#400212, Biolegend) was used as its matched isotype control. PE anti-human CD71 (#334106, Biolegend) was used to stain the CD71 receptor, PE Mouse IgG2a, K Isotype Ctrl FC (#400212, Biolegend) was used as its matched isotype control. Samples were incubated for 30 min at 4° C. on a tube roller mixer. Afterwards, the cells were washed 3× with cold PBS (Mg²⁺ and Ca²⁺ free, 2% FBS) and fixated for 20 min at room temperature using a 2% PFA solution in PBS. Cells were washed 2× with cold PBS and resuspended in 250-350 μL cold PBS for FACS analysis. Samples were analyzed with a BD FACSCanto II flow cytometry system (BD Biosciences) and FlowJo software.

TABLE 1
Expression levels of EGFR, HER2 and CD71 of various cells

| | EGFR | HER2 | CD71 |
|---|---|---|---|
| | expression | expression | expression |
| Cell line | level (MFI) | level (MFI) | level (MFI) |
| MDA-MB-468 | 1656 | 1 | 186 |
| A431 | 1593 | 10 | 322 |
| CaSki | 481 | 12 | 189 |
| SK-BR-3 | 28 | 1162 | 331 |
| JIMT-1 | 58 | 74 | 107 |
| HeLa | 91 | 7 | 312 |
| A2058 | 1 | 5 | 59 |

CMC Determination

The critical micellar concentration (CMC) of saponins derived from Saponaria Officinalis (SO) was determined by the method of DeVendittis et al. (www.sciencedirect.com/science/article/pii/0003269781900063, doi.org/10.1016/0003-2697(81)90006-3, Analytical Biochemistry, Volume 115, Issue 2, August 1981, Pages 278-286) as follows: The emission spectrum of 8-Anilinonaphthalene-1-sulfonic acid (ANS) in either purified water (MQ) or PBS (Dulbecco's PBS+/+) was determined at dry weight concentrations of saponins ranging from 1 to 1400 μM to cover the range below and above the CMC. Above the CMC, the fluorescence yield of ANS increases and the wavelength of maximum emission decreases due to portioning of the fluorescent dye into micelles. Fluorescence yields were recorded on a Fluoroskan Ascent FL (Thermo Scientific) at an excitation wavelength of 355 nm, and an emission wavelength of 460 nm. 6 μg at a concentration of 75.86 μM of ANS were used per sample and measurement.

Haemolysis Assay

Red blood cells (RBCs) were isolated from a buffy coat using a Ficoll gradient. The obtained RBC pellet (˜4-5 ml) was washed 2× with 50 ml DPBS (without Ca²⁺/Mg²⁺, PAN-Biotech GmbH). Cells were pelleted by centrifugation for 10 min, 800×g at RT. RBC were counted and resuspended at 500,000,000 c/ml in DPBS (without Ca²⁺/Mg²⁺), based on total cell count. SO1861-linker dilutions were prepared in DPBS (with Ca²⁺/Mg²⁺, PAN-Biotech GmbH), at 1.11× final strength. For positive lysis control a 0.02% Triton-X100 solution was prepared in DPBS^{+/+}. Of all compound solutions 135 μl was dispensed/well in a 96 well V-bottom plate. To this 15 μl RBC suspension was added and mixed shortly (10 sec-600 rpm). The plate was incubated 30 min at RT, with gentle agitation. Afterwards the plate was spun for 10 min at 800×g to pellet the RBC and 100-120 μl supernatant was transferred to a standard 96 wp. Subsequently, the OD at 405 nm was measured on a Thermo Scientific Multiskan FC plate reader (Thermo Scientific). For quantification the background signal of ‘DPBS^{+/+} only’ wells was subtracted from all other wells before the percentage of hemolysis was calculated in comparison to 0.02% Triton-X100, by dividing the background corrected signal of treated wells over the background corrected signal of the 0.02% Triton-X100 wells (×100).

RNA Isolation and Gene Expression Analysis

RNA from cells was isolated and analysed according to standard protocols (Biorad). The qPCR primers that were used are indicated in Table 2.

TABLE 2
Primers used in qPCR are shown below:

| Gene | Primer | Sequence (5′-3′) [SEQ ID No.] |
|---|---|---|
| HSP27 | Forward | GCAGTCCAACGAGATCACCA [SEQ ID NO. 2] |
| | Reverse | TAAGGCTTTACTTGGCGGCA [SEQ ID NO. 3] |
| HBMS | Forward | CACCCACACACAGCCTACTT [SEQ ID NO. 4] |
| (control) | Reverse | GTACCCACGCGAATCACTCT [SEQ ID NO. 5] |
| GUSB | Forward | GAAAATACGTGGTTGGAGAGCT [SEQ ID NO. 6] |
| (control) | Reverse | CCGAGTGAAGATCCCCTTTTTA [SEQ ID NO. 7] |

Synthesis

SO1861-SC-Mal (FIG. 1)

### Intermediate 1

### benzyl 4-(2-(tert-butoxycarbonyl)hydrazine-1-carbonyl)piperazine-1-carboxylate

To a stirring solution of phosgene in toluene (20%, w/w, 15.8 mL, 30.0 mmol) at 0° C. was added slowly (10 min) a solution of 1-Cbz-piperazine (1.93 mL, 10.0 mmol) in dichloromethane (25 mL) and DIPEA (3.83 mL, 22.0 mmol). The reaction mixture was stirred at room temperature. After 30 min the reaction mixture was evaporated in vacuo and co-evaporated with dichloromethane (2×50 mL). Next, the residue was dissolved in dichloromethane (100 mL) and resulting solution was stirred at 0° C. A solution of Boc hydrazine (2.33 mL, 15.0 mmol) in dichloromethane (20 mL) and DiPEA (3.83 mL, 22.0 mL) was added and the reaction mixture was stirred overnight at room temperature. The reaction mixture was diluted with dichloromethane (100 mL) and the resulting solution was washed with 0.5 N potassium bisulphate solution (2×100 mL) and brine (100 mL), dried over Na₂SO4, filtered and evaporated in vacuo. The residue was purified by flash chromatography (first by an ethyl acetate—heptane gradient, 5:95 (v/v) rising to 100% ethyl acetate followed by a flush with 10% methanol in DCM (v/v)) to give the title compound (2.42 g, 64%) as a white solid. Purity based on LC-MS 100%.

LRMS (m/z): 279/323/401 [M−99/M−55/M+23]¹⁺
LC-MS r.t. (min): 1.27¹

### Intermediate 2

### tert-butyl 2-(piperazine-1-carbonyl)hydrazine-1-carboxylate

To benzyl 4-(2-(tert-butoxycarbonyl)hydrazine-1-carbonyl)piperazine-1-carboxylate (2.42 g, 6.39 mmol) was added methanol (50 mL). The mixture was heated to obtain a clear solution. Next, palladium (10% on activated carbon, 50% wet with water, 1.50 g) was added and the resulting mixture was stirred under a hydrogen atmosphere by using a balloon. After 1 hour the reaction mixture was filtered over celite and the filtrate evaporated in vacuo to give the title product (1.56 g, quant.) as a white solid.

LRMS (m/z): 189/245 [M−55/M+1]¹⁺

### Intermediate 3

### tert-butyl 2-(4-(6-(2,5-dioxo-2,5-dihydro-1H-pyrrol-1-yl)hexanoyl)piperazine-1-carbonyl)hydrazine-1-carboxylate

6-maleimidocaproic acid (810 mg, 3.84 mmol), tert-butyl 2-(piperazine-1-carbonyl)hydrazine-1-carboxylate (781 mg, 3.20 mmol), EDCl·HCl (735 mg, 3.84 mmol) and Oxyma Pure (591 mg, 4.16 mmol) were dissolved in a mixture of dichloromethane (25 mL) and DIPEA (835 μL, 4.80 mmol) and the reaction mixture was stirred at room temperature. After 2 hours the reaction mixture was evaporated in vacuo and the residue was dissolved in ethyl acetate (50 mL). The resulting solution was washed with 0.5 N potassium bisulphate solution (50 mL), saturated sodium bicarbonate solution (2×50 mL) and brine (50 mL), dried over Na₂SO₄, filtered and evaporated in vacuo. The residue was purified by flash chromatography (DCM−10% methanol in DCM (v/v) gradient 100:0 rising to 40:60) to give the title compound (634 mg, 45%) as a white solid. Purity based on LC-MS 97%.

LRMS (m/z): 338/382/460 [M−99/M−55/M+23]¹⁺

LC-MS r.t. (min): 1.02¹

SO1861-SC-Mal (FIG. 1)

Chemical Formula: SO1861-SC-Mal: C₉₈H₁₅₁N₅O₄₉, Exact Mass: 2181.95

tert-butyl 2-(4-(6-(2,5-dioxo-2,5-dihydro-1H-pyrrol-1-yl)hexanoyl)piperazine-1-carbonyl)hydrazine-1-carboxylate (25.0 mg, 57.1 μmol) was dissolved in a mixture of dichloromethane (500 μL) and TFA (500 μL) and the reaction mixture was stirred at room temperature. After 30 min the reaction mixture was evaporated in vacuo and co-evaporated with dichloromethane (3×5 mL) and methanol (5 mL). The residue and SO1861 (21.3 mg, 11.4 μmol) were dissolved in methanol (extra dry, 1.00 mL) and the resulting mixture was shaken for 1 min and left standing at room temperature. After 4 hours the reaction mixture was subjected to to preparative MP-LC. 2 Fractions corresponding to the product were immediately pooled together, frozen and lyophilized overnight to yield the title compound (13.7 mg, 55%) as a white fluffy solid. Purity based on LC-MS 97%.

LRMS (m/z): 2181 [M−1]¹⁻

LC-MS r.t. (min): 2.13³

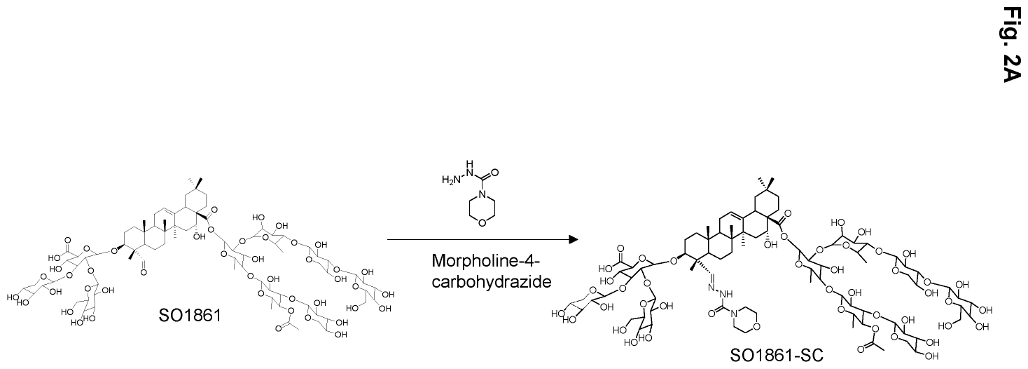

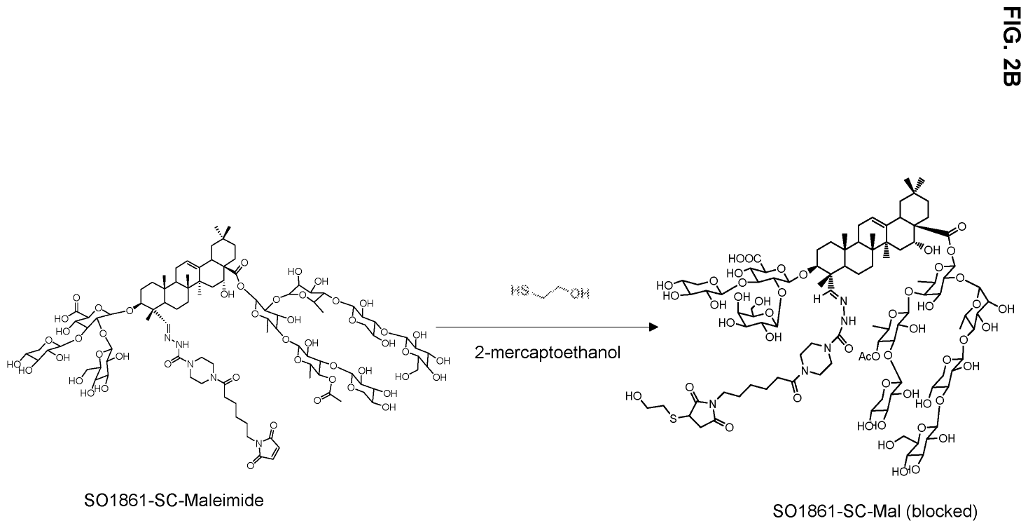

SO1861-SC (FIG. 2A)

Morpholine-4-carbohydrazide (8.1 mg, 56 μmol) and SO1861 (21.3 mg, 11.4 μmol) were dissolved in methanol (extra dry, 1.00 mL). Then 50 μLTFA was added and the resulting mixture was shaken for 1 min and left standing at room temperature. After 4 hours the reaction mixture was subjected to preparative MP-LC. 2 Fractions corresponding to the product were immediately pooled together, frozen and lyophilized overnight to yield the title compound (13.8 mg, 61%) as a white fluffy solid. Purity based on LC-MS 96%.

LRMS (m/z): 1988 [M−1]¹⁻

SO1861-SC-Mal (Blocked) (FIG. 2B)

SO1861-SC-Mal (3.63 mg, 1.66 μmol) was dissolved in a solution of 20 mM ammonium bicarbonate/acetonitrile (3:1, v/v, 500 μL). Directly afterwards 2-mercaptoethanol (0.466 μL, 6.65 μmol) was added. The reaction mixture was shaken for 1 min and left standing at room temperature. After 30 min the reaction mixture was subjected to to preparative MP-LC. 2 Fractions corresponding to the product were immediately pooled together, frozen and lyophilized overnight to yield the title compound (2.35 mg, 63%) as a white fluffy solid. Purity based on LC-MS 96%.

LRMS (m/z): 2260 [M−1]¹⁻

LC-MS r.t. (min): 3.53⁴

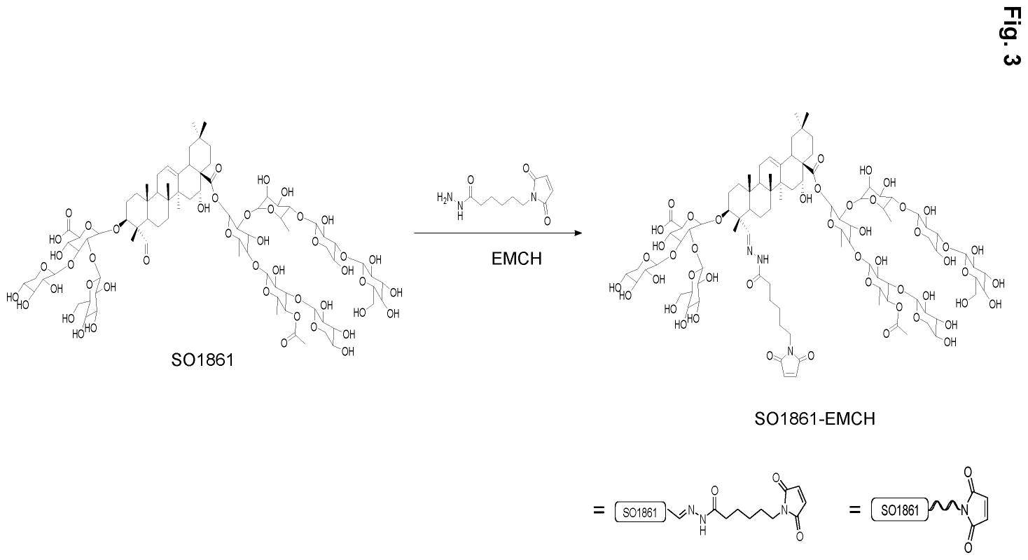

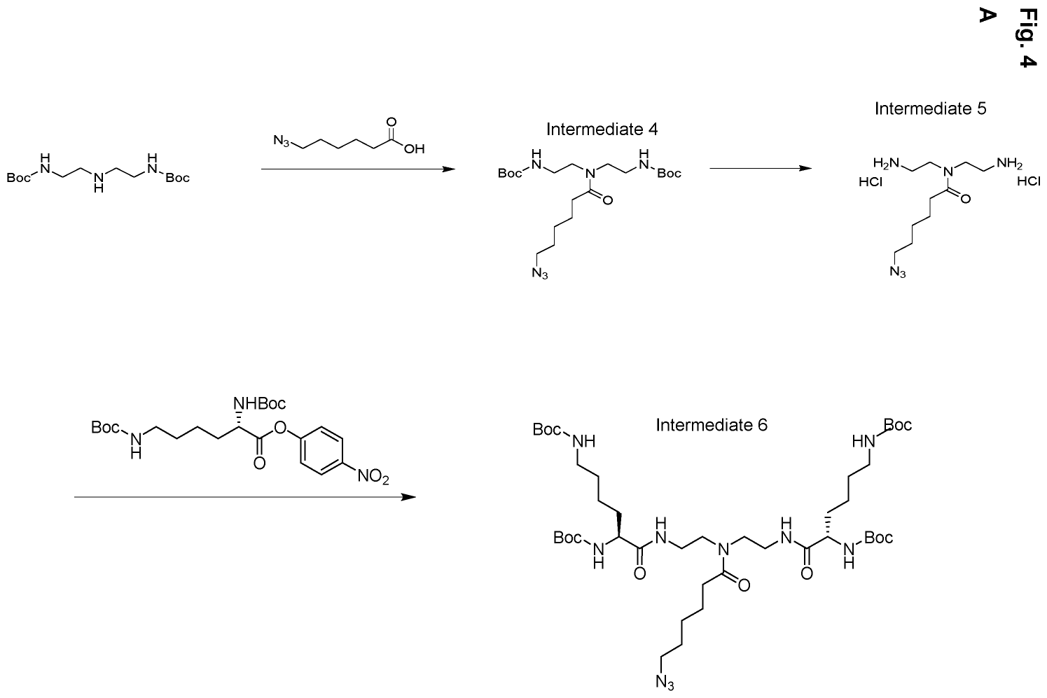

SO1861-EMCH Synthesis (FIG. 3)

To SO1861 (121 mg, 0.065 mmol) and EMCH·TFA (110 mg, 0.325 mmol) was added methanol (extra dry, 3.00 mL) and TFA (0.020 mL, 0.260 mmol). The reaction mixture stirred at room temperature. After 1.5 hours the reaction mixture was subjected to preparative MP-LC.¹ Fractions corresponding to the product were immediately pooled together, frozen and lyophilized overnight to give the title compound (120 mg, 90%) as a white fluffy solid. Purity based on LC-MS 96%.

LRMS (m/z): 2069 [M−1]¹⁻

LC-MS r.t. (min): 1.08⁴

Dendron(SO1861)4 Synthesis (FIG. 4)

### Intermediate 4: (FIG. 4a)

### di-tert-butyl (((6-azidohexanoyl)azanediyl)bis(ethane-2,1-diyl))dicarbamate

6-azidohexanoic acid (0.943 g, 6.00 mmol), EDCI.HCl (1.21 g, 6.30 mmol) and Oxyma Pure (0.938 g, 6.60 mmol) were dissolved in DMF (10.0 mL) and the mixture was stirred for 5 min. Next a solution of di-tert-butyl (azanediylbis(ethane-2,1-diyl))dicarbamate (1.82 g, 6.00 mmol) in DMF (5.00 mL) was added and the reaction mixture was stirred at room temperature. After 5 hours the reaction mixture was evaporated in vacuo and the residue was dissolved in ethyl acetate (50 mL). The resulting solution was washed with 1N potassium bisulphate solution (50 mL), saturated sodium bicarbonate solution (2×50 mL) and brine (50 mL), dried over Na₂SO4, filtered and evaporated in vacuo. The residue was purified by flash chromatography (ethyl acetate-heptane gradient, 10:90 rising to 100:0) to give the title compound (2.67 g, 100%) as a white solid. Purity based on LC-MS 98%.

LRMS (m/z): 287/343/465 [M−155/M−99/M+23]¹⁺

LC-MS r.t. (min): 2.02^{2A}

### Intermediate 5: (FIG. 4a)

### N,N-bis(2-aminoethyl)-6-azidohexanamide dihydrochloride

To di-tert-butyl (((6-azidohexanoyl)azanediyl)bis(ethane-2,1-diyl))dicarbamate (2.66 g, 6.00 mmol) was added HCl in isopropanol (5-6 N, 20.0 mL, 110 mmol) and the reaction mixture was stirred at room temperature. After 4 hours, the reaction mixture was evaporated in vacuo and the resulting crude product was co-evaporated with DCM (3×20 mL) to give the crude title product (1.49 g, 79%) as a white solid.

LRMS (m/z): 243 [M+1]¹⁺

### Intermediate 6: (FIG. 4a)

### tetra-tert-butyl ((5S,5'S)-((((6-azidohexanoyl)azanediyl)bis(ethane-2,1-diyl))bis(azanediyl))bis(6-oxohexane-6,1,5-triyl))tetracarbamate

To a solution of N,N-bis(2-aminoethyl)-6-azidohexanamide dihydrochloride (1.19 g, 3.76 mmol) in DMF (30.0 mL) and DIPEA (2.62 mL, 15.1 mmol) was added Boc-Lys(Boc)-ONp (3.69 g, 7.90 mmol) and the mixture was stirred at room temperature overnight. The reaction mixture was evaporated in vacuo and the residue was dissolved in ethyl acetate (100 mL). The resulting solution was washed with 1N potassium bisulphate solution (100 mL) and saturated sodium bicarbonate solution (5×100 mL), dried over Na₂SO₄, filtered and evaporated in vacuo. The residue was purified by flash chromatography (DCM—methanol/DCM (1/9, v/v) gradient 100:0 rising to 0:100) to give the give the title product (3.07 g, 91%) as a slightly yellowish solid. Purity based on LC-MS 94%.

LRMS (m/z): 800/900/922 [M−99/M+1/M+23]¹⁺

LC-MS r.t. (min): 2.17^{2A}

### Intermediate 7: (FIG. 4b)

### 4-nitrophenyl 3-(acetylthio)propanoate

4-Nitrophenyl trifluoroacetate (5.17 g, 22.0 mmol) and 3-(Acetylthio)propionic Acid (2.96 g, 20.0 mmol) were dissolved in DCM (50.0 mL). Next, DIPEA (6.97 mL, 40.0 mmol) was added and the reaction mixture was stirred at room temperature overnight. The reaction mixture was evaporated in vacuo and the residue was dissolved in ethyl acetate (50 mL). The resulting solution was washed with 1N potassium bisulphate solution (50 mL), saturated sodium bicarbonate solution (5×50 mL) and brine (50 mL), dried over Na₂SO₄, filtered and evaporated in vacuo. The residue was purified by flash chromatography (DCM—methanol/DCM (1/9, v/v) gradient 100:0 rising to 0:100) to give the give the title product (4.90 g, 91%) as a slightly yellowish solid. Purity based on LC-MS 99%.

LRMS (m/z): 292 [M+23]¹⁺

LC-MS r.t. (min): 1.94^{2A}

### Intermediate 8: (FIG. 4b)

### (S)-2,6-diamino-N-(2-(6-azido-N-(2-((S)-2,6-diaminohexanamido)ethyl)hexanamido)ethyl)hexanamide tetrahydrochloride

tetra-tert-butyl ((5S,5'S)-((((6-azidohexanoyl)azanediyl)bis(ethane-2,1-diyl))bis(azanediyl))bis(6-oxohexane-6,1,5-triyl))tetracarbamate (1.80 g, 2.00 mmol) was dissolved in HCl in isopropanol (5-6N, 50.0 ml, 275 mmol) and the reaction mixture was stirred at room temperature overnight. The reaction mixture was evaporated in vacuo and the resulting crude product was co-evaporated with DCM (3×20 mL) to give the crude title product as a white solid.

LRMS (m/z): 250 [M+2]²⁺, 500 [M+1]¹⁺

### Intermediate 9: (FIG. 4b)

### (2S)-2,6-bis[3-(acetylsulfanyl)propanamido]-N-[2-(6-azido-N-{2-[(2S)-2,6-bis[3-(acetylsulfanyl)propanamido]hexanamido]ethyl}hexanamido)ethyl]hexanamide

To a solution of (S)-2,6-diamino-N-(2-(6-azido-N-(2-((S)-2,6-diaminohexanamido)ethyl)hexanamido) ethyl)hexanamide tetrahydrochloride (1.29 g, 2.00 mmol) in DMF (30 mL) and DIPEA (3.48 mL, 20.0 mmol) was added 4-nitrophenyl 3-(acetylthio)propanoate (2.26 g, 8.40 mmol) and the reaction mixture was stirred at room temperature over the weekend. The reaction mixture was evaporated in vacuo and the residue was dissolved in DCM/methanol (95:5, v/v, 100 mL). The resulting solution was washed with 1N potassium bisulphate solution (100 mL), 1 N sodium hydroxide solution (3×100 mL) and brine (100 mL), dried over Na₂SO₄, filtered and evaporated in vacuo. The residue was purified by flash chromatography (DCM—methanol/DCM (1/9, v/v) gradient 100:0 rising to 0:100) to give the title product (1.33 g, 65%) as a white solid. On LC-MS an impurity (15%) was found with m/z values corresponding to the product with one deprotected thioacetate group. The impurity was formed during or after work-up. Purity based on LC-MS 85%.

LRMS (m/z): 510 [M+2]²⁺, 1019/1041 [M+1/M+23]¹⁺

LC-MS r.t. (min): 1.86^{2B}

### Intermediate 10: (FIG. 4c)

### N,N′-((9S,19S)-14-(6-aminohexanoyl)-1-mercapto-9-(3-mercaptopropanamido)-3,10,18-trioxo-4,11,14,17-tetraazatricosane-19,23-diyl)bis(3-mercaptopropanamide) formate

Intermediate 9 (102 mg, 0.100 mmol) was dissolved in methanol (1.00 ml). Next, a freshly prepared 1 N Sodium hydroxide solution (0.440 ml, 0.440 mmol) was added and the reaction mixture was stirred at room temperature. After 30 min a 1.0 M trimethylphosphine solution in THF (0.500 ml, 0.500 mmol) was added and the resulting mixture was stirred at room temperature. After 30 min the reaction mixture was evaporated in vacuo and co-evaporated with methanol (2×10 mL). The residue was dissolved in a mixture of methanol/water (9:1, v/v, 1.00 mL) and the resulting solution was subjected to preparative MP-LC. 2 Fractions corresponding to the product were immediately pooled together, frozen and lyophilized overnight to give the title compound (75.6 mg, 87%) as a colorless sticky oil. Purity based on LC-MS 96%.

LRMS (m/z): 513 [M+2]²⁺, 825 [M+1]¹⁺

LC-MS r.t. (min): 1.42^{2A}

### Intermediate 11 and 12: (FIG. 4d)

The conjugation of SO1861 to the dendron to yield Dendron(SO1861)4-amine was performed for both SO1861-EMCH and SO1861-SC-maleimide.

### Intermediate 11

Dendron(EMCH-SO1861)4-Amine (FIG. 4e)

N,N′-((9S,19S)-14-(6-aminohexanoyl)-1-mercapto-9-(3-mercaptopropanamido)-3,10,18-trioxo-4,11,14,17-tetraazatricosane-19,23-diyl)bis(3-mercaptopropanamide) formate (2.73 mg, 3.13 μmol) was dissolved in a mixture of 20 mM NH₄HCO₃ with 0.5 mM TCEP/acetonitrile (3:1, v/v, 3.00 mL). Next, SO1861-EMCH (29.2 mg, 0.014 mmol) was added and the reaction mixture was stirred at room temperature. After 1.5 hours the reaction mixture was subjected to preparative LC-MS.^{3B} Fractions corresponding to the product were immediately pooled together, frozen and lyophilized overnight to give the title compound (12.3 mg, 43%) as a white fluffy solid. Purity based on LC-MS 97%.

LRMS (m/z): 1517 [M−6]⁶⁻, 1821 [M−5]⁵⁻, 2276 [M−4]⁴⁻

LC-MS r.t. (min): 4.39^{5A}

### Intermediate 12

Dendron-(semicarbazone-SO1861) 4-amine (FIG. 4e)

N,N′-((9S,19S)-14-(6-aminohexanoyl)-1-mercapto-9-(3-mercaptopropanamido)-3,10,18-trioxo-4,11,14,17-tetraazatricosane-19,23-diyl)bis(3-mercaptopropanamide) formate (2.73 mg, 3.13 μmol, molecule 38) was dissolved in a mixture of 1M NaOH and MeOH (1:1, v/v, 3.00 mL) and stirred for 30 min. Next, 5 mL of Trimethylphosphine (9.5 mg, 125 μmol) dissolved in THF was added and the mixture was stirred for 30 min. Next, SO1861-SC-Mal (30.54 mg, 0.014 mmol, molecule 37) dissolved in 20 mM NH₄HCO₃ with acetonitrile (3:1, v/v, 3.00 mL) was added and the reaction mixture was stirred at room temperature. After 1.5 hours the reaction mixture was subjected to preparative LC-MS.^{3B} Fractions corresponding to the product were immediately pooled together, frozen and lyophilized overnight to give the title compound (19 mg, 64%) as a white fluffy solid. Purity based on LC-MS 95%.

MS (m/z): 9553 [M+H]⁺

Dendron-(SO1861)4-azide

Dendron(EMCH-SO1861)4-azide (FIG. 4f)

Dendron-(EMCH-SO1861)4-amine (6.81 mg, 0.748 μmol) and 2,5-dioxopyrrolidin-1-yl 1-azido-3,6,9,12-tetraoxapentadecan-15-oate (2.90 mg, 7.48 μmol) were dissolved in DMF(1.00 mL). Next, DIPEA (1.302 μL, 7.48 μmol) was added and the mixture was shaken for 1 min and left standing at room temperature. After 2 hours the reaction mixture was subjected to preparative LC-MS.^{3C} Fractions corresponding to the product were immediately pooled together, frozen and lyophilized overnight to give the title compound (5.86 mg, 84%) as a white fluffy solid. Purity based on LC-MS 90%.

LRMS (m/z): 2344 [M−4]⁴⁻

LC-MS r.t. (min): 4.78^{5B}

Dendron(semicarbazone-SO1861)4-azide (FIG. 4f):

Dendron-(semicarbazone-SO1861)₄-amine (7.1 mg, 0.748 μmol, molecule 39) and 2,5-dioxopyrrolidin-1-yl 1-azido-3,6,9,12-tetraoxapentadecan-15-oate (2.90 mg, 7.48 μmol, molecule 18) were dissolved in DMF (1.00 mL). Next, DIPEA (1.302 μL, 7.48 μmol) was added and the mixture was shaken for 1 min and left standing at room temperature. After 2 hours the reaction mixture was subjected to preparative LC-MS.^{3C} Fractions corresponding to the product were immediately pooled together, frozen and lyophilized overnight to give the title compound (6.4 mg, 87%) as a white fluffy solid. Purity based on LC-MS 93%.

MS (m/z): 9826 [M+H]⁺

Dendron-(SO1861)4-maleimide1 (FIG. 4g)

The synthesis of Dendron(SO1861)4-maleimide1 was performed for both saponin-dendron conjugates Dendron(EMCH-SO1861) 4-amine and Dendron(semicarbazone-SO1861)4-amine. The following synthesis is exemplary described for Dendron-(EMCH-SO1861)4-maleimide1.

Dendron-(EMCH-SO1861)4-maleimide1 (FIG. 4g)

Dendron(SO1861)4-amine (8.12 mg, 0.891 μmol) and 2,5-dioxopyrrolidin-1-yl 1-(2,5-dioxo-2,5-dihydro-1H-pyrrol-1-yl)-3,6,9,12-tetraoxapentadecan-15-oate (3.94 mg, 8.91 μmol) were dissolved in DMF(1.00 mL). Next, DIPEA (1.55 μL, 8.91 μmol) was added and the mixture was shaken for 1 min and left standing at room temperature. After 3 hours the reaction mixture was subjected to preparative LC-MS.^{3C} Fractions corresponding to the product were immediately pooled together, frozen and lyophilized overnight to give the title compound (6.76 mg, 80%) as a white fluffy solid. Purity based on LC-MS 66%.

LRMS (m/z): 2358 [M−4]⁴⁻

LC-MS r.t. (min): 2.13^{6C}

Dendron-(SO1861)4-maleimide2 (FIG. 4h)

The synthesis of Dendron(SO1861)4-maleimide1 was performed for both saponin-dendron conjugates Dendron(EMCH-SO1861)4-amine and Dendron-(semicarbazone-SO1861)4-amine. The following synthesis is exemplary described for Dendron(EMCH-SO1861)4-maleimide2.

Dendron(SO1861)4-maleimide2 (FIG. 4h)

Intermediate 9 (5.10 mg, 5.00 μmol) was dissolved in methanol (100 μL). Next, a freshly prepared 1 N Sodium hydroxide solution (22.0 μL, 22.0 μmol) was added and the mixture was shaken for 1 min and left standing at room temperature. After 30 min a 1.0 M trimethylphosphine solution in THF (25.0 μL, 25.0 μmol) was added and the resulting mixture was shaken for 1 min and left standing at room temperature. After 30 min the reaction mixture was evaporated in vacuo and co-evaporated with methanol (2×5 mL). The resulting residue was dissolved in a mixture of 20 mM NH₄HCO₃ with 0.5 mM TCEP/acetonitrile (3:1, v/v, 3.242 mL). From this solution, directly, 1000 μL was added to SO1861-EMCH (14.4 mg, 6.94 μmol, 4.5 equiv. compared to the scaffold) and the mixture was shaken for 1 min and left standing at room temperature. After 10 min the reaction mixture was lyophilized overnight. To the resulting residue 2,5-Dioxopyrrolidin-1-yl 3-(2-(2-(3-(2,5-dioxo-2h-pyrrol-1(5h)-yl)propanamido)ethoxy)ethoxy)propanoate (5.84 mg, 0.014 mmol) and DMF (1.00 mL) were added. Next, DIPEA (2.39 μL, 0.014 mmol) was added and the suspension was shaken for 1 min and left standing at room temperature. After 2 hours the reaction mixture was subjected to preparative LC-MS.^{3C} Fractions corresponding to the product were immediately pooled together, frozen and lyophilized overnight to give the title compound (10.9 mg, 85%) as a white fluffy solid. Purity based on LC-MS 80%.

LRMS (m/z): 2354 [M−4]⁴⁻

LC-MS r.t. (min): 4.16^{5B}

Dendron-(SO1861)8 Synthesis

### Intermediate 13: (FIG. 5a)

### tert-butyl N-U1S)-1-{[(1S)-1-{[2-(6-azido-N-{2-[(2S)-2,6-bis[(2S)-2,6-bis({[(tert-butoxy)carbonyl]amino})hexanamido])hexanamido]ethyl}hexanamido)ethyl]carbamoyl}-5-[(2S)-2,6-bis({[(tert-butoxy)carbonyl]amino}hexanamido]pentyl]carbamoyl}-5-{[(tert-butoxy)carbonyl]amino}pentyl]carbamate

(S)-2,6-diamino-N-(2-(6-azido-N-(2-((S)-2,6-diaminohexanamido)ethyl)hexanamido)ethyl)hexanamide tetrahydrochloride (964 mg, 1.50 mmol) was dissolved in DMF (25.0 mL) and triethylamine (2.08 mL, 15.0 mmol). Next, Boc-Lys(Boc)-ONp (3.36 g, 7.18 mmol) was added and the reaction mixture was stirred at room temperature overnight. The reaction mixture was evaporated in vacuo and the residue was purified by flash chromatography (DCM—methanol/DCM (1/9, v/v) gradient 100:0 rising to 0:100) to give the title product (2.71 g, 100%) as a white solid. Purity based on LC-MS 97%.

LRMS (m/z): 807 [M−198]²⁺

LC-MS r.t. (min): 2.35^{2B}

### Intermediate 14: (FIG. 5b)

### (2S,2'S)—N,N′-((5S,15S,22S)-22,26-diamino-10-(6-azidohexanoyl)-154(S)-2,6-diaminohexanamido)-6,14,21-trioxo-7,10,13,20-tetraazahexacosane-1,5-diyl)bis(2,6-diaminohexanamide) octahydrochloride

Intermediate 13 (2.71 g, 1.50 mmol) was dissolved in HCl in isopropanol (5-6N, 25.0 ml, 138 mmol) and the reaction mixture was stirred at room temperature overnight. Next, the reaction mixture was evaporated in vacuo and the resulting crude product was co-evaporated with DCM (3×20 mL) to give the crude title product as a white solid.

LRMS (m/z): 203/254 [M−200/M+4]⁴⁺, 338 [M+3]³⁺, 507 [M+2]²⁺, 1012 [M+1]¹⁺

### Intermediate 15: (FIG. 5c)

### (2S)-2,6-bis[3-(acetylsulfanyl)propanamido]-N-[(1S)-1-{[2-(6-azido-N-{2-[(2S)-2,6-bis[(2S)-2,6-bis[3-(acetylsulfanyl)propanamido]hexanamido]hexanamido]ethyl}hexanamido)ethyl]carbamoyl}-5-[(2S)-2,6-bis[3-(acetylsulfanyl)propanamido]hexanamido]pentypexanamide

To (2S,2'S)—N,N′-((5S,15S,22S)-22,26-diamino-10-(6-azidohexanoyl)-15-((S)-2,6-diaminohexanamido)-6,14,21-trioxo-7,10,13,20-tetraazahexacosane-1,5-diyl) bis(2,6-diaminohexanamide) octahydrochloride (300 mg, 0.230 mmol) was added DMF (20.0 mL), triethylamine (320 μl, 2.30 mmol) and 4-nitrophenyl 3-(acetylthio)propanoate (595 mg, 2.21 mmol). The resulting suspension was sonicated at 60° C. for 30 min and left stirring at room temperature overnight. The reaction mixture was evaporated in vacuo and the residue was purified by first performing flash chromatography (DCM—methanol/DCM (1/9, v/v) gradient 100:0 rising to 0:100), followed by preparative MP-LC 2 to give the title product (70 mg, 15%) as a white solid. Purity based on LC-MS 100%.

LRMS (m/z): 685 [M+3]³⁺

LC-MS r.t. (min): 1.91^{2A}

### Intermediate 16: (FIG. 5d)

### (2S)—N-[(1S)-1-{[2-(6-amino-N-{2-[(2S)-2,6-bis[(2S)-2,6-bis(3-sulfanylpropanamido)hexanamido[hexanamido]ethyl}hexanamido)ethyl]carbamoyl}-5-[(2S)-2,6-bis(3-sulfanylpropanamido)hexanamido]pentyl]-2,6-bis(3-sulfanylpropanamido)hexanamide formate

Intermediate 15 (10.0 mg, 4.87 μmol) was dissolved methanol (200 μL). Next, a freshly prepared 1 N Sodium hydroxide solution (42.9 μL, 0.043 mmol) was added and the resulting mixture was shaken for 1 min and left standing at room temperature. After 30 min a 1.0 M trimethylphosphine solution in THF (24.4 μL, 0.024 mmol) was added and the resulting mixture was shaken for 1 min and left standing at room temperature. After 30 min the reaction mixture was diluted with water (1 mL) and directly subjected to preparative MP-LC.² Fractions corresponding to the product were immediately pooled together, frozen and lyophilized overnight to give the title compound (4.02 mg, 48%) as a white fluffy solid.

LRMS (m/z): 564 [M+3]³⁺, 846 [M+2]²⁺

LC-MS r.t. (min): 1.54^{2C}

Dendron-(SO1861)8-amine (FIG. 4d)

The conjugation of SO1861 to the dendron to yield Dendron-(SO1861)8-amine was performed for both SO1861-EMCH and SO1861-SC-maleimide.

### Intermediate 17: (FIG. 5e)

Dendron(EMCH-SO1861)8-amine

Intermediate 16 (0.52 mg, 0.299 μmol) and SO1861-EMCH (29.2 mg, 0.014 mmol) were dissolved in a mixture of 20 mM NH₄HCO₃ with 0.5 mM TCEP/acetonitrile (3:1, v/v, 1.00 mL) and the resulting mixture was shaken for 1 min and left standing at room temperature. After 30 min TCEP (0.30 mg, 1.05 μmol) was added and the reaction mixture was shaken for 1 min. Next, the mixture was directly subjected to preparative LC-MS. 3B Fractions corresponding to the product were immediately pooled together, frozen and lyophilized overnight to give the title compound (2.17 mg, 40%) as a white fluffy solid. Purity based on LC-MS 97%.

LRMS (m/z): 2282 [M−8]⁸⁻, 2607 [M−7]⁷⁻

LC-MS r.t. (min): 4.41^{5A}

### Intermediate 18: (FIG. 5e)

Dendron(semicarbazone-SO1861)8-amine

(2S)—N-[(1S)-1-{[2-(6-amino-N-{2-[(2S)-2,6-bis[(2S)-2,6-bis(3-sulfanylpropanamido)hexanamido]hexanamido]ethyl}hexanamido)ethyl]carbamoyl}-5-[(2S)-2,6-bis(3-sulfanylpropanamido)hexanamido]pentyl]-2,6-bis(3-sulfanylpropanamido)hexanamide formate (0.61 mg, 0.299 μmol, molecule 43) was dissolved in a mixture of 1M NaOH and MeOH (1:1, v/v, 3.00 mL) and stirred for 30 min. Next, 5 mL of Trimethylphosphine (2 mg, 24 μmol) dissolved in THF was added and the mixture was stirred for 30 min. Next, SO1861-SC-Mal (30.54 mg, 0.014 mmol, molecule 37) dissolved in 20 mM NH₄HCO₃ with acetonitrile (3:1, v/v, 3.00 mL) was added and the reaction mixture was stirred at room temperature. After 1.5 hours the reaction mixture was subjected to preparative LC-MS.^{3B} Fractions corresponding to the product were immediately pooled together, frozen and lyophilized overnight to give the title compound (4.6 mg, 81%) as a white fluffy solid. Purity based on LC-MS 95%.

MS (m/z): 19146 [M+H]⁺

Dendron-(SO1861)8-azide (FIG. 5f):

The synthesis of Dendron(SO1861)8-azide was performed at both saponin-dendron conjugates Dendron-(EMCH-SO1861)⁸-amine and Dendron-(semicarbazone-SO1861)8-amine. The following synthesis is exemplary described for Dendron-(semicarbazone-SO1861)8-azide.

Dendron-(semicarbazone-SO1861)8-azide

Dendron-(semicarbazone-SO1861)8-amine (5.4 mg, 0.28 μmol, molecule 44) and 2,5-dioxopyrrolidin-1-yl 1-azido-3,6,9,12-tetraoxapentadecan-15-oate (1.1 mg, 2.8 μmol, molecule 18) were dissolved in DMF(1.00 mL). Next, DIPEA (1.302 μL) was added and the mixture was shaken for 1 min and left standing at room temperature. After 2 hours the reaction mixture was subjected to preparative LC-MS.^{3C} Fractions corresponding to the product were immediately pooled together, frozen and lyophilized overnight to give the title compound (4.35 mg, 80%) as a white fluffy solid. Purity based on LC-MS 93%.

MS (m/z): 19420 [M+H]⁺

Dendron(SO1861)8-maleimide1 (FIG. 5g):

The synthesis of Dendron(SO1861)8-maleimide1 was performed at both saponin-dendron conjugates Dendron(EMCH-SO1861)8-amine and Dendron(semicarbazone-SO1861)8-amine. The following synthesis is exemplary described for Dendron(semicarbazone-501861)8-maleimide1.

Dendron(SO1861)8-amine (17 mg, 0.891 μmol) and 2,5-dioxopyrrolidin-1-yl 1-(2,5-dioxo-2,5-dihydro-1H-pyrrol-1-yl)-3,6,9,12-tetraoxapentadecan-15-oate (3.94 mg, 8.91 μmol) were dissolved in DMF(1.00 mL). Next, DIPEA (1.55 μL, 8.91 μmol) was added and the mixture was shaken for 1 min and left standing at room temperature. After 3 hours the reaction mixture was subjected to preparative LC-MS. 3c Fractions corresponding to the product were immediately pooled together, frozen and lyophilized overnight to give the title compound (13.2 mg, 76%) as a white fluffy solid. Purity based on LC-MS 71%.

MS: 19473 [M+H]⁺

Dendron(SO1861)8-maleimide2 (FIG. 5h):

The synthesis of Dendron(SO1861)8-maleimide2 was performed at both saponin-dendron conjugates Dendron(EMCH-SO1861)8-amine and Dendron(semicarbazone-SO1861)8-amine. The following synthesis is exemplary described for Dendron(semicarbazone-501861)8-maleimide2.

Dendron(SO1861)8-amine (27 mg, 1.4 μmol) and 2,5-Dioxopyrrolidin-1-yl 3-(2-(2-(3-(2,5-dioxo-2h-pyrrol-1(5h)-yl)propanamido)ethoxy)ethoxy)propanoate (5.84 mg, 0.014 mmol) were dissolved om DMF (1.00 mL). Next, DIPEA (2.39 μL, 0.014 mmol) was added and the suspension was shaken for 1 min and left standing at room temperature. After 2 hours the reaction mixture was subjected to preparative LC-MS.^{3C} Fractions corresponding to the product were immediately pooled together, frozen and lyophilized overnight to give the title compound (16 mg, 60%) as a white fluffy solid. Purity based on LC-MS 81%.

MS: 19456 [M+H]⁺

Mab-Saporin Conjugates

Custom Trastuzumab-saporin, cetuximab-saporin or CD71 mab-saporin conjugates were produced and purchased from Advanced Targeting Systems (San Diego, CA).

Antibody-(SC-SO1861)ⁿ, Antibody-(EMCH-SO1861)ⁿ conjugates

Trastuzumab, Cetuximab, are referred hereafter as “Ab”. Ab was conjugated to SO18161-EMCH or SO1861-semicarbazone-Mal (SO1861-SC-Mal) via Michael-type thiol-ene conjugation reaction at DAR⁴. The SO1861-EMCH and SO1861-SC-Mal molecule obtains a labile (L) pH sensitive bond between its structure and its maleimide function generating a labile bond between the SO1861 and Ab. The procedure is exemplary described for Cetuximab-(SC-SO1861) 4: To a solution of Cetuximab (40 mg, 8.0 ml) was added 10 μl/ml each of Tris concentrate (127 mg/ml, 1.05 M), Tris·HCl concentrate (623 mg/ml, 3.95 M) and EDTA-Naz concentrate (95 mg/ml, 0.26 M) to give a 50 mM TBS, 2.5 mM EDTA buffer pH 7.5. To Cetuximab divided into four portions (each of 9.73 mg, 4.864 mg/ml, 65 nmol) was added an aliquot of freshly prepared TCEP solution (0.5-2.0 mg/ml, 1.15-7.02 mole equivalents, 75-455 nmol), the mixtures vortexed briefly then incubated for 300 minutes at 20° C. with roller-mixing. After incubation (prior to addition of SO1861-SC-Mal), a ca. 1 mg (0.210 ml) aliquot of Ab-SH was removed from each mixture and purified by gel filtration using a zeba spin desalting column into TBS pH 7.5. These aliquots were characterized by UV-vis analysis and Ellman's assay. To each of the bulk Ab-SH was added an aliquot of freshly prepared SO1861-SC-Mal solution (2 mg/ml, 1.3 mole equivalents per ‘thiol’, 0.15-0.61 μmol, 0.16-0.63 ml), the mixtures vortexed briefly then incubated for 120 minutes at 20° C. Besides each conjugation reaction, two aliquots of desalted Ab-SH (0.25 mg, 1.67 nmol) were reacted with NEM (1.3 mole equivalents per ‘thiol’, 4.3-17.4 nmol, 2.2-8.7 μl of a 0.25 mg/ml solution) or TBS pH 7.5 buffer (2.2-8.7 μl) for 120 minutes at 20° C., as positive and negative controls, respectively. After incubation (prior to addition of NEM), a 0.200 ml aliquot of Ab-SC-SO1861 mixture was removed and purified by gel filtration using zeba spin desalting column into TBS pH 7.5. This aliquot was characterized by UV-vis and alongside positive and negative controls were characterized by Ellman's assay to obtain SO1861-5C incorporations. To the bulk Ab- SC-SO1861 mixture was added an aliquot of freshly prepared NEM solution (2.5 mg/ml, 2.5-10 mole equivalents, 0.15-0.58 μmol) and the mixtures purified by zeba spin desalting columns eluting with DPBS pH 7.5 to give purified Cetuximab-(SC-SO1861) conjugates. The products were normalized to 2.5 mg/ml and filtered to 0.2 μm prior to dispensing for biological evaluation.

Antibody-(S-HSP27BNA)ⁿ

To an aliquot of Trastuzumab (5.00 mg, 3.3×10⁻⁵ mmol, 2.5 mg/ml) was added an aliquot of freshly prepared SMCC solution (2.00 mg/ml, 8.56 mole equivalents, 28.5×10⁻⁵ mmol) in DMSO, the mixture vortexed briefly then incubated for 60 minutes at 20° C. with roller-mixing. After, the reaction was quenched by the addition of an aliquot of a freshly prepared glycine solution (10.0 mg/ml, 5.0 mole equivalents, 1.4×10⁻³ mmol) in DPBS pH 7.5. Tras-SMCC (5.15 mg, 3.4×10⁻⁵ mmol, 2.127 mg/ml, Tras:SMCC=4.4) was obtained after gel filtration using a zeba 10 ml spin column eluting with TBS pH 7.5, and characterised by UV-vis spectrophotometry and SAMSA colorimetric assay. BNA lyophilisate was reconstituted to 10 mg/ml using TBS pH 7.5 and analysed by UV-vis spectrophotometry. To (protected) BNA (9.5 mg, 1.6×10⁻³ mmol, 9.59 mg/ml) was added an aliquot of freshly prepared TCEP solution (50.0 mg/ml, 10 mole equivalents, 16.3×10⁻³ mmol) in TBS pH 7.5, the mixture briefly vortexed then incubated for 60 minutes at 37° C. with roller-mixing. After, the mixture was desalted using PD10 G25 desalting column eluting with TBS pH 7.5, followed by repeated washing via diafiltration using vivaspin T4 3K MWCO filters (3,000, 20° C., ˜15 minutes) and TBS pH 7.5 (seven cycles in total) to remove residual TCEP. BNA-SH (7.90 mg, 1.3×10⁻³ mmol, 3.098 mg/ml, BNA:SH=1.2) was characterised by UV-vis spectrophotometry and Ellman's colorimetric assay. To Tras-SMCC (4.93 mg, 3.3×10⁻⁵ mmol, 2.127 mg/ml) was added an aliquot of BNA-SH (8.0 mole equivalents, 26.3×10⁻⁵ mmol, 3.098 mg/ml, 0.499 ml), the mixture vortexed briefly then incubated overnight at 20° C. After ca. 16 hours, the conjugate was purified by gel filtration using a 1.6×35 cm Sephadex G50M column eluting with DPBS pH 7.5. The conjugate was collected, pooled and concentrated to ca. 1 ml. The concentrate was analysed by BCA assay to ascertain antibody concentration and then by UV-vis spectrophotometry to ascertain a combined mass ε value for the conjugate and incorporation of BNA. The product was normalised to 2.0 mg/ml and spin-filtered to 0.2 μm. The result was Trastuzumab—BNA conjugate (2.0 mg/ml, 1.1 ml, 48%, BNA to mAB ratio=2.2, purity=99.0%).

Synthesis of Antibody(-L-SC-SO1861)4-(S-HSP27BNA)2, Antibody(-L-EMCH-SO1861)4-(S-HSP27BNA)2 conjugates

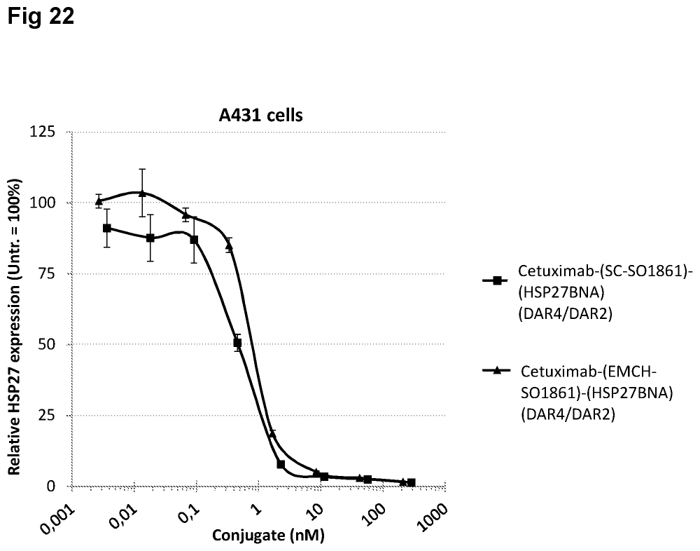

Trastuzumab, Cetuximab, are referred hereafter as “Ab”. Ab was conjugated to SO18161-EMCH or SO1861-semicarbazone-Mal (SO1861-SC-Mal) via Michael-type thiol-ene conjugation reaction at DAR⁴. The SO1861-EMCH and SO1861-SC-Mal molecule obtains a labile (L) pH sensitive bond between its structure and its maleimide function generating a labile bond between the SO1861 and Ab. The procedure is exemplary described for conjugate Cetuximab-(SC-SO1861)4-(BNA)2 (Cetuximab-(SC-SO1861)-(BNA) (DAR4/DAR2) comprising four copies of the SO1861 and two copies of the BNA. This exemplary conjugate is tested for its influence on HSP27 expression in A431 cells (See FIG. 22).

To an aliquot of Cet-(SC-SO1861)4 (24.7 mg, 1.65×10⁻⁴ mmol, 2.50 mg/ml) was added an aliquot of freshly prepared PEG4-SPDP solution (5.00 mg/ml, 3.31 mole equivalents, 5.45×10⁻⁴ mmol), the mixture vortexed briefly then incubated for 60 minutes at 20° C. with roller-mixing. After incubation, the reaction was quenched by the addition of an aliquot of a freshly prepared glycine solution (10.0 mg/ml, 16 mole equivalents, 2.64×10⁻³ mmol), the mixture vortexed briefly then incubated for >15 minutes at 20° C. with roller-mixing. The conjugate was purified by 2.6×26 cm Sephadex G50M eluting with TBS pH 7.5 and analysed by UV-vis to give purified Cet-(SC-SO1861)4-SPDP (23.7 mg, 96%, 1.32 mg/ml, SPDP per Cet ratio=2.5). Cet-(SC-S SO1861)4-SPDP was stored at 2-8° C. in the dark until required.

Separately, an aliquot of Thiol HSP27BNA (4.16 mg, 7.08×10⁻⁴ mmol, 10.80 mg/ml) reconstituted using TBS pH 7.5 was added an aliquot of freshly prepared THPP solution (50 mg/ml, 10 mole equivalents, 7.08×10⁻³ mmol, 29.5 μl), the mixture vortexed briefly then incubated for 60 minutes at 37° C. with roller-mixing. After incubation, the BNA was purified by PD10 Sephadex G25M column eluting with TBS pH 7.5, to afford BNA-SH (3.15 mg, 76%, SH to HSP27BNA ratio=0.88).

To an aliquot of Cet-(SC-SO1861)4-SPDP (10.01 mg, 6.67×10⁻⁵ mmol, 1.32 mg/ml) was added an aliquot of BNA-SH (2.10 mg/ml, 4.0 mole equivalents, 2.67×10⁻⁴ mmol, 0.747 ml), the mixture vortexed briefly then incubated for 3 hours at 20° C. then overnight at 5° C. with roller-mixing. Reaction progression was measured by PDT displacement. After ca. 18 hours, the conjugate mixture was again UV analysed to confirm PDT displacement by HSP27BNA and was then purified by 2.6×26 cm Sephadex G50M column eluting with DPBS pH 7.5 to give purified Cet-(SC-SO1861)4-HSP27BNA conjugate. The aliquot was analysed by BCA colorimetric assay and assigned a new molar absorptivity, then concentrated and normalised to 2.5 mg/ml, filtered to 0.2 μm and then dispensed into an aliquot for characterisation (retain) and two aliquots for product testing, one supplied refrigerated and the other frozen. The result was a Cet-(SC-SO1861)4-HSP27BNA conjugate (total yield=8.75 mg, 87%, HSP27BNA to Cet: ratio=1.4).

HSP27BNA, Oligonucleotide Sequences

HSP27 (5′-GGCacagccagtgGCG-3′) [SEQ ID NO: 1], more specifically BNA^{NC}, modified from Zhang et al. (2011) [Y Zhang, Z Qu, S Kim, V Shi, B Liao1, P Kraft, R Bandaru, Y Wu, L M Greenberger and I D Horak, Down-modulation of cancer targets using locked nucleic acid (LNA)-based antisense oligonucleotides without transfection, Gene Therapy (2011) 18, 326-333]), BNA^{NC} oligos were ordered with 5′-Thiol C6 linker at Bio-Synthesis Inc (Lewisville, Texas), with BNA bases in capitals, and fully phosphorothioated backbones.

hCD71mab-EMCH-SO1861

hCD71 mab was desalted into TBS pH 7.5 buffer and then normalised to 3 mg/ml. To an aliquot of hCD71mab (7.52 mg, 3.007 mg/ml, 5.1×10⁻⁵ mmol) was added an aliquot of freshly prepared TCEP solution (2.0 mg/ml, 4 mole equivalents, 20.4×10⁻⁵ mmol), the mixtures vortexed briefly then incubated for 210 minutes at 20° C. with roller-mixing. After incubation (prior to addition of SO1861-EMCH), a ca. 1 mg (0.340 ml) aliquot of hCD71 mab-SH was removed from the mixture and purified by gel filtration using a zeba spin desalting column into TBS pH 7.5. This aliquot was characterised by UV-vis analysis and Ellman's assay (SH to hCD71 ratio=3.9). To the bulk hCD71 mab-SH was added an aliquot of freshly prepared SO1861-EMCH solution (2 mg/ml, 2.0 mole equivalents per ‘thiol’, 4.1×10⁻⁴ mmol), the mixtures vortexed briefly then incubated for 120 minutes at 20° C. Besides the conjugation reaction, two aliquots of desalted hCD71mab-SH (0.25 mg, 1.67×10⁻⁶ mmol) were reacted with NEM (2.0 mole equivalents per ‘thiol’, 13×10⁻⁶ mmol, 6 μl of a 0.25 mg/ml solution) or TBS pH 7.5 buffer (6 μl) for 120 minutes at 20° C., as positive and negative controls, respectively. After incubation (prior to addition of NEM), a 0.200 ml aliquot of hCD71mab-SO1861-EMCH mixture was removed and purified by gel filtration using zeba spin desalting column into TBS pH 7.5. This aliquot was characterised by UV-vis and alongside positive and negative controls were characterised by Ellman's assay to obtain SO1861-EMCH incorporations. To the bulk hCD71mab-SO1861-EMCH mixture was added an aliquot of freshly prepared NEM solution (2.5 mg/ml, 5 mole equivalents, 2.2×10⁻⁴ mmol) and the mixtures purified by zeba spin desalting columns eluting with DPBS pH 7.5 to give purified hCD71mab-EMCH-SO1861conjugate. The product was concentrated using vivaspin T4 concentrators (3,000 g, 5 minutes, 5° C.), then normalised to 2.5 mg/ml and filtered to 0.2 μm prior to dispensing. Yield: 69%, SO1861 to hCD71 ratio: 3.8.

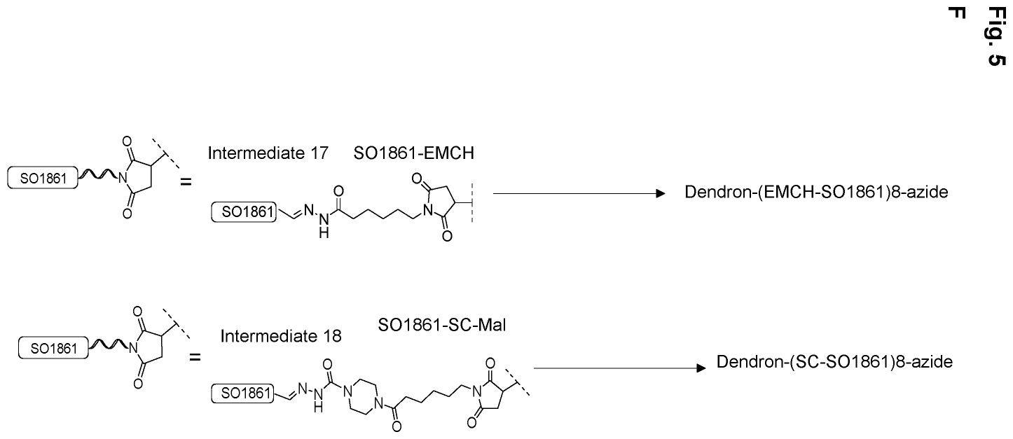

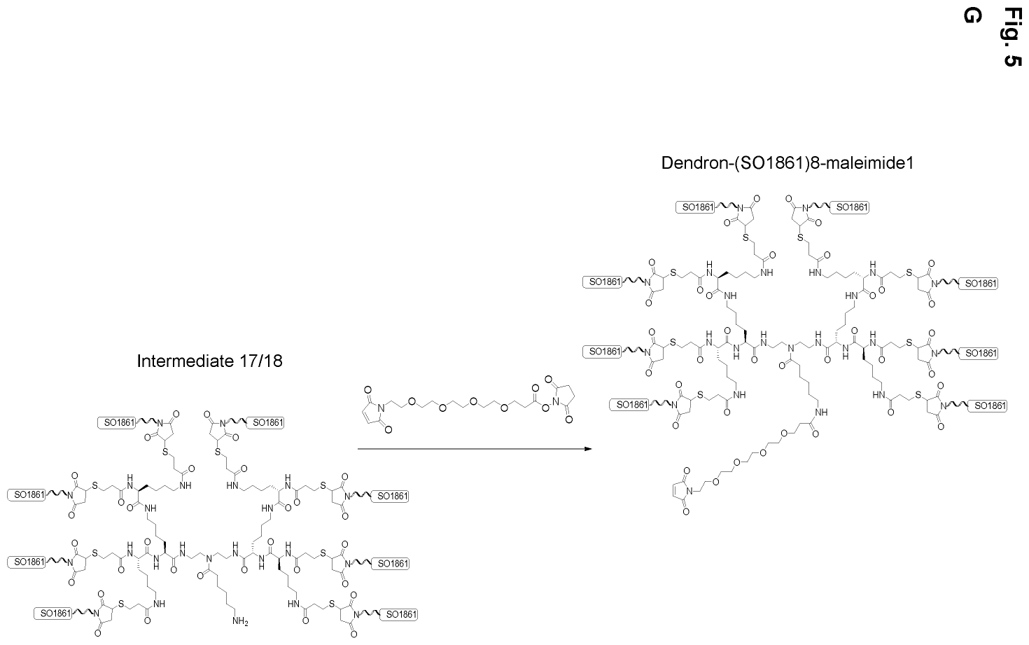

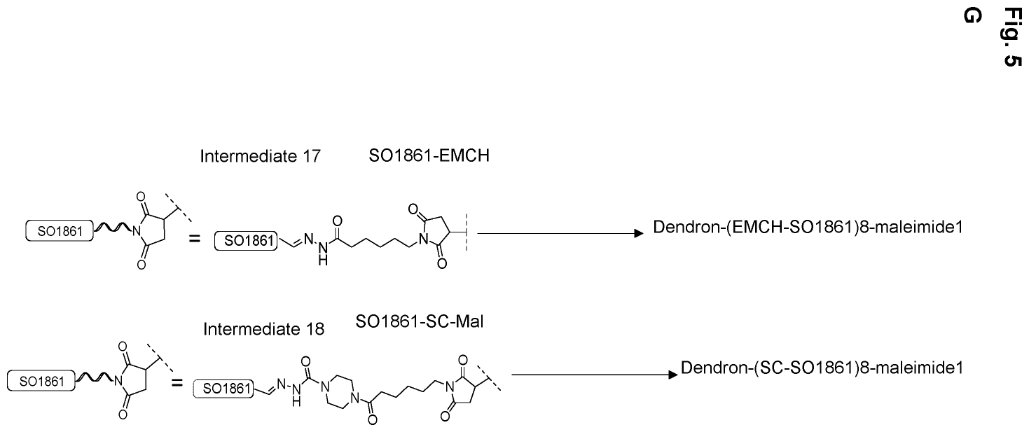

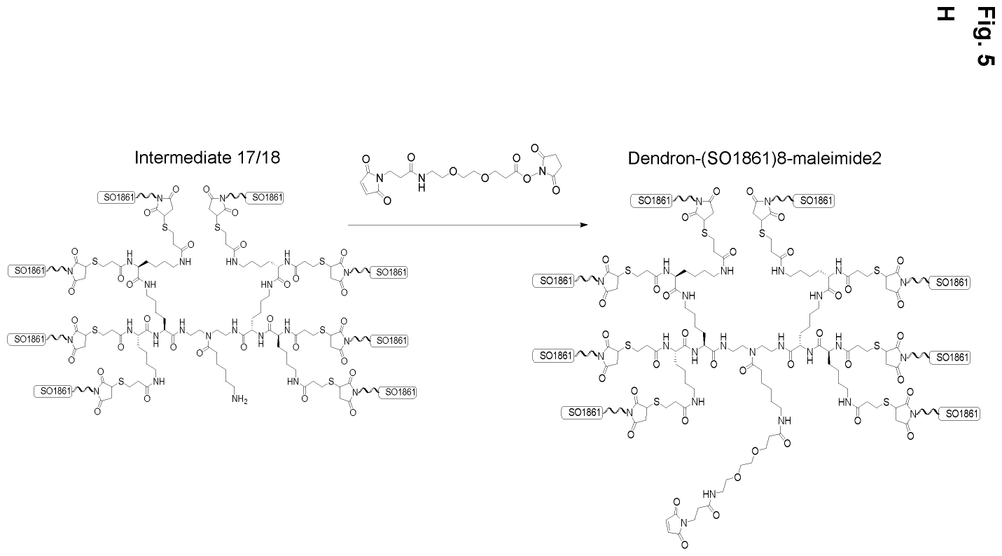

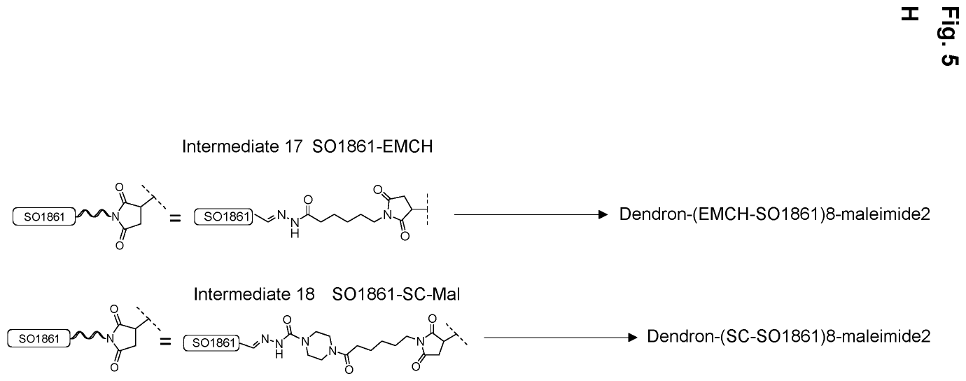

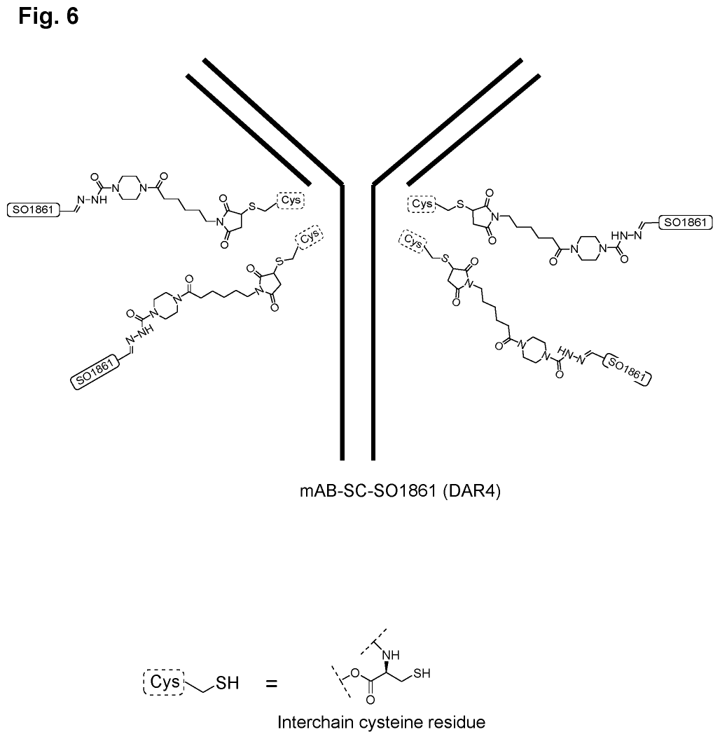

hCD71mab-SC-SO1861 (FIG. 6)

hCD71 (55.1 mg, 0.37 μmol, 8.10 mg/ml, 6.80 ml) as supplied was buffered exchanged using a zeba spin desalting column eluting with TBS pH 7.5, and normalised to 3 mg/ml. To hCD71 (50 mg, 0.33 μmol, 5.044 mg/ml) was added an aliquot of freshly prepared TCEP solution (2.00 mg/ml, 3 mole equivalents, 1 μmol), the mixture vortexed briefly then incubated for 210 minutes at 20° C. with roller-mixing. After incubation (prior to addition of SO1861-SC-Maleimide), a 1.0 mg (0.201 ml) aliquot of hCD71 solution was removed and purified by gel filtration using zeba spin desalting column into TBS pH 7.5. This aliquot was characterised by UV-vis analysis and Ellman's assay (2.23 mg/ml, thiol to hCD71 ratio=4.35). To an aliquot of bulk hCD71-SH (42 mg, 0.28 μmol, 4.978 mg/ml) was added an aliquot of freshly prepared SO1861-SC-Maleimide solution (2.0 mg/ml, 8 mole equivalents, 2.24 μmol, 2.32 ml), the mixture vortexed briefly then incubated for 120 minutes at 20° C. Besides the conjugation reaction, two aliquots of desalted hCD71-SH (0.25 mg, 0.077 ml, 1.67 nmol) were reacted with NEM (8.00 equivalents, 13.4 nmol, 6.7 μl of a 0.25 mg/ml solution) or TBS pH 7.5 buffer (6.7 μl) for 120 minutes at 20° C., as positive and negative controls, respectively. After incubation, a ca. 0.4 mg aliquot of hCD71-S08161 mixture was removed, purified by gel filtration using zeba spin desalting column into TBS pH 7.5 and characterised by Ellman's assay alongside positive and negative controls to obtain SO1861 incorporation. To the bulk hCD71-SO1861 mixture was added an aliquot of freshly prepared NEM solution (5 mole equivalents, 1.4 μmol of a 2.5 mg/ml solution) to quench the reaction. The conjugate was purified by zeba 40K MWCO spin desalting columns eluting with DPBS pH 7.5 to give purified hCD71-SO1861 conjugate. An aliquot of the product was analysed by BCA colorimetric assay to ascertain a new EC280 value. Then, the product was normalised to 2.5 mg/ml, filtered to 0.2 μm and then dispensed into aliquots for characterisation, product testing and further conjugation. The result was hCD71-SO1861 conjugate (total yield=37.8 mg, 89%, SO1861 to hCD71 ratio=4.2).

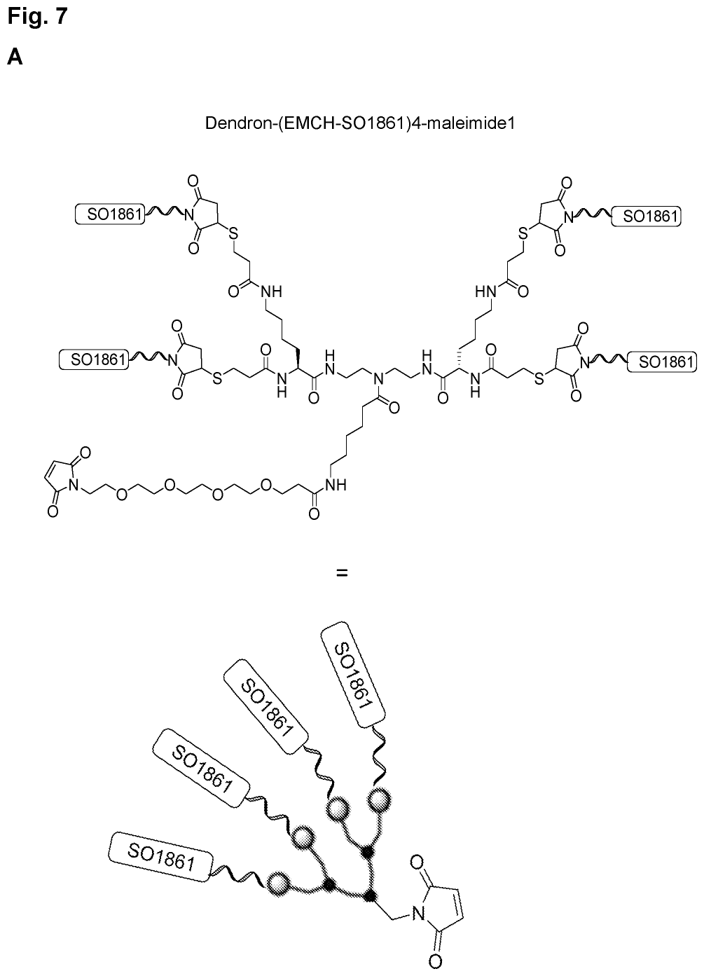

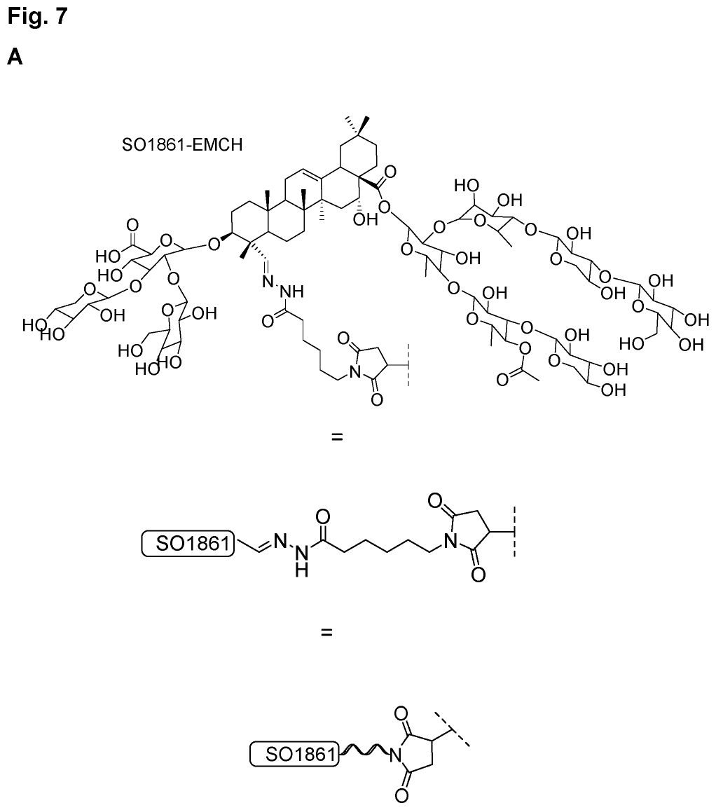

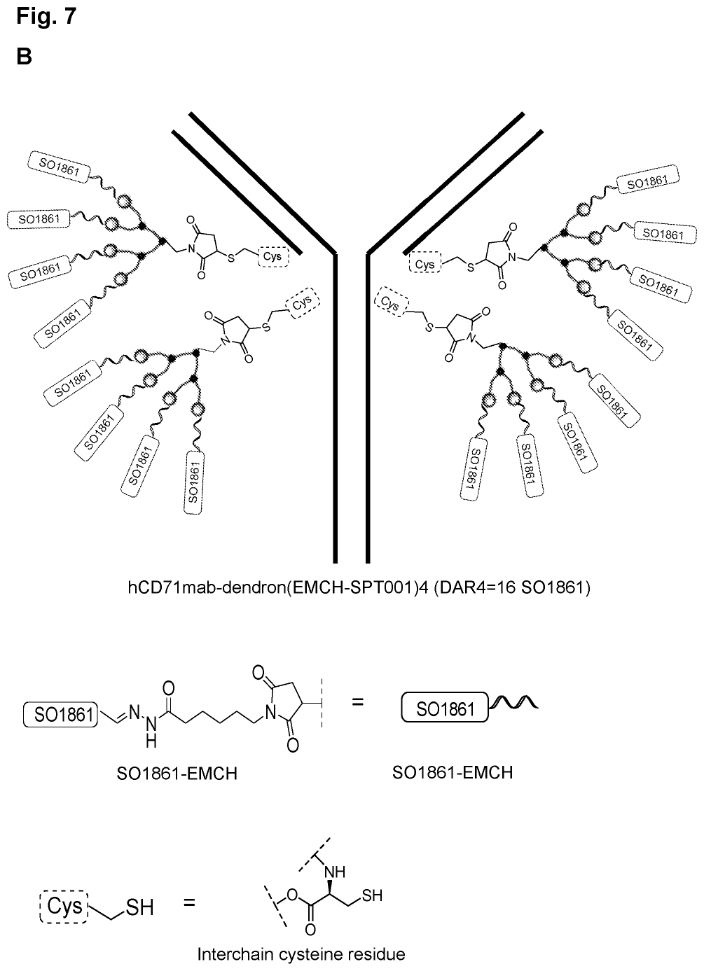

hCD71mab-dendron(EMCH-SO1861)4 (FIG. 7)

hCD71 (55.1 mg, 0.37 μmol, 8.10 mg/ml, 6.80 ml) as supplied was buffered exchanged using a zeba spin desalting column eluting with TBS pH 7.5, and normalised to 5 mg/ml. To hCD71 (50 mg, 0.33 μmol, 5.044 mg/ml) was added an aliquot of freshly prepared TCEP solution (2.00 mg/ml, 3 mole equivalents, 1 μmol), the mixture vortexed briefly then incubated for 210 minutes at 20° C. with roller-mixing. After incubation (prior to addition of Dendron-(EMCH-SO1861)4-maleimide1), a 1.0 mg (0.201 ml) aliquot of hCD71 solution was removed and purified by gel filtration using zeba spin desalting column into TBS pH 7.5. This aliquot was characterised by UV-vis analysis and Ellman's assay (2.23 mg/ml, thiol to hCD71 ratio=3.8). To an aliquot of bulk hCD71-SH (42 mg, 0.28 μmol, 4.978 mg/ml) was added an aliquot of freshly prepared Dendron-(EMCH-SO1861)4-maleimide1 solution in DMSO (2.0 mg/ml, 8 mole equivalents, 2.24 μmol, 2.32 ml), the mixture vortexed briefly then incubated for 120 minutes at 20° C. Besides the conjugation reaction, two aliquots of desalted hCD71-SH (0.25 mg, 0.077 ml, 1.67 nmol) were reacted with NEM (8.00 equivalents, 13.4 nmol, 6.7 μl of a 0.25 mg/ml solution) or TBS pH 7.5 buffer (6.7 μl) for 120 minutes at 20° C., as positive and negative controls, respectively. After incubation, a ca. 0.4 mg aliquot of hCD71-dendron(EMCH-SO8161)4 mixture was removed, purified by gel filtration using zeba spin desalting column into TBS pH 7.5 and characterised by Ellman's assay alongside positive and negative controls to obtain Dendron-(EMCH-SO1861)4 incorporation. To the bulk hCD71-Dendron-(EMCH-SO1861)4 mixture was added an aliquot of freshly prepared NEM solution (5 mole equivalents, 1.4 μmol of a 2.5 mg/ml solution) to quench the reaction. The conjugate was purified by zeba 40K MWCO spin desalting columns eluting with DPBS pH 7.5 to give purified hCD71-SO1861 conjugate. An aliquot of the product was analysed by BCA colorimetric assay to ascertain a new EC280 value. Then, the product was normalised to 2.5 mg/ml, filtered to 0.2 μm and then dispensed into aliquots for characterisation. The result was hCD71mab-dendron(EMCH-SO1861)4 conjugate (total yield=22 mg, 37%, dendron-(EMCH-SO1861)4 to hCD71 ratio=3.2).

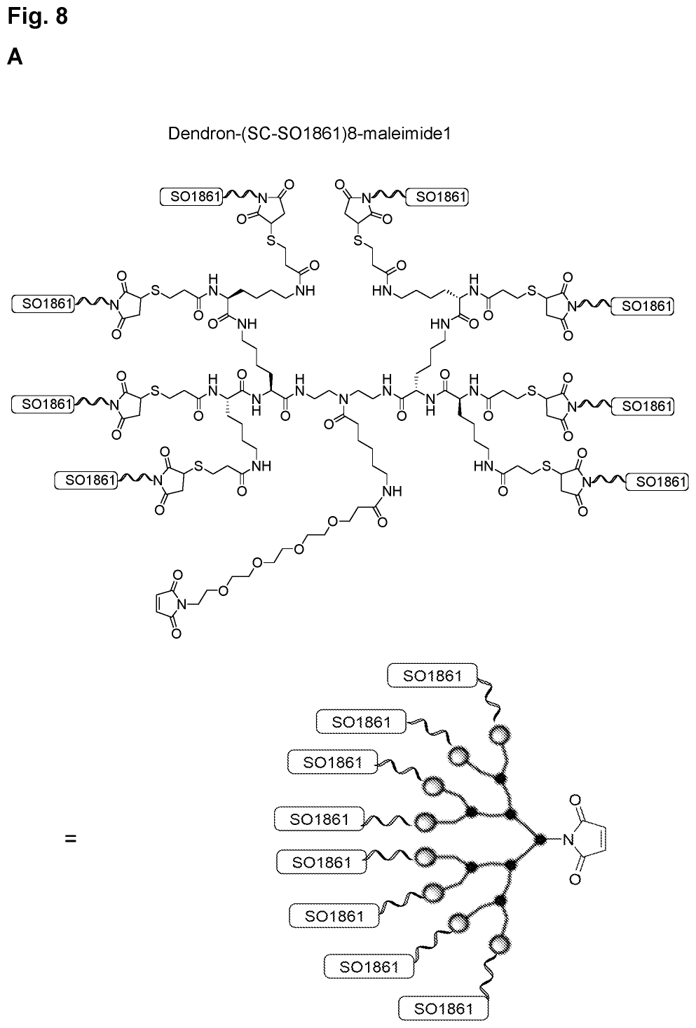

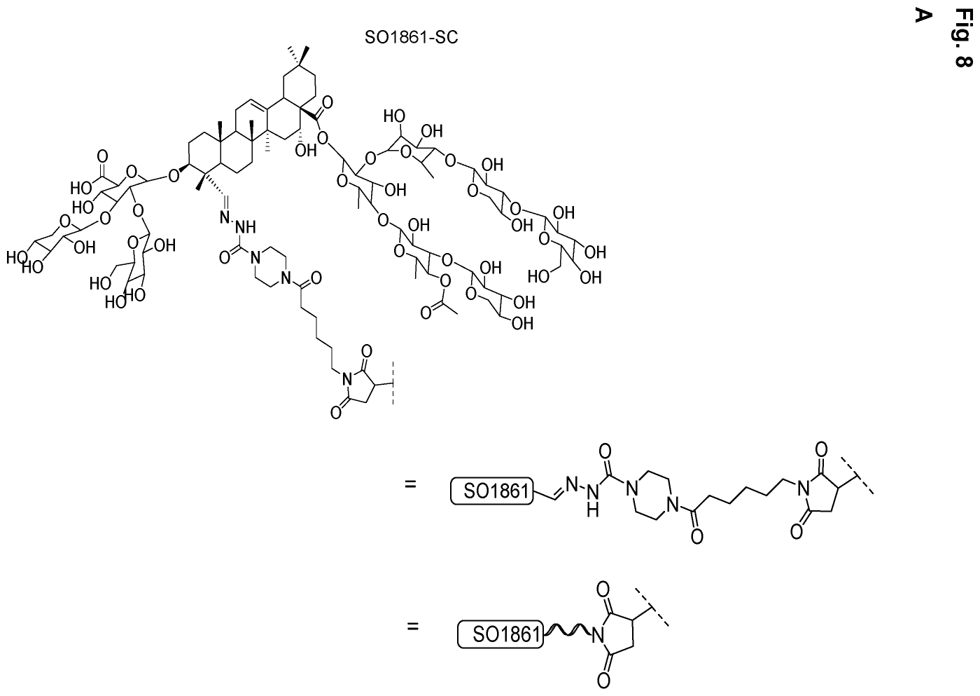

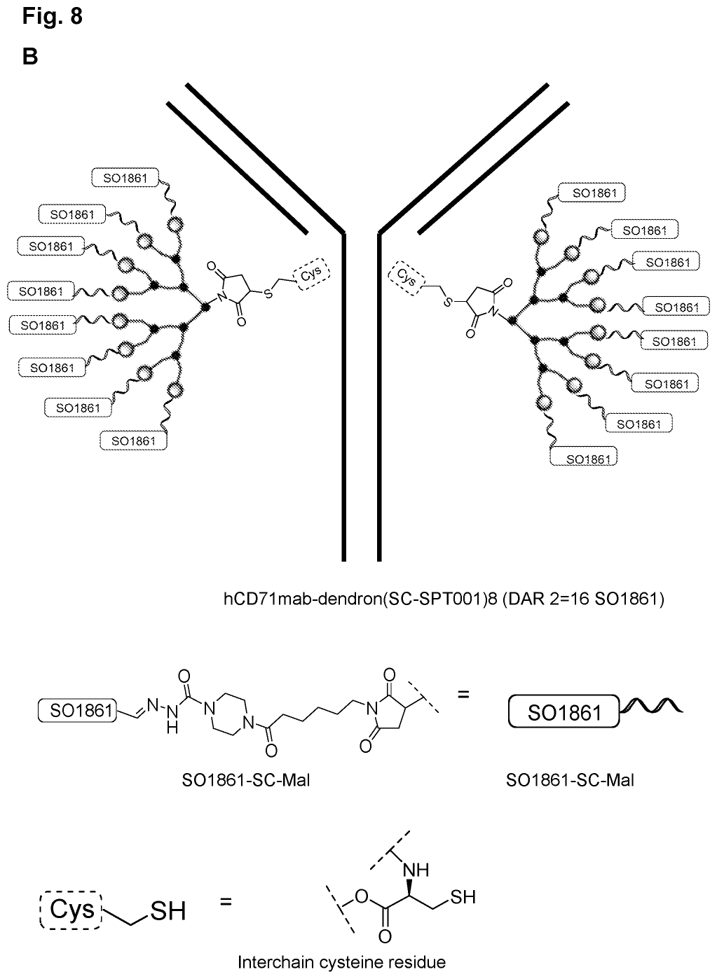

hCD71mab-Dendron(SC-SO1861)8 (FIG. 8)

hCD71 (55.1 mg, 0.37 μmol, 8.10 mg/ml, 6.80 ml) as supplied was buffered exchanged using a zeba spin desalting column eluting with TBS pH 7.5, and normalised to 3 mg/ml. To hCD71 (50 mg, 0.33 μmol, 5.044 mg/ml) was added an aliquot of freshly prepared TCEP solution (1 mg/ml, 1.3 mole equivalents, 0.43 μmol), the mixture vortexed briefly then incubated for 210 minutes at 20° C. with roller-mixing. After incubation (prior to addition of Dendron-(SC-SO1861)8-maleimide1), a 1.0 mg (0.201 ml) aliquot of hCD71 solution was removed and purified by gel filtration using zeba spin desalting column into TBS pH 7.5. This aliquot was characterised by UV-vis analysis and Ellman's assay (2.23 mg/ml, thiol to hCD71 ratio=1.6). To an aliquot of bulk hCD71-SH (42 mg, 0.28 μmol, 4.978 mg/ml) was added an aliquot of freshly prepared Dendron-(SC-SO1861)8-maleimide1 solution in DMSO (2.0 mg/ml, 3.2 mole equivalents, 0.9 μmol, 9 ml), the mixture vortexed briefly then incubated for 120 minutes at 20° C. Besides the conjugation reaction, two aliquots of desalted hCD71-SH (0.25 mg, 0.077 ml, 1.67 nmol) were reacted with NEM (8.00 equivalents, 13.4 nmol, 6.7 μl of a 0.25 mg/ml solution) or TBS pH 7.5 buffer (6.7 μl) for 120 minutes at 20° C., as positive and negative controls, respectively. After incubation, a ca. 0.4 mg aliquot of hCD71-dendron(SC-SO8161)8 mixture was removed, purified by gel filtration using zeba spin desalting column into TBS pH 7.5 and characterised by Ellman's assay alongside positive and negative controls to obtain Dendron-(SC-SO1861)8 incorporation. To the bulk hCD71-Dendron-(SC-SO1861)8 mixture was added an aliquot of freshly prepared NEM solution (5 mole equivalents, 1.4 μmol of a 2.5 mg/ml solution) to quench the reaction. The conjugate was purified by zeba 40K MWCO spin desalting columns eluting with DPBS pH 7.5 to give purified hCD71mab-dendron(SC-SO1861)8 conjugate. An aliquot of the product was analysed by BCA colorimetric assay to ascertain a new EC280 value. Then, the product was normalised to 2.5 mg/ml, filtered to 0.2 μm and then dispensed into aliquots for characterisation. The result was hCD71mab-dendron(EMCH-SSO1861)8 conjugate (total yield=24 mg, 41%, dendron-(SC-SO1861)8 to hCD71 ratio=1.5

Cetuximab-EMCH-SO1861

To Cetuximab (1100 mg) 10 μl/ml each of Tris concentrate (127 mg/ml, 1.05M), Tris·HCl concentrate (623 mg/ml, 3.95 M) and EDTA·2Na·2H₂O concentrate (95 mg/ml, 0.26 M) was added to give a 50 mM TBS, 2.5 mM EDTA buffer pH ˜7.5. To Cetuximab (1087 mg, 4.800 mg/ml, 7.2×10⁻³ mmol) was added an aliquot of freshly prepared TCEP solution (1 mg/ml, 2.72 mole equivalents, 2.0×10⁻² mmol, 5.65 mg), the mixture swirled by hand to mix then incubated for 210 minutes at 20° C. with roller-mixing. After incubation (prior to addition of SO1861-EMCH), a 2 mg (0.417 ml) aliquot of Ab-SH was removed and purified by gel filtration using zeba spin desalting column into TBS pH 7.5. This aliquot was characterised by UV-vis analysis and Ellman's assay (3.693 mg/ml, SH to Ab ratio=4.0). To the bulk Ab-SH was added an aliquot of freshly prepared SO1861-EMCH solution (2 mg/ml, 5.2 mole equivalents, 3.8×10⁻² mmol, 38.9 ml), the mixtures vortexed briefly then incubated for 120 minutes at 20° C. Besides the conjugation reaction, two aliquots of desalted Ab-SH (0.5 mg, 0.135 ml, 3.33×10⁻⁶ mmol) were reacted with NEM (8.00 equivalents, 2.66×10⁻⁶ mmol, 3.3 μg, 13.3 μl of a 0.25 mg/ml solution) or TBS pH 7.5 buffer (13.3 μl) for 120 minutes at 20° C., as positive and negative controls, respectively. After incubation (prior to addition of NEM), a ca. 2 mg (0.450 ml) aliquot of Ab-SO1861 mixture was removed and purified by gel filtration using zeba spin desalting column into TBS pH 7.5. This aliquot was characterised by UV-vis (3.271 mg/ml) and alongside positive and negative controls were characterised by Ellman's assay to obtain SO1861 incorporation. To the bulk Ab-SO1861 mixture was added an aliquot of freshly prepared NEM solution (2.5 mg/ml, 5 mole equivalents, 3.6×10⁻² mmol, 4.54 mg) and the mixture stored at 2-8° C. overnight. The conjugate was purified by 10×40 cm Sephadex G50M column eluting with DPBS pH 7.5 to give purified Cetuximab-SO1861 conjugate. The aliquot was filtered to 0.2 μm and dispensed. The result was a Cetuximab-SO1861 conjugate (total yield=1056 mg, 97%, SO1861 to Ab ratio=3.9).

Cetuximab-Dendron(EMCH-SO1861)4, DAR^{3.9}

To Cetuximab (5.95 mg, 1.19 ml) was added 30 μl/ml each of Tris/Tris·HCl/EDTA·concentrate to give a 50 mM TBS, 2.5 mM EDTA buffer pH ˜7.5. To Cetuximab (5.85 mg, 4.793 mg/ml, 3.9×10⁻⁵ mmol) was added an aliquot of freshly prepared TCEP solution (2.72 mole equivalents, 1.1×10⁻⁴ mmol, 30.4 μg, 30.4 μl of a 1 mg/ml solution), the mixture vortex mixed briefly then incubated for 210 minutes at 20° C. with roller-mixing. After incubation (prior to addition of Dendron-(EMCH-SO1861)4-maleimide1), a 1 mg (0.214 ml) aliquot of Ab-SH was removed and purified by gel filtration using zeba spin desalting column into TBS pH 7.5. This aliquot was characterised by UV-vis analysis and Ellman's assay (2.3 mg/ml, SH to Ab ratio=5.9). To the bulk Ab-SH (4.85 mg, 3.2×10⁻⁵ mmol) was added TBS buffer pH 7.5 (2.3 mL) and an aliquot of freshly prepared Dendron-(EMCH-SO1861)4-maleimide1 solution (8.0 mole equivalents, 2.6×10⁻⁴ mmol, 2 mg, 1.03 ml of 2 mg/ml solution in DMSO), the mixture vortexed briefly then incubated for 120 minutes at 20° C. Besides the conjugation reaction, two aliquots of desalted Ab-SH (0.25 mg, 0.109 ml, 1.6×10⁻⁶ mmol) were reacted with NEM (8.00 equivalents, 1.35×10⁻⁵ mmol, 1.67 μg, 6.7 μl of a 0.25 mg/ml solution) or TBS pH 7.5 buffer (6.7 μl) for 120 minutes at 20° C., as positive and negative controls, respectively. After incubation (prior to addition of NEM), a ca. 0.1 mg (0.1 ml) aliquot of Ab-Dendron-(EMCH-SO1861)4 mixture was removed and purified by gel filtration using zeba spin desalting column into TBS pH 7.5. This aliquot was characterised by UV-vis and alongside positive and negative controls were characterised by Ellman's assay to obtain Dendron-(EMCH-SO1861)4 incorporation. To the bulk Ab-Dendron-(EMCH-SO1861)4 mixture was added an aliquot of freshly prepared NEM solution (5 mole equivalents, 1.6×10⁻⁴ mmol, 0.02 mg, 81 μl of a 0.25 mg/ml solution). The conjugate was purified by 1.6×33 cm Sephadex G50M column eluting with DPBS pH 7.5 to give purified Cetuximab-Dendron-(EMCH-SO1861)4 conjugate. The product was concentrated then normalised to 2.50 mg/ml using a vivaspin T4 concentrator (3,000 g, 5° C., 30 minutes). The result was a Cetuximab-Dendron-(EMCH-SO1861)4 conjugate (total yield=3.17 mg, 54%, Dendron-(EMCH-SO1861)4 to Ab ratio=3.9).

Cetuximab-dendron(EMCH-SO1861)4, DAR1.5

To Cetuximab (80.0 mg, 16.0 ml) 10 μl/ml each of Tris concentrate (127 mg/ml, 1.05M), Tris·HCl concentrate (623 mg/ml, 3.95M) and EDTA·2Na·2H2O concentrate (95 mg/ml, 0.26M) was added to give a 50 mM TBS, 2.5 mM EDTA buffer pH ˜7.5. To Cetuximab (78.1 mg, 4.739 mg/ml, 5.2×10⁻⁴ mmol) was added an aliquot of freshly prepared TCEP solution (1.647 mole equivalents, 8.6×10⁻⁴ mmol, 245 μg, 245 μl of a 1 mg/ml solution), the mixture vortex mixed briefly then incubated for 90 minutes at 20° C. with roller-mixing. After incubation (prior to addition of Dendron-(EMCH-SO1861)4-maleimide1), a 2 mg (0.428 ml) aliquot of Ab-SH was removed and purified by gel filtration using zeba spin desalting column into TBS pH 7.5. This aliquot was characterised by UV-vis analysis and Ellman's assay (3.774 mg/ml, SH to Ab ratio=1.9). To the bulk Ab-SH (76.05 mg, 5.1×10⁻⁴ mmol) was added an aliquot of freshly prepared Dendron-(EMCH-SO1861)4-maleimide1 solution (4.0 mole equivalents, 2.0×10⁻³ mmol, 16.22 mg, 1.62 ml of 10 mg/ml solution in DMSO), the mixture vortexed briefly then incubated for 120 minutes at 20° C. Besides the conjugation reaction, two aliquots of desalted Ab-SH (0.50 mg, 0.132 ml, 3.3×10⁻⁶ mmol) were reacted with NEM (4.00 equivalents, 1.3×10⁻⁵ mmol, 1.67 μg, 6.7 μl of a 0.25 mg/ml solution) or TBS pH 7.5 buffer (6.7 μl) for 120 minutes at 20° C., as positive and negative controls, respectively. After incubation (prior to addition of NEM), a ca. 1 mg (0.235 ml) aliquot of Ab-Dendron-(EMCH-SO1861)4 mixture was removed and purified by gel filtration using zeba spin desalting column into TBS pH 7.5. This aliquot was characterised by UV-vis (2.793 mg/ml) and alongside positive and negative controls were characterised by Ellman's assay to obtain Dendron-(EMCH-SO1861)4 incorporation. To the bulk Ab-Dendron-(EMCH-SO1861)4 mixture was added an aliquot of freshly prepared NEM solution (5 mole equivalents, 2.5×10⁻³ mmol, 0.32 mg, 1.27 ml of a 0.25 mg/ml solution). The conjugate was purified by 5×50 cm Sephadex G50M column eluting with DPBS pH 7.5 to give purified Cetuximab-Dendron-(EMCH-SO1861)4 conjugate. The product was concentrated then normalised to 2.50 mg/ml using a vivaspin T4 concentrator (3,000 g, 5° C., 30 minutes). The result was a Cetuximab-Dendron-(EMCH-SO1861)4 conjugate (total yield=68.6 mg, 88%, Dendron-(EMCH-SO1861)4 to Ab ratio=1.5).

Cetuximab-Dendron(SC-SO1861)8, DAR 1.5

To Cetuximab (10 mg, 5 mg/ml, 2.0 ml) was added 30 μl/ml (60 μl) of a pre-mixed Tris/Tris·HCl/EDTA concentrate comprising Tris concentrate (127 mg/ml, 1.05M), Tris·HCl concentrate (623 mg/ml, 3.95M) and EDTA·2Na·2H2O concentrate (95 mg/ml, 0.26M) combined 1:1:1 v/v, to give a 50 mM TBS, 2.5 mM EDTA buffer pH ˜7.5. TTE mix pre-tested against histidine pH 6.0 buffer (30 μl/ml), pH 7.53, 19° C. An aliquot of Cet (10 mg, 6.70×10⁻⁵ mmol, 4.793 mg/ml) was added an aliquot of freshly prepared TCEP solution (1.00 mg/ml, 1.24 mole equivalents, 8.30×10⁻⁵ mmol, 24 μl), the mixture vortexed briefly then incubated for 210 minutes at 20° C. with roller-mixing. After incubation (prior to addition of Dendron-(SC-SO1861)8-maleimide1), a 1.0 mg (0.212 ml) aliquot of was removed and purified by gel filtration using zeba spin desalting column into TBS pH 7.5. This aliquot was characterised by UV-vis analysis and Ellman's assay (3.671 mg/ml, SH to Ab ratio=1.6). To the bulk Cet-SH (9.0 mg, 6.00×10⁻⁵ mmol, 4.739 mg/ml) was added an aliquot of freshly prepared Dendron-(SC-SO1861)8-maleimide1 solution (2.0 mg/ml, 3.2 mole equivalents, 1.92×10⁻⁴ mmol, 1.845 ml) pre-dispersed in DMSO:TBS pH 7.5 (20:80 v/v), the mixture vortexed briefly then incubated for 72 hours at 20° C. Besides the conjugation reaction, two aliquots of desalted Cet-SH (0.25 mg, 0.068 ml, 1.67×10⁻⁶ mmol) were reacted with NEM (3.2 equivalents, 5.34×10⁻⁶ mmol, 2.7 μl of a 0.25 mg/ml solution) or TBS pH 7.5 buffer (2.7 μl) for 72 hours at 20° C., as positive and negative controls, respectively. After incubation, 50 μl of the Cet-Dendron-(SC-SO1861)8 mixture was characterised by Ellman's assay alongside positive and negative controls to obtain Dendron-(SC-SO1861)8 incorporation. To the bulk Cet-Dendron-(SC-SO1861)8 mixture was added an aliquot of freshly prepared NEM solution (5 mole equivalents, 3.00×10⁻⁴ mmol, 15 μl of a 2.5 mg/ml solution) to quench the reaction. The conjugate was purified by 2.6×40 cm superdex 200 column eluting with DPBS pH 7.5 to give purified Cet-Dendron-(SC-SO1861)8 conjugate. The aliquot was concentrated by vivaspin T15 centrifugal filtration (3,000 g, 10 minute intervals, 20° C.), analysed by UV-vis spectrophotometry and BCA assay to ascertain a new EC280 and normalised to 2.5 mg/ml, filtered to 0.2 μm and then dispensed into aliquots for in-house characterisation and customer testing. The result was Cmab-Dendron-(SC-SO1861)8 conjugate (total yield=4.8 mg, 53%, Dendron-(SC-SO1861)8 to Ab ratio=1.5).

### Example 1

Results

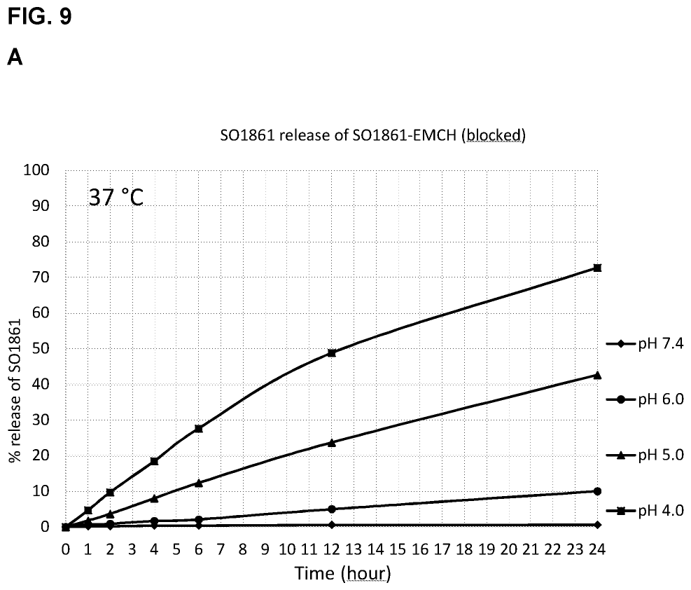

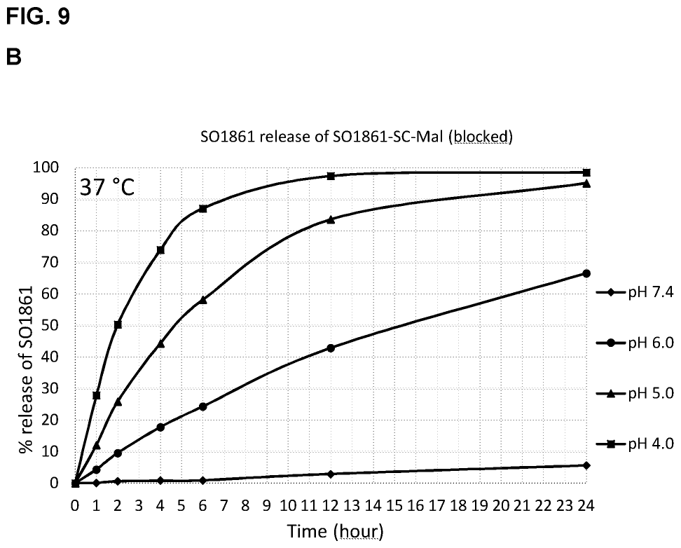

SO1861-SC-Mal (blocked) (FIG. 2B) was tested for pH dependent release with a release kinetics assay and compared with SO1861-EMCH (blocked) (FIG. 3). SO1861-EMCH (blocked) was produced in same way as in FIG. 2B. The assay was performed in buffers with pH 7.4, pH 6.0 pH, pH 5.0, pH 4.0 at 37° C. for 24 hours. This revealed that SO1861-SC-Mal (blocked) shows a 6-fold faster release compared to SO1861-EMCH (blocked) (50% release at pH 4.0=2 hours (SO1861-SC-Mal (Blocked)) vs 50% release in 12 hours (SO1861-EMCH (Blocked)) (FIG. 9).

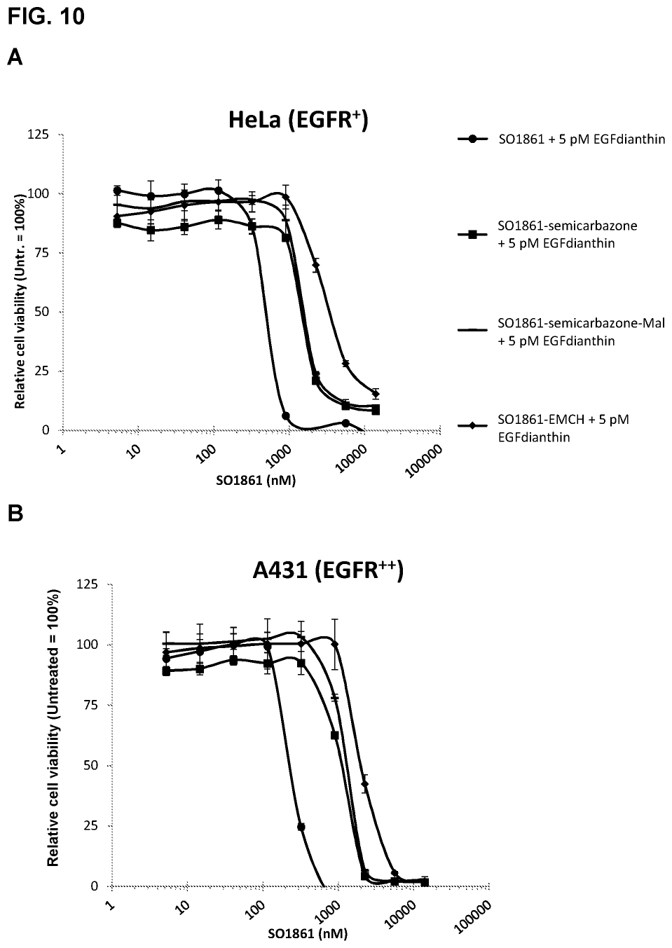

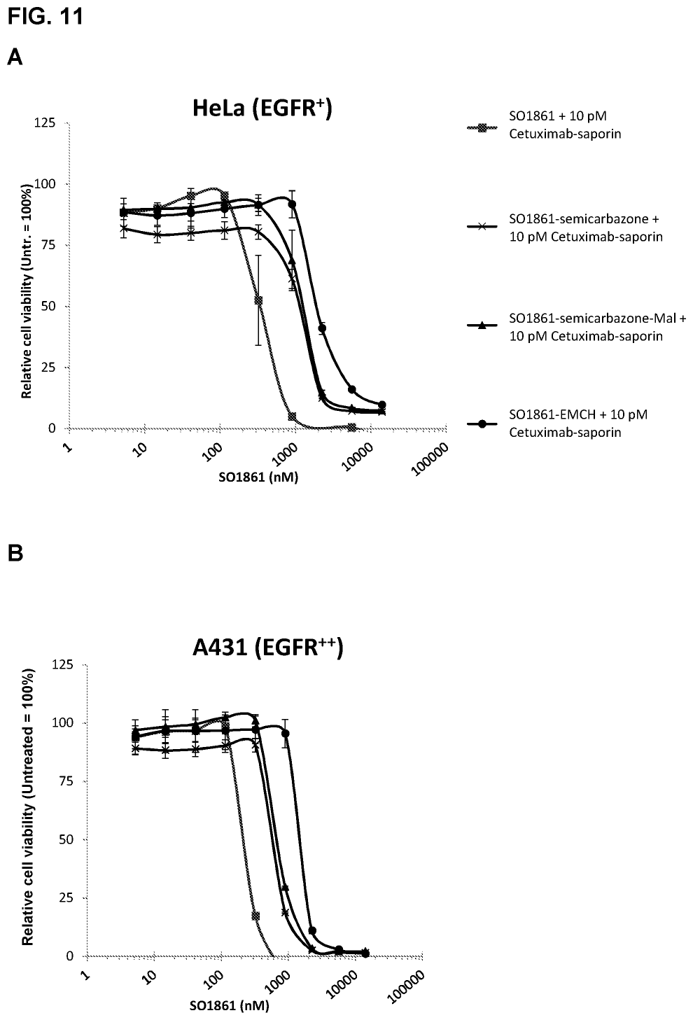

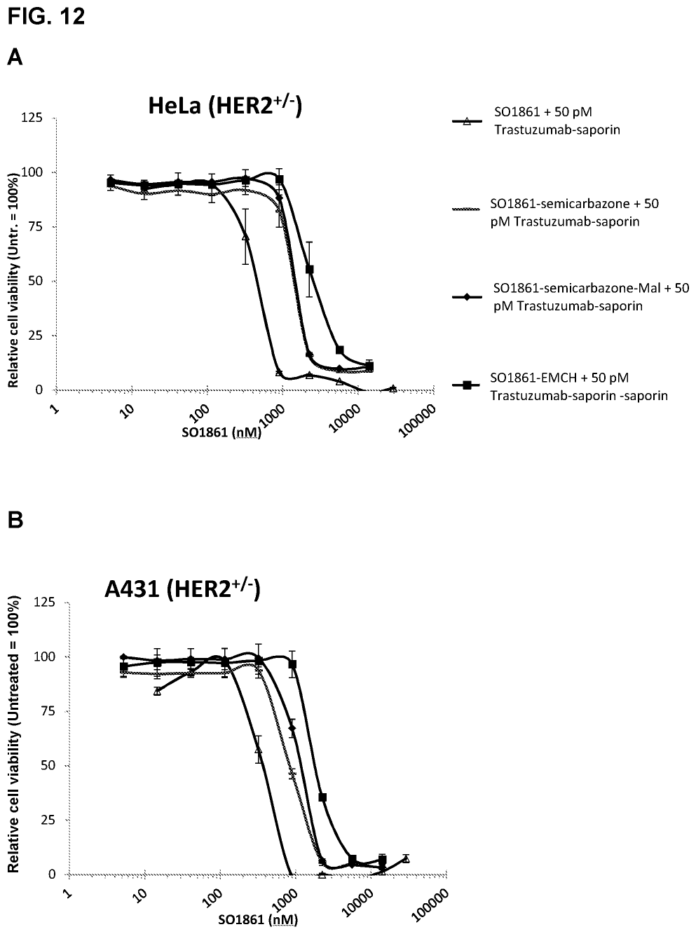

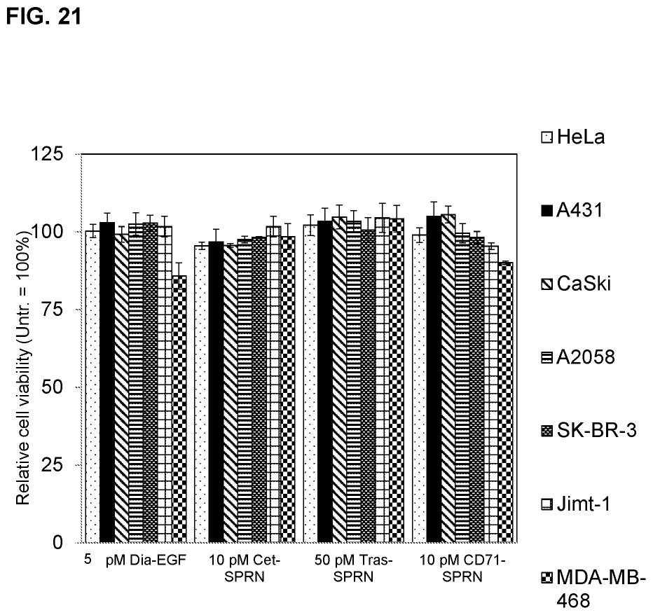

Next, SO1861-SC (FIG. 2A) and SO1861-SC-Mal (FIG. 1; FIG. 2B) were tested for endosomal escape enhancing activity. For this, SO1861, SO1861-EMCH, SO1861-SC and SO1861-SC-Mal were titrated in the presence of a non-effective, fixed concentration (FIG. 21) of 5 pM EGFdianthin (Dia-EGF), 50 pM Trastuzumab-saporin, 10 pM CD71-saporin or 10 pM Cetuximab-saporin on EGFR/HER2 expressing cells (HeLa and A431, Table 1, 3, 4). This revealed that both SO1861-SC or SO1861-SC-Mal combined with 5 pM EGFdianthin (IC50=2000 nM (HeLa); IC50=1500 nM (A431)) (FIG. 10) or 10 pM Cetuximab-saporin (IC50=1500 nM (HeLa); IC50=1000 nM (A431) (FIG. 11) or 50 pM Trastuzumab-saporin (IC50=2000 nM (HeLa); IC50=1500 nM (A431)) (FIG. 12) was more effective compared to SO1861-EMCH combined with 5 pM EGFdianthin (IC50=4000 nM (HeLa); IC50=3000 nM (A431)) (FIG. 10) or 10 pM Cetuximab-saporin (IC50=3000 nM (HeLa); IC50=2000 nM (A431)) (FIG. 11) or 50 pM Trastuzumab-saporin (IC50=3000 nM (HeLa); IC50=2500 nM (A431)) (FIG. 12). Unmodified (non-derivatized) SO1861+5 pM EGFdianthin or 10 pM cetuximab-saporin or 50 pM Trastuzumab-saporin showed strongest cell-killing activity (FIG. 10, FIG. 11, FIG. 12).

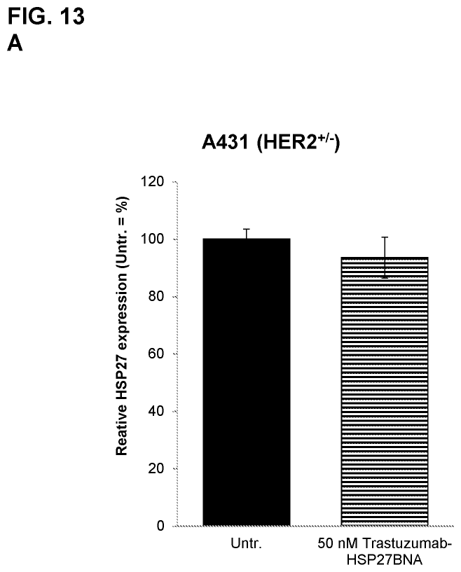

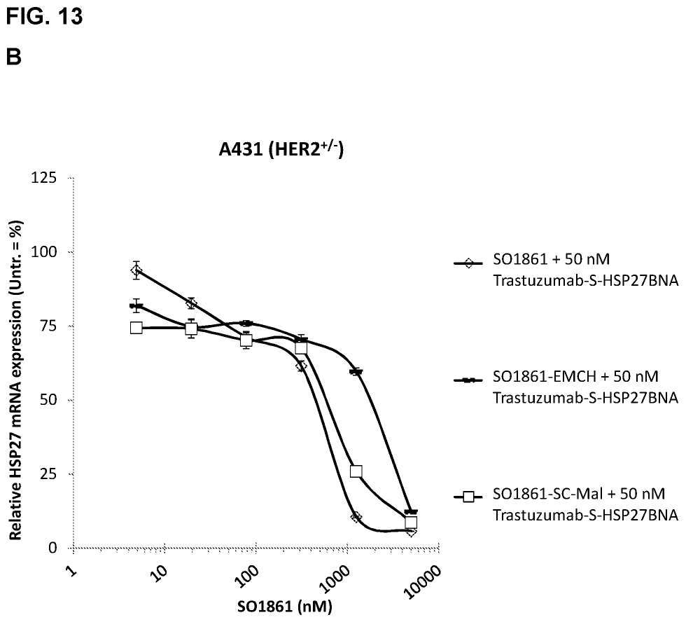

Next, the ability of SO1861-SC-Mal to induce enhanced cytoplasmic oligonucleotide delivery and targeted gene silencing was tested. For this, SO1861, SO1861-EMCH or SO1861-SC-Mal were titrated on a fixed (non-effective) concentration (determined in FIG. 13A) of 50 nM Trastuzumab-S-HSP27BNA, an antisense (BNA) oligonucleotide targeting heat-shock protein 27 (HSP27) conjugated to Trastuzumab to the lysines via a stable linker (S) with a DAR², in A431 cells (HER2^{+/-}) (Table 1, FIG. 13B) and HSP27 mRNA gene silencing activity was determined. This revealed that SO1861-SC-Mal+50 nM Trastuzumab-HSP27BNA (IC50 of 600 nM) (FIG. 13B) is more effective in gene silencing activity compared to SO1861-EMCH+50 nM Trastuzumab-HSP27BNA (IC50 of 2000 nM) in A431 cells (FIG. 13B).

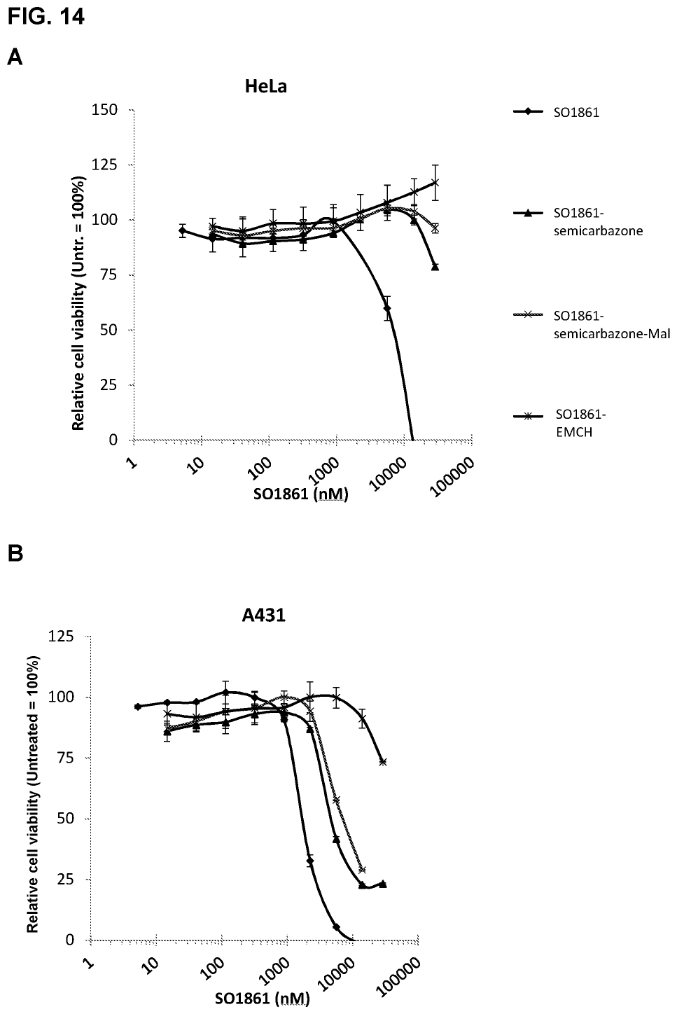

Next, cell toxicity was determined; for this, SO1861, SO1861-EMCH SO1861-SC and SO1861-SC-Mal were titrated on HeLa and A431 cells (FIG. 14, Table 3, Table 4). This revealed that unmodified SO1861 showed toxicity at IC50=9000 nM (HeLa) and IC50=2000 nM (A431), whereas in Hela cells SO1861-EMCH or SO1861-SC-Mal showed toxicity at IC50>100000 nM and in A431 cells SO1861-EMCH showed toxicity at IC50>30000 nM, whereas SO1861-SC-Mal and SO1861-SC showed toxicity at IC50=6000 nM (FIG. 14; Table 3, Table 4).

TABLE 3
IC50 overview of modified SO1861 activity (HeLa cells)/toxicity (Hela cells)/hemolysis (red blood cells)/CMC.

| | | | | | | | | | | Ratio: | Ratio: |
|---|---|---|---|---|---|---|---|---|---|---|---|
| | | IC50 nM | | | | | | | | IC50 | IC50 |
| | | (Activity; +5 | | | | | | | | toxicity/ | haemolysis/ |
| Conjugate | modified | pM | | IC50 | | IC50 | | | | IC50 | IC50 |
| Sample | group | EGFdianthin) | | (Toxicity) | | (Haemolysis) | | CMC | | activity | activity |
| SO1861 | none | 500 | nM | 9000 | nM | 10000 | nM | 120 | μM | 18 | 20 |
| SO1861-EMCH | Aldehyde | 4000 | nM | >100000 | nM | >500000 | nM | >800 | μM | >25 | >125 |
| SO1861- | Aldehyde | 2000 | nM | 30.000 | nM | 100000 | nM | n.d. | | 15 | 50 |
| Semicarbazone | | | | | | | | | | | |
| SO1861- | Aldehyde | 2000 | nM | >100000 | nM | 250000 | nM | >800 | μM | >50 | 125 |
| semicarbazone- | | | | | | | | | | | |
| Mal | | | | | | | | | | | |

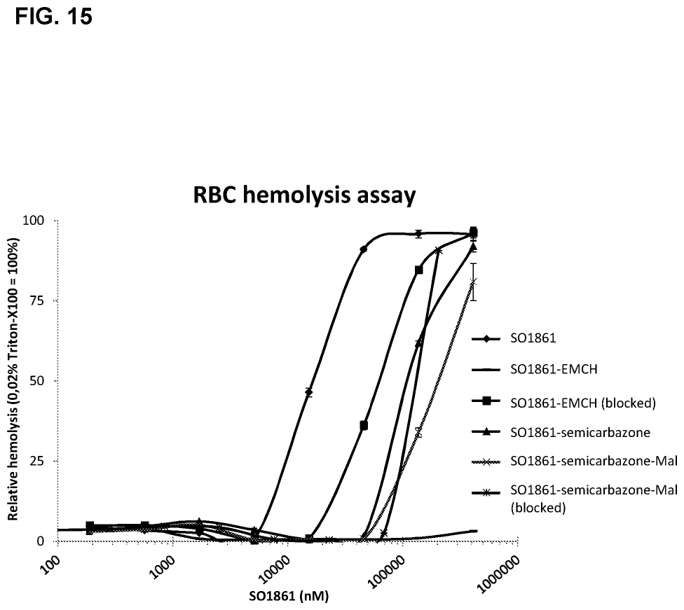

Next, a haemolysis assay was performed; for this SO1861, SO1861-EMCH, SO1861-EMCH (blocked) SO1861-SC, SO1861-SC-Mal and SO1861-SC-Mal (blocked) were tested on fresh human red blood cells (RBC) and this revealed haemolytic activity of SO1861-SC at IC50=100000 nM, SO1861-SC-Mal at IC50=250000 nM and SO1861-SC-Mal (blocked) at IC50=100000 nM (FIG. 15; Table 3, Table 4), whereas no haemolytic activity was detected with SO1861-EMCH at the range of concentrations tested (IC50>500000 nM) and SO1861-EMCH (blocked) showed haemolytic activity at IC50=6000 nM (FIG. 15; Table 3, Table 4).

Next, the critical micelle concentration (CMC) was determined for SO1861, SO1861-EMCH, SO1861-EMCH (blocked), SO1861-SC-Mal and SO1861-SC-Mal (blocked) (Table 3, Table 4). This revealed a CMC value of 129 μM for SO1861, whereas for SO1861-SC-Mal and SO1861-EMCH, a CMC could not be detected and determined within this range of concentrations, which indicates that the CMS for these two saponin derivatives is higher than 800 μM (Table 3, Table 4).

TABLE 4
IC50 overview of modified SO1861 activity (A431 cells)/toxicity
(A431 cells)/haemolysis (red blood cells)/CMC.

| | | | | | | | | | | Ratio: | Ratio: |
|---|---|---|---|---|---|---|---|---|---|---|---|
| | | IC50 nM | | | | | | | | IC50 | IC50 |
| | | (Activity; +5 | | | | | | | | toxicity/ | haemolysis/ |
| Conjugate | modified | pM | | IC50 | | IC50 | | | | IC50 | IC50 |
| Sample | group | EGFdianthin) | | (Toxicity) | | (haemolysis) | | CMC | | activity | activity |
| SO1861 | none | 300 | nM | 2000 | nM | 10000 | nM | 120 | μM | 7 | 33 |
| SO1861-EMCH | Aldehyde | 2500 | nM | >30000 | nM | >500000 | nM | >800 | μM | >12 | >200 |
| SO1861- | Aldehyde | 1500 | nM | 6000 | nM | 100000 | nM | n.d. | | 4 | 67 |
| semicarbazone | | | | | | | | | | | |
| SO1861- | Aldehyde | 1500 | nM | 6000 | nM | 250000 | nM | >800 | μM | 4 | 166 |
| Semicarbazone- | | | | | | | | | | | |
| Mal | | | | | | | | | | | |

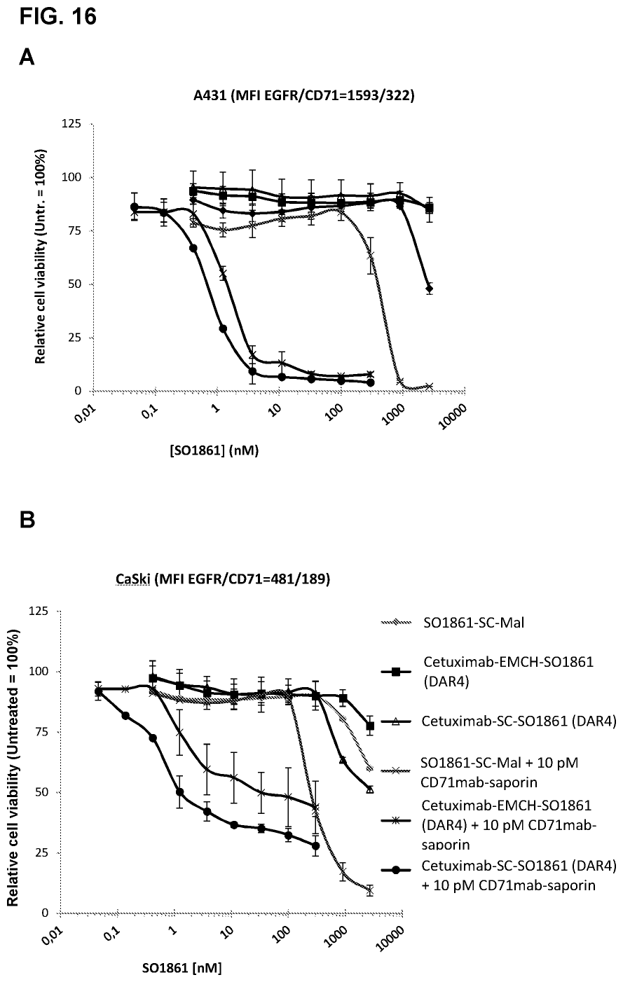

Next, SO1861-semicarbazone-Mal (SO1861-SC-Mal) was conjugated via cysteine residues (Cys) to Cetuximab (a monoclonal antibody recognizing and binding human EGFR), with a DAR 4, to yield Cetuximab-(SC-SO1861)⁴ as depicted in FIG. 6. Cetuximab-(SC-SO1861)⁴ was titrated on a fixed non-effective concentration (see FIG. 21) of 10 pM CD71mab-saporin (monoclonal antibody recognizing human CD71; clone OKT-9, conjugated to the protein toxin, saporin) and targeted protein toxin mediated cell killing on EGFR/CD71 expressing cells (Table 1; A431, EGFR⁺⁺/CD71⁺; CaSki, EGFRE⁺⁺/CD71⁺) was determined. This revealed strong cell killing at low concentrations of Cetuximab-(SC-SO1861)⁴ (A431: IC50=0.6 nM and CaSki IC50=1 nM; FIG. 16A, 16B) and compared to Cetuximab-(EMCH-SO1861)⁴ the activity was stronger (A431: IC50=2 nM and CaSki IC50=30 nM; FIG. 16A, 16B). Equivalent concentrations of Cetuximab-(SC-SO1861)⁴ or Cetuximab-(EMCH-SO1861)⁴ could not induce any cell killing activity in EGFR⁺⁺/CD71⁺ expressing cells. This shows that compared to Cetuximab-(EMCH-SO1861)⁴, Cetuximab-(SC-SO1861)⁴ more potently enhances endosomal escape of the CD71mab conjugated protein toxin (the same concentration of 10 pM the protein toxin is not effective in the absence of saponin; see FIG. 21), thereby inducing efficient cell killing of EGFR⁺⁺/CD71⁺ expressing cells. Improved acid-sensitive linker release of SO1861 from an antibody at low pH in endosomes thus improves the efficacy of mAb-SO1861 conjugates for delivery of a protein toxin such as a ribosomal inactivating protein toxin in the cytoplasm.

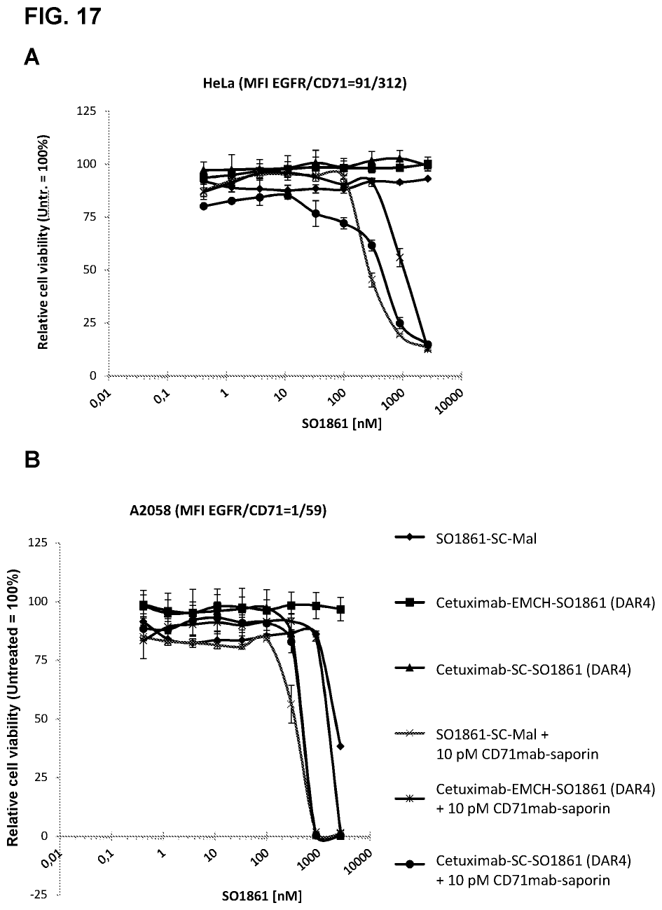

Next, Cetuximab-(SC-SO1861)⁴ was titrated on a fixed concentration of 10 pM CD71mab-saporin and targeted protein toxin-mediated cell killing on HeLa (Table 1; EGFR^{+/-}/CD71⁺) and A2058 (Table1, EGFR⁻/CD71⁺) was determined. Cetuximab-(SC-SO1861)⁴+10 pM CD71mab-saporin in HeLa cells (EGFR^{+/-}/HER2^{+/-}) could already induce partial protein toxin-mediated cell killing at lower concentrations (between 50-100 nM) of Cetuximab-(SC-SO1861)⁴ (compared to HeLa: IC50=500 nM, FIG. 17A), whereas this was not observed for Cetuximab-(EMCH-SO1861)⁴+10 pM CD71mab-saporin (HeLa: IC50=1000 nM) (FIG. 17A). In A2058 cells (that lack EGFR receptor expression), no improved activity was observed for Cetuximab-(SC-SO1861)⁴+10 pM CD71mab-saporin (A2058: IC50=1000 nM); FIG. 17B). This shows that improved release of SO1861 in acidified endosomes due to a more efficiently releasable semicarbazone linker also triggers cells with lower EGFR expression (where endosomal SO1861 threshold concentrations is reached) for the combination therapy. When EGFR expression is absent (in A2058 cells), effective SO1861 concentrations are not reached (i.e., below threshold) to induce endosomal escape and cytoplasmic delivery of the protein toxin.

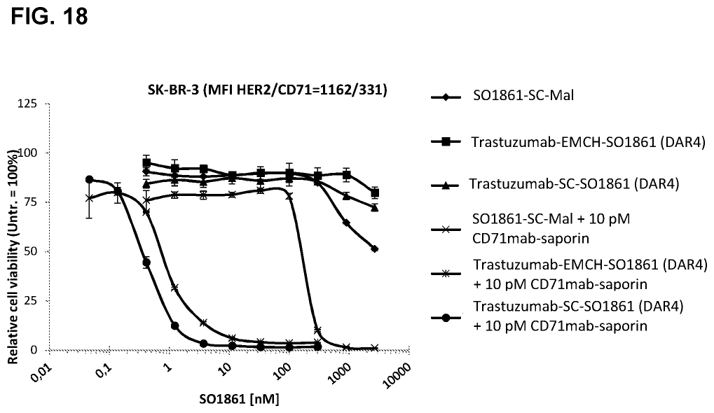

Next, SO1861-SC-Mal was conjugated via cysteine residues (Cys) to Trastuzumab (monoclonal antibody recognizing and binding human HER2), with a DAR 4, to yield Trastuzumab-(SC-SO1861)⁴. Trastuzumab-(SC-SO1861)⁴ was titrated to a fixed concentration of 10 pM CD71 mab-saporin and targeted protein toxin mediated cell killing on HER2/CD71 expressing cells (Table 1; SK-BR-3: HER2⁺⁺/CD71⁺) was determined (FIG. 18). This revealed strong cell killing at low concentrations of Trastuzumab-(SC-SO1861)⁴ (SK-BR-3: IC50=0.3 nM; FIG. 18) and compared to Trastuzumab-(EMCH-SO1861)⁴, the activity was 5-fold improved (SK-BR-3: IC50=1 nM; FIG. 18). Equivalent concentrations of Cetuximab-(SC-SO1861)⁴ or Cetuximab-(EMCH-SO1861)⁴ alone could not induce any significant cell killing activity in SK-BR-3 cells (FIG. 18). This shows that compared to Trastuzumab-(EMCH-SO1861)⁴, Trastuzumab-(SC-SO1861)⁴ more potently enhances endosomal escape of the CD71 mab conjugated protein toxin (at otherwise non-effective concentrations; FIG. 21), thereby inducing efficient cell killing of HER2⁺⁺/CD71⁺ expressing cells. Improved acid-sensitive linker release of SO1861 from an antibody at low pH in endosomes thus improves effectiveness of mAb-SO1861 conjugates for delivery of a ribosomal inactivating protein toxin in the cytoplasm.

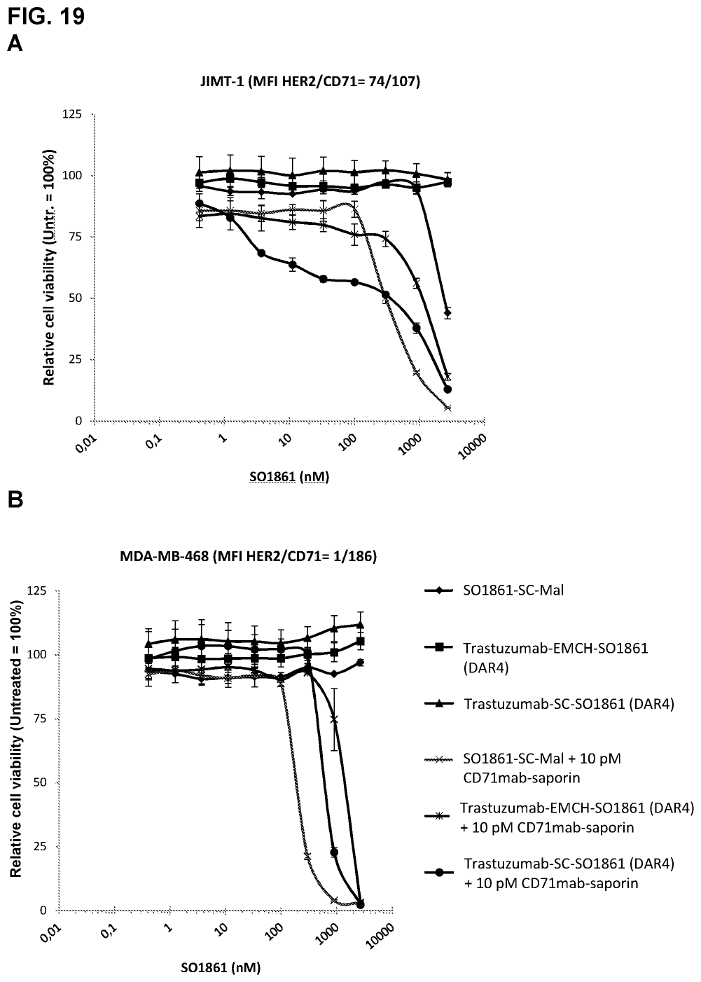

Next, Trastuzumab-(SC-SO1861)⁴ was titrated to a fixed concentration of 10 pM CD71mab-saporin and targeted protein toxin-mediated cell killing on JIMT-1 (Table1; HER2^{+/-}/CD71⁺) and MDA-MB-468 (Table 1; HER2⁻/CD71⁺) was determined. Trastuzumab-(SC-SO1861)⁴+10 pM CD71mab-saporin in JIMT-1 cells (HER2^{+/-}/CD71⁺) could already induce toxin mediated cell killing at low conjugated SO1861 concentrations (JIMT-1: IC50=100 nM; FIG. 19A) whereas this was not observed for Trastuzumab-(EMCH-SO1861)⁴+10 pM CD71mab-saporin (JIMT-1: IC50=1000 nM; FIG. 19A). In MDA-MB-468 cells (that lack HER2 receptor expression), no improved activity was observed for Trastuzumab-(SC-SO1861)⁴+10 pM CD71mab-saporin (MDA-MB-468: IC50=1000 nM; FIG. 19B). This shows that improved release of SO1861 in acidified endosomes (due to a more efficient release of semicarbazone-conjugated SO1861) also triggers cells with lower HER2 expression (i.e., that endosomal SO1861 threshold concentrations are reached) for the combination therapy. When HER2 expression is absent (in MDA-MB-468 cells), effective SO1861 concentrations are not reached (i.e. below threshold) to induce endosomal escape and cytoplasmic delivery of the protein toxin.

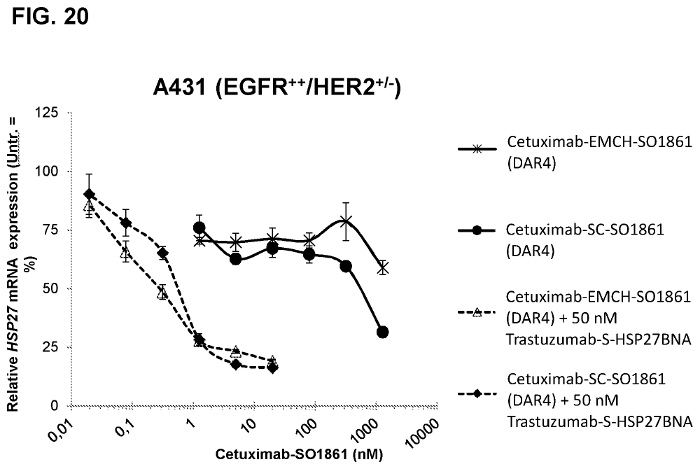

Next, Cetuximab-(SC-SO1861)⁴ was titrated to a fixed concentration of 50 pM Trastuzumab-HSP27BNA and targeted HSP27 gene silencing on A431 cells (EGFR⁺⁺/HER2^{+/-}) was determined. This revealed strong HSP27 gene silencing at low concentrations of Cetuximab-(SC-SO1861)4 (A431: IC50=0.5 nM; FIG. 20), comparable to the activity observed with Cetuximab-(EMCH-SO1861)⁴+50 pM Trastuzuzmab-HSP27BNA (A431: IC50=0.5 nM FIG. 20).

In vitro potency of Cetuximab-(SC-SO1861)-HSP27 BNA (DAR⁴/DAR2)

Cetuximab-(SC-SO1861)-(HSP27BNA) (DAR4 for the SO1861 moieties/DAR2 for the HSP27 BNA molecule) and Cetuximab-(EMCH-SO1861)-(HSP27BNA) (DAR4 for SO1861/DAR2 for the BNA) were produced and potency compared on A431 cells (Table 1; EGFR⁺⁺). This revealed that Cetuximab-(SC-SO1861)-(HSP27BNA) (DAR4/DAR2) showed improved potency (IC50=0.5 nM) compared to Cetuximab-(EMCH-SO1861)-(HSP27BNA) (DAR4/DAR2) (IC50=1 nM), indicating that SO1861 release from the antibody in the endosome enhances the potency of the Cetuximab-(SC-SO1861)-(HSP27BNA) conjugate (DAR4/DAR2) more than the EMCH-SO1861 based conjugate (FIG. 22).

In vitro potency of CD71mab-(SC-SO1861)

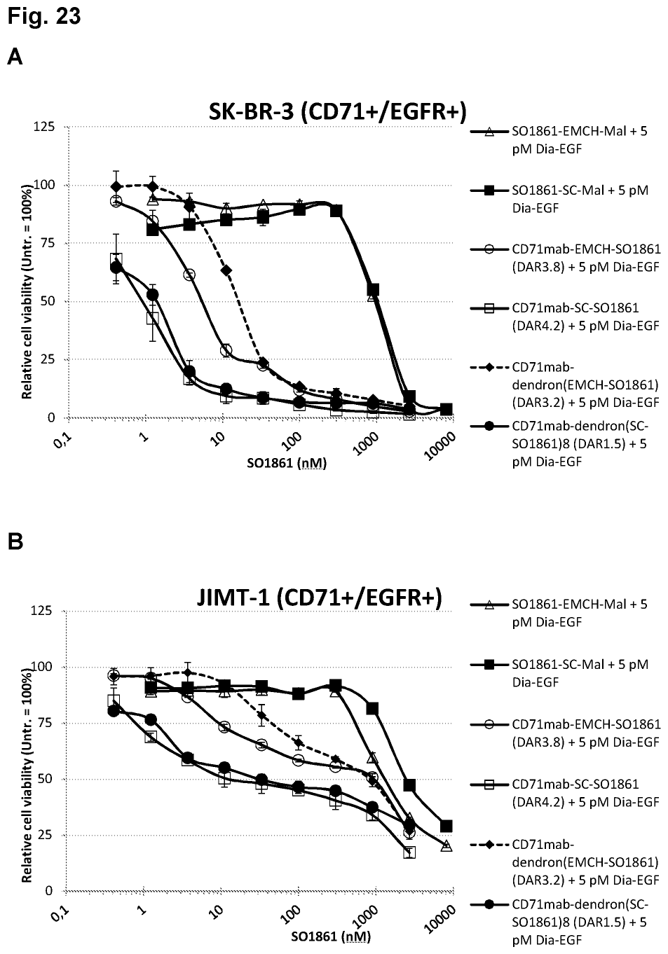

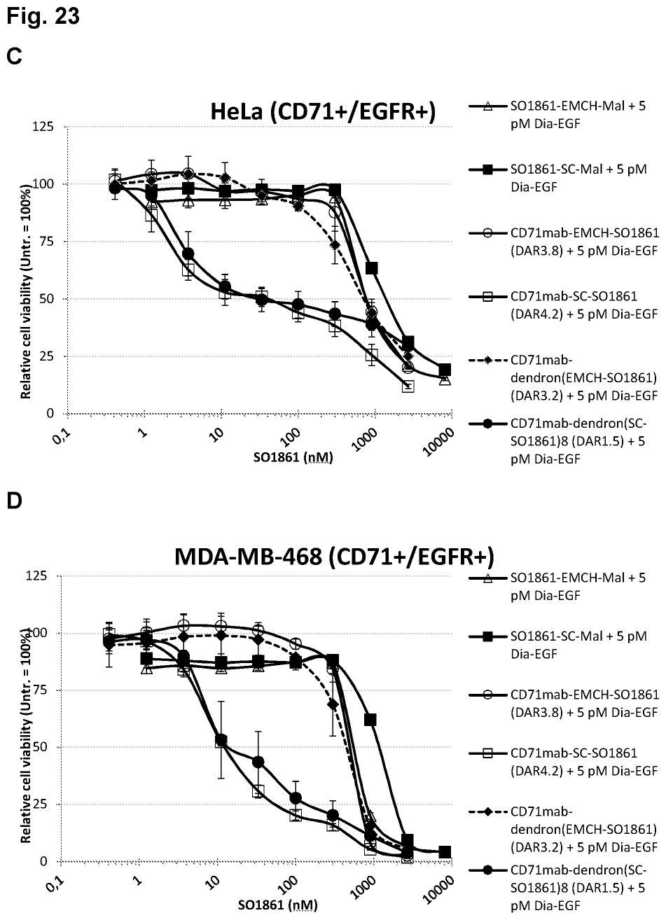

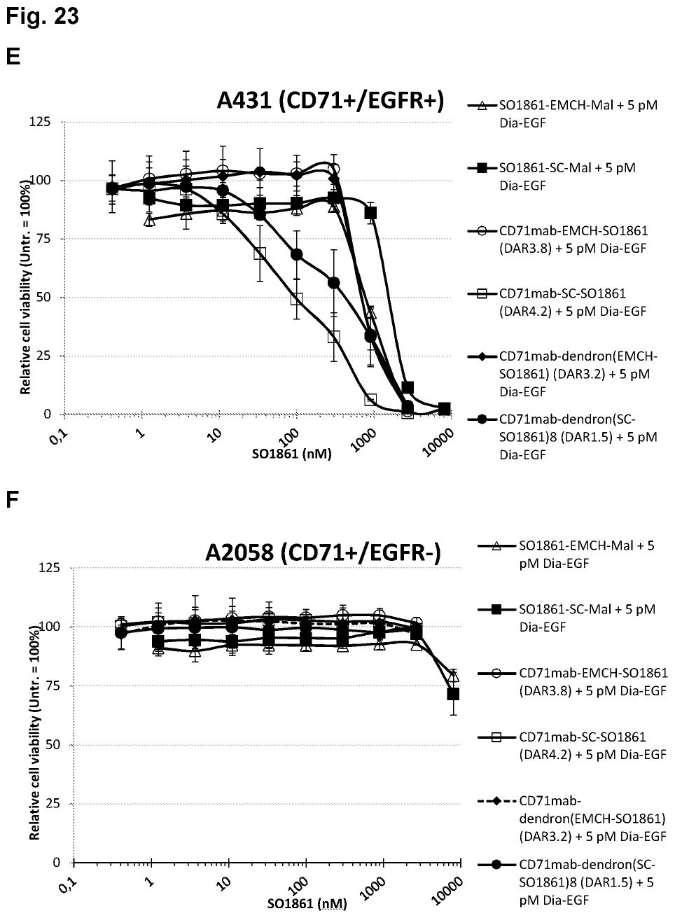

SO1861-SC-Mal or Dendron-(SC-SO1861)8-Mal was conjugated via cysteine residues (Cys) to CD71mab (a monoclonal antibody recognizing and binding human CD71) to yield CD71mab-(SC-SO1861) (DAR4.2) or CD71mab-dendron(SC-SO1861)8 (DAR^{1.5}). CD71mab-(SC-SO1861) (DAR4.2) or CD71mab-dendron(SC-SO1861)8 (DAR^{1.5}, on average 12 SO1861 molecules, as depicted in FIG. 8A, 8B) or CD71mab-(EMCH-SO1861) (DAR3.8) or CD71mab-dendron(EMCH-SO1861)4 (DAR^{3.2}, on average 12 SO1861 molecules; as depicted in FIG. 7A, 7B) was titrated on a fixed non-effective concentration (see FIG. 21) of 5 pM DianthinEGF (Dia-EGF; dianthin protein toxin fused to EGF) and targeted protein toxin mediated cell killing on CD71+/EGFR+expressing cells (Table 1; SK-BR-3 (CD71⁺/EGFR⁺), JIMT-1 (CD71⁺/EGFR⁺), HeLa (CD71⁺/EGFR⁺), MDA-MB-468 (CD71⁺/EGFR⁺), A431 (CD71⁺/EGFR⁺) as well as on A2058 (CD71⁺/EGFR⁻) was determined (FIG. 23). This revealed strong cell killing at low concentrations of SO1861 when treated with CD71mab-SC-SO1861 (DAR4.2) or CD71mab-dendron(SC-SO1861)8 (DAR^{1.5}) (SK-BR-3: IC50=1 nM; JIMT-1 IC50=10 nM; HeLa IC50=10 nM MDA-MB-468 IC50=10 nM; A431 IC50=100 nM (FIG. 23A-23E), whereas treatment with CD71 mab-(EMCH-SO1861) (DAR3.8) or CD71mab-dendron(EMCH-SO1861)4 (DAR3.2) was less potent (SK-BR-3: IC50=10-20 nM; JIMT-1 IC50=1000 nM; HeLa IC50=1000 nM; MDA-MB-468 IC50=600 nM; A431 IC50=900 nM (FIG. 23A-23E). A2058 cells, lacking EGFR expression (Table 1) were not responsive to any of the treatments; only at very high concentrations some cell-killing activity was observed (FIG. 23F). This shows that compared to CD71mab-(EMCH-SO1861), CD71mab-(SC-SO1861) is more potent, by further improving enhancing endosomal escape of the 5 pM of EGFdianthin toxin, and inducing efficient cell killing only in CD71⁺/EGFR⁺ expressing cells. Improved acid-sensitive linker release of SO1861 from a CD71 receptor targeted monoclonal antibody at low pH in endosomes thus improves the potency of mAb-SO1861 conjugates for delivery of a protein toxin such as a ribosomal inactivating protein toxin in the cytoplasm.

### Example 2

The inventors have previously established that numerous aberrant cells or diseased cells such as tumor cells can be targeted with a conjugate comprising a ligand such as EGF or an antibody and comprising a saponin such as a saponin as listed in Table A1, without wishing to be bound by any theory, resulting in the endocytosis of the conjugate and the accumulation of the saponin in the endosome of the targeted cell, facilitating the endosomal escape of a molecule such as an oligonucleotide or a proteinaceous toxin, which is present in said same cell in the same endosomes at the same time, from the endosome and into the cytosol of the targeted cell. On the target cell, typically an endocytic receptor was targeted, such as EGFR, HER2 and CD71. CD71 is preferred.

Typically, the conjugates comprised a monoclonal antibody capable of binding to such an endocytic cell-surface receptor on the target cell, or comprised at least one sdAb. Examples of suitable endocytic receptor targeting ligands and antibodies were EGF, V_{HH} capable of binding to HER2, VHH capable of binding to CD71, VHH capable of binding to EGFR, MoAb capable of binding to CD71, MoAb capable of binding to HER2 such as trastuzumab and pertuzumab, MoAb capable of binding to EGFR such as cetuximab and matuzumab.

Typically, the saponin comprised by the conjugate was any one of SO1861, SA1641, GE1741, QS-21, QS-21A, QS21-B, Quil-A, SO1832, SO1904, and SO1862.

The inventors have previously successfully established that oligonucleotides such as BNA HSP27 and proteinaceous toxins such as saporin and dianthin are efficiently endocytosed and released from endosomes into the cell cytosol under influence of a conjugate comprising a saponin according to Table A1.

In the saponin-comprising conjugates, the saponin moieties were for example linked to an antibody involving a hydrazone bond formed at the C-23 position, i.e. involving the aldehyde group at position C-23 of the saponin. Under influence of the slightly acidic pH in the endosomes of targeted cells, 35 the hydrazone bond was cleaved and the aldehyde group at position C-23 was again formed, endowing the saponin with the desired endosomal escape enhancing activity towards an effector molecule present in the same endosome, without wishing to be bound by any theory.

The inventors previously established the beneficial effects of endocytosing a saponin-bearing 40 conjugate, when cytosolic delivery of a selected effector molecule such as an oligonucleotide in said target cell is desired. Such saponin-bearing conjugate was for example co-administered with a second conjugate such as an ADC or an AOC. This is referred to as the 2-component approach. Alternatively, the saponin was covalently bound (e.g., via the hydrazone bond) to such an ADC or AOC, which also resulted in enhanced activity of the effector molecule comprised by the ADC or AOC, once present inside the target cell. This is referred to as the 1-component approach.

Illustrative examples of such 2-component combinations are for example published in international applications WO2020126627, WO2021261995, and WO2021259925.

Illustrative examples of such 1-component conjugates are for example published in international application WO2020126620.

In Table A3, references to a series of representative examples in these listed international applications are provided. These examples show that for example CD71 is a suitable target for delivery of saponin and/or effector moiety into a target cell such as an aberrant cell or diseased cell.

These examples also show that for example an oligonucleotide is a suitable effector molecule to be delivered in a target cell and into the cytosol of said cell, under influence of a saponin-bearing conjugate.

TABLE A3
published examples of a conjugate comprising a saponin and a cell-targeting ligand,
wherein the saponin is not linked with the ligand via a semicarbazone bond, said
conjugate combined with a second conjugate comprising a cell-targeting ligand
and an effector moiety, and published examples of 1-component conjugates comprising
a cell-targeting ligand, an effector moiety and a saponin, wherein the saponin
is not linked via a semicarbazone bond in the 1-component conjugate.

| | | Page(s) of | | |
|---|---|---|---|---|
| International | | the | | Remarks relating to the indicated |
| application No. | Example | description | FIG.(s) | examples |
| WO2020126627 | 3, 4, 5, | 24-26, 28, | 7, 14, 15, | Effector moiety is an oligonucleotide or |
| | 14 | 30, 31, 98, | 23, 32, | a toxin; target is CD71, HER2, EGFR; |
| | | 101-102, | 33, 46, | in vivo tumor treatment; conjugates |
| | | 104-105, | 66 | comprising bound saponin directly to |
| | | 108-109, | | the cell-targeting ligand or via a |
| | | 114, 161 | | dendron moiety; 2-component and 1- |
| | | | | component examples; hydrazone |
| | | | | bond between saponin and ligand or |
| | | | | dendron; cell-surface molecule |
| | | | | binding ligand is a monoclonal |
| | | | | antibody |
| WO2021261995 | 1-4 | 11-12, 38- | 2-9 | Effector moiety is a toxin; target is |
| | | 41, 44-45 | | HER2, EGFR; conjugates comprising |
| | | | | bound saponin directly to the cell- |
| | | | | targeting ligand; 2-component |
| | | | | examples; hydrazone bond between |
| | | | | saponin and ligand; saponin conjugate |
| | | | | and effector moiety conjugate bind to |
| | | | | the same cell receptor though to a |
| | | | | different epitope; cell-surface |
| | | | | molecule binding ligand is a |
| | | | | monoclonal antibody |
| WO2021259925 | 1-3, 5, | 14-18, 63- | 2-7, 14, | 2-component examples; Effector |
| | 12-14 | 66, 76-77, | 18, 21, | moiety is an oligonucleotide (BNA) or |
| | | 86-87, 90- | 28 | a proteinaceous toxin; target is CD71, |
| | | 91 | | HER2, EGFR; conjugates comprising |
| | | | | bound saponin directly to the cell- |
| | | | | targeting ligand; hydrazone bond |
| | | | | between saponin and ligand; cell- |
| | | | | surface molecule binding ligand is a |
| | | | | single domain antibody or an antibody |
| | | | | for the saponin-comprising conjugate; |
| | | | | ligand can be a cell receptor ligand, |
| | | | | here EGF; in vivo tumor models; cell- |
| | | | | surface molecule binding molecule of |
| | | | | the conjugate comprising the effector |
| | | | | moiety can be bivalent VHH |
| WO2020126620 | 1-3 | 26, 94-97, | 1-7 | 1-component examples; in vivo tumor |
| | | | | treatment; effector moiety is an |
| | | | | oligonucleotide or a toxin; cell-surface |
| | | | | receptors targeted by conjugates are |
| | | | | HER2, EGFR; cell-surface receptor |
| | | | | binding ligand in the conjugate is an |
| | | | | antibody |

1. A saponin conjugate comprising a first proteinaceous molecule (‘proteinaceous molecule 1’) comprising a cell-surface molecule binding-molecule comprising a first binding site for binding to a first epitope of a first cell-surface molecule and further comprising at least one thiol functional group, according to formula (X) 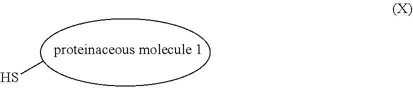
the first proteinaceous molecule covalently bound with at least one saponin derivative, wherein the at least one saponin derivative is based on a saponin comprising a triterpene aglycone core structure and at least one of a first saccharide chain ‘R¹’ and a second saccharide chain ‘R²’ linked to the aglycone core structure, wherein the saponin derivative comprises an aglycone core structure comprising an aldehyde group, wherein the aldehyde group is transformed into a semicarbazone functional group according to formula (I) 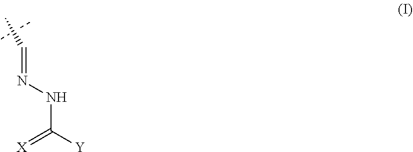
wherein R¹ and R² are independently selected from hydrogen, a monosaccharide, a linear oligosaccharide and a branched oligosaccharide,
X=O, P or S, and
Y= 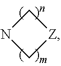
wherein n and m each are an integer independently selected from 1, 2, or 3,
Z=NR⁵, and
wherein R⁵ represents a maleimide moiety according to formula (II) 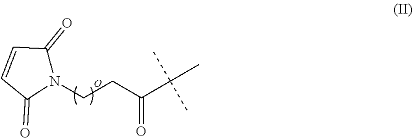
wherein o is an integer selected from 0-10, preferably 2-7, more preferably 4-6,
and wherein the maleimide moiety (II) of the saponin derivative is further transformed into a thioether bond through reaction
either, with the at least one thiol functional group of the first proteinaceous molecule,
or, with at least one thiol functional group of an oligomeric molecule which oligomeric molecule comprises a maleimide moiety that is transformed into a thioether bond through reaction with the at least one thiol functional group of the first proteinaceous molecule.

2. Saponin conjugate according to claim 1, wherein the saponin is a mono-desmosidic triterpene saponin or bi-desmosidic triterpene saponin belonging to the type of a 12,13-dehydrooleanane with the aldehyde group in position C-23 and optionally comprising a glucuronic acid group in a carbohydrate substituent at the C-3beta-OH group of the saponin, preferably a bi-desmosidic triterpene saponin belonging to the type of a 12,13-dehydrooleanane with the aldehyde group in position C-23 and comprising a glucuronic acid group in a carbohydrate substituent at the C-3beta-OH group of the saponin.

3. Saponin conjugate according to claim 1 or 2, wherein the triterpene aglycone core structure is selected from quillaic acid and gypsogenin, preferably the triterpene aglycone core structure is quillaic acid.

4. Saponin conjugate according to any one of the claims 1-3, wherein the saponin derivative is according to formula (IV): 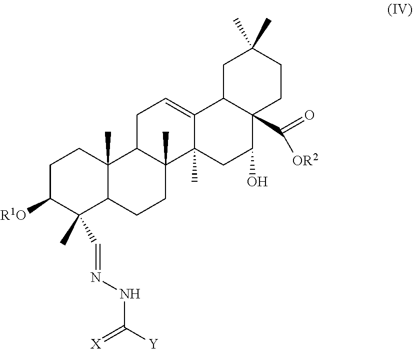
wherein R¹ and R² are independently selected from hydrogen, a monosaccharide, a linear oligosaccharide and a branched oligosaccharide,
X=O, P or S, and
Y= 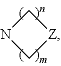
wherein n and m each are an integer independently selected from 1, 2 or 3;
Z=NR⁵, and
wherein R⁵ represents a maleimide moiety according to formula (II) 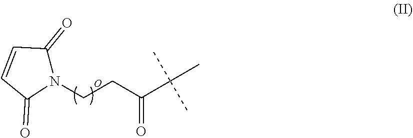
wherein o is an integer selected from 0-10, preferably 2-7, more preferably 4-6.

5. Saponin conjugate according to any one of the claims 1-4, wherein X=O.

6. Saponin conjugate according to any one of the claims 1-5,
wherein Y= 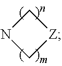
n and m each are an integer independently selected from 1, 2 or 3; and
Z=NR⁵; and
wherein R⁵ represents a maleimide moiety according to formula (II) 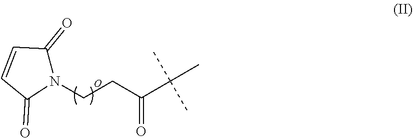
wherein o is an integer selected from 0-10, preferably 2-7, more preferably 4-6.

7. Saponin conjugate according to any one of the claims 1-6,
wherein n=1 and m=1, or
n=2 and m=1, or
n=2 and m=2, or
n=3 and m=2, or
n=3 and m=3;
preferably wherein n=2 and m=2.

8. Saponin conjugate according to any one of the claims 1-7, wherein
the first saccharide chain R¹ is selected from:
H,
GlcA-,
Glc-,
GaI-,
Rha-(1→2)-Ara-,
GaI-(1→2)-[XyI-(1→3)]-GlcA-,
Glc-(1→2)-[Glc-(1→4)]-GlcA-,
Glc-(1→2)-Ara-(1→3)-[GaI-(1→2)]-GlcA-,
XyI-(1→2)-Ara-(1→3)-[GaI-(1→2)]-GlcA-,
Glc-(1→3)-GaI-(1→2)-[XyI-(1→3)]-Glc-(1→4)-GaI-,
Rha-(1→2)-GaI-(1→3)-[Glc-(1→2)]-GlcA-,
Ara-(1→4)-Rha-(1→2)-Glc-(1→2)-Rha-(1→2)-GlcA-,
Ara-(1→4)-Fuc-(1→2)-Glc-(1→2)-Rha-(1→2)-GlcA-,
Ara-(1→4)-Rha-(1→2)-GaI-(1→2)-Rha-(1→2)-GlcA-,
Ara-(1→4)-Fuc-(1→2)-GaI-(1→2)-Rha-(1→2)-GlcA-,
Ara-(1→4)-Rha-(1→2)-Glc-(1→2)-Fuc-(1→2)-GlcA-,
Ara-(1→4)-Fuc-(1→2)-Glc-(1→2)-Fuc-(1→2)-GlcA-,
Ara-(1→4)-Rha-(1→2)-GaI-(1→2)-Fuc-(1→2)-GlcA-,
Ara-(1→4)-Fuc-(1→2)-GaI-(1→2)-Fuc-(1→2)-GlcA-,
XyI-(1→4)-Rha-(1→2)-Glc-(1→2)-Rha-(1→2)-GlcA-,
XyI-(1→4)-Fuc-(1→2)-Glc-(1→2)-Rha-(1→2)-GlcA-,
XyI-(1→4)-Rha-(1→2)-GaI-(1→2)-Rha-(1→2)-GlcA-,
XyI-(1→4)-Fuc-(1→2)-GaI-(1→2)-Rha-(1→2)-GlcA-,
XyI-(1→4)-Rha-(1→2)-Glc-(1→2)-Fuc-(1→2)-GlcA-,
XyI-(1→4)-Fuc-(1→2)-Glc-(1→2)-Fuc-(1→2)-GlcA-,
XyI-(1→4)-Rha-(1→2)-GaI-(1→2)-Fuc-(1→2)-GlcA-,
XyI-(1→4)-Fuc-(1→2)-GaI-(1→2)-Fuc-(1→2)-GlcA-, and
derivatives thereof, and
wherein the second saccharide chain R² is selected from:
H,
Glc-,
GaI-,
Rha-(1→2)-[XyI-(1→4)]-Rha-,
Rha-(1→2)-[Ara-(1→3)-XyI-(1→4)]-Rha-,
Ara-,
XyI-,
XyI-(1→4)-Rha-(1→2)-[R1-(→4)]-Fuc- wherein R1 is 4E-Methoxycinnamic acid,
XyI-(1→4)-Rha-(1→2)-[R2-(→-4)]-Fuc- wherein R2 is 4Z-Methoxycinnamic acid,
XyI-(1→4)-[GaI-(1→3)]-Rha-(1→2)-4-OAc-Fuc-,
XyI-(1→4)-[Glc-(1→3)]-Rha-(1→2)-3,4-di-OAc-Fuc-,
XyI-(1→4)-[Glc-(1→3)]-Rha-(1→2)-[R⁶-(→4)]-3-OAc-Fuc- wherein R⁶ is 4E-Methoxycinnamic acid,
Glc-(1→3)-XyI-(1→4)-[Glc-(1→3)]-Rha-(1→2)-4-OAc-Fuc-,
Glc-(1→3)-XyI-(1→4)-Rha-(1→2)-4-OAc-Fuc-,
(Ara- or XyI-)(1→3)-(Ara- or XyI-)(1→4)-(Rha- or Fuc-)(1→2)-[4-OAc-(Rha- or Fuc-)(1→4)]-(Rha- or Fuc-),
XyI-(1→3)-XyI-(1→4)-Rha-(1→2)-[Qui-(1→4)]-Fuc-,
Api-(1→3)-XyI-(1→4)-[Glc-(1→3)]-Rha-(1→2)-Fuc-,
XyI-(1→4)-[GaI-(1→3)]-Rha-(1→2)-Fuc-,
XyI-(1→4)-[Glc-(1→3)]-Rha-(1→2)-Fuc-,
Ara/XyI-(1→4)-Rha/Fuc-(1→4)-[Glc/GaI-(1→2)]-Fuc-,
Api-(1→3)-XyI-(1→4)-[Glc-(1→3)]-Rha-(1→2)-[R⁷-(→4)]-Fuc- wherein R⁷ is 5-O-[5-O-Ara/Api-3,5-dihydroxy-6-methyl-octanoyl]-3,5-dihydroxy-6-methyl-octanoic acid),
Api-(1→3)-XyI-(1→4)-Rha-(1→2)-[R⁸-(→4)]-Fuc- wherein R⁸ is 5-O-[5-O-Ara/Api-3,5-dihydroxy-6-methyl-octanoyl]-3,5-dihydroxy-6-methyl-octanoic acid),
Api-(1→3)-XyI-(1→4)-Rha-(1→2)-[Rha-(1→3)]-4-OAc-Fuc-,
Api-(1→3)-XyI-(1→4)-[Glc-(1→3)]-Rha-(1→2)-[Rha-(1→3)]-4-OAc-Fuc-,
6-OAc-Glc-(1→3)-XyI-(1→4)-Rha-(1→2)-[3-OAc-Rha-(1→3)]-Fuc-,
Glc-(1→3)-XyI-(1→4)-Rha-(1→2)-[3-OAc-Rha-(1→3)]-Fuc-,
XyI-(1→3)-XyI-(1→4)-Rha-(1→2)-[Qui-(1→4)]-Fuc-,
Glc-(1→3)-[XyI-(1→4)]-Rha-(1→2)-[Qui-(1→4)]-Fuc-,
Glc-(1→3)-XyI-(1→4)-Rha-(1→2)-[XyI-(1→3)-4-OAc-Qui-(1→4)]-Fuc-,
XyI-(1→3)-XyI-(1→4)-Rha-(1→2)-[3,4-di-OAc-Qui-(1→4)]-Fuc-,
Glc-(1→3)-[XyI-(1→4)]-Rha-(1→2)-Fuc-,
6-OAc-Glc-(1→3)-[XyI-(1→4)]-Rha-(1→2)-Fuc-,
Glc-(1→3)-[XyI-(1→3)-XyI-(1→4)]-Rha-(1→2)-Fuc-,
XyI-(1→3)-XyI-(1→4)-Rha-(1→2)-[XyI-(1→3)-4-OAc-Qui-(1→4)]-Fuc-,
Api/XyI-(1→3)-XyI-(1→4)-[Glc-(1→3)]-Rha-(1→2)-[Rha-(1→3)]-4OAc-Fuc-,
Api-(1→3)-XyI-(1→4)-[Glc-(1→3)]-Rha-(1→2)-[Rha-(1→3)]-4OAc-Fuc-,
Api/XyI-(1→3)-XyI-(1→4)-[Glc-(1→3)]-Rha-(1→2)-[R⁹-(→4)]-Fuc- wherein R⁹ is 5-O-[5-O-Rha-(1→2)-Ara/Api-3,5-dihydroxy-6-methyl-octanoyl]-3,5-dihydroxy-6-methyl-octanoic acid),
Api/XyI-(1→3)-XyI-(1→4)-[Glc-(1→3)]-Rha-(1→2)-[R¹⁰-(→4)]-Fuc- wherein R¹⁰ is 5-O-[5-O-Ara/Api-3,5-dihydroxy-6-methyl-octanoyl]-3,5-dihydroxy-6-methyl-octanoic acid),
Api/XyI-(1→3)-XyI-(1→4)-[Glc-(1→3)]-Rha-(1→2)-[R¹¹-(→4)]-Fuc- wherein R¹¹ is 5-O-[5-O-Ara/Api-3,5-dihydroxy-6-methyl-octanoyl]-3,5-dihydroxy-6-methyl-octanoic acid),
Api-(1→3)-XyI-(1→4)-Rha-(1→2)-[R¹²-(→4)]-Fuc- wherein R¹² is 5-O-[5-O-Ara/Api-3,5-dihydroxy-6-methyl-octanoyl]-3,5-dihydroxy-6-methyl-octanoic acid),
XyI-(1→3)-XyI-(1→4)-Rha-(1→2)-[R¹³-(→4)]-Fuc- wherein R¹³ is 5-O-[5-O-Ara/Api-3,5-dihydroxy-6-methyl-octanoyl]-3,5-dihydroxy-6-methyl-octanoic acid),
Api-(1→3)-XyI-(1→4)-Rha-(1→2)-[R¹⁴-(→3)]-Fuc- wherein R¹⁴ is 5-O-[5-O-Ara/Api-3,5-dihydroxy-6-methyl-octanoyl]-3,5-dihydroxy-6-methyl-octanoic acid),
XyI-(1→3)-XyI-(1→4)-Rha-(1→2)-[R¹⁵-(→3)]-Fuc- wherein R¹⁵ is 5-O-[5-O-Ara/Api-3,5-dihydroxy-6-methyl-octanoyl]-3,5-dihydroxy-6-methyl-octanoic acid)
Glc-(1→3)-[Glc-(1→6)]-GaI-, and
derivatives thereof.

9. Saponin conjugate according to any one of the claims 1-8, wherein
the first saccharide chain R¹ is GaI-(1→2)-[XyI-(1→3)]-GlcA-; and
wherein the second saccharide chain R² is selected from:
Glc-(1→3)-XyI-(1→4)-Rha-(1→2)-[XyI-(1→3)-4-OAc-Qui-(1→4)]-Fuc-,
XyI-(1→3)-XyI-(1→4)-Rha-(1→2)-[3,4-di-OAc-Qui-(1→4)]-Fuc-,
Api-(1→3)-XyI-(1→4)-Rha-(1→2)-[R¹²-(→4)]-Fuc- wherein R¹² is 5-O-[5-O-Ara/Api-3,5-dihydroxy-6-methyl-octanoyl]-3,5-dihydroxy-6-methyl-octanoic acid,
XyI-(1→3)-XyI-(1→4)-Rha-(1→2)-[R¹³-(→4)]-Fuc- wherein R¹³ is 5-O-[5-O-Ara/Api-3,5-dihydroxy-6-methyl-octanoyl]-3,5-dihydroxy-6-methyl-octanoic acid,
Api-(1→3)-XyI-(1→4)-Rha-(1→2)-[R¹⁴-(→3)]-Fuc- wherein R¹⁴ is 5-O-[5-O-Ara/Api-3,5-dihydroxy-6-methyl-octanoyl]-3,5-dihydroxy-6-methyl-octanoic acid, and
XyI-(1→3)-XyI-(1→4)-Rha-(1→2)-[R¹⁵-(→3)]-Fuc- wherein R¹⁵ is 5-O-[5-O-Ara/Api-3,5-dihydroxy-6-methyl-octanoyl]-3,5-dihydroxy-6-methyl-octanoic acid,
preferably, R¹ is GaI-(1→2)-[XyI-(1→3)]-GlcA- and R² is Glc-(1→3)-XyI-(1→4)-Rha-(1→2)-[XyI-(1→3)-4-OAc-Qui-(1→4)]-Fuc-.

10. Saponin conjugate of any one of the claims 1-9, wherein the at least one saponin on which the saponin derivative is based is any one or more of:
a) saponin selected from any one or more of list A:
Quillaja saponaria saponin mixture, or a saponin isolated from Quillaja saponaria, for example Quil-A, QS-17-api, QS-17-xyl, QS-21, QS-21A, QS-21B, QS-7-xyl;
Saponinum album saponin mixture, or a saponin isolated from Saponinum album;
Saponaria officinalis saponin mixture, or a saponin isolated from Saponaria officinalis; and
Quillaja bark saponin mixture, or a saponin isolated from Quillaja bark, for example Quil-A, QS-17-api, QS-17-xyl, QS-21, QS-21A, QS-21B, QS-7-xyl; or
b) a saponin comprising a gypsogenin aglycone core structure, selected from list B:
SA1641, gypsoside A, NP-017772, NP-017774, NP-017777, NP-017778, NP-018109, NP-017888, NP-017889, NP-018108, SO1658 and Phytolaccagenin; or
c) a saponin comprising a quillaic acid aglycone core structure, selected from list C:
AG1856, AG1, AG2, Agrostemmoside E, GE1741, Gypsophila saponin 1 (Gyp1), NP-017674, NP-017810, NP-003881, NP-017676, NP-017677, NP-017705, NP-017706, NP-017773, NP-017775, SA1657, Saponarioside B, SO1542, SO1584, SO1674, SO1700, SO1730, SO1772, SO1832, SO1861, SO1862, SO1904, QS1861, QS1862, QS-7, QS-7 api, QS-17, QS-18, QS-21 A-apio, QS-21 A-xylo, QS-21 B-apio and QS-21 B-xylo,
preferably, the at least one saponin is any one or more of a saponin selected from list B or C, more preferably from list C.

11. Saponin conjugate of any one of the claims 1-10, wherein the at least one saponin on which the saponin derivative is based is any one or more of
AG1856, GE1741, a saponin isolated from Quillaja saponaria, Quil-A, QS-17, QS-21, QS-7, SA1641, a saponin isolated from Saponaria officinalis, Saponarioside B, SO1542, SO1584, SO1658, SO1674, SO1700, SO1730, SO1772, SO1832, SO1861, SO1862 and SO1904, preferably the at least one saponin is any one or more of QS-21, SO1832, SO1861, SA1641 and GE1741, more preferably the at least one saponin is QS-21, SO1832 or SO1861, even more preferably the at least one saponin is SO1861 or SO1832.

12. Saponin conjugate of any one of the claims 1-11, wherein the at least one saponin on which the saponin derivative is based is a saponin isolated from Saponaria officinalis, preferably the at least one saponin is any one or more of Saponarioside B, SO1542, SO1584, SO1658, SO1674, SO1700, SO1730, SO1772, SO1832, SO1861, SO1862 and SO1904, more preferably the at least one saponin is any one or more of SO1832, SO1861 and SO1862, even more preferably SO1832 and SO1861, even more preferably the at least one saponin is SO1861.

13. Saponin conjugate according to any one of the claims 1-12, wherein the saponin derivative is according to formula (VII)

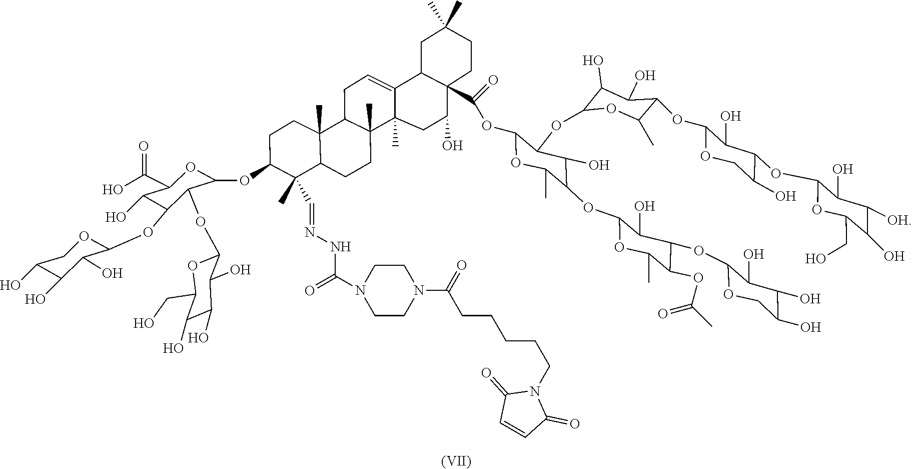

14. Saponin conjugate according to any one of the claims 1-13, wherein the saponin conjugate is according to formula (XI) 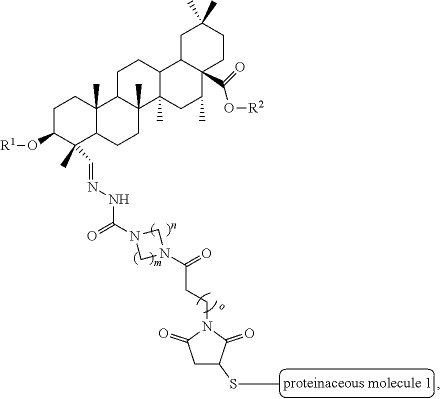
wherein R¹ and R² are as defined in any one of claims 1-13;
n and m each are an integer independently selected from 1, 2 or 3; and
o is an integer selected from 0-10, preferably 2-7, more preferably 4-6.

15. Saponin conjugate according to any one of the claims 1-14, wherein the saponin conjugate is according to formula (XII)

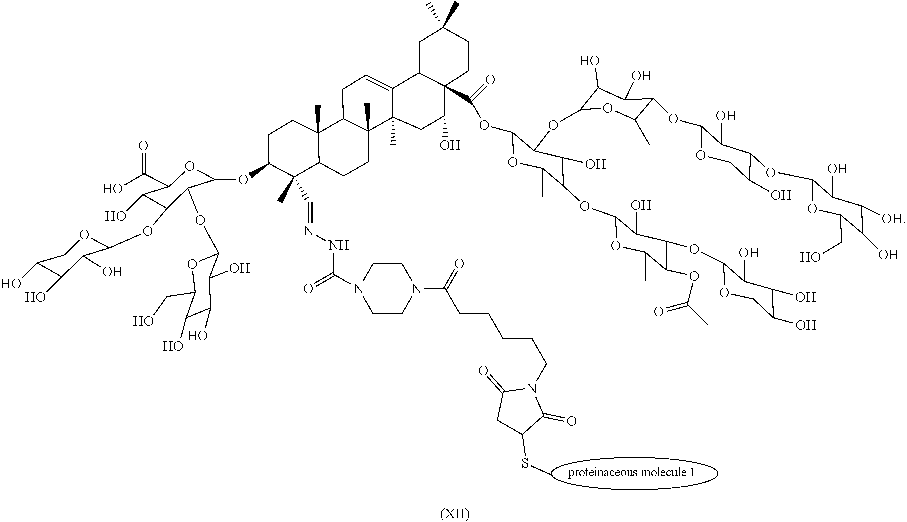

16. Saponin conjugate of any one of the claims 1-13, wherein the oligomeric molecule to which the at least one saponin derivative is covalently bound, is selected from: a dendron, a poly-ethylene glycol such as any one of PEG₃-PEG₃₀, preferably any one of PEG₄-PEG₁₂, preferably the oligomeric molecule is a dendron such as a poly-amidoamine (PAMAM) dendrimer.

17. Saponin conjugate of any one of the claim 1-13 or 16, wherein the oligomeric molecule is a dendron, preferably a G2 dendron, a G3 dendron, a G4 dendron or a G5 dendron, more preferably a G2 dendron or a G3 dendron.

18. Saponin conjugate of any one of the claims 1-17, wherein the semicarbazone functional group 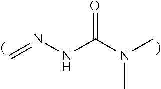
is hydrolysable under acidic conditions, preferably at pH 4.0-6.5, wherein hydrolysis of said semicarbazone functional group provides the aldehyde group on the aglycone core structure of the saponin on which the saponin derivative is based,
and/or
wherein the semicarbazone functional group is subject to cleavage in vivo under acidic conditions such as for example present in endosomes and/or lysosomes of a mammalian cell, preferably a human cell such as a diseased cell, an aberrant cell or a tumor cell, preferably at pH 4.0-6.5, and more preferably at pH≤5.5, wherein hydrolysis of said semicarbazone functional group provides the aldehyde group on the aglycone core structure of the saponin on which the saponin derivative is based.

19. Saponin conjugate of any one of the previous claims, comprising more than one copy of the saponin, preferably any number of saponin copies selected from 1-64 copies of the saponin, more preferably 2-32 copies of the saponin, even more preferably 3-16 copies of the saponin, even more preferably 4-12 copies of the saponin, such as 1, 2, 3, 4, 5, 6, 7, 8, 9, 10, 11, 12, 13, 14, 15 or 16 copies of the saponin, preferably 2, 4 or 8 copies of the saponin.

20. Saponin conjugate according to any one of the claim 1-13 or 16-19, wherein the saponin conjugate is according to formula (XII)a

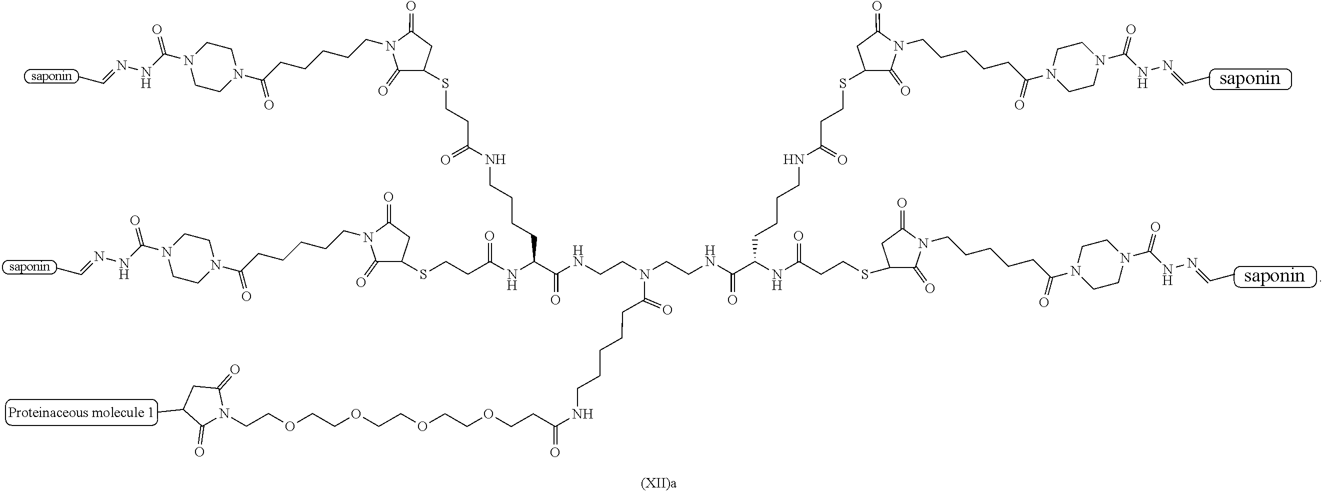 [SMILES: C/C=N/NC(=O)N1CCN(C(=O)CCCCCN2C(=O)CC(SCCC(=O)NCCCC[C@H](NC(=O)CCSC3CC(=O)N(CCCCCC(=O)N4CCN(C(=O)N/N=C/C)CC4)C3=O)C(=O)NCCN(CCNC(=O)[C@H](CCCCNC(=O)CCSC3CC(=O)N(CCCCCC(=O)N4CCN(C(=O)N/N=C/C)CC4)C3=O)NC(=O)CCSC3CC(=O)N(CCCCCC(=O)N4CCN(C(=O)N/N=C/C)CC4)C3=O)C(=O)CCCCCNC(=O)CCOCCOCCOCCOCCN3C(=O)CC(C)C3=O)C2=O)CC1]

21. Saponin conjugate according to any one of the claim 1-13 or 16-19, wherein the saponin conjugate is according to formula (XII)b

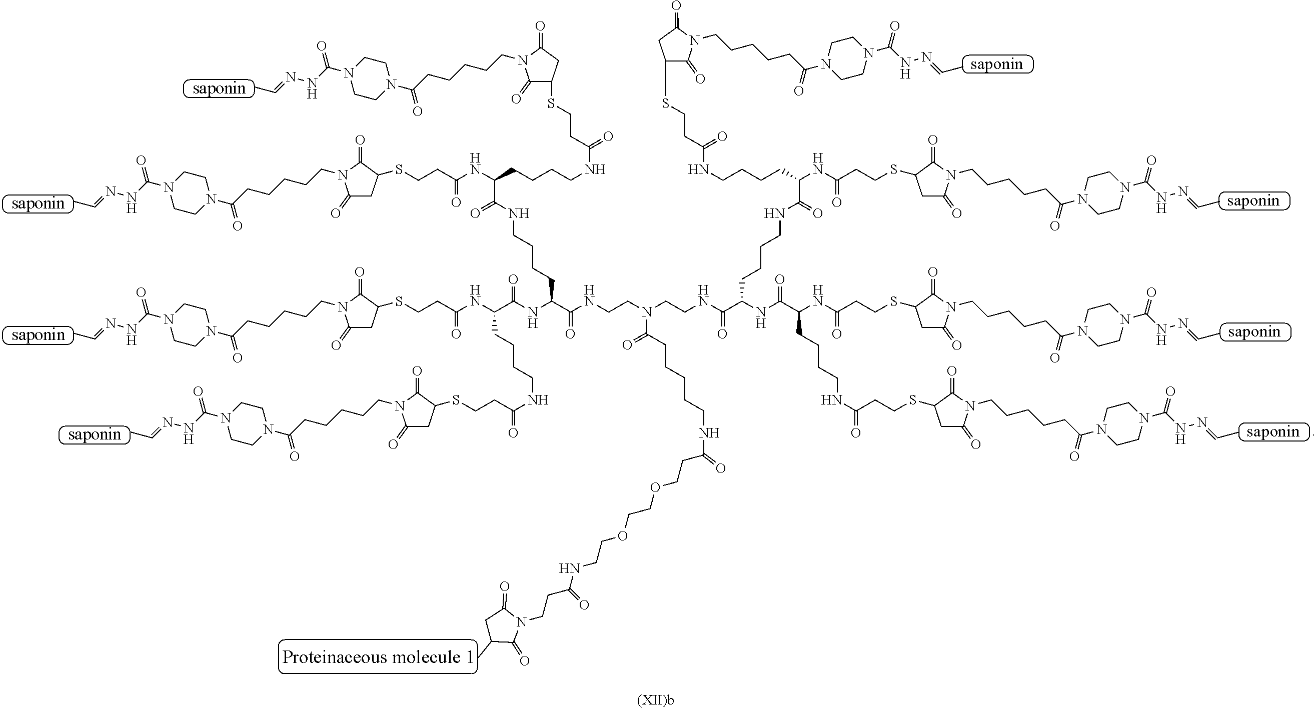

22. Saponin conjugate according to any one of the claims 1-21, wherein the first binding site of the proteinaceous molecule 1 is or comprises any one or more of: an amino acid, a peptide, a protein, an antibody such as an IgG, preferably a monoclonal antibody, or a binding derivative of said antibody or binding fragment of said antibody or binding domain of said antibody such as a F(ab′)2 fragment, Fab′ fragment, Fab fragment, scFv, dsFv, scFv-Fc, reduced IgG (rIgG), minibody, diabody, triabody, tetrabody, Fc fusion protein, nanobody, variable V domain, a single-domain antibody (sdAb), wherein the sdAb preferably is a V_{HH}, for example a camelid V_{H}, or wherein said first binding site is or comprises a ligand for a cell-surface molecule preferably a receptor, preferably wherein the ligand is a proteinaceous ligand such as EGF or a cytokine, an adnectin, an affibody, an anticalin, or binding molecules comprising one or more of any of these cell-surface molecule binding-molecules.

23. Saponin conjugate according to any one of the claims 1-22, wherein the first binding site of the proteinaceous molecule 1 is or comprises an antibody, preferably a monoclonal antibody, such as an IgG, or a binding derivative of said antibody or binding fragment of said antibody or binding domain of said antibody, preferably the first binding site is an antibody.

24. Saponin conjugate according to any one of the claims 1-22, wherein the proteinaceous molecule 1 comprises a cell-surface molecule binding-molecule comprising a first binding site for binding to a first epitope of a first cell-surface molecule, wherein said first binding site is or comprises any one or more of: a single-domain antibody (sdAb), preferably a V_{H} domain derived from a heavy chain of an antibody, preferably of immunoglobulin G origin, preferably of human origin, a V_{L} domain derived from a light chain of an antibody, preferably of immunoglobulin G origin, preferably of human origin, a V_{HH} domain such as derived from a heavy-chain only antibody (HCAb) such as from Camelidae origin or Ig-NAR origin such as a variable heavy chain new antigen receptor (VNAR) domain, preferably the HCAb is from Camelidae origin, preferably the sdAb is a V_{HH} domain derived from an HCAb from Camelidae origin (camelid V_{H}) such as derived from an HCAb from camel, lama, alpaca, dromedary, vicuna, guanaco and Bactrian camel.

25. Saponin conjugate according to any one of the claims 1-24, wherein the first epitope of the first cell-surface molecule is any one or more of: a diseased cell specific first epitope of a cell-surface receptor, a first epitope of a cell-surface receptor over-expressed on a diseased cell, an aberrant cell specific first epitope of a cell-surface receptor, a first epitope of a cell-surface receptor overexpressed on an aberrant cell, a tumor-cell specific first epitope of a first tumor-cell surface receptor, preferably of a first tumor-cell surface receptor specifically present on a tumor cell and/or overexpressed on the tumor cell.

26. Saponin conjugate of any one of the claims 1-25, wherein the first cell surface molecule is a first cell surface receptor, preferably an endocytic cell-surface receptor, preferably a diseased cell specific receptor or an aberrant cell specific receptor or a tumor-cell specific receptor, or a receptor overexpressed at a diseased cell, aberrant cell or tumor cell, more preferably the cell surface molecule is selected from any one or more of: CD71, CA125, EpCAM(17-1A), CD52, CEA, CD44v6, FAP, EGF-IR, integrin, syndecan-1, vascular integrin alpha-V beta-3, HER2, EGFR, CD20, CD22, Folate receptor 1, CD146, CD56, CD19, CD138, CD27L receptor, prostate specific membrane antigen (PSMA), CanAg, integrin-alphaV, CA6, CD33, mesothelin, Cripto, CD3, CD30, CD239, CD70, CD123, CD352, DLL3, CD25, ephrinA4, MUC-1, Trop2, CEACAM5, CEACAM6, HER3, CD74, PTK7, Notch3, FGF2, C4.4A, FLT3, CD38, FGFR3, CD7, PD-L1, CTLA-4, CD52, PDGFRA, VEGFR1, VEGFR2, c-Met (HGFR), EGFR1, RANKL, ADAMTS5, CD16, CXCR7 (ACKR3), glucocorticoid-induced TNFR-related protein (GITR), even more preferably the cell surface molecule is selected from: CD71, HER2, c-Met, VEGFR2, CXCR7, CD71, EGFR and EGFR1, even more preferably the cell surface molecule is any one of CD71, HER2 and EGFR, most preferably the cell surface molecule is cell surface receptor CD71.

27. Saponin conjugate of any one of the claims 22-26, wherein the antibody is selected from, or the sdAb is derived from or based on, any one or more of immunoglobulins: an anti-CD71 antibody such as IgG type OKT-9, an anti-HER2 antibody such as trastuzumab (Herceptin), pertuzumab, an anti-CD20 antibody such as rituximab, ofatumumab, tositumomab, obinutuzumab ibritumomab, an anti-CA125 antibody such as oregovomab, an anti-EpCAM (17-1A) antibody such as edrecolomab, an anti-EGFR antibody such as cetuximab, matuzumab, panitumumab, nimotuzumab, an anti-CD30 antibody such as brentuximab, an anti-CD33 antibody such as gemtuzumab, huMy9-6, an anti-vascular integrin alpha-v beta-3 antibody such as etaracizumab, an anti-CD52 antibody such as alemtuzumab, an anti-CD22 antibody such as epratuzumab, pinatuzumab, binding fragment (Fv) of anti-CD22 antibody moxetumomab, humanized monoclonal antibody inotuzumab, an anti-CEA antibody such as labetuzumab, an anti-CD44v6 antibody such as bivatuzumab, an anti-FAP antibody such as sibrotuzumab, an anti-CD19 antibody such as huB4, an anti-CanAg antibody such as huC242, an anti-CD56 antibody such as huN901, an anti-CD38 antibody such as daratumumab, OKT-10 anti-CD38 monoclonal antibody, an anti-CA6 antibody such as DS6, an anti-IGF-1R antibody such as cixutumumab, 3B7, an anti-integrin antibody such as CNTO 95, an anti-syndecan-1 antibody such as B-134, an anti-CD79b such as polatuzumab, an anti-HIVgp41 antibody, or an immunoglobulin with at least 95% amino-acid sequence identity with any one of these immunoglobulins, preferably at least 96%, more preferably at least 97%, even more preferably at least 98%, even more preferably at least 99%,
preferably the antibody is selected from, or the sdAb is derived from or based on any one or more of immunoglobulins: an anti-HIVgp41 antibody, an anti-CD71 antibody, an anti-HER2 antibody and an anti-EGFR antibody, or an immunoglobulin with at least 95% amino-acid sequence identity with any one of these immunoglobulins, preferably at least 96%, more preferably at least 97%, even more preferably at least 98%, even more preferably at least 99%, more preferably the antibody is, or the sdAb is derived from or based on any one or more of:
trastuzumab, pertuzumab, cetuximab, matuzumab, an anti-CD71 antibody, OKT-9, or an immunoglobulin with at least 95% amino-acid sequence identity with any one of these immunoglobulins, preferably at least 96%, more preferably at least 97%, even more preferably at least 98%, even more preferably at least 99%,
even more preferably the antibody is, or the sdAb is derived from or based on any one or more of: an anti-CD71 antibody, trastuzumab, cetuximab, the anti-CD71 antibody OKT-9, or an immunoglobulin with at least 95% amino-acid sequence identity with any one of these immunoglobulins, preferably at least 96%, more preferably at least 97%, even more preferably at least 98%, even more preferably at least 99%,
more preferably the antibody is, or the sdAb is derived from or based on any one or more of:
an anti-CD71 antibody such as OKT-9, or an immunoglobulin with at least 95% amino-acid sequence identity with such immunoglobulin, preferably at least 96%, more preferably at least 97%, even more preferably at least 98%, even more preferably at least 99%, and preferably the proteinaceous molecule 1 is a monoclonal antibody, preferably an anti-CD71-antibody.

28. Composition comprising the saponin conjugate of any one of the previous claims, and optionally a pharmaceutically acceptable diluent and/or a pharmaceutically acceptable excipient.

29. First pharmaceutical combination comprising:
(a) the composition of claim 28; and
(b) a first pharmaceutical composition comprising a covalently bound conjugate comprising a cell-surface molecule binding-molecule, such as a second proteinaceous molecule (‘proteinaceous molecule 2’), and an effector moiety, wherein the proteinaceous molecule 2 is the same or different from the proteinaceous molecule 1 present in the saponin conjugate, and if the proteinaceous molecule 2 is different from the proteinaceous molecule 1, the proteinaceous molecule 2 comprising a second binding site for binding to a second epitope of a second cell-surface molecule, wherein the second cell-surface molecule is the same as or different from the first cell surface molecule, and if the second cell-surface molecule is different from the first cell surface molecule, the second cell-surface molecule and the first cell surface molecule are preferably present on the same cell,
the first pharmaceutical composition optionally further comprising a pharmaceutically acceptable excipient and/or a pharmaceutically acceptable diluent.

30. First pharmaceutical combination of claim 29, wherein the second proteinaceous molecule is selected from the proteinaceous molecules according to any one of the claims 22-27.

31. Second pharmaceutical combination, comprising:
(a) the composition of claim 28; and
(b) a second pharmaceutical composition comprising a covalently bound conjugate comprising a cell-surface molecule binding-molecule, such as a third proteinaceous molecule (‘proteinaceous molecule 3’), and an effector moiety, wherein the proteinaceous molecule 3 comprises the first binding site for binding to the first epitope on the cell-surface molecule according to any one of the claims 1-27, the second pharmaceutical composition optionally further comprising a pharmaceutically acceptable excipient and/or a pharmaceutically acceptable diluent,
wherein the first binding site of the proteinaceous molecule 1 and the first binding site of the proteinaceous molecule 3 are the same, and wherein the first cell-surface molecule and the first epitope on the first cell-surface molecule, to which the proteinaceous molecule 1 can bind, and the first cell-surface molecule and the first epitope on the first cell-surface molecule, to which the proteinaceous molecule 3 can bind, are the same.

32. Second pharmaceutical combination of claim 31, wherein the third proteinaceous molecule is selected from the proteinaceous molecules according to any one of the claims 22-27.

33. Third pharmaceutical composition comprising:
(a) the saponin conjugate of any one of the claims 1-27;
and comprising
either
(b1) the conjugate of claim 29 or 30 comprising proteinaceous molecule 2 and an effector moiety,
or
(b2) the conjugate of claim 31 or 32 comprising proteinaceous molecule 3 and an effector moiety,
and the third pharmaceutical composition optionally comprising a pharmaceutically acceptable excipient and/or a pharmaceutically acceptable diluent.

34. First pharmaceutical combination of claim 29 or 30, second pharmaceutical combination of claim 31 or 32, or third pharmaceutical composition of claim 33, wherein the effector moiety is an oligonucleotide.

35. First pharmaceutical combination of claim 34, second pharmaceutical combination of claim 34, or third pharmaceutical composition of claim 34, wherein the effector moiety is an oligonucleotide selected from deoxyribonucleic acid (DNA) oligomer, ribonucleic acid (RNA) oligomer, anti-sense oligonucleotide (ASO, AON), short interfering RNA (siRNA), microRNA (miRNA), anti-microRNA (anti-miRNA), DNA aptamer, RNA aptamer, mRNA, mini-circle DNA, peptide nucleic acid (PNA), phosphoramidate morpholino oligomer (PMO), locked nucleic acid (LNA), bridged nucleic acid (BNA), 2′-deoxy-2′-fluoroarabino nucleic acid (FANA), 2′-O-methoxyethyl-RNA (MOE), 3′-fluoro hexitol nucleic acid (FHNA), glycol nucleic acid (GNA), xeno nucleic acid oligonucleotide and threose nucleic acid (TNA).

36. First pharmaceutical combination of claim 34 or 35, second pharmaceutical combination of claim 34 or 35, or third pharmaceutical composition of claim 34 or 35, wherein the oligonucleotide is selected from any one or more of a(n): short interfering RNA (siRNA), short hairpin RNA (shRNA), anti-hairpin-shaped microRNA (miRNA), single-stranded RNA, aptamer RNA, double-stranded RNA (dsRNA), microRNA (miRNA), anti-microRNA (anti-miRNA, anti-miR), antisense oligonucleotide (ASO), mRNA, DNA, antisense DNA, locked nucleic acid (LNA), bridged nucleic acid (BNA), 2′-O,4′-aminoethylene bridged nucleic Acid (BNA Nc), BNA-based siRNA, and BNA-based antisense oligonucleotide (BNA-AON).

37. First pharmaceutical combination of any one of the claims 34-36, second pharmaceutical combination of any one of the claims 34-36, or third pharmaceutical composition of any one of the claims 34-36, wherein the effector moiety is an oligonucleotide selected from any one of an anti-miRNA, a BNA-AON or an siRNA, such as BNA-based siRNA, preferably selected from chemically modified siRNA, metabolically stable siRNA and chemically modified, metabolically stable siRNA.

38. First pharmaceutical combination of any one of the claims 34-37, second pharmaceutical combination of any one of the claims 34-37, or third pharmaceutical composition of any one of the claims 34-37, wherein the oligonucleotide is an oligonucleotide that is capable of silencing a gene, when present in a cell comprising such gene, and/or is capable of targeting an aberrant miRNA when present in a cell comprising such aberrant miRNA.

39. First pharmaceutical combination of any one of the claims 34-38, second pharmaceutical combination of any one of the claims 34-38, or third pharmaceutical composition of any one of the claims 34-38, wherein the oligonucleotide is an oligonucleotide that is capable of targeting an mRNA, when present in a cell comprising such mRNA, or wherein the oligonucleotide is an oligonucleotide that is capable of antagonizing or restoring an miRNA function such as inhibiting an oncogenic miRNA (onco-miR) or suppression of expression of an onco-miR, when present in a cell comprising such an miRNA.

40. First pharmaceutical combination of claim 29 or 30, second pharmaceutical combination of claim 31 or 32, or third pharmaceutical composition of claim 33, wherein the effector moiety is a toxin.

41. First pharmaceutical combination of claim 40, second pharmaceutical combination of claim 40, or third pharmaceutical composition of claim 40, wherein the toxin is selected from: a viral toxin, a bacterial toxin, a plant toxin including ribosome-inactivating proteins and the A chain of type 2 ribosome-inactivating proteins, an animal toxin, a human toxin and a fungal toxin, more preferably the toxin is a plant toxin including ribosome-inactivating proteins and the A chain of type 2 ribosome-inactivating proteins.

42. First pharmaceutical combination of claim 40 or 41, second pharmaceutical combination of claim 40 or 41, or third pharmaceutical composition of claim 40 or 41, wherein the toxin is selected from the list consisting of: apoptin, Shiga toxin, Shiga-like toxin, Pseudomonas aeruginosa exotoxin (PE), full-length or truncated diphtheria toxin (DT), cholera toxin, alpha-sarcin, dianthin, saporin, bouganin, de-immunized derivative debouganin of bouganin, shiga-like toxin A, pokeweed antiviral protein, ricin, ricin A chain, modeccin, modeccin A chain, abrin, abrin A chain, volkensin, volkensin A chain, viscumin, viscumin A chain, frog RNase, granzyme B, human angiogenin; preferably the toxin is dianthin and/or saporin.

43. First pharmaceutical combination of any one of the claims 40-42, second pharmaceutical combination of any one of the claims 40-42, or third pharmaceutical composition of any one of the claims 40-42, wherein the toxin is selected from: a toxin targeting ribosomes, a toxin targeting elongation factors, a toxin targeting tubulin, a toxin targeting DNA and a toxin targeting RNA, more preferably the toxin is selected from the list consisting of: emtansine, pasudotox, maytansinoid derivative DM1, maytansinoid derivative DM4, monomethyl auristatin E (MMAE, vedotin), monomethyl auristatin F (MMAF, mafodotin), a Calicheamicin, N-Acetyl-γ-calicheamicin, a pyrrolobenzodiazepine (PBD) dimer, a benzodiazepine, a CC-1065 analogue, a duocarmycin, Doxorubicin, paclitaxel, docetaxel, cisplatin, cyclophosphamide, etoposide, docetaxel, 5-fluorouracyl (5-FU), mitoxantrone, a tubulysin, an indolinobenzodiazepine, AZ13599185, a cryptophycin, rhizoxin, methotrexate, an anthracycline, a camptothecin analogue, SN-38, DX-8951f, exatecan mesylate, truncated form of Pseudomonas aeruginosa exotoxin (PE38), a Duocarmycin derivative, an amanitin, α-amanitin, a spliceostatin, a thailanstatin, ozogamicin, tesirine, Amberstatin269 and soravtansine.

44. First pharmaceutical combination of claim 29 or 30, second pharmaceutical combination of claim 31 or 32, or third pharmaceutical composition of claim 33, wherein the effector moiety is an enzyme, such as urease or Cre-recombinase.

45. First pharmaceutical combination of claim 29 or 30, second pharmaceutical combination of claim 31 or 32, or third pharmaceutical composition of claim 33, wherein the effector molecule is a drug molecule.

46. First pharmaceutical combination of any one of the claim 29-30 or 34-45, second pharmaceutical combination of any one of the claim 31-32 or 34-45, or third pharmaceutical composition of any one of the claims 33-45, wherein the covalently bound conjugate comprises 1-16 effector moieties, preferably oligonucleotide(s), preferably 1-4 effector moieties, most preferably 1 effector moiety, wherein the effector moiety is preferably covalently bound in the conjugate via a cleavable bond, preferably an acid-labile cleavable bond that is cleaved under acidic conditions such as for example present in endosomes and/or lysosomes of mammalian cells, preferably human cells such as a diseased cell, an aberrant cell and a tumor cell, preferably at pH 4.0-6.5, and more preferably at pH≤5.5, wherein preferably the cleavable bond is a hydrazone bond or a semicarbazone bond, more preferably a semicarbazone bond.

47. First pharmaceutical combination of any one of the claim 29-30 or 34-46, second pharmaceutical combination of any one of the claim 31-32 or 34-46, or third pharmaceutical composition of any one of the claims 33-46, for use as a medicament, preferably in a human patient.

48. First pharmaceutical combination of any one of the claim 29-30 or 34-46, second pharmaceutical combination of any one of the claim 31-32 or 34-46, or third pharmaceutical composition of any one of the claims 33-46, for use in the treatment or prevention of a disease or health problem related to presence of the diseased cell of any one of the claims 18, 25, 26 and 46, preferably in a human patient, preferably wherein the disease or health problem related to presence of the diseased cell is related to a gene defect in the diseased cell and/or is related to expression or overexpression of a protein in the diseased cell.

49. First pharmaceutical combination of any one of the claim 29-30 or 34-46 or 48, second pharmaceutical combination of any one of the claim 31-32 or 34-46 or 48, or third pharmaceutical composition of any one of the claim 33-46 or 48, for use in the treatment or prevention of a disease or health problem related to the presence of the aberrant cell of any one of the claims 18, 25, 26 and 46, preferably in a human patient, preferably wherein the disease or health problem related to presence of the aberrant cell is related to a gene defect in the aberrant cell and/or is related to expression or overexpression of a protein in the aberrant cell.

50. First pharmaceutical combination of any one of the claim 29-30 or 34-46 or 48-49, second pharmaceutical combination of any one of the claim 31-32 or 34-46 or 48-49, or third pharmaceutical composition of any one of the claim 33-46 or 48-49, for use in the treatment or prevention of a cancer, preferably in a human patient, such as a cancer selected from any one or more of a carcinoma and a melanoma, for example selected from any one or more of: a breast cancer such as a breast carcinoma such as adenocarcinoma or metastatic adenocarcinoma in the breast; a cervical cancer such as a cervical carcinoma such as cervical epidermoid carcinoma; a skin cancer such as a skin carcinoma such as epidermoid carcinoma, or such as skin melanoma, preferably wherein the cancer is related to a gene defect in a tumor cell and/or is related to expression or overexpression of a protein in a tumor cell.

51. First pharmaceutical combination of any one of the claim 29-30 or 34-46 or 48-50, second pharmaceutical combination of any one of the claim 31-32 or 34-46 or 48-50, or third pharmaceutical composition of any one of the claim 33-46 or 48-50, for use in the treatment or prevention of an autoimmune disease such as rheumatoid arthritis, preferably in a human patient, preferably wherein the autoimmune disease is related to a gene defect in an aberrant cell and/or is related to expression or overexpression of a protein in an aberrant cell.

52. First pharmaceutical combination of any one of the claim 29-30 or 34-46 or 48-51, second pharmaceutical combination of any one of the claim 31-32 or 34-46 or 48-51, or third pharmaceutical composition of any one of the claim 33-46 or 48-51, for use in the treatment or prevention of a disease or health problem relating to any one or more of: expression or over-expression of a protein, presence of a mutant gene, a gene defect, a mutant protein, absence of a functional protein, presence of a dys-functional protein and a functional protein deficiency.

53. First pharmaceutical combination of any one of the claim 29-30 or 34-46 or 48-52, second pharmaceutical combination of any one of the claim 31-32 or 34-46 or 48-52, or third pharmaceutical composition of any one of the claim 33-46 or 48-52, for use according to any one of the claims 47-52, preferably in a human patient, wherein the first cell surface molecule and the third cell surface molecule are CD71 and/or the second cell surface molecule is CD71, and/or the first proteinaceous molecule and the third proteinaceous molecule are a monoclonal antibody capable of binding to CD71 or at least one sdAb capable of binding to CD71, and/or the second proteinaceous molecule is a monoclonal antibody capable of binding to CD71 or at least one sdAb capable of binding to CD71, and/or the effector moiety is an oligonucleotide,
preferably, the first, second and third cell surface molecule is CD71, the first, second and third proteinaceous molecule is a monoclonal antibody capable of binding to CD71 or at least one sdAb capable of binding to CD71, and the effector moiety is an oligonucleotide.

54. An antibody-drug conjugate, antibody-oligonucleotide conjugate, ligand-drug conjugate or ligand-oligonucleotide conjugate, comprising the saponin conjugate of any one of the claim 1-27 or 53 and an effector moiety of any one of the claim 34-46 or 53, preferably an antibody-oligonucleotide conjugate comprising the saponin conjugate of any one of the claim 1-27 or 53 and an effector moiety of any one of the claim 34-39 or 46 or 53.

55. The antibody-drug conjugate, antibody-oligonucleotide conjugate, ligand-drug conjugate or ligand-oligonucleotide conjugate, preferably the antibody-oligonucleotide conjugate, according to claim 54, for use as a medicament.

56. The antibody-drug conjugate, antibody-oligonucleotide conjugate, ligand-drug conjugate or ligand-oligonucleotide conjugate, preferably the antibody-oligonucleotide conjugate, according to claim 54, for use according to any one of the claims 48-53, preferably in a human patient.

57. An in vitro or ex vivo method for transferring a molecule from outside a cell to inside said cell, preferably into the cytosol of said cell, comprising the steps of:
a) providing a cell, preferably selected from: an aberrant cell, a diseased cell, a tumor cell and an auto-immune cell;
b) providing the molecule for transferring from outside the cell into the cell provided in step a), the molecule preferably selected from any one of the effector molecules of claims 34-46, preferably an oligonucleotide, wherein preferably the molecule for transferring from outside the cell into the cell is provided as a conjugate according to any one of the claims 29-32;
c) providing a saponin conjugate according to any one of the claims 1-27;
d) contacting the cell of step a) in vitro or ex vivo with the molecule of step b) and the saponin conjugate of step c), therewith establishing the transfer of the molecule from outside the cell into said cell.


SEQUENCE LISTING (7 sequences)

SEQ ID NO: 1 - DNA, 16 bp, Artificial Sequence
  Antisense BNA (HSP27) oligo nucleic acid sequence
ggcacagccagtggcg

SEQ ID NO: 2 - DNA, 20 bp, Artificial Sequence
  HSP27 primer forward sequence
gcagtccaacgagatcacca

SEQ ID NO: 3 - DNA, 20 bp, Artificial Sequence
  HSP27 primer reverse sequence
taaggctttacttggcggca

SEQ ID NO: 4 - DNA, 20 bp, Artificial Sequence
  HBMS primer forward sequence
cacccacacacagcctactt

SEQ ID NO: 5 - DNA, 20 bp, Artificial Sequence
  HBMS primer reverse sequence
gtacccacgcgaatcactct

SEQ ID NO: 6 - DNA, 22 bp, Artificial Sequence
  GUSB primer forward sequence
gaaaatacgtggttggagagct

SEQ ID NO: 7 - DNA, 22 bp, Artificial Sequence
  GUSB primer reverse sequence
ccgagtgaagatccccttttta